(19) United States
(12) Patent Application Publication
(10) Pub. No.: US 2026/0347486 A1
(43) Pub. Date: Nov. 5, 2026

(54) IL-18 MIMETICS

(71) Applicant: Merck Patent GmbH, Darmstadt (DE)

(72) Inventors: Stefan Zielonka, Darmstadt (DE); Lukas Pekar, Darmstadt (DE); Paul Arras, Darmstadt (DE); Britta Lipinski, Darmstadt (DE); Laura Unmuth, Darmstadt (DE)

(73) Assignee: Merck Patent GmbH, Darmstadt (DE)

(21) Appl. No.: 19/491,108

(22) PCT Filed: Jun. 7, 2024
(86) PCT No.: PCT/EP2024/065796

§ 371 Date: Dec. 8, 2025

(30) Foreign Application Priority Data
Jun. 9, 2023 (EP) 23178542.9

(51) Int. Cl. C07K 16/28

(52) U.S. Cl. CPC C07K 16/2866; C07K 2317/22; C07K 2317/31; C07K 2317/53; C07K 2317/565; C07K 2317/569; C07K 2317/732; C07K 2317/92

(57) ABSTRACT

Novel, VHH-based binders of IL-18Rα and/or IL-18Rβ show favorable characteristics. Pharmaceutical compositions have such a compound.

### FIELD OF THE INVENTION

The present disclosure relates to novel, VHH-based binders of IL-18Rα and/or IL-18Rβ with favorable characteristics. Moreover, the present disclosure relates to pharmaceutical compositions comprising such a compound.

### BACKGROUND OF THE INVENTION

Cytokines are potent immunomodulatory proteins comprising huge therapeutic potential. Consequently, several different molecules were granted marketing approval for the treatment of different diseases (Propper and Balkwill, 2022; Pires et al., 2021; Berraondo et al., 2019).

However, the pleiotropic mechanism of action of many cytokines, their short half-life combined with often dose-limiting toxicities when administered systemically hamper their therapeutic applicability (Pires et al., 2021; Amin et al., 2014).

To address these inherent limitations, “next-generation cytokines” were generated (Zheng et al., 2022). For instance, muteins of IL-2 were engineered, which do not target the a-subunit of the IL-2 receptor (CD25) in order to eliminate the intrinsic bias of this cytokine for activating regulatory T cells (Tregs) (Klein et al., 2017; Dolgin, 2022). To optimize the poor pharmacokinetics of cytokines, fusions with the (effector silenced or attenuated) Fc portion of IgGs were engineered (Jazayeri and Carroll, 2008) as well as conjugates with polyethylene glycol (Eliason, 2001). Besides, bifunctional antibody cytokine fusion proteins were constructed aiming at accumulating the immunomodulatory function of cytokines at the site of disease (Murer and Neri, 2019; Neri and Sondel, 2016).

Another interesting approach regarding “next-generation cytokines” relies on exploiting antibody-derivatives that mimic the function of cytokines, referred to as cytokine mimetics or surrogate cytokines (Saxton et al., 2023). In this respect, bispecific antibody derivatives (diabodies) have been developed that dimerize the Erythropoietin receptor (EpoR) (Moraga et al., 2015). Intriguingly, different diabodies induced differential EpoR phosphorylation ranging from weak and partial agonism to full agonism. In addition to this, single domain antibody (sdAb)-based bispecifics were generated that activate signalling through the heterodimeric IL-2 receptor γγ (IL-2Rβγ). These bispecific antibodies (bsAbs) mimic the function of IL-2 but without preferential activation of Tregs via IL-2 receptor a binding (Harris et al., 2021). Garcia and co-workers recently described the engineering of VHH-derived surrogate agonists targeting IL-2Rβγ as well as type I IFN-mimicking bsAbs (Yen et al., 2022). Importantly, the group constructed surrogate agonists displaying functional diversification in terms of receptor downstream signalling compared to the natural cytokine. Moreover, also bsAbs were constructed that trigger agonistic activity through binding to IL-2Rβ and IL-10Rβ, a receptor heterodimer that naturally does not exist. This is clearly demonstrating that the modular assembly of bsAbs enables the generation of cytokine mimetics with tailor-made functionalities which might be versatile building blocks for drug discovery.

IL-18 is a proinflammatory cytokine belonging to the IL-1 family of cytokines that mediates signalling through heterodimerization of the receptor subunits IL-18Rα and IL-18Rβ (Yasuda et al., 2019; Dinarello et al., 2013). IL-18 stimulates IFN-γ production in innate lymphoid cells as well as antigen-experienced T cells in synergy with IL-12 (Nakamura et al., 2020). Recombinant (r)IL-18 has been assessed in clinical trials and demonstrated a favorable toxicity profile but limited efficacy as monotherapy (Atallah-Yunes and Robertson, 2022; Robertson et al., 2006; Tarhini et al., 2009). Physiologically, the activity of IL-18 is balanced via the high-affinity neutralizing and naturally occurring IL-18 binding protein (IL-18BP) (Dinarello et al., 2013; Nakamura et al., 2020). After treatment with (r)IL-18, substantially elevated levels of IL-18BP were found in the serum of patients (Robertson et al., 2006; Robertson et al. 2008). Ring and colleagues were able to show that IL18BP is expressed in the tumor microenvironment of several tumor types (Zhou et al., 2020). Moreover, the authors found increased IL-18BP concentrations in the serum of patients with non-small cell lung cancer which were further elevated post PD-1 or PD-L1 treatment. In the same study, the group engineered a decoy-resistant IL-18 mutein that still triggered IL-18 receptor activation. This next-generation IL-18 derivative showed superior antitumor efficacy in preclinical models as monotherapy as well as in combination with immune checkpoint inhibition (Zhou et al., 2020).

IL-18 is a proinflammatory cytokine promoting NK cell activation as well as effector T cell maturation and function (Holder et al., 2022). Consequently, recombinant human (rh) IL-18 emerged as promising potential therapeutic inducing antitumor immunity. (rh) IL-18 was assessed in several clinical trials either as single agent or as combination therapy (see e.g. Tarhini et al., 2009; Robertson et al., 2008; Robertson et al., 2013; Simpkins et al., 2013; Robertson et al., 2018). (rh) IL-18 therapy has been well tolerated, however, clinical efficacy has been limited. A possible explanation for the lack of therapeutic efficacy relies in the inhibition of IL-18 by the IL-18BP decoy receptor (Dinarello et al., 2013). IL-18BP binds to an overlapping site on IL-18 with IL-18Rα albeit with much higher affinities. Accordingly, it antagonizes signalling of IL-18 by blocking the IL-18Rα interaction with IL-18. Recently, Ring and colleagues described the generation of a decoy resistant mutein of IL-18 showing great promise for anti-cancer therapy in preclinical models (Zhou et al., 2020). This entity is currently in clinical investigations (NCT04787042).

Despite the relevance of IL-18 in the formation of tumors and the potential in targeting IL-18 for cancer treatment, at present approaches that allow to mimic IL-18 and its functions in a taylor-made fashion are still lacking.

Accordingly, there is a need in the art for improved IL-18 receptor agonists. Moreover, there is a need in the art for improved ways to mimic the function of IL-18 in a taylor-made fashion, e.g. specifically with respect to different potencies and magnitudes of activation of IL-18R receptor activation, different potencies and magnitudes for the release of IFN-γ or resistance to inhibition by IL-18BP.

The present disclosure overcomes the above-described problems and addresses the above-described needs.

### SUMMARY OF THE INVENTION

The present disclosure addresses the needs described above in the section “Background of the Invention” by the different aspects and embodiments described below.

The present invention is, in part, based on the surprising observation that novel bispecific surrogate agonists can be obtained that are based on sdAbs (sdAb: single-domain antibody, also referred to herein as V_{H}H or VHH), target both IL-18Rα and IL-18Rβ and mimic the functionality of IL-18. Moreover, it was found that it is possible to obtain such IL-18 mimics that at the same time are resistant to the decoy receptor IL-18BP. Moreover, it was surprisingly observed that molecules in which the overall design architecture with respect to valencies as well as to the spatial orientation of individual VHH-based paratopes is engineered within the molecule allow to obtain IL-18 mimetics in which the agonistic activities are tailor-made, e.g. with respect to their potency, the magnitude of the induced activation of IL-18R and IFN-γ release or the tolerance to inhibition by IL-18BP receptor decoy.

In an aspect, the present disclosure relates to a VHH antibody domain or a fragment thereof, wherein
(a) said VHH antibody domain or fragment thereof comprises the complementarity determining regions CDR1, CDR2 and CDR3 of one VHH selected from the group consisting of VHH1, VHH2, VHH3, VHH4, VHH5, VHH6, VHH8, VHH9, VHH10 and VHH11 as shown in the Table of CDRs;
(b) said VHH antibody domain or fragment thereof comprises the complementarity determining regions CDR1, CDR2 and CDR3 as defined in (a) with modification, wherein the modification is that the sequence of at least one of CDR1, CDR2 and CDR3 is humanized; or (c) said VHH antibody domain or fragment thereof comprises the complementarity determining regions CDR1, CDR2 and CDR3 as defined in (a) with modification, wherein the modification is
   the replacement, addition or deletion of up to three amino acids in CDR1,
   the replacement, addition or deletion of up to three amino acids in CDR2 and/or
   the replacement, addition or deletion of up to three amino acids in CDR3;

Table of CDRs:

| Description | SEQ ID NO | Amino acid sequence |
|---|---|---|
| CDR1 of VHH1 | SEQ ID NO: 23 | GFTENSYA |
| CDR2 of VHH1 | SEQ ID NO: 24 | HIGSGGSTR |
| CDR3 of VHH1 | SEQ ID NO: 25 | NVYGATLRS |
| CDR1 of VHH2 | SEQ ID NO: 26 | GFTLLDSYD |
| CDR2 of VHH2 | SEQ ID NO: 27 | ISSSGGKSTY |
| CDR3 of VHH2 | SEQ ID NO: 28 | AADGAEGFWSCQSGGYDY |
| CDR1 of VHH3 | SEQ ID NO: 29 | GFTLGAYA |
| CDR2 of VHH3 | SEQ ID NO: 30 | ITSSGRSAY |
| CDR3 of VHH3 | SEQ ID NO: 31 | AAGAACAAAGTMNY |
| CDR1 of VHH4 | SEQ ID NO: 32 | GFTLGAYA |
| CDR2 of VHH4 | SEQ ID NO: 33 | ITSSGGSAY |
| CDR3 of VHH4 | SEQ ID NO: 34 | AAGGACAAAETMNY |
| CDR1 of VHH5 | SEQ ID NO: 35 | GRTFSNLA |
| CDR2 of VHH5 | SEQ ID NO: 36 | IDWNGDNRL |
| CDR3 of VHH5 | SEQ ID NO: 37 | AADSTYSTDWYLNP |
| | | LNRTRYDY |
| CDR1 of VHH6 | SEQ ID NO: 38 | KSIFSINA |
| CDR2 of VHH6 | SEQ ID NO: 39 | FTSSGRIN |
| CDR3 of VHH6 | SEQ ID NO: 40 | NVGLWEDGLWYDY |
| CDR1 of VHH7 | SEQ ID NO: 41 | GHTENNLA |
| CDR2 of VHH7 | SEQ ID NO: 42 | IDWNGDNRL |
| CDR3 of VHH7 | SEQ ID NO: 43 | AADSTYSTNWYLD |
| | | PLNRTTYNY |
| CDR1 of VHH8 | SEQ ID NO: 44 | GRTFSNDA |
| CDR2 of VHH8 | SEQ ID NO: 45 | VSWSLGMTY |
| CDR3 of VHH8 | SEQ ID NO: 46 | AAGPDLSDELYGYDY |
| CDR1 of VHH9 | SEQ ID NO: 47 | RSDFSRYT |
| CDR2 of VHH9 | SEQ ID NO: 48 | RITSGGIPH |
| CDR3 of VHH9 | SEQ ID NO: 49 | NTVGVALWA |
| CDR1 of VHH10 | SEQ ID NO: 50 | GLPFSSYA |
| CDR2 of VHH10 | SEQ ID NO: 51 | ITWSGSSIY |
| CDR3 of VHH10 | SEQ ID NO: 52 | AAARRRVGLPLADPREYVY |
| CDR1 of VHH11 | SEQ ID NO: 53 | GSPIRFSV |
| CDR2 of VHH11 | SEQ ID NO: 54 | RITSGGIVY |
| CDR3 of VHH11 | SEQ ID NO: 55 | NRLGGALFD |
| CDR1 of VHH12 | SEQ ID NO: 56 | GVGFSIYT |
| CDR2 of VHH12 | SEQ ID NO: 57 | VTTGGHIN |
| CDR3 of VHH12 | SEQ ID NO: 58 | NAIYFGODI |
| CDR1 of VHH13 | SEQ ID NO: 59 | GISFRVNV |
| CDR2 of VHH13 | SEQ ID NO: 60 | ISSGDSTN |
| CDR3 of VHH13 | SEQ ID NO: 61 | NARIFARDN |
| CDR1 of VHH14 | SEQ ID NO: 62 | DRTVSKGA |
| CDR2 of VHH14 | SEQ ID NO: 63 | IGRSSGITY |
| CDR3 of VHH14 | SEQ ID NO: 64 | AADSSGIIRTTSWAYDY |
| CDR1 of VHH15 | SEQ ID NO: 65 | GRFFTINA |
| CDR2 of VHH15 | SEQ ID NO: 66 | ITSDGSTT |
| CDR3 of VHH15 | SEQ ID NO: 67 | NADGRENDNGYVKNFNI |
| CDR1 of VHH16 | SEQ ID NO: 68 | GFTTDDYI |
| CDR2 of VHH16 | SEQ ID NO: 69 | ISTTDGSAW |
| CDR3 of VHH16 | SEQ ID NO: 70 | AEVSTGTGVLYCREEF |
| | | YFGMDS |
| CDR1 of VHH17 | SEQ ID NO: 71 | GFALDEIA |
| CDR2 of VHH17 | SEQ ID NO: 72 | ISSSDGSTL |
| CDR3 of VHH17 | SEQ ID NO: 73 | AAVRGSCFGRTTH |
| CDR1 of VHH18 | SEQ ID NO: 74 | GRTFSSYA |
| CDR2 of VHH18 | SEQ ID NO: 75 | ISWSGGSTY |
| CDR3 of VHH18 | SEQ ID NO: 76 | AADRSLSGSYFGATIDY |
| CDR1 of VHH19 | SEQ ID NO: 77 | GIRENPER |
| CDR2 of VHH19 | SEQ ID NO: 78 | IDSGGSTD |
| CDR3 of VHH19 | SEQ ID NO: 79 | NTQIFARDY |
| CDR1 of VHH20 | SEQ ID NO: 80 | VSAFRLKV |
| CDR2 of VHH20 | SEQ ID NO: 81 | IDSGDTKS |
| CDR3 of VHH20 | SEQ ID NO: 82 | NAVLFGRDY |
| CDR1 of VHH21 | SEQ ID NO: 83 | GITFSPYV |
| CDR2 of VHH21 | SEQ ID NO: 84 | MVNGGSTN |
| CDR3 of VHH21 | SEQ ID NO: 85 | HIMIFGRAS |
| CDR1 of VHH22 | SEQ ID NO: 86 | GFALEYYA |
| CDR2 of VHH22 | SEQ ID NO: 87 | VSSSRSGGSTV |
| CDR3 of VHH22 | SEQ ID NO: 88 | AGAPAYRSHYCRLQADDYTY |

In another aspect, the present disclosure relates to a VHH antibody domain or a fragment thereof, wherein
(A) said VHH antibody domain comprises the amino acid sequence of a VHH selected from the group consisting of VHH1, VHH2, VHH3, VHH4, VHH5, VHH6, VHH8, VHH9, VHH10 and VHH11 as shown in the Table of VHH Sequences;
(B) said VHH antibody domain comprises a VHH sequence as defined in (A) with modification, wherein the modification is that said sequence is humanized;
(C) said VHH antibody domain comprises a VHH sequence as defined in (A) with modification, wherein the modification is the replacement, addition or deletion of up to 25 amino acids; or
(D) said VHH antibody domain comprises a VHH sequence that is at least 75% identical to a VHH sequence referred to in (A);

Table of VHH Sequences:

| VHH | SEQ ID NO | Amino acid sequence |
|---|---|---|
| VHH1 | SEQ ID NO: 1 | DVPLQESGGGLVQTGGSLRLSCAASGFTENSYAMGW |
| | | YQQAPGKKYRELVAHIGSGGSTRYIDSVKGRFTISR |
| | | DNAKNTVYLQMNSLKPEDTAVYYCNVYGATLRSWGQ |
| | | GTQVTVSS |
| VHH2 | SEQ ID NO: 2 | DVQLQESGGGLVQAGGSLRLSCAASGFTLLDSYDIG |
| | | WFRQAPGKKREEVSCISSSGGKSTYYADSVKGRFTI |
| | | SRDNANNTVYLQMNSLKPEDTAVYYCAADGAEGEWS |
| | | CQSGGYDYWGQGTQVTVSS |
| VHH3 | SEQ ID NO: 3 | DVQLQESGGGLVQAGGSLRLSCAASGFTLGAYAINW |
| | | FRQAPGKERARAWDACITSSGRSAYYADSVKGRFTV |
| | | SRDHAKNTVYLQMNSLKPEDTAFYYCAAGAACAAAG |
| | | TMNYWGKGTLVTVSS |
| VHH4 | SEQ ID NO: 4 | DVQLQESGGGLVQAGGSLRLSCAASGFTLGAYAINW |
| | | FQQAPGKERARAWVLCITSSGGSAYYVDSVKGRFTV |
| | | SRDNAKNTVYLQMNSLKPEDTAFYYCAAGGACAAAE |
| | | TMNYWGKGTLVTVSS |
| VHH5 | SEQ ID NO: 5 | DVQLQESGGGLVQAGGSLRLSCAASGRTFSNLAMAW |
| | | FRQAPGKEREFVAAIDWNGDNRLYAASVKGRFTISR |
| | | DNAKSTMYLQMNSLKPEDTAVYYCAADSTYSTDWYL |
| | | NPLNRTRYDYWGQGTQVTVSS |
| VHH6 | SEQ ID NO: 6 | DVPLQESGGGSVQPGGSLRLSCAASKSIFSINAMRW |
| | | HRQAPGKQRELVATFTSSGRINYADSVKGRFTISTD |
| | | NAKNTIYLQMNSLNPEDTAVYRCNVGLWEDGLWYDY |
| | | WGQGTQVTVSS |
| VHH7 | SEQ ID NO: 7 | DVQLQESGGGLVQAGGSLRLSCAASGHTENNLAMAW |
| | | CRQAPGKEREFVSAIDWNGDNRLYAYSVKGRFTISR |
| | | DNAKSTMYLQMNSLKPEDTAVYYCAADSTYSTNWYL |
| | | DPLNRTTYNYWGQGTPVTVSS |
| VHH8 | SEQ ID NO: 8 | DVQLQESGGGLVQAGGSLRLSCAASGRTFSNDAMGW |
| | | FRQAPGKEREFVAAVSWSLGMTYNADSVKGRFTISR |
| | | DNAKNTVYLQMNSLKPDDTAVYYCAAGPDLSDELYG |
| | | YDYWGQGTQVTVSS |
| VHH9 | SEQ ID NO: 9 | DVQLQESGGGLVQPGESLRLSCTASRSDESRYTMGW |
| | | YRQAPGKWRELVARITSGGIPHYADSVKGRFTISRD |
| | | NAKNTVTLQMNTLQFEDSAVYYCNTVGVALWAWGQG |
| | | TLVTVSS |
| VHH10 | SEQ ID NO: 10 | DVQLQESGGGLVQAGGSLRLSCAASGLPFSSYAMGW |
| | | FRQAPGKDREFVAGITWSGSSIYYRDSVKGRFTISR |
| | | DNAKNTVWLQMNDLKPEDTAVYYCAAARRRVGLPLA |
| | | DPREYVYWGQGTQVTVSS |
| VHH11 | SEQ ID NO: 11 | DVPLQESGGGLVQPGQSLRLSCAASGSPIRFSVMGW |
| | | FQQAPGKGRDLIARITSGGIVYYTDSVKGRFTISKD |
| | | NAKNTLYLQMNSLNLEDTGVYYCNRLGGALEDWGQG |
| | | TQVTVSS |
| VHH12 | SEQ ID NO: 12 | DVPLQESGGGLVQPGGSLRLSCRASGVGESIYTMGW |
| | | YRQAPGEQRHLVATVTTGGHINYGDSVQGRFTISRE |
| | | NNGNVVELQMHSLKPEDTAVYFCNAIYFGQDIWGOG |
| | | TQVTVSS |
| VHH13 | SEQ ID NO: 13 | DVQLQESGGGLVQPGGSLRLSCAASGISFRVNVLGW |
| | | YRQAPGKPRELVASISSGDSTNYADSVKGRFTISRD |
| | | NAKSTVYLQMNNLKPEDTAVYYCNARIFARDNWGQG |
| | | TQVTVSS |
| VHH14 | SEQ ID NO: 14 | DVPLQESGGGLVQAGGSLRLSCAASDRTVSKGAMGW |
| | | FRQAPGKEREFVATIGRSSGITYYADSVKGRFTISR |
| | | DNAKNTWYLQMNSLRPEDTAVYYCAADSSGIIRTTS |
| | | WAYDYWGQGTQVTVSS |
| VHH15 | SEQ ID NO: 15 | DVQLQESGGGLVQPGGSLRLSCAASGRFFTINAMGW |
| | | YRQTPGKQRELVAAITSDGSTTYQDSVKGRFTISRD |
| | | NANRTANLYMNSLKSEDTAVYYCNADGRENDNGYVK |
| | | NENIWGQGTQVTVSS |
| VHH16 | SEQ ID NO: 16 | DVQLQESGGGSVPAGGSLRLSCSASGFTTDDYIIGW |
| | | FRQAPGKKREGVSCISTTDGSAWYADSVKGRFTISS |
| | | DNANNTVTLQMNSLLPEDTSVYYCAEVSTGTGVLYC |
| | | REEFYFGMDSWGKGTPVTVSS |
| VHH17 | SEQ ID NO: 17 | DVPLQESGGGLVQAGGSLRLSCVASGFALDEIAVGW |
| | | FRQAPGKEREGLSCISSSDGSTLYADSVKGRFTISS |
| | | DNAKNTVYLQMNSLKPEDTAVYLCAAVRGSCFGRTT |
| | | HWGQGTQVTVSS |
| VHH18 | SEQ ID NO: 18 | DVQLQESGGGLVQAGGSLRLSCAASGRTFSSYAMGW |
| | | FRQAPGMVREFVAAISWSGGSTYYADSVQGRFTISR |
| | | DNAKNTVYLQMNSLKPEDTAVYYCAADRSLSGSYFG |
| | | ATIDYWGQGTQVTVSS |
| VHH19 | SEQ ID NO: 20 | DVQLQESGGGLVQAGGSLRLSCAASGIRENPERMAW |
| | | YRQAPGKQRELVAAIDSGGSTDYADSVRGRFTISRD |
| | | NAKITVSLQMNSLKPEDTAVYYCNTQIFARDYWGQG |
| | | SQVTVSS |
| VHH20 | SEQ ID NO: 21 | DVQLQESGGGLVQAGGSLRLSCAASVSAFRLKVMGW |
| | | YRQAPGKEREFVAAIDSGDTKSYTNAVKGRFTISGG |
| | | AATNTVVLQMNSLKPEDTAFYYCNAVLFGRDYWGQG |
| | | TQVTVSS |
| VHH21 | SEQ ID NO: 22 | DVQLQESGGGLVQAGGSLRLSCAASGITFSPYVMGW |
| | | YRQAPGKQRELVADMVNGGSTNYADSVKGRFTIERD |
| | | TAANTVTLQMNNLKPDDTAVYYCHIMIFGRASWGQG |
| | | TQVTVSS |
| VHH22 | SEQ ID NO: 22 | DVPLQESGGGLVQPGGSLRLSCAAPGFALEYYAIGW |
| | | FRQAPGKEREGLSCVSSSRSGGSTVYADSVKGRFTI |
| | | SRDNAKNTVYLQIDSLKVEDTAVYYCAGAPAYRSHY |
| | | CRLQADDYTYWGQGTQVTVSS |

In another aspect, aspect, the present disclosure relates to a VHH antibody domain or a fragment thereof, wherein
(A) said VHH antibody domain consists of the amino acid sequence of a VHH selected from the group consisting of VHH1, VHH2, VHH3, VHH4, VHH5, VHH6, VHH8, VHH9, VHH10 and VHH11 as shown in the Table of VHH Sequences;
(B) said VHH antibody domain consists of a VHH sequence as defined in (A) with modification, wherein the modification is that said sequence is humanized;
(C) said VHH antibody domain consists of a VHH sequence as defined in (A) with modification, wherein the modification is the replacement, addition or deletion of up to 25 amino acids; or
(D) said VHH antibody domain consists of a VHH sequence that is at least 7500 identical to a VHH sequence referred to in (A).

In another aspect, aspect, the present disclosure relates to a VHH antibody domain or a fragment thereof, wherein
(d) said VHH antibody domain or fragment thereof comprises the complementarity determining regions CDR1, CDR2 and CDR3 of one VHH selected from the group consisting of VHH12, VHH13, VHH14, VHH15, VHH16, VHH17, VHH18, VHH20, VHH21 and VHH22 as shown in the Table of CDRs;
(e) said VHH antibody domain or fragment thereof comprises the complementarity determining regions CDR1, CDR2 and CDR3 as defined in (d) with modification, wherein the modification is that the sequence of at least one of CDR1, CDR2 and CDR3 is humanized; or
(f) said VHH antibody domain or fragment thereof comprises the complementarity determining regions CDR1, CDR2 and CDR3 as defined in (d) with modification, wherein the modification is
   the replacement, addition or deletion of up to three amino acids in CDR1,
   the replacement, addition or deletion of up to three amino acids in CDR2 and/or
   the replacement, addition or deletion of up to three amino acids in CDR3.

In another aspect, aspect, the present disclosure relates to a VHH antibody domain or a fragment thereof, wherein
(E) said VHH antibody domain comprises the amino acid sequence of a VHH selected from the group consisting of VHH12, VHH13, VHH14, VHH15, VHH16, VHH17, VHH18, VHH20, VHH21 and VHH22 as shown in the Table of VHH Sequences;
(F) said VHH antibody domain comprises a VHH sequence as defined in (E) with modification, wherein the modification is that said sequence is humanized;
(G) said VHH antibody domain comprises a VHH sequence as defined in (E) with modification, wherein the modification is the replacement, addition or deletion of up to 25 amino acids; or
(H) said VHH antibody domain comprises a VHH sequence that is at least 75% identical to a VHH sequence referred to in (E).

In another aspect, aspect, the present disclosure relates to a VHH antibody domain or a fragment thereof, wherein
(E) said VHH antibody domain consists of the amino acid sequence of a VHH selected from the group consisting of VHH12, VHH13, VHH14, VHH15, VHH16, VHH17, VHH18, VHH20, VHH21 and VHH22 as shown in the Table of VHH Sequences;
(F) said VHH antibody domain consists of a VHH sequence as defined in (E) with modification, wherein the modification is that said sequence is humanized;
(G) said VHH antibody domain consists of a VHH sequence as defined in (E) with modification, wherein the modification is the replacement, addition or deletion of up to 25 amino acids; or
(H) said VHH antibody domain consists of a VHH sequence that is at least 75% identical to a VHH sequence referred to in (E).

In another aspect, aspect, the present disclosure relates to a compound comprising
a VHH antibody domain or fragment thereof according to any one of embodiments 1 to

In another aspect, aspect, the present disclosure relates to a compound comprising
a VHH antibody domain or fragment thereof according to any one of claims 189 to 374.

In another aspect, aspect, the present disclosure relates to a compound comprising
a first binding module which is a VHH antibody domain or fragment thereof, wherein
   (a) said VHH antibody domain or fragment thereof comprises the complementarity determining regions CDR1, CDR2 and CDR3 of one VHH selected from the group consisting of VHH1, VHH2, VHH3, VHH4, VHH5, VHH6, VHH8, VHH9, VHH10 and VHH11 as shown in the Table of CDR;
   (b) said VHH antibody domain or fragment thereof comprises the complementarity determining regions CDR1, CDR2 and CDR3 as defined in (a) with modification, wherein the modification is that the sequence of at least one of CDR1, CDR2 and CDR3 is humanized; or
   (c) said VHH antibody domain or fragment thereof comprises the complementarity determining regions CDR1, CDR2 and CDR3 as defined in (a) with modification, wherein the modification is
      the replacement, addition or deletion of up to three amino acids in CDR1,
      the replacement, addition or deletion of up to three amino acids in CDR2 and/or
      the replacement, addition or deletion of up to three amino acids in CDR3;
a second binding module which is a VHH antibody domain or a fragment thereof, wherein
   (d) said VHH antibody domain or fragment thereof comprises the complementarity determining regions CDR1, CDR2 and CDR3 of one VHH selected from the group consisting of VHH12, VHH13, VHH14, VHH15, VHH16, VHH17, VHH18, VHH20, VHH21 and VHH22 as shown in the Table of CDRs;
   (e) said VHH antibody domain or fragment thereof comprises the complementarity determining regions CDR1, CDR2 and CDR3 as defined in (d) with modification, wherein the modification is that the sequence of at least one of CDR1, CDR2 and CDR3 is humanized; or
   (f) said VHH antibody domain or fragment thereof comprises the complementarity determining regions CDR1, CDR2 and CDR3 as defined in (d) with modification, wherein the modification is
   the replacement, addition or deletion of up to three amino acids in CDR1,
   the replacement, addition or deletion of up to three amino acids in CDR2 and/or
   the replacement, addition or deletion of up to three amino acids in CDR3.

In another aspect, aspect, the present disclosure relates to a compound comprising
a first binding module which is a VHH antibody domain or fragment thereof, wherein
   (A) said VHH antibody domain comprises the amino acid sequence of a VHH selected from the group consisting of VHH1, VHH2, VHH3, VHH4, VHH5, VHH6, VHH8, VHH9, VHH10 and VHH11 as shown in the Table of VHH Sequences;
   (B) said VHH antibody domain comprises a VHH sequence as defined in (A) with modification, wherein the modification is that said sequence is humanized;
   (C) said VHH antibody domain comprises a VHH sequence as defined in (A) with modification, wherein the modification is the replacement, addition or deletion of up to 25 amino acids; or
   (D) said VHH antibody domain comprises a VHH sequence that is at least 75% identical to a VHH sequence referred to in (A);
a second binding module which is a VHH antibody domain or a fragment thereof, wherein
   (E) said VHH antibody domain comprises the amino acid sequence of a VHH selected from the group consisting of VHH12, VHH13, VHH14, VHH15, VHH16, VHH17, VHH18, VHH20, VHH21 and VHH22 as shown in the Table of VHH Sequences;
   (F) said VHH antibody domain comprises a VHH sequence as defined in (E) with modification, wherein the modification is that said sequence is humanized;
   (G) said VHH antibody domain comprises a VHH sequence as defined in (E) with modification, wherein the modification is the replacement, addition or deletion of up to 25 amino acids; or
   (H) said VHH antibody domain comprises a VHH sequence that is at least 75% identical to a VHH sequence referred to in (E).

In another aspect, aspect, the present disclosure relates to a compound comprising
a first binding module which is a VHH antibody domain or fragment thereof, wherein
   (A) said VHH antibody domain consists of the amino acid sequence of a VHH selected from the group consisting of VHH1, VHH2, VHH3, VHH4, VHH5, VHH6, VHH8, VHH9, VHH10 and VHH11 as shown in the Table of VHH Sequences;
   (B) said VHH antibody domain consists of a VHH sequence as defined in (A) with modification, wherein the modification is that said sequence is humanized;
   (C) said VHH antibody domain consists of a VHH sequence as defined in (A) with modification, wherein the modification is the replacement, addition or deletion of up to 25 amino acids; or
   (D) said VHH antibody domain consists of a VHH sequence that is at least 75% identical to a VHH sequence referred to in (A);
a second binding module which is a VHH antibody domain or a fragment thereof, wherein
   (E) said VHH antibody domain consists of the amino acid sequence of a VHH selected from the group consisting of VHH12, VHH13, VHH14, VHH15, VHH16, VHH17, VHH18, VHH20, VHH21 and VHH22 as shown in the Table of VHH Sequences;
   (F) said VHH antibody domain consists of a VHH sequence as defined in (E) with modification, wherein the modification is that said sequence is humanized;
   (G) said VHH antibody domain consists of a VHH sequence as defined in (E) with modification, wherein the modification is the replacement, addition or deletion of up to 25 amino acids; or
   (H) said VHH antibody domain consists of a VHH sequence that is at least 75% identical to a VHH sequence referred to in (E).

In another aspect, the present disclosure relates to a compound which is a bispecific antibody molecule comprising an IL-18Rα-binding VHH with an amino acid sequence as defined in the present disclosure below (First binding module) and an IL18Rα-binding VHH with an amino acid sequence as defined in the present disclosure below (Second binding module).

In another aspect, the present disclosure relates to a bispecific antibody molecule prepared by the SEED (strand-exchange engineered domain) technology, said molecule comprising
an AG chain linked to an IL-18Rα-binding VHH, wherein said AG chain linked to said IL-18Rα-binding VHH has an amino acid sequence as defined in the present disclosure below (“VHH-SEED-AG-1”) and
a GA chain linked to an IL18Rβ-binding VHH, wherein said GA chain linked to said IL18Rβ-binding VHH has an amino acid sequence as defined in the present disclosure below (“VHH-SEED-GA-1”).

In another aspect, the present disclosure relates to a pharmaceutical composition comprising the compound or bispecific antibody molecule according to the present disclosure.

In another aspect, the present disclosure relates to the use of a compound or bispecific antibody molecule according to the present disclosure for activating IL-18R receptor.

In another aspect, the present disclosure relates to the use of a compound or bispecific antibody molecule according to the present disclosure for inducing IL-7 release from cells carrying an IL-18R receptor.

### BRIEF DESCRIPTION OF THE FIGS

In the following, reference is made to the figures. All methods referred to in the FIG. descriptions below were carried out as described in detail in the examples.

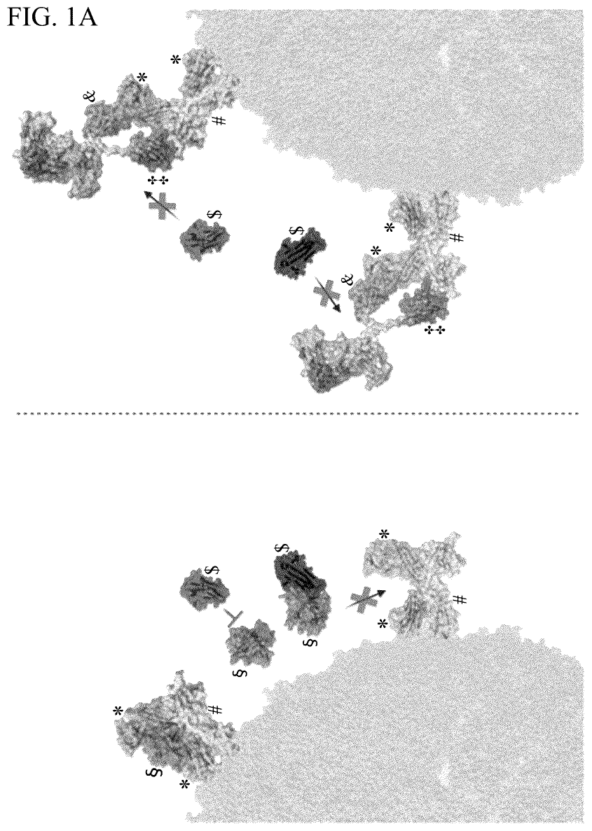

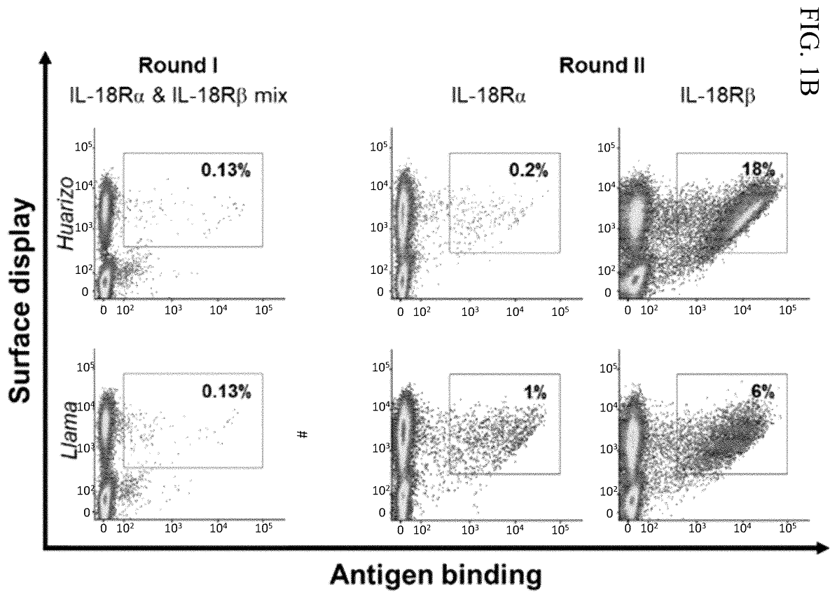

FIG. 1 summarizes the overall strategy applied in the present disclosure for the generation of tailor-made cytokine mimetics based on sdAb-derived bispecifics and YSD-enabled antibody discovery. (A) Bottom/Left panel: IL-18 (blue, §) triggers IL-18R downstream signalling by consecutive binding to IL-18Rα (yellow, *; the two “lobes” of IL-18Rα are each labelled with *) followed by IL-18Rβ (lime, #) recruitment. IL-18BP (dark grey, $) inhibits IL-18 by high-affinity binding and hence, by blocking the IL-18/IL-18Rα interaction. Top/Right panel: Upon reformatting camelid-derived sdAbs with IL-18Rα-targeting functionality (orange, &) and IL-18Rβ-targeting functionality (green, ‡) into an IgG-like bispecific, resulting cytokine mimetics cross-link the IL18R subunits IL-18Rα (yellow, *; the two “lobes” of IL-18Rα are each labelled with *) and IL-18Rβ (lime, #) and hence elicit down-stream signalling. Essentially, generated IL-18 mimetics are resistant to inhibition by IL-18BP (dark grey, $). Generated with PyMOL software version 2.3.0 based on PDB entries YYY and ZZZ and modified using www.biorender.com (B) Immunization of camelids followed by YSD facilitate the enrichment of sdAbs specific to (rh) IL-18Rα and (rh) IL-18Rβ. One sublibrary was generated for each specimen (huarizo and llama) and sorted separately. In the first round of selection, a mixture of both (rh) receptor subunits was exploited at a concentration of 250 nM. In the subsequent second sorting round, enriched libraries were sorted separately against each antigen at 250 nM. A two-dimensional sorting strategy was applied to select for full-length VHH display in addition to antigen binding. Percentage of cells in sorting gates are shown. Plots show 5×10⁴ events. (C) Graphical alignments of 55 unique sdAb clones addressing IL-18Rα (top) as well as 101 independent clones targeting IL-18Rβ (bottom) retrieved from YSD library sorting. Complementarity-determining regions (CDRs) are highlighted. Red bars indicate high sequence diversity at the amino acid level and green bars represent high sequence conservation at a given position. Alignment conducted with MUSCLE alignment tool using Geneious Prime 2021.1.1.

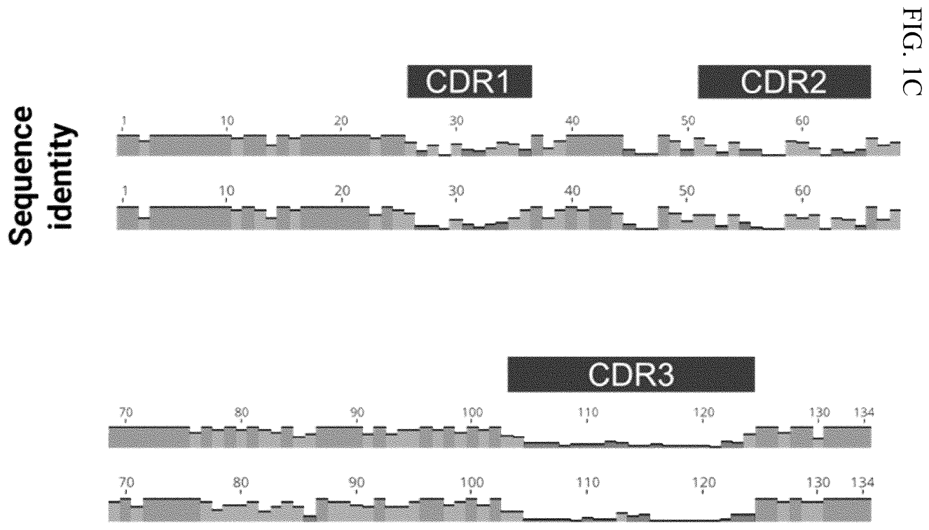

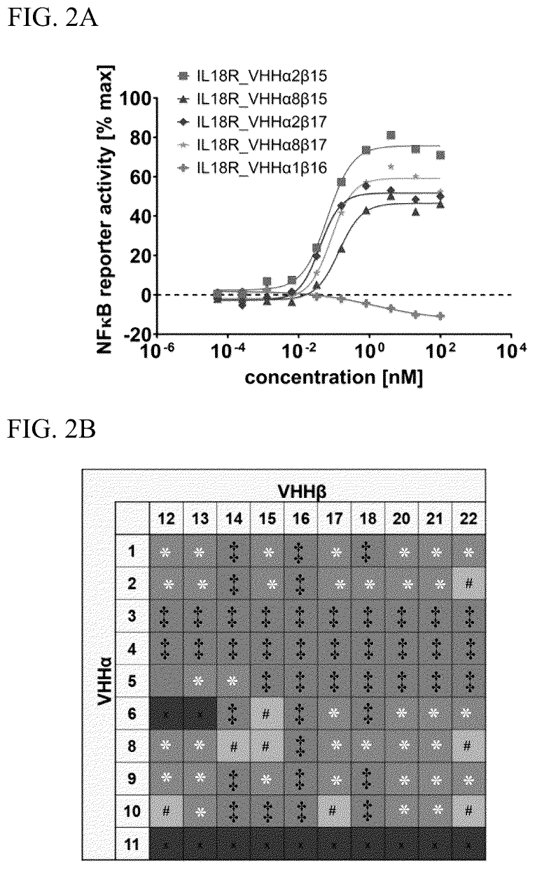

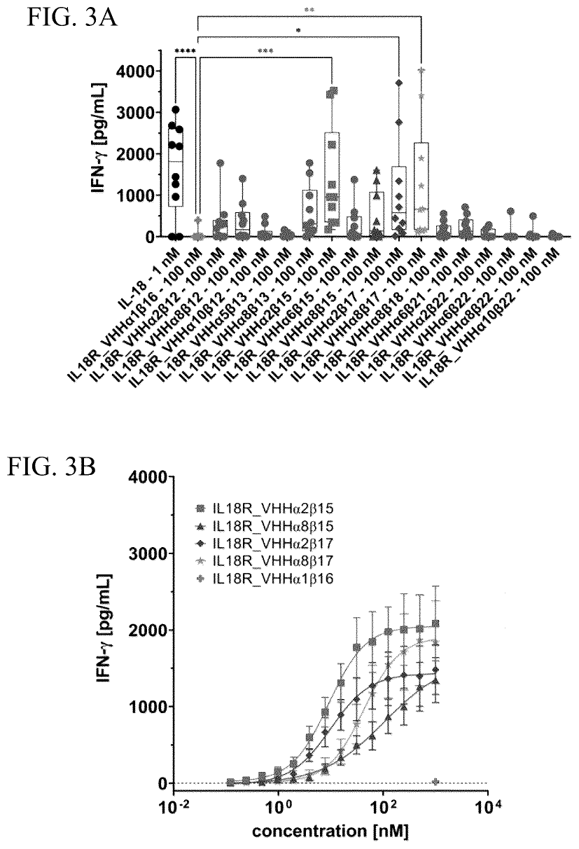

FIG. 2 shows data obtained with a reporter assay, confirming that combinatorial reformatting of monospecific (1+0) SEEDbodies into strictly monovalent (1+1) bsAbs enables the identification of IL-18 mimetics with attenuated capacities to trigger NFκB reporter activity on IL-18 reporter cells. (A) HEK-Blue™ reporter cells were incubated with increasing concentrations of reformatted bsAbs, as exemplarily shown for IL18R_VHHα2β15, IL18R_VHHα8β15, IL18R_VHHα2β17 and IL18R_VHHα8β17. Secreted embryonic alkaline phosphatase activity was monitored by determining the OD₆₄₀. Reporter activity was normalized to maximal IL-18 read-out. (B) Heatmap of NFκB reporter activation elicited by combinatorial reformatted (1+1) bsAbs. Molecules failing initial quality control (target monomer peak in SEC <96% post protein A purification given in dark grey, functionally inactive bsAbs shown in red (‡), minimally active surrogate agonists (NFκB reporter activation <50% compared to (rh) IL-18at 1 nM or EC50≥0.1 nM) in yellow (#) and moderately active IL-18 mimetics (NFκB reporter activation ≥50% compared to (rh) IL-18at 1 nM or EC50<0.1 nM) depicted in green (*). FIG. 3 depicts data from an IFN-γ release assay, confirming that bispecific (1+1) surrogate agonists trigger IFN-γ release on PBMCs isolated from healthy donors. (A) IFN-γ production of PBMCs stimulated with bsAbs at a fixed concentration of 100 nM or with (rh) IL-18 at 1 nM. Experiments were performed in the presence of 10 ng/ml (rh) IL-12. Graph shows box and whisker plots as superimpositions with dot plots of IFN-γ release of ten different donors. ****p<0.0001,***p<0.001, **p<0.01, *p<0.05. IL18R_VHHα1β16 was used as negative control (given in red). Four leading candidates used for further characterization shown in green, purple, blue and orange. (B) TOP4 candidates evoke a dose dependent IFN-γ read-out on PBMCs in the presence of low dose (rh) IL-12 (10 ng/ml). IL18R_VHHα1β16 as negative control shown in red was used at a fixed concentration of 1 μM. Mean values ±SEM of ten independent experiments are shown.

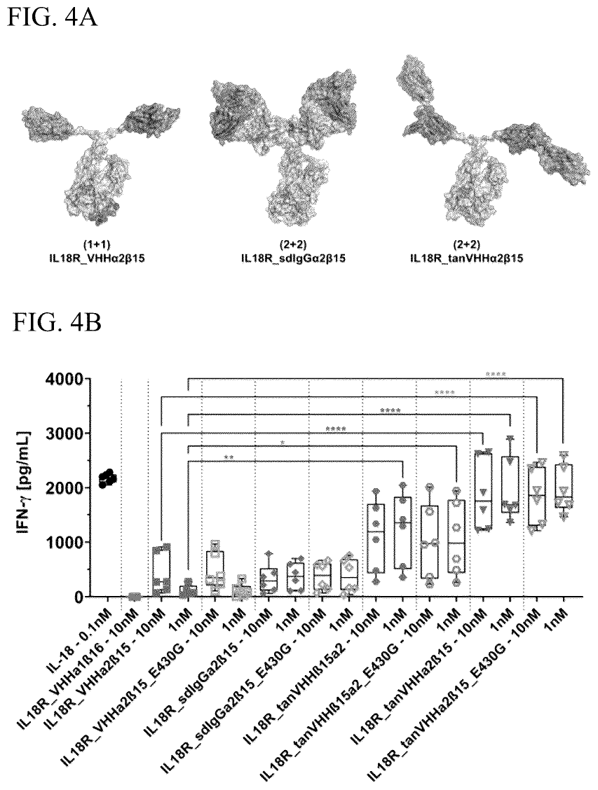

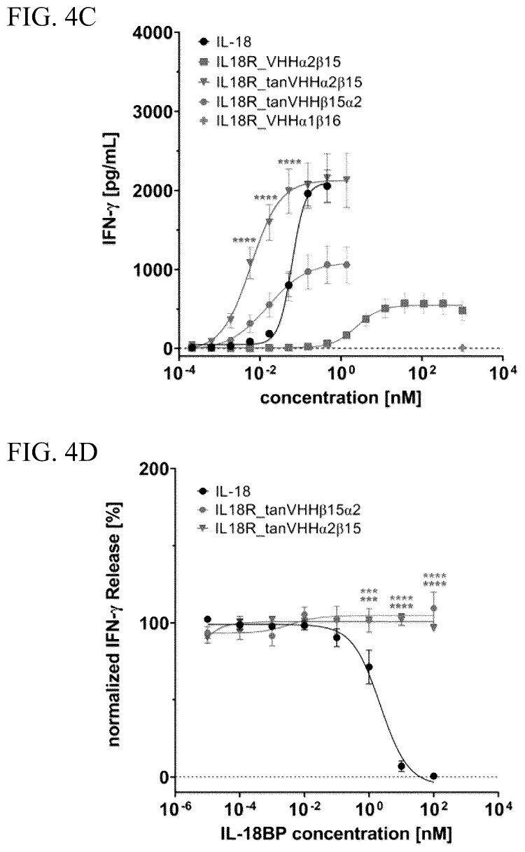

FIG. 4 shows schematic depictions of the architecture of antibodies prepared and summarizes data to characterize such antibodies. These data confirm that antibody engineering enables the generation of IL-18 mimetics with augmented agonism capacities. (A) Schematic depiction of main different bispecific antibody architectures that were constructed within this work. Fusion of an anti-IL18Rα VHHα2 to the hinge region of the AG chain of the SEEDbody as well as engraftment of an anti-IL18Rβ VHHβ15 onto the GA chain results in the initially generated (1+1) format IL18R_VHHα2β15. Replacing the VH and VLλ of an effector silenced IgG by VHHα2 and VHHβ15, respectively facilitates the generation of the IL18R_sdIgGα2β15 architecture (2+2). Within the IL18R_tanVHHα2β15 design (2+2), VHHα2 and VHHβ15 are arranged in tandem (from N-terminus to C-terminus) and separated by a five amino acid Gly₄Ser linker. The tandem is fused to the hinge region of an effector silenced IgG1 Fc fragment. Of note, also the opposite orientation was constructed (VHHβ15 followed by VHHα2, IL18R_tanVHHβ15α2). In addition, all four molecules were also produced harboring the E430G mutation for on-target hexamerization. Structure models generated with PyMOL software version 2.3.0. (B) Distinct surrogate agonist formats of the same paratopes (VHHα2 and VHHβ15) display differential properties in eliciting a functional IFN-γ response on human PBMCs isolated from healthy donors at fixed concentrations. Experiments were performed at two different concentrations (10 nM and 1 nM) in the presence of 10 ng/ml (rh) IL-12. Graph shows box and whisker plots as superimpositions with dot plots of IFN-γ release of six different donors. ****p<0.0001,***p<0.001, **p<0.01, *p<0.05. (C) Surrogate agonists arranged 407 in tandem (IL18R_tanVHHα2β15 and IL18R_tanVHHβ15α2) elicit enhanced IFN-γ production in terms of potencies and magnitude on PBMCs isolated from healthy donors, resulting in a variant with increased potencies compared with (rh) IL-18. All experiments were performed in the presence of low dose (rh) IL-12 (10 ng/ml). IL18R_VHHα1 β16 as negative control shown in red was used at a fixed concentration of 1 μM. Mean values ±SEM of 13 independent experiments are shown. (D) Potency augmented tandem IL-18 mimetics are resistant to inhibition by (rh) IL-18BP, whereas (rh) IL-18 is efficiently blocked from signalling. PBMCs of healthy human donors were stimulated either with (rh) IL-18 or IL18R_tanVHHα2β15 and IL18R tanVHHβ15α2 at a fixed concentration of 0.5 nM in the presence of (rh) IL-12 (10 ng/ml) and different concentrations of (rh) IL-18BP. Five independent experiments were performed and mean values ±SEM are shown.

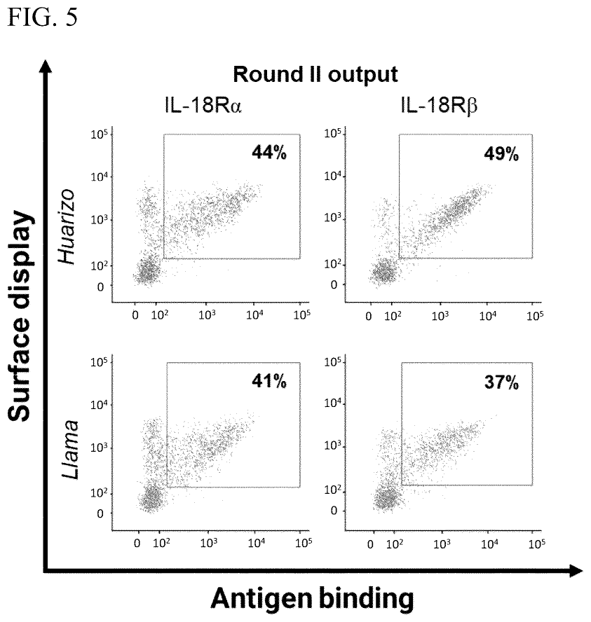

FIG. 5 summarizes the sorting procedure applied to obtain sdABs. YSD enables the enrichment of sdAbs targeting (rh) IL-18Rα and (rh) IL-18Rβ. Sorting output after two sorting rounds of each library against (rh) IL-18Rα and (rh) IL-18Rβ. A two-dimensional sorting strategy was applied do detect full-length VHH display simultaneous to the binding functionality at a concentration of 250 nM. Plots show 5×10³ events of the corresponding sorting output to visualize enrichment.

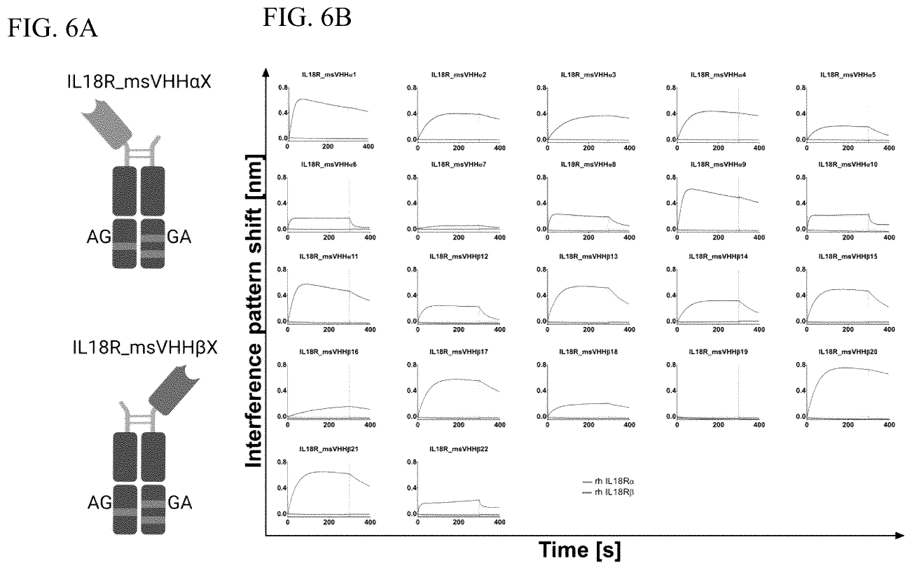

FIG. 6 depicts the results of binding experiments examining if monospecific (1+0) VHH SEEDbodies show specific binding to (rh) IL-18Rα (red) or IL-18Rβ (green). (A) Schematic depiction of monospecific VHHs targeting (rh) IL-18Rα (red) or (rh) IL-18Rβ (green) engrafted onto the hinge region of the SEED backbone. sdAbs targeting IL-18Rα were fused to the hinge region of the AG chain (msVHHαX), while VHHs addressing the IL-18Rβ subunit were grafted onto the GA chain (msVHHβX). (B) Binding of respective monospecific VHH SEEDbodies to (rh) IL-18Rα (red) or (rh) IL-18Rβ (green) as determined by BLI. Either (rh) IL-18Rα or (rh) IL-18Rβ were loaded to H1S1K biosensors at a concentration of 3 μg/ml followed by a first association of monospecific VHH SEEDbodies at 100 nM for 300 s. Subsequently, dissociation was measured for 100 s in kinetics buffer. Schemes generated via www.biorender.com.

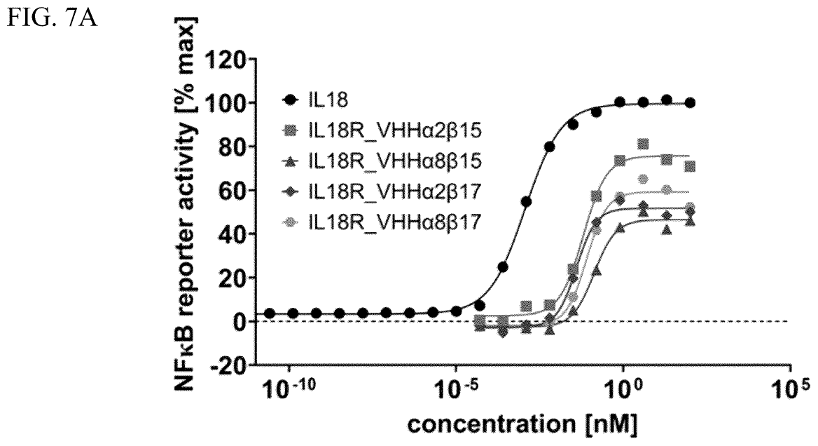

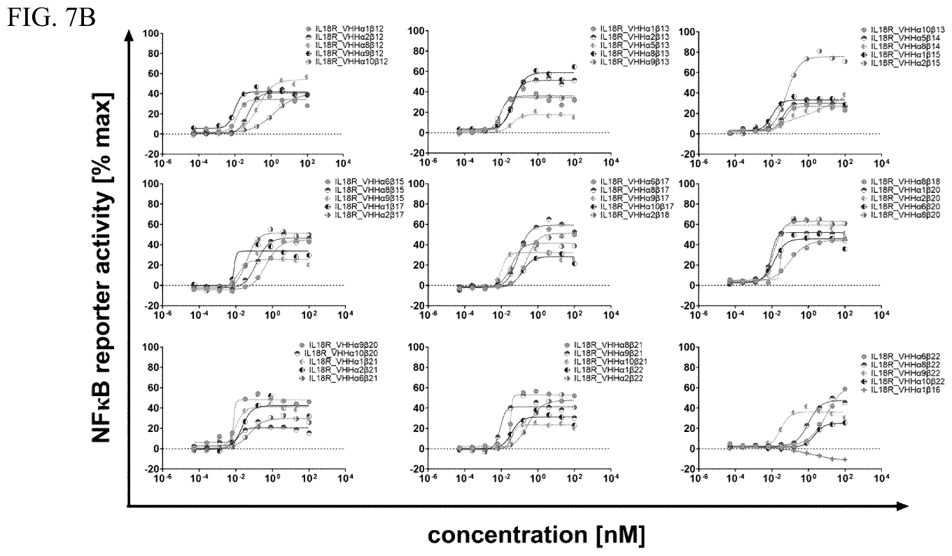

FIG. 7 presents data from a reporter assay suggesting that combinatorial reformatting of monospecific (1+0) SEEDbodies into strictly monovalent (1+1) bsAbs enables the identification of IL-18 mimetics with attenuated capacities to trigger NFκB reporter activity on IL-18 reporter cells. HEK-Blue™ reporter cells were incubated with increasing concentrations of reformatted bsAbs or (rh) IL-18 and secreted embryonic alkaline phosphatase activity was monitored by determining the OD₆₄₀. Reporter activity was normalized to maximal IL-18 read-out. (A) Exemplary dose-dependent IL-18R agonism triggered by surrogate agonists IL18R_VHHα2β15, IL18R_VHHα8β15, IL18R_VHHα2β17 and IL18R_VHHα8β17 in direct comparison to (rh) IL-18. (B) Dose-dependent receptor agonism mediated by all 44 cytokine mimetics.

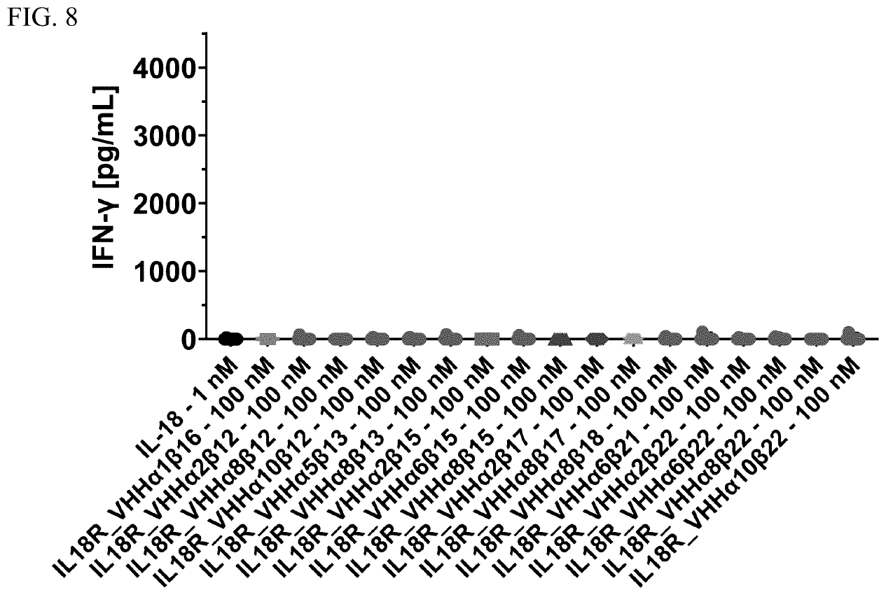

FIG. 8 depicts data from an IFN-γ release assay, showing that bispecific (1+1) surrogate agonists do not elicit IFN-γ release on PBMCs isolated from healthy donors without the presence of low dose (rh) IL-12. IFN-γ production of PBMCs stimulated with bsAbs at a fixed concentration of 100 nM or with (rh) IL-18 at 1 nM in the absence of (rh)IL-12. Graph dot plots of IFN-γ release of eight different donors. IL18R_VHHα1β16 was used as negative control (given in red). Four leading candidates used for further characterization shown in green, purple, blue and orange.

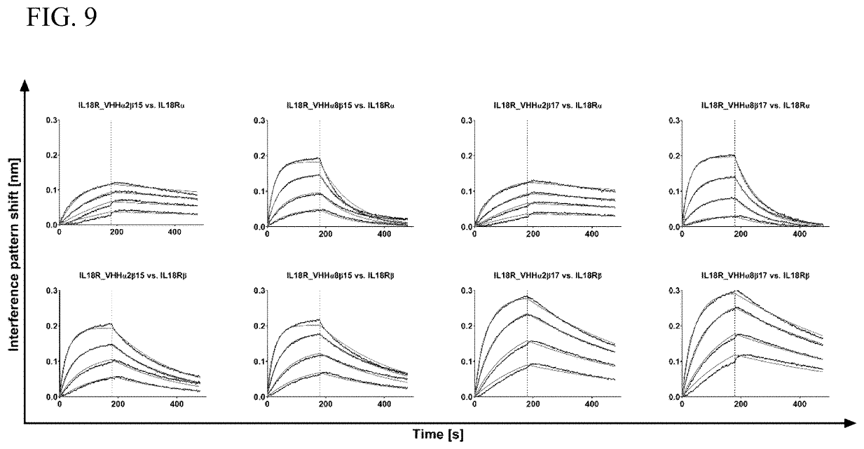

FIG. 9 shows binding kinetics data obtained with bispecific (1+1) TOP4 surrogate agonists against receptor subunits IL-18Rα and IL-18Rβ. IL-18 mimetics were loaded on AHC biosensor tips at a concentration of 5 μg/ml. Interactions against both receptor subunits were measured at a concentration of 100 nM, 50 nM, 25 nM and 12.5 nM for 180 s followed by a dissociation in kinetics buffer for 300 s.

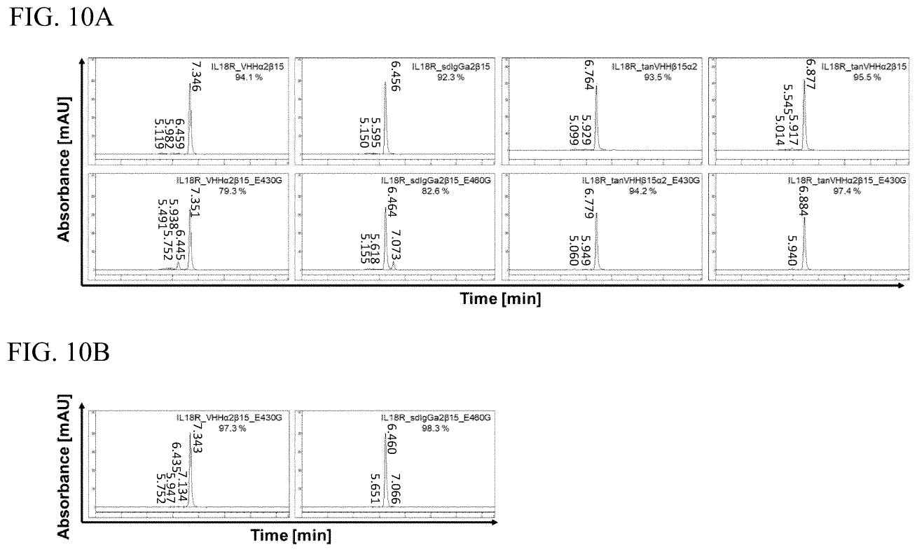

FIG. 10 summarizes analytical size exclusion chromatography profiles of the generated surrogate agonist formats, indicating adequate purities following downstream purification. SEC-HPLC profiles of different formats as well as the corresponding target monomer peaks are shown. Absorbance at 214 nm was used for purity determination. (A) SEC profiles of all eight formats post protein A purification, indicating purities below 90% target species for IL18R_VHHα2β15_E430G and IL18R_sdIgGα2β15_E430G. (B) Analysis of IL18R_VHHα2β15_E430G and IL18R_sdIgGα2β15_E430G following second step SEC purification.

FIG. 11 depicts data obtained by differential scanning fluorimetry to characterize the thermal unfolding of IL-18R agonistic bsAbs in different formats. Overlays of the melting curves for different formats of IL-18R agonists were recorded utilizing a temperature gradient from 20 °C to 95 °C at a slope of 1 °C/min. First derivatives of 350 nm/330 nm curves are shown.

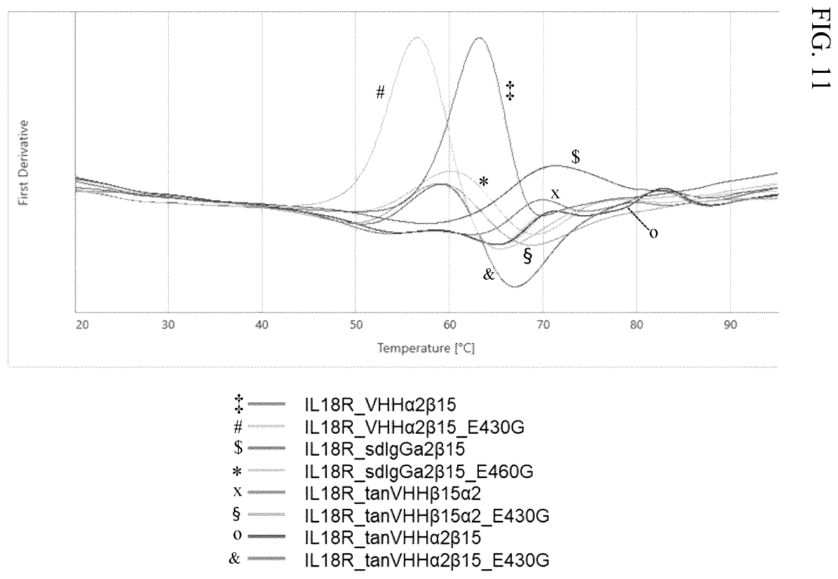

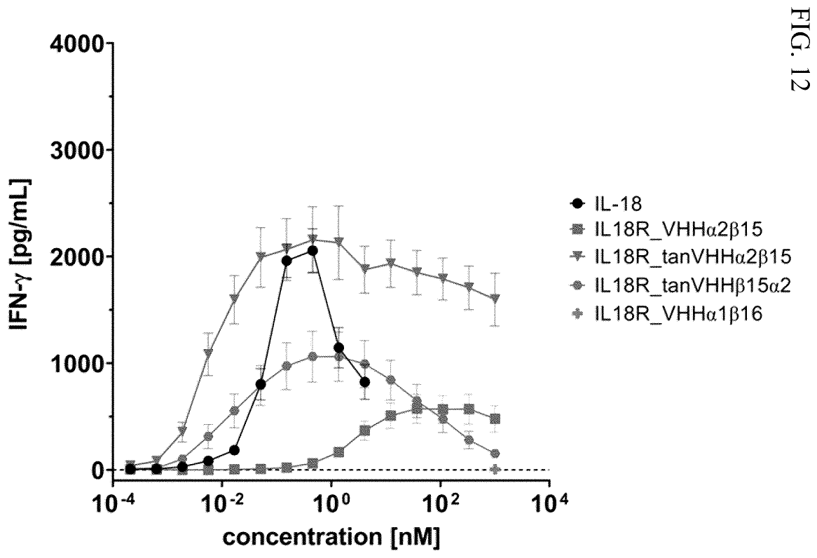

FIG. 12 shows data obtained upon stimulation of PBMCs with the full dose-range of compound concentrations, indicating a strong hooking effect for (rh) IL18 while for the different surrogate agonist formats it is not as pronounced. IFN-γ read-out on PBMCs in the presence of low dose (rh) IL-12 (10 ng/ml). IL18R_VHHα1β16 as negative control shown in red was used at a fixed concentration of 1 μM. Mean values ±SEM of 13 independent experiments are shown.

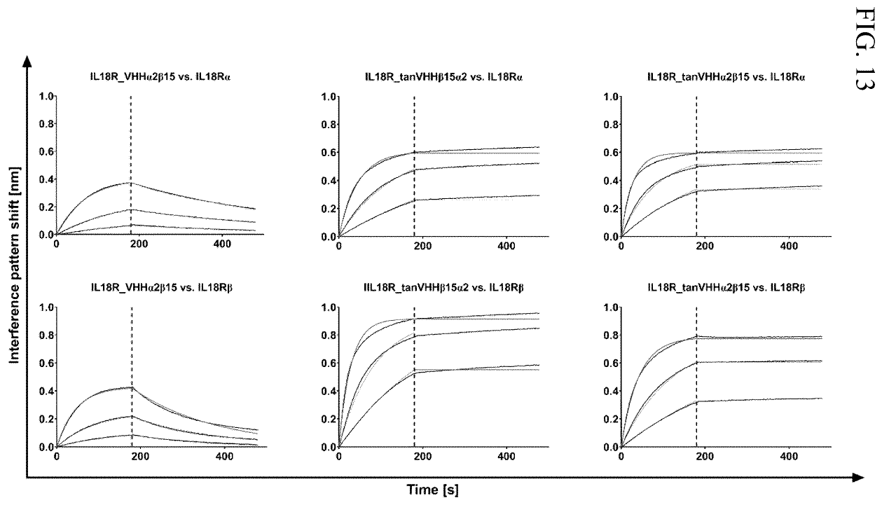

FIG. 13 shows data examining the binding of engineered tandem IL-18 mimetics. Such engineered tandem IL-18 mimetics exhibit strong binding to both receptor subunits in an avidity driven setting. (rh) IL-18Rα or (rh) IL-18Rβ were loaded onto HIS1K sensor tips at 5 μg/ml. A first association step was performed with different surrogate agonist formats at 100 nM, 33.3 nM and 11.1 nM for 180 s followed by a dissociation step in kinetics buffer fpr 300 s.

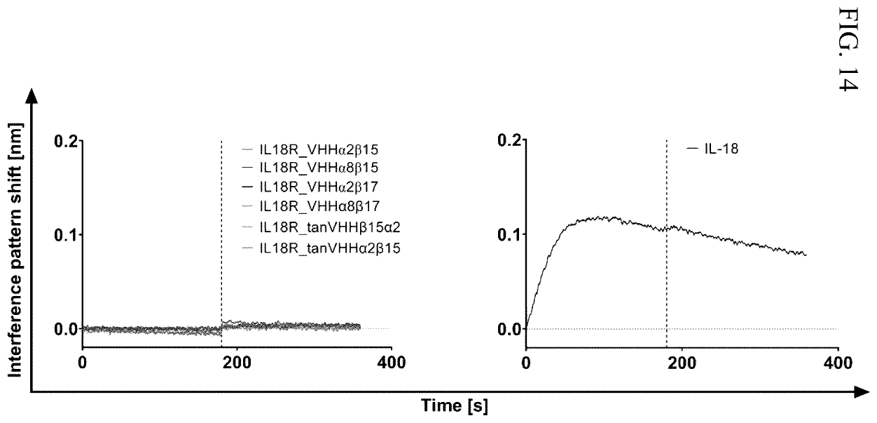

FIG. 14 shows biolayer interferometry (BLI) binding data for the binding of bispecific surrogate agonists to (rh)IL-18BP. Bispecific surrogate agonists do not bind to (rh)IL-18BP as determined, whereas (rh) IL-18BP binds to (rh)IL-18 with high affinities. BsAbs were loaded on AHC biosensor tips at 5 μg/ml. Subsequently, association was monitored using 1 M IL-18BP for 180 s, followed by dissociation in kinetics buffer for 180 s.

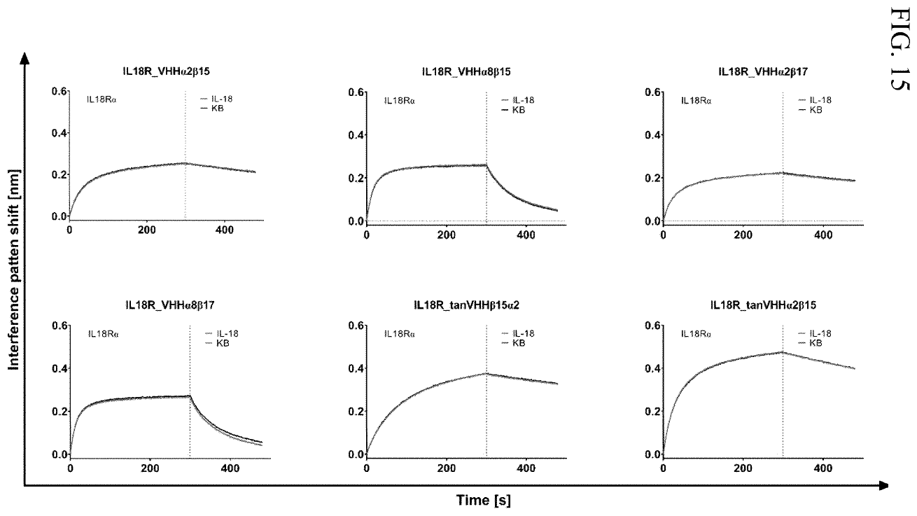

FIG. 15 summarizes binding data showing that bispecific cytokine mimetics compete with (rh)IL-18 for binding to (rh)IL-18Rα. Surrogate agonists were loaded on AHC biosensor tips, followed by association using IL-18Rα at 200 nM for 300 s. Subsequently, a second association step was performed for 180 s using either (rh)IL-18 at 100 nM (red) or kinetics buffer (black).

### SUMMARY OF SEQUENCES

(in case of any difference between the below table and the co-filed WIPO ST.26 compliant sequence listing, the information of the below table shall prevail)

| | Identifier/ | |
|---|---|---|
| SEQ ID NO | Description | Amino acid sequence |
| SEQ ID NO: 1 | VHH1 | DVPLQESGGGLVQTGGSLRLSCAASGFTENSYA |
| | | MGWYQQAPGKKYRELVAHIGSGGSTRYIDSVKG |
| | | RFTISRDNAKNTVYLQMNSLKPEDTAVYYCNVY |
| | | GATLRSWGQGTQVTVSS |
| SEQ ID NO: 2 | VHH2 | DVQLQESGGGLVQAGGSLRLSCAASGFTLLDSY |
| | | DIGWFRQAPGKKREEVSCISSSGGKSTYYADSV |
| | | KGRFTISRDNANNTVYLOMNSLKPEDTAVYYCA |
| | | ADGAEGFWSCQSGGYDYWGQGTQVTVSS |
| SEQ ID NO: 3 | VHH3 | DVQLQESGGGLVQAGGSLRLSCAASGFTLGAYA |
| | | INWFRQAPGKERARAWDACITSSGRSAYYADSV |
| | | KGRFTVSRDHAKNTVYLQMNSLKPEDTAFYYCA |
| | | AGAACAAAGTMNYWGKGTLVTVSS |
| SEQ ID NO: 4 | VHH4 | DVQLQESGGGLVQAGGSLRLSCAASGFTLGAYA |
| | | INWFQQAPGKERARAWVLCITSSGGSAYYVDSV |
| | | KGRFTVSRDNAKNTVYLQMNSLKPEDTAFYYCA |
| | | AGGACAAAETMNYWGKGTLVTVSS |
| SEQ ID NO: 5 | VHH5 | DVQLQESGGGLVQAGGSLRLSCAASGRTESNLA |
| | | MAWFRQAPGKEREFVAAIDWNGDNRLYAASVKG |
| | | RFTISRDNAKSTMYLQMNSLKPEDTAVYYCAAD |
| | | STYSTDWYLNPLNRTRYDYWGQGTQVTVSS |
| SEQ ID NO: 6 | VHH6 | DVPLQESGGGSVQPGGSLRLSCAASKSIFSINA |
| | | MRWHRQAPGKQRELVATFTSSGRINYADSVKGR |
| | | FTISTDNAKNTIYLQMNSLNPEDTAVYRCNVGL |
| | | WEDGLWYDYWGQGTQVTVSS |
| SEQ ID NO: 7 | VHH7 | DVQLQESGGGLVQAGGSLRLSCAASGHTENNLA |
| | | MAWCRQAPGKEREFVSAIDWNGDNRLYAYSVKG |
| | | RFTISRDNAKSTMYLQMNSLKPEDTAVYYCAAD |
| | | STYSTNWYLDPLNRTTYNYWGQGTPVTVSS |
| SEQ ID NO: 8 | VHH8 | DVQLQESGGGLVQAGGSLRLSCAASGRTFSNDA |
| | | MGWFRQAPGKEREFVAAVSWSLGMTYNADSVKG |
| | | RFTISRDNAKNTVYLQMNSLKPDDTAVYYCAAG |
| | | PDLSDELYGYDYWGQGTQVTVSS |
| SEQ ID NO: 9 | VHH9 | DVQLQESGGGLVOPGESLRLSCTASRSDESRYT |
| | | MGWYRQAPGKWRELVARITSGGIPHYADSVKGR |
| | | FTISRDNAKNTVTLQMNTLQFEDSAVYYCNTVG |
| | | VALWAWGQGTLVTVSS |
| SEQ ID NO: 10 | VHH10 | DVQLQESGGGLVQAGGSLRLSCAASGLPFSSYA |
| | | MGWFRQAPGKDREFVAGITWSGSSIYYRDSVKG |
| | | RFTISRDNAKNTVWLQMNDLKPEDTAVYYCAAA |
| | | RRRVGLPLADPREYVYWGOGTQVTVSS |
| SEQ ID NO: 11 | VHH11 | DVPLQESGGGLVQPGQSLRLSCAASGSPIRFSV |
| | | MGWFQQAPGKGRDLIARITSGGIVYYTDSVKGR |
| | | FTISKDNAKNTLYLOMNSLNLEDTGVYYCNRLG |
| | | GALFDWGQGTQVTVSS |
| SEQ ID NO: 12 | VHH12 | DVPLQESGGGLVQPGGSLRLSCRASGVGFSIYT |
| | | MGWYRQAPGEQRHLVATVTTGGHINYGDSVQGR |
| | | FTISRENNGNVVELQMHSLKPEDTAVYFCNAIY |
| | | FGQDIWGQGTQVTVSS |
| SEQ ID NO: 13 | VHH13 | DVQLQESGGGLVQPGGSLRLSCAASGISFRVNV |
| | | LGWYRQAPGKPRELVASISSGDSTNYADSVKGR |
| | | FTISRDNAKSTVYLOMNNLKPEDTAVYYCNARI |
| | | FARDNWGQGTQVTVSS |
| SEQ ID NO: 14 | VHH14 | DVPLQESGGGLVQAGGSLRLSCAASDRTVSKGA |
| | | MGWFRQAPGKEREFVATIGRSSGITYYADSVKG |
| | | RFTISRDNAKNTWYLQMNSLRPEDTAVYYCAAD |
| | | SSGIIRTTSWAYDYWGQGTQVTVSS |
| SEQ ID NO: 15 | VHH15 | DVQLQESGGGLVQPGGSLRLSCAASGRFFTINA |
| | | MGWYRQTPGKQRELVAAITSDGSTTYQDSVKGR |
| | | FTISRDNANRTANLYMNSLKSEDTAVYYCNADG |
| | | RFNDNGYVKNFNIWGQGTQVTVSS |
| SEQ ID NO: 16 | VHH16 | DVQLQESGGGSVPAGGSLRLSCSASGFTTDDYI |
| | | IGWFRQAPGKKREGVSCISTTDGSAWYADSVKG |
| | | RFTISSDNANNTVTLQMNSLLPEDTSVYYCAEV |
| | | STGTGVLYCREEFYFGMDSWGKGTPVTVSS |
| SEQ ID NO: 17 | VHH17 | DVPLQESGGGLVQAGGSLRLSCVASGFALDEIA |
| | | VGWFRQAPGKEREGLSCISSSDGSTLYADSVKG |
| | | RFTISSDNAKNTVYLOMNSLKPEDTAVYLCAAV |
| | | RGSCFGRTTHWGQGTQVTVSS |
| SEQ ID NO: 18 | VHH18 | DVQLQESGGGLVQAGGSLRLSCAASGRTFSSYA |
| | | MGWFRQAPGMVREFVAAISWSGGSTYYADSVQG |
| | | RFTISRDNAKNTVYLOMNSLKPEDTAVYYCAAD |
| | | RSLSGSYFGATIDYWGQGTQVTVSS |
| SEQ ID NO: 19 | VHH19 | DVQLQESGGGLVQAGGSLRLSCAASGIRENPER |
| | | MAWYRQAPGKQRELVAAIDSGGSTDYADSVRGR |
| | | FTISRDNAKITVSLQMNSLKPEDTAVYYCNTQI |
| | | FARDYWGQGSQVTVSS |
| SEQ ID NO: 20 | VHH20 | DVQLQESGGGLVOAGGSLRLSCAASVSAFRLKV |
| | | MGWYRQAPGKEREFVAAIDSGDTKSYTNAVKGR |
| | | FTISGGAATNTVVLOMNSLKPEDTAFYYCNAVL |
| | | FGRDYWGQGTQVTVSS |
| SEQ ID NO: 21 | VHH21 | DVQLQESGGGLVQAGGSLRLSCAASGITFSPYV |
| | | MGWYRQAPGKQRELVADMVNGGSTNYADSVKGR |
| | | FTIFRDTAANTVTLQMNNLKPDDTAVYYCHIMI |
| | | FGRASWGQGTQVTVSS |
| SEQ ID NO: 22 | VHH22 | DVPLQESGGGLVQPGGSLRLSCAAPGFALEYYA |
| | | IGWFRQAPGKEREGLSCVSSSRSGGSTVYADSV |
| | | KGRFTISRDNAKNTVYLQIDSLKVEDTAVYYCA |
| | | GAPAYRSHYCRLQADDYTYWGQGTQVTVSS |
| SEQ ID NO: 23 | CDR1 of VHH1 | GFTFNSYA |
| SEQ ID NO: 24 | CDR2 of VHH1 | HIGSGGSTR |
| SEQ ID NO: 25 | CDR3 of VHH1 | NVYGATLRS |
| SEQ ID NO: 26 | CDR1 of VHH2 | GFTLLDSYD |
| SEQ ID NO: 27 | CDR2 of VHH2 | ISSSGGKSTY |
| SEQ ID NO: 28 | CDR3 of VHH2 | AADGAEGFWSCQSGGYDY |
| SEQ ID NO: 29 | CDR1 of VHH3 | GFTLGAYA |
| SEQ ID NO: 30 | CDR2 of VHH3 | ITSSGRSAY |
| SEQ ID NO: 31 | CDR3 of VHH3 | AAGAACAAAGTMNY |
| SEQ ID NO: 32 | CDR1 of VHH4 | GFTLGAYA |
| SEQ ID NO: 33 | CDR2 of VHH4 | ITSSGGSAY |
| SEQ ID NO: 34 | CDR3 of VHH4 | AAGGACAAAETMNY |
| SEQ ID NO: 35 | CDR1 of VHH5 | GRTFSNLA |
| SEQ ID NO: 36 | CDR2 of VHH5 | IDWNGDNRL |
| SEQ ID NO: 37 | CDR3 of VHH5 | AADSTYSTDWYLNPLNRTRYDY |
| SEQ ID NO: 38 | CDR1 of VHH6 | KSIFSINA |
| SEQ ID NO: 39 | CDR2 of VHH6 | FTSSGRIN |
| SEQ ID NO: 40 | CDR3 of VHH6 | NVGLWEDGLWYDY |
| SEQ ID NO: 41 | CDR1 of VHH7 | GHTENNLA |
| SEQ ID NO: 42 | CDR2 of VHH7 | IDWNGDNRL |
| SEQ ID NO: 43 | CDR3 of VHH7 | AADSTYSTNWYLDPLNRTTYNY |
| SEQ ID NO: 44 | CDR1 of VHH8 | GRTFSNDA |
| SEQ ID NO: 45 | CDR2 of VHH8 | VSWSLGMTY |
| SEQ ID NO: 46 | CDR3 of VHH8 | AAGPDLSDELYGYDY |
| SEQ ID NO: 47 | CDR1 of VHH9 | RSDFSRYT |
| SEQ ID NO: 48 | CDR2 of VHH9 | RITSGGIPH |
| SEQ ID NO: 49 | CDR3 of VHH9 | NTVGVALWA |
| SEQ ID NO: 50 | CDR1 of | GLPFSSYA |
| | VHH10 | |
| SEQ ID NO: 51 | CDR2 of | ITWSGSSIY |
| | VHH10 | |
| SEQ ID NO: 52 | CDR3 of | AAARRRVGLPLADPREYVY |
| | VHH10 | |
| SEQ ID NO: 53 | CDR1 of | GSPIRFSV |
| | VHH11 | |
| SEQ ID NO: 54 | CDR2 of | RITSGGIVY |
| | VHH11 | |
| SEQ ID NO: 55 | CDR3 of | NRLGGALFD |
| | VHH11 | |
| SEQ ID NO: 56 | CDR1 of | GVGFSIYT |
| | VHH12 | |
| SEQ ID NO: 57 | CDR2 of | VTTGGHIN |
| | VHH12 | |
| SEQ ID NO: 58 | CDR3 of | NAIYFGQDI |
| | VHH12 | |
| SEQ ID NO: 59 | CDR1 of | GISFRVNV |
| | VHH13 | |
| SEQ ID NO: 60 | CDR2 of | ISSGDSTN |
| | VHH13 | |
| SEQ ID NO: 61 | CDR3 of | NARIFARDN |
| | VHH13 | |
| SEQ ID NO: 62 | CDR1 of | DRTVSKGA |
| | VHH14 | |
| SEQ ID NO: 63 | CDR2 of | IGRSSGITY |
| | VHH14 | |
| SEQ ID NO: 64 | CDR3 of | AADSSGIIRTTSWAYDY |
| | VHH14 | |
| SEQ ID NO: 65 | CDR1 of | GRFFTINA |
| | VHH15 | |
| SEQ ID NO: 66 | CDR2 of | ITSDGSTT |
| | VHH15 | |
| SEQ ID NO: 67 | CDR3 of | NADGRENDNGYVKNFNI |
| | VHH15 | |
| SEQ ID NO: 68 | CDR1 of | GFTTDDYI |
| | VHH16 | |
| SEQ ID NO: 69 | CDR2 of | ISTTDGSAW |
| | VHH16 | |
| SEQ ID NO: 70 | CDR3 of | AEVSTGTGVLYCREEFYFGMDS |
| | VHH16 | |
| SEQ ID NO: 71 | CDR1 of | GFALDEIA |
| | VHH17 | |
| SEQ ID NO: 72 | CDR2 of | ISSSDGSTL |
| | VHH17 | |
| SEQ ID NO: 73 | CDR3 of | AAVRGSCFGRTTH |
| | VHH17 | |
| SEQ ID NO: 74 | CDR1 of | GRTFSSYA |
| | VHH18 | |
| SEQ ID NO: 75 | CDR2 of | ISWSGGSTY |
| | VHH18 | |
| SEQ ID NO: 76 | CDR3 of | AADRSLSGSYFGATIDY |
| | VHH18 | |
| SEQ ID NO: 77 | CDR1 of | GIRENPFR |
| | VHH19 | |
| SEQ ID NO: 78 | CDR2 of | IDSGGSTD |
| | VHH19 | |
| SEQ ID NO: 79 | CDR3 of | NTQIFARDY |
| | VHH19 | |
| SEQ ID NO: 80 | CDR1 of | VSAFRLKV |
| | VHH20 | |
| SEQ ID NO: 81 | CDR2 of | IDSGDTKS |
| | VHH20 | |
| SEQ ID NO: 82 | CDR3 of | NAVLFGRDY |
| | VHH20 | |
| SEQ ID NO: 83 | CDR1 of | GITESPYV |
| | VHH21 | |
| SEQ ID NO: 84 | CDR2 of | MVNGGSTN |
| | VHH21 | |
| SEQ ID NO: 85 | CDR3 of | HIMIFGRAS |
| | VHH21 | |
| SEQ ID NO: 86 | CDR1 of | GFALEYYA |
| | VHH22 | |
| SEQ ID NO: 87 | CDR2 of | VSSSRSGGSTV |
| | VHH22 | |
| SEQ ID NO: 88 | CDR3 of | AGAPAYRSHYCRLQADDYTY |
| | VHH22 | |
| SEQ ID NO: 89 | VHH-SEED- | DVPLQESGGGLVQTGGSLRLSCAASGFTENSYA |
| | AG-1 | MGWYQQAPGKKYRELVAHIGSGGSTRYIDSVKG |
| | | RFTISRDNAKNTVYLOMNSLKPEDTAVYYCNVY |
| | | GATLRSWGQGTQVTVS |
| SEQ ID NO: 90 | VHH-SEED- | DVQLQESGGGLVQAGGSLRLSCAASGFTLLDSY |
| | AG-2 | DIGWFRQAPGKKREEVSCISSSGGKSTYYADSV |
| | | KGRFTISRDNANNTVYLOMNSLKPEDTAVYYCA |
| | | ADGAEGFWSCQSGGYDYWGQGTQVTVS |
| SEQ ID NO: 91 | VHH-SEED- | DVQLQESGGGLVQAGGSLRLSCAASGFTLGAYA |
| | AG-3 | INWFRQAPGKERARAWDACITSSGRSAYYADSV |
| | | KGRFTVSRDHAKNTVYLQMNSLKPEDTAFYYCA |
| | | AGAACAAAGTMNYWGKGTLVTVS |
| SEQ ID NO: 92 | VHH-SEED- | DVQLQESGGGLVQAGGSLRLSCAASGFTLGAYA |
| | AG-4 | INWFQQAPGKERARAWVLCITSSGGSAYYVDSV |
| | | KGRFTVSRDNAKNTVYLQMNSLKPEDTAFYYCA |
| | | AGGACAAAETMNYWGKGTLVTVS |
| SEQ ID NO: 93 | VHH-SEED- | DVQLQESGGGLVQAGGSLRLSCAASGRTFSNLA |
| | AG-5 | MAWFRQAPGKEREFVAAIDWNGDNRLYAASVKG |
| | | RFTISRDNAKSTMYLQMNSLKPEDTAVYYCAAD |
| | | STYSTDWYLNPLNRTRYDYWGQGTQVTVS |
| SEQ ID NO: 94 | VHH-SEED- | DVPLQESGGGSVQPGGSLRLSCAASKSIFSINA |
| | AG-6 | MRWHRQAPGKQRELVATFTSSGRINYADSVKGR |
| | | FTISTDNAKNTIYLQMNSLNPEDTAVYRCNVGL |
| | | WEDGLWYDYWGQGTQVTVS |
| SEQ ID NO: 95 | VHH-SEED- | DVQLQESGGGLVQAGGSLRLSCAASGHTENNLA |
| | AG-7 | MAWCRQAPGKEREFVSAIDWNGDNRLYAYSVKG |
| | | RFTISRDNAKSTMYLQMNSLKPEDTAVYYCAAD |
| | | STYSTNWYLDPLNRTTYNYWGQGTPVTVS |
| SEQ ID NO: 96 | VHH-SEED- | DVQLQESGGGLVQAGGSLRLSCAASGRTFSNDA |
| | AG-8 | MGWFRQAPGKEREFVAAVSWSLGMTYNADSVKG |
| | | RFTISRDNAKNTVYLQMNSLKPDDTAVYYCAAG |
| | | PDLSDELYGYDYWGQGTQVTVS |
| SEQ ID NO: 97 | VHH-SEED- | DVQLQESGGGLVQPGESLRLSCTASRSDESRYT |
| | AG-9 | MGWYRQAPGKWRELVARITSGGIPHYADSVKGR |
| | | FTISRDNAKNTVTLQMNTLQFEDSAVYYCNTVG |
| | | VALWAWGQGTLVTVS |
| SEQ ID NO: 98 | VHH-SEED- | DVQLQESGGGLVQAGGSLRLSCAASGLPFSSYA |
| | AG-10 | MGWFRQAPGKDREFVAGITWSGSSIYYRDSVKG |
| | | RFTISRDNAKNTVWLQMNDLKPEDTAVYYCAAA |
| | | RRRVGLPLADPREYVYWGQGTQVTVS |
| SEQ ID NO: 99 | VHH-SEED- | DVPLQESGGGLVOPGQSLRLSCAASGSPIRFSV |
| | AG-11 | MGWFQQAPGKGRDLIARITSGGIVYYTDSVKGR |
| | | FTISKDNAKNTLYLQMNSLNLEDTGVYYCNRLG |
| | | GALFDWGQGTQVTVS |
| SEQ ID NO: 100 | VHH-SEED- | DVPLQESGGGLVQPGGSLRLSCRASGVGESIYT |
| | GA-1 | MGWYRQAPGEQRHLVATVTTGGHINYGDSVQGR |
| | | FTISRENNGNVVELQMHSLKPEDTAVYFCNAIY |
| | | FGQDIWGQGTQVTVS |
| SEQ ID NO: 101 | VHH-SEED- | DVQLQESGGGLVQPGGSLRLSCAASGISFRVNV |
| | GA-2 | LGWYRQAPGKPRELVASISSGDSTNYADSVKGR |
| | | FTISRDNAKSTVYLOMNNLKPEDTAVYYCNARI |
| | | FARDNWGQGTQVTVS |
| SEQ ID NO: 102 | VHH-SEED- | DVPLQESGGGLVQAGGSLRLSCAASDRTVSKGA |
| | GA-3 | MGWFRQAPGKEREFVATIGRSSGITYYADSVKG |
| | | RFTISRDNAKNTWYLOMNSLRPEDTAVYYCAAD |
| | | SSGIIRTTSWAYDYWGOGTQVTVS |
| SEQ ID NO: 103 | VHH-SEED- | DVQLQESGGGLVQPGGSLRLSCAASGRFFTINA |
| | GA-4 | MGWYRQTPGKQRELVAAITSDGSTTYQDSVKGR |
| | | FTISRDNANRTANLYMNSLKSEDTAVYYCNADG |
| | | RENDNGYVKNFNIWGQGTQVTVS |
| SEQ ID NO: 104 | VHH-SEED- | DVQLQESGGGSVPAGGSLRLSCSASGFTTDDYI |
| | GA-5 | IGWFRQAPGKKREGVSCISTTDGSAWYADSVKG |
| | | RFTISSDNANNTVTLQMNSLLPEDTSVYYCAEV |
| | | STGTGVLYCREEFYFGMDSWGKGTPVTVS |
| SEQ ID NO: 105 | VHH-SEED- | DVPLQESGGGLVQAGGSLRLSCVASGFALDEIA |
| | GA-6 | VGWFRQAPGKEREGLSCISSSDGSTLYADSVKG |
| | | RFTISSDNAKNTVYLQMNSLKPEDTAVYLCAAV |
| | | RGSCFGRTTHWGQGTQVTVS |
| SEQ ID NO: 106 | VHH-SEED- | DVQLQESGGGLVQAGGSLRLSCAASGRTFSSYA |
| | GA-7 | MGWFRQAPGMVREFVAAISWSGGSTYYADSVQG |
| | | RFTISRDNAKNTVYLQMNSLKPEDTAVYYCAAD |
| | | RSLSGSYFGATIDYWGQGTQVTVS |
| SEQ ID NO: 107 | VHH-SEED- | DVQLQESGGGLVQAGGSLRLSCAASGIRENPFR |
| | GA-8 | MAWYRQAPGKQRELVAAIDSGGSTDYADSVRGR |
| | | FTISRDNAKITVSLQMNSLKPEDTAVYYCNTQI |
| | | FARDYWGQGSQVTVS |
| SEQ ID NO: 108 | VHH-SEED- | DVQLQESGGGLVQAGGSLRLSCAASVSAFRLKV |
| | GA-9 | MGWYRQAPGKEREFVAAIDSGDTKSYTNAVKGR |
| | | FTISGGAATNTVVLQMNSLKPEDTAFYYCNAVL |
| | | FGRDYWGQGTQVTVS |
| SEQ ID NO: 109 | VHH-SEED- | DVQLQESGGGLVQAGGSLRLSCAASGITESPYV |
| | GA-10 | MGWYRQAPGKQRELVADMVNGGSTNYADSVKGR |
| | | FTIFRDTAANTVTLQMNNLKPDDTAVYYCHIMI |
| | | FGRASWGQGTQVTVS |
| SEQ ID NO: 110 | VHH-SEED- | DVPLQESGGGLVQPGGSLRLSCAAPGFALEYYA |
| | GA-11 | IGWFRQAPGKEREGLSCVSSSRSGGSTVYADSV |
| | | KGRFTISRDNAKNTVYLQIDSLKVEDTAVYYCA |
| | | GAPAYRSHYCRLQADDYTYWGQGTQVTVS |
| SEQ ID NO: 111 | human IL- | MNCRELPLTLWVLISVSTAESCTSRPHITVVEG |
| | 18Ra | EPFYLKHCSCSLAHEIETTTKSWYKSSGSQEHV |
| | | ELNPRSSSRIALHDCVLEFWPVELNDTGSYFFQ |
| | | MKNYTQKWKLNVIRRNKHSCFTERQVTSKIVEV |
| | | KKFFQITCENSYYQTLVNSTSLYKNCKKLLLEN |
| | | NKNPTIKKNAEFEDQGYYSCVHFLHHNGKLENI |
| | | TKTFNITIVEDRSNIVPVLLGPKLNHVAVELGK |
| | | NVRLNCSALLNEEDVIYWMFGEENGSDPNIHEE |
| | | KEMRIMTPEGKWHASKVLRIENIGESNLNVLYN |
| | | CTVASTGGTDTKSFILVRKADMADIPGHVFTRG |
| | | MIIAVLILVAVVCLVTVCVIYRVDLVLFYRHLT |
| | | RRDETLTDGKTYDAFVSYLKECRPENGEEHTFA |
| | | VEILPRVLEKHFGYKLCIFERDVVPGGAVVDEI |
| | | HSLIEKSRRLIIVLSKSYMSNEVRYELESGLHE |
| | | ALVERKIKIILIEFTPVTDFTFLPQSLKLLKSH |
| | | RVLKWKADKSLSYNSRFWKNLLYLMPAKTVKPG |
| | | RDEPEVLPVLSES |
| SEQ ID NO: 112 | human IL- | CTSRPHITVVEGEPFYLKHCSCSLAHEIETTTK |
| | 18Ra ECD | SWYKSSGSQEHVELNPRSSSRIALHDCVLEFWP |
| | | VELNDTGSYFFQMKNYTQKWKLNVIRRNKHSCF |
| | | TERQVTSKIVEVKKFFQITCENSYYQTLVNSTS |
| | | LYKNCKKLLLENNKNPTIKKNAEFEDQGYYSCV |
| | | HFLHHNGKLFNITKTENITIVEDRSNIVPVLLG |
| | | PKLNHVAVELGKNVRLNCSALLNEEDVIYWMFG |
| | | EENGSDPNIHEEKEMRIMTPEGKWHASKVLRIE |
| | | NIGESNLNVLYNCTVASTGGTDTKSFILVRKAD |
| | | MADIPGHVFTR |
| SEQ ID NO: 113 | human IL- | MLCLGWIFLWLVAGERIKGENISGCSTKKLLWT |
| | 18RB | YSTRSEEEFVLFCDLPEPQKSHFCHRNRLSPKQ |
| | | VPEHLPFMGSNDLSDVOWYQQPSNGDPLEDIRK |
| | | SYPHIIQDKCTLHELTPGVNNSGSYICRPKMIK |
| | | SPYDVACCVKMILEVKPQTNASCEYSASHKODL |
| | | LLGSTGSISCPSLSCOSDAQSPAVTWYKNGKLL |
| | | SVERSNRIVVDEVYDYHQGTYVCDYTQSDTVSS |
| | | WTVRAVVQVRTIVGDTKLKPDILDPVEDTLEVE |
| | | LGKPLTISCKARFGFERVENPVIKWYIKDSDLE |
| | | WEVSVPEAKSIKSTLKDEIIERNIILEKVTORD |
| | | LRRKFVCFVQNSIGNTTQSVQLKEKRGVVLLYI |
| | | LLGTIGTLVAVLAASALLYRHWIEIVLLYRTYQ |
| | | SKDQTLGDKKDFDAFVSYAKWSSFPSEATSSLS |
| | | EEHLALSLFPDVLENKYGYSLCLLERDVAPGGV |
| | | YAEDIVSIIKRSRRGIFILSPNYVNGPSIFELQ |
| | | AAVNLALDDQTLKLILIKFCYFQEPESLPHLVK |
| | | KALRVLPTVTWRGLKSVPPNSRFWAKMRYHMPV |
| | | KNSQGFTWNQLRITSRIFQWKGLSRTETTGRSS |
| | | QPKEW |
| SEQ ID NO: 114 | human IL- | FNISGCSTKKLLWTYSTRSEEEFVLFCDLPEPQ |
| | 18RB ECD | KSHFCHRNRLSPKQVPEHLPFMGSNDLSDVQWY |
| | | QQPSNGDPLEDIRKSYPHIIQDKCTLHFLTPGV |
| | | NNSGSYICRPKMIKSPYDVACCVKMILEVKPQT |
| | | NASCEYSASHKQDLLLGSTGSISCPSLSCOSDA |
| | | QSPAVTWYKNGKLLSVERSNRIVVDEVYDYHQG |
| | | TYVCDYTQSDTVSSWTVRAVVQVRTIVGDTKLK |
| | | PDILDPVEDTLEVELGKPLTISCKARFGFERVE |
| | | NPVIKWYIKDSDLEWEVSVPEAKSIKSTLKDEI |
| | | IERNIILEKVTQRDLRRKFVCFVONSIGNTTQS |
| | | VQLKEKR |
| SEQ ID NO: 115 | human IL-18 | MAAEPVEDNCINFVAMKFIDNTLYFIAEDDENL |
| | | ESDYFGKLESKLSVIRNLNDQVLFIDQGNRPLF |
| | | EDMTDSDCRDNAPRTIFIISMYKDSQPRGMAVT |
| | | ISVKCEKISTLSCENKIISFKEMNPPDNIKDTK |
| | | SDIIFFQRSVPGHDNKMQFESSSYEGYFLACEK |
| | | ERDLFKLILKKEDELGDRSIMFTVQNED |
| SEQ ID NO: 116 | human IL- | MTMRHNWTPDLSPLWVLLLCAHVVTLLVRATPV |
| | 18BP | SQTTTAATASVRSTKDPCPSQPPVFPAAKQCPA |
| | | LEVTWPEVEVPLNGTLSLSCVACSRFPNESILY |
| | | WLGNGSFIEHLPGRLWEGSTSRERGSTGTQLCK |
| | | ALVLEQLTPALHSTNFSCVLVDPEQVVQRHVVL |
| | | AQLWAGLRATLPPTQEALPSSHSSPQQQG |

SEQ ID NO: 1 to 11 provide the amino acid sequences of VHH1 to VHH11 (corresponding to α1 to α11 according to the present disclosure). The CDRs of these VHHs are provided in SEQ ID NO: 23 to 55.

SEQ ID NO: 12 to 22 provide the amino acid sequences of VHH12 to VHH22 (corresponding to 312 to 022 according to the present disclosure). The CDRs of these VHHs are provided in SEQ ID NO: 56 to 88.

SEQ ID NO: 89 to 99 provide the amino acid sequences of SEED AG chains based on VHH1 to VHH11 (α1 to α11) according to the present disclosure. SEQ ID NO: 100 to 110 provide the amino acid sequences of SEED GA chains based on VHH12 to VHH22 (312 to 322) according to the present disclosure.

### DETAILED DESCRIPTION OF CERTAIN EMBODIMENTS

Although the present disclosure is described in detail above and below, it is to be understood that this disclosure is not limited to the particular methodologies, protocols and reagents described by the present disclosure, as these may vary. It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by one of ordinary skill in the art.

In the following, certain elements of the present disclosure will be described in more detail, including the description of specific embodiments. However, the variously described examples and preferred embodiments should not be construed to limit the present disclosure to only the explicitly described embodiments. This description should be understood to support and encompass embodiments which combine the explicitly described embodiments with any number of the disclosed and/or preferred elements and in any manner. Furthermore, any permutations and combinations of all described elements in this application should be considered disclosed by the description of the present application except for where this leads to logical contradictions or the context indicates otherwise.

Unless defined otherwise herein, scientific and technical terms used in connection with the present disclosure shall have the meanings that are commonly understood by those of ordinary skill in the art. Generally, nomenclatures and techniques referred to in the present disclosure, e.g. nomenclatures and techniques of organic chemistry, chemical synthesis, biology, medicinal and pharmaceutical chemistry, medicine, pharmacology or toxicology, are those well-known and commonly used in the art. The methods and techniques of the present disclosure are generally performed according to conventional methods well-known in the art and as described in the references cited and discussed throughout the present disclosure unless otherwise indicated.

First aspect of the present disclosure (also referred to as “Embodiment 1”): According to a first aspect, the present disclosure relates to a VHH antibody domain or a fragment thereof, wherein
(a) said VHH antibody domain or fragment thereof comprises the complementarity determining regions CDR1, CDR2 and CDR3 of one VHH selected from the group consisting of VHH1, VHH2, VHH3, VHH4, VHH5, VHH6, VHH8, VHH9, VHH10 and VHH11 as shown in the Table of CDRs;
(b) said VHH antibody domain or fragment thereof comprises the complementarity determining regions CDR1, CDR2 and CDR3 as defined in (a) with modification, wherein the modification is that the sequence of at least one of CDR1, CDR2 and CDR3 is humanized; or
(c) said VHH antibody domain or fragment thereof comprises the complementarity determining regions CDR1, CDR2 and CDR3 as defined in (a) with modification, wherein the modification is
   the replacement, addition or deletion of up to three amino acids in CDR1,
   the replacement, addition or deletion of up to three amino acids in CDR2 and/or
   the replacement, addition or deletion of up to three amino acids in CDR3;

TABLE of
CDRs:

| Description | SEQ ID NO | Amino acid sequence |
|---|---|---|
| CDR1 of VHH1 | SEQ ID NO: 23 | GFTFNSYA |
| CDR2 of VHH1 | SEQ ID NO: 24 | HIGSGGSTR |
| CDR3 of VHH1 | SEQ ID NO: 25 | NVYGATLRS |
| CDR1 of VHH2 | SEQ ID NO: 26 | GFTLLDSYD |
| CDR2 of VHH2 | SEQ ID NO: 27 | ISSSGGKSTY |
| CDR3 of VHH2 | SEQ ID NO: 28 | AADGAEGFWSCQSGGYDY |
| CDR1 of VHH3 | SEQ ID NO: 29 | GFTLGAYA |
| CDR2 of VHH3 | SEQ ID NO: 30 | ITSSGRSAY |
| CDR3 of VHH3 | SEQ ID NO: 31 | AAGAACAAAGTMNY |
| CDR1 of VHH4 | SEQ ID NO: 32 | GFTLGAYA |
| CDR2 of VHH4 | SEQ ID NO: 33 | ITSSGGSAY |
| CDR3 of VHH4 | SEQ ID NO: 34 | AAGGACAAAETMNY |
| CDR1 of VHH5 | SEQ ID NO: 35 | GRTFSNLA |
| CDR2 of VHH5 | SEQ ID NO: 36 | IDWNGDNRL |
| CDR3 of VHH5 | SEQ ID NO: 37 | AADSTYSTDWYLNPLNR |
| | | TRYDY |
| CDR1 of VHH6 | SEQ ID NO: 38 | KSIFSINA |
| CDR2 of VHH6 | SEQ ID NO: 39 | FTSSGRIN |
| CDR3 of VHH6 | SEQ ID NO: 40 | NVGLWEDGLWYDY |
| CDR1 of VHH7 | SEQ ID NO: 41 | GHTFNNLA |
| CDR2 of VHH7 | SEQ ID NO: 42 | IDWNGDNRL |
| CDR3 of VHH7 | SEQ ID NO: 43 | AADSTYSTNWYLDPLNR |
| | | TTYNY |
| CDR1 of VHH8 | SEQ ID NO: 44 | GRTFSNDA |
| CDR2 of VHH8 | SEQ ID NO: 45 | VSWSLGMTY |
| CDR3 of VHH8 | SEQ ID NO: 46 | AAGPDLSDELYGYDY |
| CDR1 of VHH9 | SEQ ID NO: 47 | RSDFSRYT |
| CDR2 of VHH9 | SEQ ID NO: 48 | RITSGGIPH |
| CDR3 of VHH9 | SEQ ID NO: 49 | NTVGVALWA |
| CDR1 of VHH10 | SEQ ID NO: 50 | GLPFSSYA |
| CDR2 of VHH10 | SEQ ID NO: 51 | ITWSGSSIY |
| CDR3 of VHH10 | SEQ ID NO: 52 | AAARRRVGLPLADPREY |
| | | VY |
| CDR1 of VHH11 | SEQ ID NO: 53 | GSPIRFSV |
| CDR2 of VHH11 | SEQ ID NO: 54 | RITSGGIVY |
| CDR3 of VHH11 | SEQ ID NO: 55 | NRLGGALFD |
| CDR1 of VHH12 | SEQ ID NO: 56 | GVGFSIYT |
| CDR2 of VHH12 | SEQ ID NO: 57 | VTTGGHIN |
| CDR3 of VHH12 | SEQ ID NO: 58 | NAIYFGQDI |
| CDR1 of VHH13 | SEQ ID NO: 59 | GISFRVNV |
| CDR2 of VHH13 | SEQ ID NO: 60 | ISSGDSTN |
| CDR3 of VHH13 | SEQ ID NO: 61 | NARIFARDN |
| CDR1 of VHH14 | SEQ ID NO: 62 | DRTVSKGA |
| CDR2 of VHH14 | SEQ ID NO: 63 | IGRSSGITY |
| CDR3 of VHH14 | SEQ ID NO: 64 | AADSSGIIRTTSWAYDY |
| CDR1 of VHH15 | SEQ ID NO: 65 | GRFFTINA |
| CDR2 of VHH15 | SEQ ID NO: 66 | ITSDGSTT |
| CDR3 of VHH15 | SEQ ID NO: 67 | NADGRFNDNGYVKNFNI |
| CDR1 of VHH16 | SEQ ID NO: 68 | GFTTDDYI |
| CDR2 of VHH16 | SEQ ID NO: 69 | ISTTDGSAW |
| CDR3 of VHH16 | SEQ ID NO: 70 | AEVSTGTGVLYCREEFY |
| | | FGMDS |
| CDR1 of VHH17 | SEQ ID NO: 71 | GFALDEIA |
| CDR2 of VHH17 | SEQ ID NO: 72 | ISSSDGSTL |
| CDR3 of VHH17 | SEQ ID NO: 73 | AAVRGSCFGRTTH |
| CDR1 of VHH18 | SEQ ID NO: 74 | GRTFSSYA |
| CDR2 of VHH18 | SEQ ID NO: 75 | ISWSGGSTY |
| CDR3 of VHH18 | SEQ ID NO: 76 | AADRSLSGSYFGATIDY |
| CDR1 of VHH19 | SEQ ID NO: 77 | GIRFNPER |
| CDR2 of VHH19 | SEQ ID NO: 78 | IDSGGSTD |
| CDR3 of VHH19 | SEQ ID NO: 79 | NTQIFARDY |
| CDR1 of VHH20 | SEQ ID NO: 80 | VSAFRLKV |
| CDR2 of VHH20 | SEQ ID NO: 81 | IDSGDTKS |
| CDR3 of VHH20 | SEQ ID NO: 82 | NAVLFGRDY |
| CDR1 of VHH21 | SEQ ID NO: 83 | GITFSPYV |
| CDR2 of VHH21 | SEQ ID NO: 84 | MVNGGSTN |
| CDR3 of VHH21 | SEQ ID NO: 85 | HIMIFGRAS |
| CDR1 of VHH22 | SEQ ID NO: 86 | GFALEYYA |
| CDR2 of VHH22 | SEQ ID NO: 87 | VSSSRSGGSTV |
| CDR3 of VHH22 | SEQ ID NO: 88 | AGAPAYRSHYCRLQADD |
| | | YTY |

An “antibody” is a polypeptide substantially encoded by an immunoglobulin gene or immunoglobulin genes, or antigen binding fragment thereof, which specifically binds and recognizes an analyte (antigen). Immunoglobulin genes include the kappa, lambda, alpha, gamma, delta, epsilon and mu constant region genes, as well as the myriad immunoglobulin variable region genes.

In primates such as humans, a heavy and the light chain variable domain of an antibody combine to specifically bind the antigen. Generally, a naturally occurring primate (e.g., human) or murine immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE. Primate antibodies can be class switched.

Certain IgG antibodies from members of the camel and dromedary (Camelus bactrianus and Calelus dromaderius) family including new world members such as llama species (Lama paccos, Lama glama and Lama vicugna) of mammals as found in nature lack light chains, and are thus structurally distinct from the typical four chain quaternary structure having two heavy and two light chains, for antibodies from other animals. See PCT/EP93/02214 (WO 94/04678 published 3 Mar. 1994). Such IgG subtypes of the llamas lack the light chains and the CH1 domain and are called heavy chain antibodies. These naturally occurring camelid antibodies consisting of only a heavy chain are functional and stable in the absence of light chain. The antigen-binding site of these heavy chain antibodies is formed only by a single domain, referred to as “VHH” (Könning et al., 2017) or, used synonymously herein, “VHH antibody domain”.

Each light and heavy chain of an antibody, contains constant domains and variable domains. References to “VH” or “VH” refer to the variable region of an immunoglobulin heavy chain, including that of an antibody fragment. References to “VL” or “VL” refer to the variable region of an immunoglobulin light chain, such as in a primate antibody. The variable domain of a heavy chain antibody is called VHH. The VHH is composed of only one polypeptide chain of 15 kDa and is considered the smallest known natural domain with full antigen-binding capacity. Light and heavy chain variable domains contain a “framework” region interrupted by three hypervariable regions, also called “complementarity determining regions” or “CDRs” (see, e.g., Kabat et al., Sequences of Proteins of Immunological Interest, U.S. Department of Health and Human Services, 1991). The sequences of the framework regions of different light or heavy chains are relatively conserved within a species. The framework region of an antibody, that is the combined framework regions of the constituent light and heavy chains, serves to position and align the CDRs in three-dimensional space. The CDRs are primarily responsible for antigen binding.

The CDRs are typically referred to as CDR1, CDR2, and CDR3 (from the N-terminus to C-terminus), and are also typically identified by the chain in which the particular CDR is located. Thus, a VH CDR3 is located in the variable domain of the heavy chain of the antibody in which it is found, whereas a VL CDR1 is the CDR1 from the variable domain of the light chain of the antibody in which it is found. Light chain CDRs are sometimes referred to as CDR L1, CDR L2, and CDR L3. Heavy chain CDRs are sometimes referred to as CDR H1, CDR H2, and CDR H3. VHH monoclonal antibodies have only a heavy chain, and thus include only one CDR1, CDR2 and CDR3. Generally, the CDR3 is primarily responsible for antigen specificity.

A VHH includes in an N- to C-direction, the following structural regions: N-FR1-CDR1-FR2-CDR2-FR3-CDR3-FR4-C, wherein FR denotes a framework region amino acid sequence and CDR denotes a complementary determining region amino acid sequence (see, e.g., Kabat et al., Sequences of Proteins of Immunological Interest, U.S. Department of Health and Human Services, 1991).

As used in the present disclosure, a “VHH antibody domain” refers to the domain formed by the complete VHH, including (in an N- to C-direction) N-FR1-CDR1-FR2-CDR2-FR3-CDR3-FR4-C. A VHH antibody domain can be part of a larger molecule to which the VHH antibody domain is covalently linked. Alternative, a VHH antibody domain can be a molecule of its own that is not covalently linked to any other molecular structure.

Since resulting paratopes are devoid of light chains, sdAbs afford the benefit of multiple reformatting options in a ‘plug-and-play’ manner involving beads-on-string assemblies or facile combinations with existing Fab-based paratopes for the generation of bispecifics (Lipinski et al., 2023; Chanier and Chames, 2019).

The extent of the framework region and CDRs have been defined (see, Kabat et al., Sequences of Proteins of Immunological Interest, U.S. Department of Health and Human Services, 1991). The CDRs of the heavy chain variable domain are located at residues 31-35 (CDR-H1), residues 50-65 (CDR-H2) and residues 95-102 (CDR-H3) according to the Kabat numbering system. In antibodies (such as primate antibodies) that include a light chain, such as a primate antibody, the CDRs of the light chain variable domain are located at residues 24-34 (CDR-L1), residues 50-56 (CDR-L2) and residues 89-97 (CDR-L3) according to the Kabat numbering system. The Kabat database is now maintained online. The location of camelid CDRs can also be determined (see, for example, Sircar et al., J. Immunol. 186: 6357-6367, 2011); a program to determine camelid antibody structure, the Rosetta Antibody program, is available on the internet.

A “monoclonal antibody” is an antibody produced by a single clone of B-lymphocytes or by a cell into which the heavy chain gene (and optionally a light chain gene, such as for a primate antibody) of a single antibody have been transfected. Monoclonal antibodies may be obtained using a variety of techniques known to those skilled in the art, including standard hybridoma technology (see e.g. Kohler and Milstein, Eur. J. Immunol. (1976), vol. 5, p. 511-519; Antibodies: A Laboratory Manual, 2nd edition (2014), editor Greenfield, Cold Spring Harbor Laboratory Press (USA); Immunobiology, 5th ed. (2001), editors Janeway et al., Garland Publishing (USA)) and e.g. expression from a eukaryotic host cell transfected with a DNA molecule coding for the homogeneous antibody or from a prokaryotic host cell transfected with a DNA molecule coding for the homogeneous antibody.

VHH antibody domains can be obtained by genetic engineering to yield a small protein having high affinity for a target, resulting in a low molecular weight antibody derived protein. See e.g. Sellmann et al., 2020; U.S. Pat. No. 5,759,808, issued Jun. 2, 1998; see also Dumoulin et al., (2003); Pleschberger et al., (2003); Cortez-Retamozo et al., (2002); and Lauwereys et al., (1998).

In some embodiments, the VHH molecules can be produced as recombinant monoclonal antibodies or antigen binding fragments in different expression platforms, avoiding the use of hybridomas and mice. VHH monoclonal antibodies can be humanized monoclonal antibodies. In some embodiments, monoclonal antibodies can be chimeric antibodies.

Without being bound by theory, a VHH monoclonal antibody has a molecular weight approximately one-tenth that of a human IgG molecule, and the protein has a physical diameter of only a few nanometers.

One consequence of the small size is the ability of the VHH monoclonal antibody to bind to antigenic sites that are functionally invisible to larger antibody proteins, such that VHH monoclonal antibodies are useful as reagents to detect antigens that are otherwise cryptic using classical immunological techniques, and thus are of use as therapeutic agents. Thus, yet another consequence of small size is that a camelid VHH monoclonal antibody can inhibit as a result of binding to a specific site in a groove or narrow cleft of a target protein, and hence can serve in a capacity that more closely resembles the function of a classical low molecular weight drug than that of a classical antibody.

Without being bound by theory, low molecular weight and compact size further result in camelid VHH monoclonal antibodies being extremely thermostable, stable to extreme pH and to proteolytic digestion, and poorly antigenic. Further, these molecules can be fully expressed in prokaryotic cells such as E. coli and are expressed as fusion proteins with bacteriophage and are functional.

“Humanizing” an antibody/antibody sequence, as used herein, refers to the process of “germlining” where a non-human (such as camelid, llama or synthetic) antibody sequence is adapted to be more similar to a human antibody sequence by replacing one or more individual amino acids with the corresponding amino acids of a human antibody sequence. Typically, as human antibody sequence will be selected that is particularly close (i.e. has a high degree of sequence homology) to the non-human sequence. Such a human antibody sequence can be identified e.g. by a BLAST search. The corresponding amino acids can then be identified by a pairwise sequence alignment between the selected human antibody sequence and the non-human antibody sequence to be humanized. After humanization, the humanized antibody still binds to the same antigen as the original non-human antibody before humanization. Humanized immunoglobulins can be constructed by means of genetic engineering. A VHH antibody domain is easily humanized based on the human VH domain, which has a sequence that is highly homologous to the sequence of the VHH antibody domain.

Furthermore, a VHH sequence can be adapted to reduce sequence liabilities. This may comprise, e.g., replacing amino acids susceptible for glycosylation, deamination, oxidization or isomerization by exchanging the respective amino acid(s) in the VHH sequence with the corresponding amino acid in the closest human germline sequence or with another amino acid (e.g. alanine). Similarly, the hydrophobic patches at the cell surface can be reduced by exchanging the respective amino acid(s) with less hydrophobic amino acid(s). Again, the amino acid replacements are chosen such that after the adaption the VHH sequence still binds to the same antigen and with similar characteristics as the VHH sequence before adaption.

VHHs can be used as modular building blocks for generating multivalent and/or multispecific antibody constructs, whereby “multivalent” means that the construct encompasses more than one single domain antibody and “multispecific” means that it encompasses single domain antibodies of more than one binding specificity.

At some occasions, the present disclosure states that a certain protein/amino acid sequence A is a “fragment” of another protein/amino acid sequence B. This means that, compared to protein/amino acid B, the protein/amino acid sequence A lacks one or more amino acids at the N-terminus and/or one or more amino acids at the C-terminus. Whether a protein/amino acid sequence lacks, compared to another protein/amino acid sequence, one or more amino acids at the N-terminus and/or one or more amino acids at the C-terminus can for example readily be determined upon forming a sequence alignment e.g. with the BLAST family of programs.

As the skilled person understands, when the present disclosure refers to an “VHH antibody domain or a fragment thereof”, said fragment is an antigen-binding fragment. Thus, said fragment binds to the same antigen as the “full-length” VHH antibody domain according to the present disclosure from which said fragment is derived (i.e. to IL-18Rα resp. IL-18Rβ). Preferably, said fragment of said VHH antibody domain is a C-terminal fragment. This means that compared to the “complete” VHH antibody domain sequence said fragment lacks amino acids at the N-terminus.

When the present disclosure indicates that a VHH antibody domain or fragment thereof “of one VHH selected from the group consisting of VHH1, VHH2 and VHH3 as shown in the Table of CDRs” (or a corresponding wording), this means that said VHH antibody domain comprises the combination of CDRs or either VHH1 or VHH2 or VHH3, but not a mixture of CDRs selected from different of the listed VHHs. Thus, said VHH antibody domain or fragment thereof includes e.g. the combination of CDR1, CDR2 and CDR3 of VHH1 (SEQ ID NO: 23, 24 and 25), the combination of CDR1, CDR2 and CDR3 of VHH2 (SEQ ID NO: 26, 27, and 28), or the combination of CDR1, CDR2 and CDR3 of VHH3 (SEQ ID NO: 29, 30, and 31) etc., but not the combination of CDR1 and CDR2 of VHH1 and CDR3 of VHH2 (SEQ ID NO: 23, 24 and 28).

When the present disclosure defines that a VHH antibody domain or fragment thereof comprises the complementarity determining regions CDR1, CDR2 and CDR3 as defined in e.g. (a) with modification, wherein the modification is e.g. that the sequence of at least one of CDR1, CDR2 and CDR3 is humanized, the skilled person is aware that this humanization exists compared to the corresponding sequence in the Table of CDRs that provides the combinations of CDR sequences without modification.

If the present disclosure indicates the existence of a modification which is “replacement, addition or deletion” of a certain number of amino acids (e.g. up to three), the skilled person understands that this is an individual replacement, addition or deletion of the indicated number of amino acids. Thus, the replaced, added or deleted amino acids may be at neighboring positions or at independent, isolated positions within the amino acid sequence. Moreover, as above, the skilled person is aware that this definition indicates the replacement, addition or deletion compared to the unmodified sequence in the Table of CDRs.

Embodiment 2: The VHH antibody domain or fragment thereof according to embodiment 1, wherein the modification in (b) is that the sequence of CDR1 and/or CDR2, but not the sequence of CDR3 is humanized.

Embodiment 3: The VHH antibody domain or fragment thereof according to any one of embodiments 1 or 2, wherein the modification in (b) is that the sequence of CDR1 is humanized, but not the sequence of CDR2 and CDR3.

Embodiment 4: The VHH antibody domain or fragment thereof according to any one of embodiments 1 or 2, wherein the modification in (b) is that the sequence of CDR2 is humanized, but not the sequence of CDR1 and CDR3.

Embodiment 5: The VHH antibody domain or fragment thereof according to any one of embodiments 1 to 4, wherein the modification in (b) is that the sequence of one, but not more than one of CDR1, CDR2 and CDR3 is humanized.

Embodiment 6: The VHH antibody domain or fragment thereof according to any one of embodiments 1 to 5, wherein said humanization of said CDR(s) is by replacing at least one amino acid in the sequence of said CDR by the corresponding amino acid of a human VH domain.

Embodiment 7: The VHH antibody domain or fragment thereof according to any one of embodiments 1 to 6, wherein said humanization of said CDR(s) is by replacing up to three amino acids in the sequence of said CDR by the corresponding amino acid of a human VH domain.

Embodiment 8: The VHH antibody domain or fragment thereof according to any one of embodiments 1 to 6, wherein said humanization of said CDR(s) is by replacing up to three amino acids in the sequence of CDR1 and/or CDR2 and up to one amino acid in the sequence of CDR3 by the corresponding amino acid of a human VH domain.

Embodiment 9: The VHH antibody domain or fragment thereof according to any one of embodiments 1 to 6, wherein said humanization of said CDR(s) is by replacing up to two amino acids in the sequence of said CDR by the corresponding amino acid of a human VH domain.

Embodiment 10: The VHH antibody domain or fragment thereof according to any one of embodiments 1 to 6, wherein said humanization of said CDR(s) is by replacing up to two amino acids in the sequence of CDR1 and/or CDR2 and up to one amino acid in the sequence of CDR3 by the corresponding amino acid of a human VH domain.

Embodiment 11: The VHH antibody domain or fragment thereof according to any one of embodiments 1 to 10, wherein said humanization of said CDR(s) is by replacing one amino acid in the sequence of said CDR by the corresponding amino acid of a human VH domain.

Embodiment 12: The VHH antibody domain or fragment thereof according to any one of embodiments 1 to 11, wherein the modification in (c) is
the replacement, addition or deletion of up to three amino acids in CDR1,
the replacement, addition or deletion of up to three amino acids in CDR2 and/or
the replacement, addition or deletion of up to one amino acid in CDR3.

Embodiment 13: The VHH antibody domain or fragment thereof according to any one of embodiments 1 to 11, wherein the modification in (c) is
the replacement, addition or deletion of up to two amino acids in CDR1,
the replacement, addition or deletion of up to two amino acids in CDR2 and/or
the replacement, addition or deletion of up to two amino acids in CDR3;

Embodiment 14: The VHH antibody domain or fragment thereof according to any one of embodiments 1 to 11, wherein the modification in (c) is
the replacement, addition or deletion of up to two amino acids in CDR1;
the replacement, addition or deletion of up to two amino acids in CDR2; and/or
the replacement, addition or deletion of up to one amino acid in CDR3.

Embodiment 15: The VHH antibody domain or fragment thereof according to any one of embodiments 1 to 11, wherein the modification in (c) is
the replacement, addition or deletion of up to two amino acids in CDR1 and/or
the replacement, addition or deletion of up to two amino acids in CDR2; wherein the sequence of CDR3 is unmodified.

As a skilled person understands, the indication that the “sequence of CDR3 is unmodified” means that the sequence is unmodified compared to the sequence provided for CDR3 for the VHH at issue in the Table of CDRs.

Embodiment 16: The VHH antibody domain or fragment thereof according to any one of embodiments 1 to 11, wherein the modification in (c) is
the replacement, addition or deletion of up to two amino acids in CDR1, wherein the sequence of CDR2 and CDR3 is unmodified.

Embodiment 17: The VHH antibody domain or fragment thereof according to any one of embodiments 1 to 11, wherein the modification in (c) is
the replacement, addition or deletion of up to two amino acids in CDR2, wherein the sequence of CDR1 and CDR3 is unmodified.

Embodiment 18: The VHH antibody domain or fragment thereof according to any one of embodiments 1 to 11, wherein the modification in (c) is
the replacement, addition or deletion of up to one amino acid in CDR1;
the replacement, addition or deletion of up to one amino acid in CDR2; and/or
the replacement, addition or deletion of up to one amino acid in CDR3.

Embodiment 19: The VHH antibody domain or fragment thereof according to any one of embodiments 1 to 11, wherein the modification in (c) is
the replacement, addition or deletion of one amino acid in CDR1 and/or
the replacement, addition or deletion of one amino acid in CDR2; wherein the sequence of CDR3 is unmodified.

Embodiment 20: The VHH antibody domain or fragment thereof according to any one of embodiments 1 to 11, wherein the modification in (c) is
the replacement, addition or deletion of one amino acid in CDR1, wherein the sequence of CDR2 and CDR3 is unmodified.

Embodiment 21: The VHH antibody domain or fragment thereof according to any one of embodiments 1 to 11, wherein the modification in (c) is
the replacement, addition or deletion of one amino acid in CDR2, wherein the sequence of CDR1 and CDR3 is unmodified.

Embodiment 22: The VHH antibody domain or fragment thereof according to any one of embodiments 1 to 21, wherein the modification in (c) comprises only the replacement, but not the addition or deletion of amino acids.

Embodiment 23: The VHH antibody domain or fragment thereof according to any one of embodiments 1 to 22, wherein said replacement is a conservative amino acid replacement.

As used herein, a “conservative amino acid replacement” refers to the replacement of an amino acid by another, biologically similar amino acid. Conservative replacements are not likely to change the shape or characteristics of a protein/amino acid sequence. Examples of conservative replacements include the replacement of one hydrophobic residue such as isoleucine, valine, leucine or methionine for another, or the substitution of one polar residue for another, such as the substitution of arginine for lysine, glutamic for aspartic acid, or glutamine for asparagine.

Second aspect of the present disclosure (also referred to as “Embodiment 24”): According to a second aspect, the present disclosure relates to a VHH antibody domain or a fragment thereof, wherein
(A) said VHH antibody domain comprises the amino acid sequence of a VHH selected from the group consisting of VHH1, VHH2, VHH3, VHH4, VHH5, VHH6, VHH8, VHH9, VHH10 and VHH11 as shown in the Table of VHH Sequences;
(B) said VHH antibody domain comprises a VHH sequence as defined in (A) with modification, wherein the modification is that said sequence is humanized;
(C) said VHH antibody domain comprises a VHH sequence as defined in (A) with modification, wherein the modification is the replacement, addition or deletion of up to 25 amino acids; or
(D) said VHH antibody domain comprises a VHH sequence that is at least 75% identical to a VHH sequence referred to in (A);

TABLE of
VHH Sequences:

| VHH | SEQ ID NO | Amino acid sequence |
|---|---|---|
| VHH1 | SEQ ID NO: 1 | DVPLQESGGGLVQTGGSLRLSCAASGFTFNSYAMGW |
| | | YQQAPGKKYRELVAHIGSGGSTRYIDSVKGRFTISR |
| | | DNAKNTVYLQMNSLKPEDTAVYYCNVYGATLRSWGQ |
| | | GTQVTVSS |
| VHH2 | SEQ ID NO: 2 | DVQLQESGGGLVQAGGSLRLSCAASGFTLLDSYDIG |
| | | WFRQAPGKKREEVSCISSSGGKSTYYADSVKGRFTI |
| | | SRDNANNTVYLQMNSLKPEDTAVYYCAADGAEGFWS |
| | | CQSGGYDYWGQGTQVTVSS |
| VHH3 | SEQ ID NO: 3 | DVQLQESGGGLVQAGGSLRLSCAASGFTLGAYAINW |
| | | FRQAPGKERARAWDACITSSGRSAYYADSVKGRFTV |
| | | SRDHAKNTVYLQMNSLKPEDTAFYYCAAGAACAAAG |
| | | TMNYWGKGTLVTVSS |
| VHH4 | SEQ ID NO: 4 | DVQLQESGGGLVQAGGSLRLSCAASGFTLGAYAINW |
| | | FQQAPGKERARAWVLCITSSGGSAYYVDSVKGRFTV |
| | | SRDNAKNTVYLQMNSLKPEDTAFYYCAAGGACAAAE |
| | | TMNYWGKGTLVTVSS |
| VHH5 | SEQ ID NO: 5 | DVQLQESGGGLVQAGGSLRLSCAASGRTFSNLAMAW |
| | | FRQAPGKEREFVAAIDWNGDNRLYAASVKGRFTISR |
| | | DNAKSTMYLQMNSLKPEDTAVYYCAADSTYSTDWYL |
| | | NPLNRTRYDYWGQGTQVTVSS |
| VHH6 | SEQ ID NO: 6 | DVPLQESGGGSVQPGGSLRLSCAASKSIFSINAMRW |
| | | HRQAPGKQRELVATFTSSGRINYADSVKGRFTISTD |
| | | NAKNTIYLQMNSLNPEDTAVYRCNVGLWEDGLWYDY |
| | | WGQGTQVTVSS |
| VHH7 | SEQ ID NO: 7 | DVQLQESGGGLVQAGGSLRLSCAASGHTFNNLAMAW |
| | | CRQAPGKEREFVSAIDWNGDNRLYAYSVKGRFTISR |
| | | DNAKSTMYLQMNSLKPEDTAVYYCAADSTYSTNWYL |
| | | DPLNRTTYNYWGQGTPVTVSS |
| VHH8 | SEQ ID NO: 8 | DVQLQESGGGLVQAGGSLRLSCAASGRTFSNDAMGW |
| | | FRQAPGKEREFVAAVSWSLGMTYNADSVKGRFTISR |
| | | DNAKNTVYLQMNSLKPDDTAVYYCAAGPDLSDELYG |
| | | YDYWGQGTQVTVSS |
| VHH9 | SEQ ID NO: 9 | DVQLQESGGGLVQPGESLRLSCTASRSDFSRYTMGW |
| | | YRQAPGKWRELVARITSGGIPHYADSVKGRFTISRD |
| | | NAKNTVTLQMNTLQFEDSAVYYCNTVGVALWAWGQG |
| | | TLVTVSS |
| VHH10 | SEQ ID NO: 10 | DVQLQESGGGLVQAGGSLRLSCAASGLPFSSYAMGW |
| | | FRQAPGKDREFVAGITWSGSSIYYRDSVKGRFTISR |
| | | DNAKNTVWLQMNDLKPEDTAVYYCAAARRRVGLPLA |
| | | DPREYVYWGQGTQVTVSS |
| VHH11 | SEQ ID NO: 11 | DVPLQESGGGLVQPGQSLRLSCAASGSPIRFSVMGW |
| | | FQQAPGKGRDLIARITSGGIVYYTDSVKGRFTISKD |
| | | NAKNTLYLQMNSLNLEDTGVYYCNRLGGALEDWGQG |
| | | TQVTVSS |
| VHH12 | SEQ ID NO: 12 | DVPLQESGGGLVQPGGSLRLSCRASGVGFSIYTMGW |
| | | YRQAPGEQRHLVATVTTGGHINYGDSVQGRFTISRE |
| | | NNGNVVELQMHSLKPEDTAVYFCNAIYFGQDIWGQG |
| | | TQVTVSS |
| VHH13 | SEQ ID NO: 13 | DVQLQESGGGLVQPGGSLRLSCAASGISFRVNVLGW |
| | | YRQAPGKPRELVASISSGDSTNYADSVKGRFTISRD |
| | | NAKSTVYLQMNNLKPEDTAVYYCNARIFARDNWGQG |
| | | TQVTVSS |
| VHH14 | SEQ ID NO: 14 | DVPLQESGGGLVQAGGSLRLSCAASDRTVSKGAMGW |
| | | FRQAPGKEREFVATIGRSSGITYYADSVKGRFTISR |
| | | DNAKNTWYLQMNSLRPEDTAVYYCAADSSGIIRTTS |
| | | WAYDYWGQGTQVTVSS |
| VHH15 | SEQ ID NO: 15 | DVQLQESGGGLVQPGGSLRLSCAASGRFFTINAMGW |
| | | YRQTPGKQRELVAAITSDGSTTYQDSVKGRFTISRD |
| | | NANRTANLYMNSLKSEDTAVYYCNADGRENDNGYVK |
| | | NFNIWGQGTQVTVSS |
| VHH16 | SEQ ID NO: 16 | DVQLQESGGGSVPAGGSLRLSCSASGFTTDDYIIGW |
| | | FRQAPGKKREGVSCISTTDGSAWYADSVKGRFTISS |
| | | DNANNTVTLQMNSLLPEDTSVYYCAEVSTGTGVLYC |
| | | REEFYFGMDSWGKGTPVTVSS |
| VHH17 | SEQ ID NO: 17 | DVPLQESGGGLVQAGGSLRLSCVASGFALDEIAVGW |
| | | FRQAPGKEREGLSCISSSDGSTLYADSVKGRFTISS |
| | | DNAKNTVYLQMNSLKPEDTAVYLCAAVRGSCFGRTT |
| | | HWGQGTQVTVSS |
| VHH18 | SEQ ID NO: 18 | DVQLQESGGGLVQAGGSLRLSCAASGRTFSSYAMGW |
| | | FRQAPGMVREFVAAISWSGGSTYYADSVQGRFTISR |
| | | DNAKNTVYLQMNSLKPEDTAVYYCAADRSLSGSYFG |
| | | ATIDYWGQGTQVTVSS |
| VHH19 | SEQ ID NO: 20 | DVQLQESGGGLVQAGGSLRLSCAASGIRFNPERMAW |
| | | YRQAPGKQRELVAAIDSGGSTDYADSVRGRFTISRD |
| | | NAKITVSLQMNSLKPEDTAVYYCNTQIFARDYWGQG |
| | | SQVTVSS |
| VHH20 | SEQ ID NO: 21 | DVQLQESGGGLVQAGGSLRLSCAASVSAFRLKVMGW |
| | | YRQAPGKEREFVAAIDSGDTKSYTNAVKGRFTISGG |
| | | AATNTVVLQMNSLKPEDTAFYYCNAVLFGRDYWGQG |
| | | TQVTVSS |
| VHH21 | SEQ ID NO: 22 | DVQLQESGGGLVQAGGSLRLSCAASGITFSPYVMGW |
| | | YRQAPGKQRELVADMVNGGSTNYADSVKGRFTIFRD |
| | | TAANTVTLQMNNLKPDDTAVYYCHIMIFGRASWGQG |
| | | TQVTVSS |
| VHH22 | SEQ ID NO: 22 | DVPLQESGGGLVQPGGSLRLSCAAPGFALEYYAIGW |
| | | FRQAPGKEREGLSCVSSSRSGGSTVYADSVKGRFTI |
| | | SRDNAKNTVYLQIDSLKVEDTAVYYCAGAPAYRSHY |
| | | CRLQADDYTYWGQGTQVTVSS |

Third aspect of the present disclosure (also referred to as “Embodiment 25”): According to a third aspect, the present disclosure relates to a VHH antibody domain or a fragment thereof, wherein
(A) said VHH antibody domain consists of the amino acid sequence of a VHH selected from the group consisting of VHH1, VHH2, VHH3, VHH4, VHH5, VHH6, VHH8, VHH9, VHH10 and VHH11 as shown in the Table of VHH Sequences;
(B) said VHH antibody domain consists of a VHH sequence as defined in (A) with modification, wherein the modification is that said sequence is humanized;
(C) said VHH antibody domain consists of a VHH sequence as defined in (A) with modification, wherein the modification is the replacement, addition or deletion of up to 25 amino acids; or
(D) said VHH antibody domain consists of a VHH sequence that is at least 75% identical to a VHH sequence referred to in (A).

To the second and third aspects of the present disclosure and the embodiments referring thereto, the same explanations and definitions apply accordingly as for the first aspect of the present disclosure and the embodiments referring thereto.

As the skilled person is aware, if the present disclosure indicates that a VHH antibody domain comprises e.g. “the VHH sequence of a VHH selected from the group consisting of VHH1, VHH2, VHH3, VHH4, VHH5, VHH6, VHH8, VHH9, VHH10 and VHH11 as shown in the Table of VHH Sequences”, this means that said VHH antibody domain comprises one and (not multiple or all) of the sequences listed in the Table of VHH Sequences.

Thus, if the present disclosure indicates that a VHH antibody domain or fragment thereof comprises/consists e.g. of a VHH sequence as defined in (A) “with modification, wherein the modification is that said sequence is humanized”, the skilled person is aware that this means that this humanization exists compared to the corresponding sequence in the Table of VHH Sequences that provides the sequences without modification.

If the present disclosure states that a certain sequence A “is at least x % identical” to another sequence B, this is synonymous to the statement that sequence A “has x % identity” to sequence B. The statement reflects a relationship between the two polypeptide sequences A and B determined by comparing the sequences. In general, identity refers to an exact amino acid to amino acid correspondence of the two polypeptide sequences, respectively, over the length of the sequences being compared. For sequences where there is not an exact correspondence, a percentage to which the two sequences are identical may be determined. In general, the two sequences to be compared are aligned to give a maximum correlation between the sequences. This may include inserting “gaps” in either one or both sequences, to enhance the degree of alignment. A % identity may be determined over the whole length of each of the sequences being compared (so-called global alignment), that is particularly suitable for sequences of the same or very similar length, or over shorter, defined lengths (so-called local alignment), that is more suitable for sequences of unequal length.

Methods for comparing the identity of two or more sequences are well known in the art. Thus, for instance, programs available in the Wisconsin Sequence Analysis Package, version 9.1 (Devereux J et al., 1984), for example the programs BESTFIT and GAP, may be used to determine the % identity between two polynucleotides and the % identity between two polypeptide sequences. BESTFIT uses the “local homology” algorithm of Smith and Waterman (1981) and finds the best single region of similarity between two sequences. Other programs for determining identity sequences are also known in the art, for instance the BLAST family of programs (Altschul S F et al, 1990, Altschul S F et al, 1997, accessible through the home page of the NCBI at www.ncbi.nlm.nih.gov) and FASTA (Pearson W R, 1990). Preferably, % identity according to the present disclosure is determined according to the BLAST family of programs (Altschul S F et al, 1990, Altschul S F et al, 1997, accessible through the home page of the NCBI at www.ncbi.nlm.nih.gov).

Embodiment 26: The VHH antibody domain or fragment thereof according to any one of embodiments 24 or 25, wherein said fragment of said VHH antibody domain comprises at least 75% of the amino acids of the sequence of said VHH antibody domain. As the skilled person understands, this means that that fragment lacks up to a quarter of the total number of amino acids of said VHH antibody domain, wherein, compared to the “complete” VHH antibody domain sequence said amino acids are lacking either at the N-terminus or at the C-terminus.

Embodiment 27: The VHH antibody domain or fragment thereof according to any one of embodiments 24 or 25, wherein said fragment of said VHH antibody domain comprises at least 80% of the amino acids of the sequence of said VHH antibody domain.

Embodiment 28: The VHH antibody domain or fragment thereof according to any one of embodiments 24 or 25, wherein said fragment of said VHH antibody domain comprises at least 85% of the amino acids of the sequence of said VHH antibody domain.

Embodiment 29: The VHH antibody domain or fragment thereof according to any one of embodiments 24 or 25, wherein said fragment of said VHH antibody domain comprises at least 90% of the amino acids of the sequence of said VHH antibody domain.

Embodiment 30: The VHH antibody domain or fragment thereof according to any one of embodiments 24 or 25, wherein said fragment of said VHH antibody domain comprises at least 95% of the amino acids of the sequence of said VHH antibody domain.

Embodiment 31: The VHH antibody domain or fragment thereof according to any one of embodiments 24 or 25, wherein said fragment of said VHH antibody domain comprises at least 98% of the amino acids of the sequence of said VHH antibody domain.

Embodiment 32: The VHH antibody domain or fragment thereof according to any one of embodiments 24 or 25, wherein said fragment of said VHH antibody domain comprises at least 99% of the amino acids of the sequence of said VHH antibody domain.

Embodiment 33: The VHH antibody domain or fragment thereof according to any one of embodiments 24 to 32, wherein said fragment of said VHH antibody domain comprises the complementarity determining regions CDR1, CDR2 and CDR3.

Embodiment 34: The VHH antibody domain or fragment thereof according to any one of embodiments 24 to 33, wherein said fragment of said VHH antibody domain comprises at least the sequence from the N-terminus of CDR1 to the C-terminus of CDR3 of said VHH antibody domain.

Embodiment 35: The VHH antibody domain or fragment thereof according to any one of embodiments 24 to 34, wherein in (A) said fragment of said VHH antibody domain comprises all the complementarity determining regions (CDRs) of said VHH antibody domain.

Embodiment 36: The VHH antibody domain or fragment thereof according to any one of embodiments 24 to 35, wherein in (B) said humanization of said sequence is by replacing at least one amino acid of said sequence by the corresponding amino acid of a human VH (variable heavy) domain.

Embodiment 37: The VHH antibody domain or fragment thereof according to any one of embodiments 24 to 36, wherein in (B) said humanization of said sequence is by (individually) replacing up to 25 amino acids of said sequence by the corresponding amino acids of a human VH domain.

As the skilled person understands, if the present disclosure refers to the replacement of an amino acid of a certain sequence/domain A by the “corresponding” amino acid of a certain sequence/domain B, this designates that said amino acid of sequence/domain A is replaced by the amino acid in sequence/domain B that in an alignment of the two sequences aligns with said amino acid of sequence/domain A.

Embodiment 38: The VHH antibody domain or fragment thereof according to any one of embodiments 24 to 36, wherein in (B) said humanization of said sequence is by replacing up to 20 amino acids of said sequence by the corresponding amino acids of a human VH domain.

Embodiment 39: The VHH antibody domain or fragment thereof according to any one of embodiments 24 to 36, wherein in (B) said humanization of said sequence is by replacing up to 15 amino acids of said sequence by the corresponding amino acids of a human VH domain.

Embodiment 40: The VHH antibody domain or fragment thereof according to any one of embodiments 24 to 36, wherein in (B) said humanization of said sequence is by replacing up to 10 amino acids of said sequence by the corresponding amino acids of a human VH domain.

Embodiment 41: The VHH antibody domain or fragment thereof according to any one of embodiments 24 to 36, wherein in (B) said humanization of said sequence is by replacing up to 5 amino acids of said sequence by the corresponding amino acids of a human VH domain.

Embodiment 42: The VHH antibody domain or fragment thereof according to any one of embodiments 24 to 36, wherein in (B) said humanization of said sequence is by replacing up to 3 amino acids of said sequence by the corresponding amino acids of a human VH domain.

Embodiment 43: The VHH antibody domain or fragment thereof according to any one of embodiments 24 to 36, wherein in (B) said humanization of said sequence is by replacing up to 2 amino acids of said sequence by the corresponding amino acids of a human VH domain.

Embodiment 44: The VHH antibody domain or fragment thereof according to any one of embodiments 24 to 36, wherein in (B) said humanization of said sequence is by replacing one amino acid of said sequence by the corresponding amino acid of a human VH domain.

Embodiment 45: The VHH antibody domain or fragment thereof according to any one of embodiments 24 to 44, wherein in (B) said humanization is within the framework regions of said VHH antibody domain and/or within the CDRs of said VHH antibody domain.

Embodiment 46: The VHH antibody domain or fragment thereof according to any one of embodiments 24 to 44, wherein in (B) said humanization is within the framework regions of said VHH antibody domain, but not within the CDRs of said VHH antibody domain.

Embodiment 47: The VHH antibody domain or fragment thereof according to any one of embodiments 24 to 36, wherein in (B) said humanization is within the CDRs of said VHH antibody domain, but not within the framework regions of said VHH antibody domain.

Embodiment 48: The VHH antibody domain or fragment thereof according to any one of embodiments 24 to 45 or 47, wherein in (B) said humanization within the CDRs of said VHH antibody domain is within CDR1, CDR2 and/or CDR3.

Embodiment 49: The VHH antibody domain or fragment thereof according to any one of embodiments 24 to 45 or 47 to 48, wherein in (B) said humanization within the CDRs of said VHH antibody domain is within CDR1 and/or CDR2.

Embodiment 50: The VHH antibody domain or fragment thereof according to any one of embodiments 24 to 45 or 47 to 49, wherein in (B) said humanization within the CDRs of said VHH antibody domain is within CDR1.

Embodiment 51: The VHH antibody domain or fragment thereof according to any one of embodiments 24 to 45 or 47 to 50, wherein in (B) said humanization within the CDRs of said VHH antibody domain is within CDR2.

Embodiment 52: The VHH antibody domain or fragment thereof according to any one of embodiments 24 to 51, wherein in (B) said humanization within the CDRs of said VHH antibody domain is not within CDR3.

Embodiment 53: The VHH antibody domain or fragment thereof according to any one of embodiments 24 to 52, wherein in (C) the modification is the replacement, addition or deletion of up to 20 amino acids.

Embodiment 54: The VHH antibody domain or fragment thereof according to any one of embodiments 24 to 52, wherein in (C) the modification is the replacement, addition or deletion of up to 15 amino acids.

Embodiment 55: The VHH antibody domain or fragment thereof according to any one of embodiments 24 to 52, wherein in (C) the modification is the replacement, addition or deletion of up to 10 amino acids.

Embodiment 56: The VHH antibody domain or fragment thereof according to any one of embodiments 24 to 52, wherein in (C) the modification is the replacement, addition or deletion of up to 5 amino acids.

Embodiment 57: The VHH antibody domain or fragment thereof according to any one of embodiments 24 to 52, wherein in (C) the modification is the replacement, addition or deletion of up to 3 amino acids.

Embodiment 58: The VHH antibody domain or fragment thereof according to any one of embodiments 24 to 52, wherein in (C) the modification is the replacement, addition or deletion of up to 2 amino acids.

Embodiment 59: The VHH antibody domain or fragment thereof according to any one of embodiments 24 to 52, wherein in (C) the modification is the replacement, addition or deletion of one amino acid.

Embodiment 60: The VHH antibody domain or fragment thereof according to any one of embodiments 24 to 59, wherein the modification in (C) comprises only the replacement, but not the addition or deletion of amino acids.

Embodiment 61: The VHH antibody domain or fragment thereof according to any one of embodiments 24 to 60, wherein in (A) said VHH antibody domain or fragment thereof comprises the complementarity determining regions CDR1, CDR2 and CDR3 of one VHH selected from the group consisting of VHH1, VHH2, VHH3, VHH4, VHH5, VHH6, VHH8, VHH9, VHH10 and VHH11 as shown in the Table of CDRs.

Embodiment 62: The VHH antibody domain or fragment thereof according to any one of embodiments 24 to 61, wherein in (B) to (D)
(a) said VHH antibody domain or fragment thereof comprises the complementarity determining regions CDR1, CDR2 and CDR3 of one VHH selected from the group consisting of VHH1, VHH2, VHH3, VHH4, VHH5, VHH6, VHH8, VHH9, VHH10 and VHH11 as shown in the Table of CDRs;
(b) said VHH antibody domain or fragment thereof comprises the complementarity determining regions CDR1, CDR2 and CDR3 as defined in (a) with modification, wherein the modification is that the sequence of at least one of CDR1, CDR2 and CDR3 is humanized;
(c) said VHH antibody domain or fragment thereof comprises the complementarity determining regions CDR1, CDR2 and CDR3 as defined in (a) with modification, wherein the modification is
   the replacement, addition or deletion of up to three amino acids in CDR1,
   the replacement, addition or deletion of up to three amino acids in CDR2 and/or
   the replacement, addition or deletion of up to three amino acids in CDR3.

Embodiment 63: The VHH antibody domain or fragment thereof according to embodiment 62, wherein the modification in (b) is that the sequence of CDR1 and/or CDR2, but not the sequence of CDR3 is humanized.

Embodiment 64: The VHH antibody domain or fragment thereof according to any one of embodiments 62 or 63, wherein the modification in (b) is that the sequence of CDR1 is humanized, but not the sequence of CDR2 and CDR3.

Embodiment 65: The VHH antibody domain or fragment thereof according to any one of embodiments 62 or 63, wherein the modification in (b) is that the sequence of CDR2 is humanized, but not the sequence of CDR1 and CDR3.

Embodiment 66: The VHH antibody domain or fragment thereof according to any one of embodiments 62 to 65, wherein the modification in (b) is that the sequence of one, but not more than one of CDR1, CDR2 and CDR3 is humanized.

Embodiment 67: The VHH antibody domain or fragment thereof according to any one of embodiments 62 to 66, wherein said humanization of said CDR(s) is by replacing at least one amino acid in the sequence of said CDR by the corresponding amino acid of a human VH domain.

Embodiment 68: The VHH antibody domain or fragment thereof according to any one of embodiments 62 to 67, wherein said humanization of said CDR(s) is by replacing up to three amino acids in the sequence of said CDR by the corresponding amino acid of a human VH domain.

Embodiment 69: The VHH antibody domain or fragment thereof according to any one of embodiments 62 to 67, wherein said humanization of said CDR(s) is by replacing up to three amino acids in the sequence of CDR1 and/or CDR2 and up to one amino acid in the sequence of CDR3 by the corresponding amino acid of a human VH domain.

Embodiment 70: The VHH antibody domain or fragment thereof according to any one of embodiments 62 to 67, wherein said humanization of said CDR(s) is by replacing up to two amino acids in the sequence of said CDR by the corresponding amino acid of a human VH domain.

Embodiment 71: The VHH antibody domain or fragment thereof according to any one of embodiments 62 to 67, wherein said humanization of said CDR(s) is by replacing up to two amino acids in the sequence of CDR1 and/or CDR2 and up to one amino acid in the sequence of CDR3 by the corresponding amino acid of a human VH domain.

Embodiment 72: The VHH antibody domain or fragment thereof according to any one of embodiments 62 to 71, wherein said humanization of said CDR(s) is by replacing one amino acid in the sequence of said CDR by the corresponding amino acid of a human VH domain.

Embodiment 73: The VHH antibody domain or fragment thereof according to any one of embodiments 62 to 72, wherein the modification in (c) is
the replacement, addition or deletion of up to three amino acids in CDR1,
the replacement, addition or deletion of up to three amino acids in CDR2 and/or
the replacement, addition or deletion of up to two amino acids in CDR3.

Embodiment 74: The VHH antibody domain or fragment thereof according to any one of embodiments 62 to 72, wherein the modification in (c) is
the replacement, addition or deletion of up to three amino acids in CDR1,
the replacement, addition or deletion of up to three amino acids in CDR2 and/or
the replacement, addition or deletion of up to one amino acid in CDR3.

Embodiment 75: The VHH antibody domain or fragment thereof according to any one of embodiments 62 to 72, wherein the modification in (c) is
the replacement, addition or deletion of up to three amino acids in CDR1, and/or
the replacement, addition or deletion of up to three amino acids in CDR2, wherein the sequence of CDR3 is unmodified.

Embodiment 76: The VHH antibody domain or fragment thereof according to any one of embodiments 62 to 72, wherein the modification in (c) is
the replacement, addition or deletion of up to two amino acids in CDR1,
the replacement, addition or deletion of up to two amino acids in CDR2 and/or
the replacement, addition or deletion of up to two amino acids in CDR3;

Embodiment 77: The VHH antibody domain or fragment thereof according to any one of embodiments 62 to 72, wherein the modification in (c) is
the replacement, addition or deletion of up to two amino acids in CDR1;
the replacement, addition or deletion of up to two amino acids in CDR2; and/or
the replacement, addition or deletion of up to one amino acid in CDR3.

Embodiment 78: The VHH antibody domain or fragment thereof according to any one of embodiments 62 to 72, wherein the modification in (c) is
the replacement, addition or deletion of up to two amino acids in CDR1 and/or
the replacement, addition or deletion of up to two amino acids in CDR2; wherein the sequence of CDR3 is unmodified.

As the skilled person is aware, “unmodified” means unmodified compared to the sequence in the Table of CDRs.

Embodiment 79: The VHH antibody domain or fragment thereof according to any one of embodiments 62 to 72, wherein the modification in (c) is
the replacement, addition or deletion of up to two amino acids in CDR1, wherein the sequence of CDR2 and CDR3 is unmodified.

Embodiment 80: The VHH antibody domain or fragment thereof according to any one of embodiments 62 to 72, wherein the modification in (c) is
the replacement, addition or deletion of up to two amino acids in CDR2, wherein the sequence of CDR1 and CDR3 is unmodified.

Embodiment 81: The VHH antibody domain or fragment thereof according to any one of embodiments 62 to 72, wherein the modification in (c) is
the replacement, addition or deletion of up to one amino acid in CDR1;
the replacement, addition or deletion of up to one amino acid in CDR2; and/or
the replacement, addition or deletion of up to one amino acid in CDR3.

Embodiment 82: The VHH antibody domain or fragment thereof according to any one of embodiments 62 to 72, wherein the modification in (c) is
the replacement, addition or deletion of one amino acid in CDR1 and/or
the replacement, addition or deletion of one amino acid in CDR2; wherein the sequence of CDR3 is unmodified.

Embodiment 83: The VHH antibody domain or fragment thereof according to any one of embodiments 62 to 72, wherein the modification in (c) is
the replacement, addition or deletion of one amino acid in CDR1, wherein the sequence of CDR2 and CDR3 is unmodified.

Embodiment 84: The VHH antibody domain or fragment thereof according to any one of embodiments 62 to 72, wherein the modification in (c) is
the replacement, addition or deletion of one amino acid in CDR2, wherein the sequence of CDR1 and CDR3 is unmodified.

Embodiment 85: The VHH antibody domain or fragment thereof according to any one of embodiments 62 to 84, wherein the modification in (c) comprises only the replacement, but not the addition or deletion of amino acids.

Embodiment 86: The VHH antibody domain or fragment thereof according to any one of embodiments 62 to 85, wherein said replacement is a conservative amino acid replacement.

Embodiment 87: The VHH antibody domain or fragment thereof according to any one of embodiments 1 to 86, wherein said fragment consists of at least 100 amino acids.

Embodiment 88: The VHH antibody domain or fragment thereof according to any one of embodiments 1 to 86, wherein said fragment consists of at least 105 amino acids.

Embodiment 89: The VHH antibody domain or fragment thereof according to any one of embodiments 1 to 86, wherein said fragment consists of at least 110 amino acids.

Embodiment 90: The VHH antibody domain or fragment thereof according to any one of embodiments 1 to 86, wherein said fragment consists of at least 115 amino acids.

Embodiment 91: The VHH antibody domain or fragment thereof according to any one of embodiments 1 to 90, wherein said VHH antibody domain is an anti-IL-18Rα VHH antibody domain.

Embodiment 92: The VHH antibody domain or fragment thereof according to any one of embodiments 1 to 91, wherein said VHH antibody domain is specific for IL-18Rα.

Embodiment 93: The VHH antibody domain or fragment thereof according to any one of embodiments 1 to 92, wherein said VHH antibody domain or fragment thereof binds to IL-18Rα.

Embodiment 94: The VHH antibody domain or fragment thereof according to any one of embodiments 1 to 93, wherein said VHH antibody domain or fragment thereof binds specifically to IL-18Rα.

Embodiment 95: The VHH antibody domain or fragment thereof according to any one of embodiments 1 to 94, wherein said VHH antibody domain or fragment thereof does not bind to IL-18Rβ.

Embodiment 96: The VHH antibody domain or fragment thereof according to any one of embodiments 1 to 95, wherein said VHH antibody domain or fragment thereof is capable of specifically binding to IL-18Rα ECD.

In some embodiments, if the present disclosure states that a first molecule/molecular group (e.g. an antibody/antibody component) “is capable of specifically binding”/“specifically binds” to a second molecule/molecular group (e.g. an antigen of interest), this means that the first molecule/molecular group (in this example the antibody) binds to said second molecule/molecular group (in this example the antigen of interest) with an affinity that is at least ten-fold greater than its affinity for other molecules/molecular groups, in particular other molecule/molecular group in the human body (in this example at least ten-fold greater than its affinity for binding to non-specific antigens (e.g., BSA, casein) other than said antigen of interest (or closely related antigens)). In a preferred embodiment, a first molecule/molecular group (e.g. an antibody/antibody component) that “specifically binds” to a second molecule/molecular group (e.g. an antigen of interest) binds to said antigen with an affinity that is at least 100-fold greater than its affinity for other molecules/molecular groups, in particular other molecule/molecular group in the human body (in this example at least 100-fold greater than its affinity for binding to non-specific antigens other than said antigen of interest (or closely related antigens)). Typically said binding will be determined under physiological conditions. A first molecule/molecular group that “specifically binds” to a second molecule/molecular group may bind to that second molecule/molecular group with a KD of 1×10⁻⁷ M or stronger.

Embodiment 97: The VHH antibody domain or fragment thereof according to any one of embodiments 1 to 96, wherein said VHH antibody domain or fragment thereof binds to recombinant human IL-18Rα ECD with a KD of 1×10⁻⁶ M or stronger.

Embodiment 98: The VHH antibody domain or fragment thereof according to any one of embodiments 1 to 97, wherein said VHH antibody domain or fragment thereof binds to recombinant human IL-18Rα ECD with a KD of 1×10⁻⁷ M or stronger.

Embodiment 99: The VHH antibody domain or fragment thereof according to any one of embodiments 1 to 97, wherein said VHH antibody domain or fragment thereof binds to recombinant human IL-18Rα ECD with a KD of 5×10⁻⁸ M or stronger.

Embodiment 100: The VHH antibody domain or fragment thereof according to any one of embodiments 1 to 97, wherein said VHH antibody domain or fragment thereof binds to recombinant human IL-18Rα ECD with a KD of 1.5×10⁻⁸ M or stronger.

Embodiment 101: The VHH antibody domain or fragment thereof according to any one of embodiments 1 to 96, wherein said VHH antibody domain or fragment thereof binds to recombinant human IL-18Rα ECD with a KD of 1×10⁻⁸ M or stronger.

Embodiment 102: The VHH antibody domain or fragment thereof according to any one of embodiments 1 to 96, wherein said VHH antibody domain or fragment thereof binds to recombinant human IL-18Rα ECD with a KD of 5×10⁻⁹ M or stronger.

Embodiment 103: The VHH antibody domain or fragment thereof according to any one of embodiments 1 to 96, wherein said VHH antibody domain or fragment thereof binds to recombinant human IL-18Rα ECD with a KD of 1×10⁻⁹ M or stronger.

Embodiment 104: The VHH antibody domain or fragment thereof according to any one of embodiments 1 to 103, wherein said fragment of said VHH antibody domain binds to recombinant human IL-18Rα ECD with at least 90% of the affinity (as determined by KD value) with which said VHH antibody domain binds to recombinant human IL-18Rα ECD.

Embodiment 105: The VHH antibody domain or fragment thereof according to any one of embodiments 1 to 104, wherein said fragment of said VHH antibody domain binds to IL-18Rα ECD with an affinity (as determined from the KD value) that is at least equal to the affinity with which VHH2 (SEQ ID NO: 2) binds to IL-18Rα ECD or stronger.

Such binding can be determined by in vitro binding experiments as described in Example 1 below (by biolayer interferometry).

Embodiment 106: The VHH antibody domain or fragment thereof according to any one of embodiments 1 to 23 or 62 to 105, wherein in (b) and (c) the VHH antibody domain or fragment thereof binds to recombinant human IL-18Rα ECD with an affinity (KD value) that is by not more than a factor of 10 weaker than the binding of the corresponding VHH antibody domain without modification.

Embodiment 107: The VHH antibody domain or fragment thereof according to any one of embodiments 1 to 23 or 62 to 105, wherein in (b) and (c) the VHH antibody domain or fragment thereof binds to recombinant human IL-18Rα ECD with an affinity (KD value) that is by not more than a factor of 5 weaker than the binding of the corresponding VHH antibody domain without modification.

Embodiment 108: The VHH antibody domain or fragment thereof according to any one of embodiments 1 to 23 or 62 to 105, wherein in (b) and (c) the VHH antibody domain or fragment thereof binds to recombinant human IL-18Rα ECD with an affinity (KD value) that is by not more than a factor of 2 weaker than the binding of the corresponding VHH antibody domain without modification.

Embodiment 109: The VHH antibody domain or fragment thereof according to any one of embodiments 1 to 23 or 62 to 105, wherein in (b) and (c) the VHH antibody domain or fragment thereof binds to recombinant human IL-18Rα ECD with an affinity (KD value) that is by not more than a factor of 1.5 weaker than the binding of the corresponding VHH antibody domain without modification.

Embodiment 110: The VHH antibody domain or fragment thereof according to any one of embodiments 1 to 23 or 62 to 109, wherein in (b) and (c) the VHH antibody domain or fragment thereof binds to recombinant human IL-18Rα ECD with an affinity (KD value) that is by not more than a factor of 10 stronger than the binding of the corresponding VHH antibody domain without modification.

Embodiment 111: The VHH antibody domain or fragment thereof according to any one of embodiments 1 to 23 or 62 to 109, wherein in (b) and (c) the VHH antibody domain or fragment thereof binds to recombinant human IL-18Rα ECD with an affinity (KD value) that is by not more than a factor of 5 stronger than the binding of the corresponding VHH antibody domain without modification.

Embodiment 112: The VHH antibody domain or fragment thereof according to any one of embodiments 1 to 23 or 62 to 109, wherein in (b) and (c) the VHH antibody domain or fragment thereof binds to recombinant human IL-18Rα ECD with an affinity (KD value) that is by not more than a factor of 2 stronger than the binding of the corresponding VHH antibody domain without modification.

Embodiment 113: The VHH antibody domain or fragment thereof according to any one of embodiments 1 to 23 or 62 to 109, wherein in (b) and (c) the VHH antibody domain or fragment thereof binds to recombinant human IL-18Rα ECD with an affinity (KD value) that is by not more than a factor of 1.5 stronger than the binding of the corresponding VHH antibody domain without modification.

Embodiment 114: The VHH antibody domain or fragment thereof according to any one of embodiments 24 to 113, wherein in (B) and (C) the VHH antibody domain binds to recombinant human IL-18Rα ECD with an affinity (KD value) that is by not more than a factor of 10 weaker than the binding of the corresponding VHH antibody domain without modification.

Embodiment 115: The VHH antibody domain or fragment thereof according to any one of embodiments 24 to 113, wherein in (B) and (C) the VHH antibody domain binds to recombinant human IL-18Rα ECD with an affinity (KD value) that is by not more than a factor of 5 weaker than the binding of the corresponding VHH antibody domain without modification.

Embodiment 116: The VHH antibody domain or fragment thereof according to any one of embodiments 24 to 113, wherein in (B) and (C) the VHH antibody domain binds to recombinant human IL-18Rα ECD with an affinity (KD value) that is by not more than a factor of 2 weaker than the binding of the corresponding VHH antibody domain without modification.

Embodiment 117: The VHH antibody domain or fragment thereof according to any one of embodiments 24 to 113, wherein in (B) and (C) the VHH antibody domain binds to recombinant human IL-18Rα ECD with an affinity (KD value) that is by not more than a factor of 1.5 weaker than the binding of the corresponding VHH antibody domain without modification.

Embodiment 118: The VHH antibody domain or fragment thereof according to any one of embodiments 24 to 117, wherein in (B) and (C) the VHH antibody domain binds to recombinant human IL-18Rα ECD with an affinity (KD value) that is by not more than a factor of 10 stronger than the binding of the corresponding VHH antibody domain without modification.

Embodiment 119: The VHH antibody domain or fragment thereof according to any one of embodiments 24 to 117, wherein in (B) and (C) the VHH antibody domain binds to recombinant human IL-18Rα ECD with an affinity (KD value) that is by not more than a factor of 5 stronger than the binding of the corresponding VHH antibody domain without modification.

Embodiment 120: The VHH antibody domain or fragment thereof according to any one of embodiments 24 to 117, wherein in (B) and (C) the VHH antibody domain binds to recombinant human IL-18Rα ECD with an affinity (KD value) that is by not more than a factor of 2 stronger than the binding of the corresponding VHH antibody domain without modification.

Embodiment 121: The VHH antibody domain or fragment thereof according to any one of embodiments 24 to 117, wherein in (B) and (C) the VHH antibody domain binds to recombinant human IL-18Rα ECD with an affinity (KD value) that is by not more than a factor of 1.5 stronger than the binding of the corresponding VHH antibody domain without modification.

Embodiment 122: The VHH antibody domain or fragment thereof according to any one of embodiments 24 to 121, wherein in (D) the affinity (KD value) of the binding of the VHH antibody domain to recombinant human IL-18Rα ECD is by not more than a factor of 10 weaker than the affinity (KD value) of the binding to recombinant human IL-18Rα ECD of a VHH antibody domain consisting of the sequence from the Table of VHH Sequences that has the highest degree of sequence identity with the sequence of said VHH antibody domain of (D). The degree of sequence identity can be determined by sequence alignment.

Embodiment 123: The VHH antibody domain or fragment thereof according to any one of embodiments 24 to 121, wherein in (D) the affinity (KD value) of the binding of the VHH antibody domain to recombinant human IL-18Rα ECD is by not more than a factor of 5 weaker than the affinity (KD value) of the binding to recombinant human IL-18Rα ECD of a VHH antibody domain consisting of the sequence from the Table of VHH Sequences that has the highest degree of sequence identity with the sequence of said VHH antibody domain of (D). The degree of sequence identity can be determined by sequence alignment.

Embodiment 124: The VHH antibody domain or fragment thereof according to any one of embodiments 24 to 121, wherein in (D) the affinity (KD value) of the binding of the VHH antibody domain to recombinant human IL-18Rα ECD is by not more than a factor of 2 weaker than the affinity (KD value) of the binding to recombinant human IL-18Rα ECD of a VHH antibody domain consisting of the sequence from the Table of VHH Sequences that has the highest degree of sequence identity with the sequence of said VHH antibody domain of (D).

Embodiment 125: The VHH antibody domain or fragment thereof according to any one of embodiments 24 to 121, wherein in (D) the affinity (KD value) of the binding of the VHH antibody domain to recombinant human IL-18Rα ECD is by not more than a factor of 1.5 weaker than the affinity (KD value) of the binding to recombinant human IL-18Rα ECD of a VHH antibody domain consisting of the sequence from the Table of VHH Sequences that has the highest degree of sequence identity with the sequence of said VHH antibody domain of (D).

Embodiment 126: The VHH antibody domain or fragment thereof according to any one of embodiments 24 to 125, wherein in (D) the affinity (KD value) of the binding of the VHH antibody domain to recombinant human IL-18Rα ECD is by not more than a factor of 10 stronger than the affinity (KD value) of the binding to recombinant human IL-18Rα ECD of a VHH antibody domain consisting of the sequence from the Table of VHH Sequences that has the highest degree of sequence identity with the sequence of said VHH antibody domain of (D).

Embodiment 127: The VHH antibody domain or fragment thereof according to any one of embodiments 24 to 125, wherein in (D) the affinity (KD value) of the binding of the VHH antibody domain to recombinant human IL-18Rα ECD is by not more than a factor of 5 stronger than the affinity (KD value) of the binding to recombinant human IL-18Rα ECD of a VHH antibody domain consisting of the sequence from the Table of VHH Sequences that has the highest degree of sequence identity with the sequence of said VHH antibody domain of (D).

Embodiment 128: The VHH antibody domain or fragment thereof according to any one of embodiments 24 to 125, wherein in (D) the affinity (KD value) of the binding of the VHH antibody domain to recombinant human IL-18Rα ECD is by not more than a factor of 2 stronger than the affinity (KD value) of the binding to recombinant human IL-18Rα ECD of a VHH antibody domain consisting of the sequence from the Table of VHH Sequences that has the highest degree of sequence identity with the sequence of said VHH antibody domain of (D).

Embodiment 129: The VHH antibody domain or fragment thereof according to any one of embodiments 24 to 125, wherein in (D) the affinity (KD value) of the binding of the VHH antibody domain to recombinant human IL-18Rα ECD is by not more than a factor of 1.5 stronger than the affinity (KD value) of the binding to recombinant human IL-18Rα ECD of a VHH antibody domain consisting of the sequence from the Table of VHH Sequences that has the highest degree of sequence identity with the sequence of said VHH antibody domain of (D).

Embodiment 130: The VHH antibody domain or fragment thereof according to any one of embodiments 91 to 129, wherein said KD value is determined in in vitro binding experiments through biolayer interferometry.

Embodiment 131: The VHH antibody domain or fragment thereof according to any one of embodiments 91 to 130, wherein said KD value is measured by kinetic measurements by biolayer interferometry at 25 °C and 1000 rpm in KB Buffer (PBS+0.1% Tween-20+1% BSA).

Embodiment 132: The VHH antibody domain or fragment thereof according to any one of embodiments 24 to 131, wherein said VHH antibody domain of (A) comprises one VHH sequence selected from the group consisting of VHH1, VHH2, VHH3, VHH4, VHH5, VHH6, VHH8, VHH9 and VHH10 shown in the Table of VHH Sequences.

Embodiment 133: The VHH antibody domain or fragment thereof according to any one of embodiments 24 to 131, wherein said VHH antibody domain of (A) comprises one VHH sequence selected from the group consisting of VHH1, VHH2, VHH5, VHH6, VHH8, VHH9 and VHH10 shown in the Table of VHH Sequences.

Embodiment 134: The VHH antibody domain or fragment thereof according to any one of embodiments 24 to 131, wherein said VHH antibody domain of (A) comprises one VHH sequence selected from the group consisting of VHH2, VHH6, VHH8 and VHH10 shown in the Table of VHH Sequences.

Embodiment 135: The VHH antibody domain or fragment thereof according to any one of embodiments 24 to 131, wherein said VHH antibody domain of (A) comprises one VHH sequence selected from the group consisting of VHH6 and VHH11 shown in the Table of VHH Sequences.

Embodiment 136: The VHH antibody domain or fragment thereof according to any one of embodiments 24 to 131, wherein said VHH antibody domain of (A) comprises one VHH sequence selected from the group consisting of VHH1, VHH2, VHH5, VHH6, VHH8 and VHH10 shown in the Table of VHH Sequences.

Embodiment 137: The VHH antibody domain or fragment thereof according to any one of embodiments 24 to 131, wherein said VHH antibody domain of (A) comprises one VHH sequence selected from the group consisting of VHH2, VHH6, VHH8 and VHH10 shown in the Table of VHH Sequences.

Embodiment 138: The VHH antibody domain or fragment thereof according to any one of embodiments 24 to 131, wherein said VHH antibody domain of (A) comprises one VHH sequence selected from the group consisting of VHH2 and VHH8 shown in the Table of VHH Sequences.

Embodiment 139: The VHH antibody domain or fragment thereof according to any one of embodiments 24 to 131, wherein said VHH antibody domain of (A) comprises the VHH sequence VHH1 shown in the Table of VHH Sequences.

Embodiment 140: The VHH antibody domain or fragment thereof according to any one of embodiments 24 to 131, wherein said VHH antibody domain of (A) comprises the VHH sequence VHH2 shown in the Table of VHH Sequences.

Embodiment 141: The VHH antibody domain or fragment thereof according to any one of embodiments 24 to 131, wherein said VHH antibody domain of (A) comprises the VHH sequence VHH3 shown in the Table of VHH Sequences.

Embodiment 142: The VHH antibody domain or fragment thereof according to any one of embodiments 24 to 131, wherein said VHH antibody domain of (A) comprises the VHH sequence VHH4 shown in the Table of VHH Sequences.

Embodiment 143: The VHH antibody domain or fragment thereof according to any one of embodiments 24 to 131, wherein said VHH antibody domain of (A) comprises the VHH sequence VHH5 shown in the Table of VHH Sequences.

Embodiment 144: The VHH antibody domain or fragment thereof according to any one of embodiments 24 to 131, wherein said VHH antibody domain of (A) comprises the VHH sequence VHH6 shown in the Table of VHH Sequences.

Embodiment 145: The VHH antibody domain or fragment thereof according to any one of embodiments 24 to 131, wherein said VHH antibody domain of (A) comprises the VHH sequence VHH8 shown in the Table of VHH Sequences.

Embodiment 146: The VHH antibody domain or fragment thereof according to any one of embodiments 24 to 131, wherein said VHH antibody domain of (A) comprises the VHH sequence VHH9 shown in the Table of VHH Sequences.

Embodiment 147: The VHH antibody domain or fragment thereof according to any one of embodiments 24 to 131, wherein said VHH antibody domain of (A) comprises the VHH sequence VHH10 shown in the Table of VHH Sequences.

Embodiment 148: The VHH antibody domain or fragment thereof according to any one of embodiments 24 to 131, wherein said VHH antibody domain of (A) comprises the VHH sequence VHH11 shown in the Table of VHH Sequences.

Embodiment 149: The VHH antibody domain or fragment thereof according to any one of embodiments 1 to 23 or 62 to 148, wherein in (a) said VHH antibody domain or fragment thereof comprises the complementarity determining regions CDR1, CDR2 and CDR3 of one VHH selected from the group consisting of VHH1, VHH2, VHH3, VHH4, VHH5, VHH6, VHH8, VHH9 and VHH10 as shown in the Table of CDRs.

Embodiment 150: The VHH antibody domain or fragment thereof according to any one of embodiments 1 to 23 or 62 to 148, wherein in (a) said VHH antibody domain or fragment thereof comprises the complementarity determining regions CDR1, CDR2 and CDR3 of one VHH selected from the group consisting of VHH1, VHH2, VHH5, VHH6, VHH8, VHH9 and VHH10 as shown in the Table of CDRs.

Embodiment 151: The VHH antibody domain or fragment thereof according to any one of embodiments 1 to 23 or 62 to 148, wherein in (a) said VHH antibody domain or fragment thereof comprises the complementarity determining regions CDR1, CDR2 and CDR3 of VHH2, VHH6, VHH8 and VHH10 as shown in the Table of CDRs.

Embodiment 152: The VHH antibody domain or fragment thereof according to any one of 1592 embodiments 1 to 23 or 62 to 148, wherein in (a) said VHH antibody domain or fragment thereof comprises the complementarity determining regions CDR1, CDR2 and CDR3 of one VHH selected from the group consisting of VHH6 and VHH11.

Embodiment 153: The VHH antibody domain or fragment thereof according to any one of embodiments 1 to 23 or 62 to 148, wherein in (a) said VHH antibody domain or fragment thereof comprises the complementarity determining regions CDR1, CDR2 and CDR3 of one VHH selected from the group consisting of VHH1, VHH2, VHH5, VHH6, VHH8 and VHH10 as shown in the Table of CDRs.

Embodiment 154: The VHH antibody domain or fragment thereof according to any one of embodiments 1 to 23 or 62 to 148, wherein in (a) said VHH antibody domain or fragment thereof comprises the complementarity determining regions CDR1, CDR2 and CDR3 of one VHH selected from the group consisting of VHH2, VHH6, VHH8, VHH10 as shown in the Table of CDRs.

Embodiment 155: The VHH antibody domain or fragment thereof according to any one of embodiments 1 to 23 or 62 to 148, wherein in (a) said VHH antibody domain or fragment thereof comprises the complementarity determining regions CDR1, CDR2 and CDR3 of one VHH selected from the group consisting of VHH2 or VHH8 as shown in the Table of CDRs.

Embodiment 156: The VHH antibody domain or fragment thereof according to any one of embodiments 1 to 23 or 62 to 148, wherein in (a) said VHH antibody domain or fragment thereof comprises the complementarity determining regions CDR1, CDR2 and CDR3 of VHH1 as shown in the Table of CDRs.

Embodiment 157: The VHH antibody domain or fragment thereof according to any one of embodiments 1 to 23 or 62 to 148, wherein in (a) said VHH antibody domain or fragment thereof comprises the complementarity determining regions CDR1, CDR2 and CDR3 of VHH2 as shown in the Table of CDRs.

Embodiment 158: The VHH antibody domain or fragment thereof according to any one of embodiments 1 to 23 or 62 to 148, wherein in (a) said VHH antibody domain or fragment thereof comprises the complementarity determining regions CDR1, CDR2 and CDR3 of VHH3 as shown in the Table of CDRs.

Embodiment 159: The VHH antibody domain or fragment thereof according to any one of embodiments 1 to 23 or 62 to 148, wherein in (a) said VHH antibody domain or fragment thereof comprises the complementarity determining regions CDR1, CDR2 and CDR3 of VHH4 as shown in the Table of CDRs.

Embodiment 160: The VHH antibody domain or fragment thereof according to any one of embodiments 1 to 23 or 62 to 148, wherein in (a) said VHH antibody domain or fragment thereof comprises the complementarity determining regions CDR1, CDR2 and CDR3 of VHH5 as shown in the Table of CDRs.

Embodiment 161: The VHH antibody domain or fragment thereof according to any one of embodiments 1 to 23 or 62 to 148, wherein in (a) said VHH antibody domain or fragment thereof comprises the complementarity determining regions CDR1, CDR2 and CDR3 of VHH6 as shown in the Table of CDRs.

Embodiment 162: The VHH antibody domain or fragment thereof according to any one of embodiments 1 to 23 or 62 to 148, wherein in (a) said VHH antibody domain or fragment thereof comprises the complementarity determining regions CDR1, CDR2 and CDR3 of VHH8 as shown in the Table of CDRs.

Embodiment 163: The VHH antibody domain or fragment thereof according to any one of embodiments 1 to 23 or 62 to 148, wherein in (a) said VHH antibody domain or fragment thereof comprises the complementarity determining regions CDR1, CDR2 and CDR3 of VHH9 as shown in the Table of CDRs.

Embodiment 164: The VHH antibody domain or fragment thereof according to any one of embodiments 1 to 23 or 62 to 148, wherein in (a) said VHH antibody domain or fragment thereof comprises the complementarity determining regions CDR1, CDR2 and CDR3 of VHH10 as shown in the Table of CDRs.

Embodiment 165: The VHH antibody domain or fragment thereof according to any one of embodiments 1 to 23 or 62 to 148, wherein in (a) said VHH antibody domain or fragment thereof comprises the complementarity determining regions CDR1, CDR2 and CDR3 of VHH11 as shown in the Table of CDRs.

Embodiment 166: The VHH antibody domain or fragment thereof according to any one of embodiments 1 to 165, wherein said VHH antibody domain or fragment thereof competes with VHH2 for binding to recombinant human IL-18Rα ECD.

Embodiment 167: The VHH antibody domain or fragment thereof according to any one of embodiments 1 to 165, wherein said VHH antibody domain or fragment thereof partially competes with VHH2 for binding to recombinant human IL-18Rα ECD.

Embodiment 168: The VHH antibody domain or fragment thereof according to any one of embodiments 1 to 167, wherein said VHH antibody domain or fragment thereof does not compete with VHH15 for binding to recombinant human IL-18RβECD.

Embodiment 169: The VHH antibody domain or fragment thereof according to any one of embodiments 1 to 168, wherein said humanization is by germlining.

Embodiment 170: The VHH antibody domain or fragment thereof according to embodiment 169, wherein said germlining involves
identifying the human VH germline sequence that is closest to the sequence to be humanized with respect to its sequence similarity; and
replacing amino acid(s) in the sequence to be humanized by the amino acid(s) at the corresponding sequence position(s) in the closest human VH germline sequence.

Sequence similarity and corresponding sequence positions can be determined by sequence alignment as described above.

Embodiment 171: The VHH antibody domain or fragment thereof according to embodiment 169, wherein said germlining consists of
identifying the human VH germline sequence that is closest to the sequence to be humanized with respect to its sequence similarity; and
replacing amino acid(s) in the sequence to be humanized by the amino acid(s) at the corresponding sequence position(s) in the closest human VH germline sequence.

Embodiment 172: The VHH antibody domain or fragment thereof according to any one of embodiments 1 to 171, wherein said VHH antibody domain (resp. fragment thereof) with humanization binds to human IL-18Rα ECD with an affinity (KD value) that is not weaker by a factor of more than 10 compared to the binding of a corresponding VHH antibody domain (resp. fragment thereof) without humanization to human IL-18Rα ECD.

As a skilled person understands, the term “corresponding VHH antibody domain without humanization” refers to a VHH antibody domain that differs only by the amino acid changes introduced in order to carry out said humanization, but that is otherwise identical.

Embodiment 173: The VHH antibody domain or fragment thereof according to any one of embodiments 1 to 171, wherein said VHH antibody domain (resp. fragment thereof) with humanization binds to human IL-18Rα ECD with an affinity (KD value) that is not weaker by a factor of more than 5 compared to the binding of a corresponding VHH antibody domain (resp. fragment thereof) without humanization to human IL-18Rα ECD.

Embodiment 174: The VHH antibody domain or fragment thereof according to any one of embodiments 1 to 171, wherein said VHH antibody domain (resp. fragment thereof) with humanization binds to human IL-18Rα ECD with an affinity (KD value) that is not weaker by a factor of more than 2 compared to the binding of a corresponding VHH antibody domain (resp. fragment thereof) without humanization to human IL-18Rα ECD.

Embodiment 175: The VHH antibody domain or fragment thereof according to any one of embodiments 1 to 171, wherein said VHH antibody domain (resp. fragment thereof) with humanization binds to human IL-18Rα ECD with an affinity (KD value) that is not weaker than the binding of a corresponding VHH antibody domain (resp. fragment thereof) without humanization to human IL-18Rα ECD.

Embodiment 176: The VHH antibody domain or fragment thereof according to any one of embodiments 172 to 175, wherein said affinity is determined by KD measurement.

Embodiment 177: The VHH antibody domain or fragment thereof according to any one of embodiments 24 to 176, wherein said VHH antibody domain comprises a VHH antibody domain according to (A), (B) or (C)/said fragment of a VHH antibody domain comprises a fragment of a VHH antibody domain according to (A), (B) or (C).

Embodiment 178: The VHH antibody domain or fragment thereof according to any one of embodiments 24 to 176, wherein said VHH antibody domain comprises a VHH antibody domain according to (A), (B) or (D)/said fragment of a VHH antibody domain comprises a fragment of a VHH antibody domain according to (A), (B) or (D).

Embodiment 179: The VHH antibody domain or fragment thereof according to any one of embodiments 24 to 176, wherein said VHH antibody domain comprises a VHH antibody domain according to (A), (C) or (D)/said fragment of a VHH antibody domain comprises a fragment of a VHH antibody domain according to (A), (C) or (D).

Embodiment 180: The VHH antibody domain or fragment thereof according to any one of embodiments 24 to 176, wherein said VHH antibody domain comprises a VHH antibody domain according to (A) or (B)/said fragment of a VHH antibody domain comprises a fragment of a VHH antibody domain according to (A) or (B).

Embodiment 181: The VHH antibody domain or fragment thereof according to any one of embodiments 24 to 176, wherein said VHH antibody domain comprises a VHH antibody domain according to (A) or (C)/said fragment of a VHH antibody domain comprises a fragment of a VHH antibody domain according to (A) or (C).

Embodiment 182: The VHH antibody domain or fragment thereof according to any one of embodiments 24 to 176, wherein said VHH antibody domain comprises a VHH antibody domain according to (A) or (D)/said fragment of a VHH antibody domain comprises a fragment of a VHH antibody domain according to (A) or (D).

Embodiment 183: The VHH antibody domain or fragment thereof according to any one of embodiments 24 to 176, wherein said VHH antibody domain comprises a VHH antibody domain according to (A)/said fragment of a VHH antibody domain comprises a fragment of a VHH antibody domain according to (A).

Embodiment 184: The VHH antibody domain or fragment thereof according to any one of embodiments 24 to 176, wherein said VHH antibody domain comprises a VHH antibody domain according to (B)/said fragment of a VHH antibody domain comprises a fragment of a VHH antibody domain according to (B).

Embodiment 185: The VHH antibody domain or fragment thereof according to any one of embodiments 1 to 23 or 62 to 184, wherein said VHH antibody domain or fragment thereof comprises complementarity determining regions according to (a) or (b).

Embodiment 186: The VHH antibody domain or fragment thereof according to any one of embodiments 1 to 23 or 62 to 184, wherein said VHH antibody domain or fragment thereof comprises complementarity determining regions according to (a) or (c).

Embodiment 187: The VHH antibody domain or fragment thereof according to any one of embodiments 1 to 23 or 62 to 184, wherein said VHH antibody domain or fragment thereof comprises complementarity determining regions according to (a).

Embodiment 188: The VHH antibody domain or fragment thereof according to any one of embodiments 1 to 23 or 62 to 184, wherein said VHH antibody domain or fragment thereof comprises complementarity determining regions according to (b). Fourth aspect of the present disclosure (also referred to as “Embodiment 189”): According to a fourth aspect, the present disclosure relates to a VHH antibody domain or a fragment thereof, wherein
(d) said VHH antibody domain or fragment thereof comprises the complementarity determining regions CDR1, CDR2 and CDR3 of one VHH selected from the group consisting of VHH12, VHH13, VHH14, VHH15, VHH16, VHH17, VHH18, VHH20, VHH21 and VHH22 as shown in the Table of CDRs;
(e) said VHH antibody domain or fragment thereof comprises the complementarity determining regions CDR1, CDR2 and CDR3 as defined in (d) with modification, wherein the modification is that the sequence of at least one of CDR1, CDR2 and CDR3 is humanized; or
(f) said VHH antibody domain or fragment thereof comprises the complementarity determining regions CDR1, CDR2 and CDR3 as defined in (d) with modification, wherein the modification is
   the replacement, addition or deletion of up to three amino acids in CDR1,
   the replacement, addition or deletion of up to three amino acids in CDR2 and/or
   the replacement, addition or deletion of up to three amino acids in CDR3;

TABLE of
CDRs:

| Description | SEQ ID NO | Amino acid sequence |
|---|---|---|
| CDR1 of VHH1 | SEQ ID NO: 23 | GFTFNSYA |
| CDR2 of VHH1 | SEQ ID NO: 24 | HIGSGGSTR |
| CDR3 of VHH1 | SEQ ID NO: 25 | NVYGATLRS |
| CDR1 of VHH2 | SEQ ID NO: 26 | GFTLLDSYD |
| CDR2 of VHH2 | SEQ ID NO: 27 | ISSSGGKSTY |
| CDR3 of VHH2 | SEQ ID NO: 28 | AADGAEGFWSCQSGGYDY |
| CDR1 of VHH3 | SEQ ID NO: 29 | GFTLGAYA |
| CDR2 of VHH3 | SEQ ID NO: 30 | ITSSGRSAY |
| CDR3 of VHH3 | SEQ ID NO: 31 | AAGAACAAAGTMNY |
| CDR1 of VHH4 | SEQ ID NO: 32 | GFTLGAYA |
| CDR2 of VHH4 | SEQ ID NO: 33 | ITSSGGSAY |
| CDR3 of VHH4 | SEQ ID NO: 34 | AAGGACAAAETMNY |
| CDR1 of VHH5 | SEQ ID NO: 35 | GRTFSNLA |
| CDR2 of VHH5 | SEQ ID NO: 36 | IDWNGDNRL |
| CDR3 of VHH5 | SEQ ID NO: 37 | AADSTYSTDWYLNPLNR |
| | | TRYDY |
| CDR1 of VHH6 | SEQ ID NO: 38 | KSIFSINA |
| CDR2 of VHH6 | SEQ ID NO: 39 | FTSSGRIN |
| CDR3 of VHH6 | SEQ ID NO: 40 | NVGLWEDGLWYDY |
| CDR1 of VHH7 | SEQ ID NO: 41 | GHTFNNLA |
| CDR2 of VHH7 | SEQ ID NO: 42 | IDWNGDNRL |
| CDR3 of VHH7 | SEQ ID NO: 43 | AADSTYSTNWYLDPLNR |
| | | TTYNY |
| CDR1 of VHH8 | SEQ ID NO: 44 | GRTFSNDA |
| CDR2 of VHH8 | SEQ ID NO: 45 | VSWSLGMTY |
| CDR3 of VHH8 | SEQ ID NO: 46 | AAGPDLSDELYGYDY |
| CDR1 of VHH9 | SEQ ID NO: 47 | RSDFSRYT |
| CDR2 of VHH9 | SEQ ID NO: 48 | RITSGGIPH |
| CDR3 of VHH9 | SEQ ID NO: 49 | NTVGVALWA |
| CDR1 of VHH10 | SEQ ID NO: 50 | GLPFSSYA |
| CDR2 of VHH10 | SEQ ID NO: 51 | ITWSGSSIY |
| CDR3 of VHH10 | SEQ ID NO: 52 | AAARRRVGLPLADPREY |
| | | VY |
| CDR1 of VHH11 | SEQ ID NO: 53 | GSPIRFSV |
| CDR2 of VHH11 | SEQ ID NO: 54 | RITSGGIVY |
| CDR3 of VHH11 | SEQ ID NO: 55 | NRLGGALFD |
| CDR1 of VHH12 | SEQ ID NO: 56 | GVGFSIYT |
| CDR2 of VHH12 | SEQ ID NO: 57 | VTTGGHIN |
| CDR3 of VHH12 | SEQ ID NO: 58 | NAIYFGQDI |
| CDR1 of VHH13 | SEQ ID NO: 59 | GISFRVNV |
| CDR2 of VHH13 | SEQ ID NO: 60 | ISSGDSTN |
| CDR3 of VHH13 | SEQ ID NO: 61 | NARIFARDN |
| CDR1 of VHH14 | SEQ ID NO: 62 | DRTVSKGA |
| CDR2 of VHH14 | SEQ ID NO: 63 | IGRSSGITY |
| CDR3 of VHH14 | SEQ ID NO: 64 | AADSSGIIRTTSWAYDY |
| CDR1 of VHH15 | SEQ ID NO: 65 | GRFFTINA |
| CDR2 of VHH15 | SEQ ID NO: 66 | ITSDGSTT |
| CDR3 of VHH15 | SEQ ID NO: 67 | NADGRENDNGYVKNFNI |
| CDR1 of VHH16 | SEQ ID NO: 68 | GFTTDDYI |
| CDR2 of VHH16 | SEQ ID NO: 69 | ISTTDGSAW |
| CDR3 of VHH16 | SEQ ID NO: 70 | AEVSTGTGVLYCREEFY |
| | | FGMDS |
| CDR1 of VHH17 | SEQ ID NO: 71 | GFALDEIA |
| CDR2 of VHH17 | SEQ ID NO: 72 | ISSSDGSTL |
| CDR3 of VHH17 | SEQ ID NO: 73 | AAVRGSCFGRTTH |
| CDR1 of VHH18 | SEQ ID NO: 74 | GRTFSSYA |
| CDR2 of VHH18 | SEQ ID NO: 75 | ISWSGGSTY |
| CDR3 of VHH18 | SEQ ID NO: 76 | AADRSLSGSYFGATIDY |
| CDR1 of VHH19 | SEQ ID NO: 77 | GIRFNPER |
| CDR2 of VHH19 | SEQ ID NO: 78 | IDSGGSTD |
| CDR3 of VHH19 | SEQ ID NO: 79 | NTQIFARDY |
| CDR1 of VHH20 | SEQ ID NO: 80 | VSAFRLKV |
| CDR2 of VHH20 | SEQ ID NO: 81 | IDSGDTKS |
| CDR3 of VHH20 | SEQ ID NO: 82 | NAVLFGRDY |
| CDR1 of VHH21 | SEQ ID NO: 83 | GITFSPYV |
| CDR2 of VHH21 | SEQ ID NO: 84 | MVNGGSTN |
| CDR3 of VHH21 | SEQ ID NO: 85 | HIMIFGRAS |
| CDR1 of VHH22 | SEQ ID NO: 86 | GFALEYYA |
| CDR2 of VHH22 | SEQ ID NO: 87 | VSSSRSGGSTV |
| CDR3 of VHH22 | SEQ ID NO: 88 | AGAPAYRSHYCRLQADD |
| | | YTY |

To the fourth aspect of the present disclosure and the embodiments referring thereto, the same explanations and definitions apply accordingly as for the first to third aspects of the present disclosure and the embodiments referring thereto.

Embodiment 190: The VHH antibody domain or fragment thereof according to embodiment 189, wherein the modification in (e) is that the sequence of CDR1 and/or CDR2, but not the sequence of CDR3 is humanized.

Embodiment 191: The VHH antibody domain or fragment thereof according to any one of embodiments 189 or 190, wherein the modification in (e) is that the sequence of CDR1 is humanized, but not the sequence of CDR2 and CDR3.

Embodiment 192: The VHH antibody domain or fragment thereof according to any one of embodiments 189 or 190, wherein the modification in (e) is that the sequence of CDR2 is humanized, but not the sequence of CDR1 and CDR3.

Embodiment 193: The VHH antibody domain or fragment thereof according to any one of embodiments 189 to 192, wherein the modification in (e) is that the sequence of one, but not more than one of CDR1, CDR2 and CDR3 is humanized.

Embodiment 194: The VHH antibody domain or fragment thereof according to any one of embodiments 189 to 193, wherein said humanization of said CDR(s) is by replacing at least one amino acid in the sequence of said CDR by the corresponding amino acid of a human VH domain.

Embodiment 195: The VHH antibody domain or fragment thereof according to any one of embodiments 189 to 194, wherein said humanization of said CDR(s) is by replacing up to three amino acids in the sequence of said CDR by the corresponding amino acid of a human VH domain.

Embodiment 196: The VHH antibody domain or fragment thereof according to any one of embodiments 189 to 194, wherein said humanization of said CDR(s) is by replacing up to three amino acids in the sequence of CDR1 and/or CDR2 and up to one amino acid in the sequence of CDR3 by the corresponding amino acid of a human VH domain.

Embodiment 197: The VHH antibody domain or fragment thereof according to any one of embodiments 189 to 194, wherein said humanization of said CDR(s) is by replacing up to two amino acids in the sequence of said CDR by the corresponding amino acid of a human VH domain.

Embodiment 198: The VHH antibody domain or fragment thereof according to any one of embodiments 189 to 194, wherein said humanization of said CDR(s) is by replacing up to two amino acids in the sequence of CDR1 and/or CDR2 and up to one amino acid in the sequence of CDR3 by the corresponding amino acid of a human VH domain.

Embodiment 199: The VHH antibody domain or fragment thereof according to any one of embodiments 189 to 198, wherein said humanization of said CDR(s) is by replacing one amino acid in the sequence of said CDR by the corresponding amino acid of a human VH domain.

Embodiment 200: The VHH antibody domain or fragment thereof according to any one of embodiments 189 to 199, wherein the modification in (f) is
the replacement, addition or deletion of up to three amino acids in CDR1,
the replacement, addition or deletion of up to three amino acids in CDR2 and/or
the replacement, addition or deletion of up to one amino acid in CDR3.

Embodiment 201: The VHH antibody domain or fragment thereof according to any one of embodiments 189 to 199, wherein the modification in (f) is
the replacement, addition or deletion of up to two amino acids in CDR1,
the replacement, addition or deletion of up to two amino acids in CDR2 and/or
the replacement, addition or deletion of up to two amino acids in CDR3;

Embodiment 202: The VHH antibody domain or fragment thereof according to any one of embodiments 189 to 199, wherein the modification in (f) is
the replacement, addition or deletion of up to two amino acids in CDR1;
the replacement, addition or deletion of up to two amino acids in CDR2; and/or
the replacement, addition or deletion of up to one amino acid in CDR3.

Embodiment 203: The VHH antibody domain or fragment thereof according to any one of embodiments 189 to 199, wherein the modification in (f) is
the replacement, addition or deletion of up to two amino acids in CDR1 and/or
the replacement, addition or deletion of up to two amino acids in CDR2; wherein the sequence of CDR3 is unmodified.

As a skilled person understands, the indication that the “sequence of CDR3 is unmodified” means that the sequence is unmodified compared to the sequence provided for CDR3 for the VHH at issue in the Table of CDRs.

Embodiment 204: The VHH antibody domain or fragment thereof according to any one of embodiments 189 to 199, wherein the modification in (f) is
the replacement, addition or deletion of up to two amino acids in CDR1, wherein the sequence of CDR2 and CDR3 is unmodified.

Embodiment 205: The VHH antibody domain or fragment thereof according to any one of embodiments 189 to 199, wherein the modification in (f) is
the replacement, addition or deletion of up to two amino acids in CDR2, wherein the sequence of CDR1 and CDR3 is unmodified.

Embodiment 206: The VHH antibody domain or fragment thereof according to any one of embodiments 189 to 199, wherein the modification in (f) is
the replacement, addition or deletion of up to one amino acid in CDR1;
the replacement, addition or deletion of up to one amino acid in CDR2; and/or
the replacement, addition or deletion of up to one amino acid in CDR3.

Embodiment 207: The VHH antibody domain or fragment thereof according to any one of embodiments 189 to 199, wherein the modification in (f) is
the replacement, addition or deletion of one amino acid in CDR1 and/or
the replacement, addition or deletion of one amino acid in CDR2; wherein the sequence of CDR3 is unmodified.

Embodiment 208: The VHH antibody domain or fragment thereof according to any one of embodiments 189 to 199, wherein the modification in (f) is
the replacement, addition or deletion of one amino acid in CDR1, wherein the sequence of CDR2 and CDR3 is unmodified.

Embodiment 209: The VHH antibody domain or fragment thereof according to any one of embodiments 189 to 199, wherein the modification in (f) is
the replacement, addition or deletion of one amino acid in CDR2, wherein the sequence of CDR1 and CDR3 is unmodified.

Embodiment 210: The VHH antibody domain or fragment thereof according to any one of embodiments 189 to 209, wherein the modification in (f) comprises only the replacement, but not the addition or deletion of amino acids.

Embodiment 211: The VHH antibody domain or fragment thereof according to any one of embodiments 189 to 210, wherein said replacement is a conservative amino acid replacement. Fifth aspect of the present disclosure (also referred to as “Embodiment 212”): According to a fifth aspect, the present disclosure relates to a VHH antibody domain or a fragment thereof, wherein
(E) said VHH antibody domain comprises the amino acid sequence of a VHH selected from the group consisting of VHH12, VHH13, VHH14, VHH15, VHH16, VHH17, VHH18, VHH20, VHH21 and VHH22 as shown in the Table of VHH Sequences;
(F) said VHH antibody domain comprises a VHH sequence as defined in (E) with modification, wherein the modification is that said sequence is humanized;
(G) said VHH antibody domain comprises a VHH sequence as defined in (E) with modification, wherein the modification is the replacement, addition or deletion of up to 25 amino acids; or
(H) said VHH antibody domain comprises a VHH sequence that is at least 75% identical to a VHH sequence referred to in (E);

TABLE of
VHH Sequences:

| VHH | SEQ ID NO | Amino acid sequence |
|---|---|---|
| VHH1 | SEQ ID NO: 1 | DVPLQESGGGLVQTGGSLRLSCAASGFTFNSYAMGW |
| | | YQQAPGKKYRELVAHIGSGGSTRYIDSVKGRFTISR |
| | | DNAKNTVYLQMNSLKPEDTAVYYCNVYGATLRSWGQ |
| | | GTQVTVSS |
| VHH2 | SEQ ID NO: 2 | DVQLQESGGGLVQAGGSLRLSCAASGFTLLDSYDIG |
| | | WFRQAPGKKREEVSCISSSGGKSTYYADSVKGRETI |
| | | SRDNANNTVYLQMNSLKPEDTAVYYCAADGAEGEWS |
| | | CQSGGYDYWGQGTQVTVSS |
| VHH3 | SEQ ID NO: 3 | DVQLQESGGGLVQAGGSLRLSCAASGFTLGAYAINW |
| | | FRQAPGKERARAWDACITSSGRSAYYADSVKGRFTV |
| | | SRDHAKNTVYLQMNSLKPEDTAFYYCAAGAACAAAG |
| | | TMNYWGKGTLVTVSS |
| VHH4 | SEQ ID NO: 4 | DVQLQESGGGLVQAGGSLRLSCAASGFTLGAYAINW |
| | | FQQAPGKERARAWVLCITSSGGSAYYVDSVKGRFTV |
| | | SRDNAKNTVYLQMNSLKPEDTAFYYCAAGGACAAAE |
| | | TMNYWGKGTLVTVSS |
| VHH5 | SEQ ID NO: 5 | DVQLQESGGGLVQAGGSLRLSCAASGRTFSNLAMAW |
| | | FRQAPGKEREFVAAIDWNGDNRLYAASVKGRFTISR |
| | | DNAKSTMYLQMNSLKPEDTAVYYCAADSTYSTDWYL |
| | | NPLNRTRYDYWGQGTQVTVSS |
| VHH6 | SEQ ID NO: 6 | DVPLQESGGGSVQPGGSLRLSCAASKSIFSINAMRW |
| | | HRQAPGKQRELVATFTSSGRINYADSVKGRFTISTD |
| | | NAKNTIYLQMNSLNPEDTAVYRCNVGLWEDGLWYDY |
| | | WGQGTQVTVSS |
| VHH7 | SEQ ID NO: 7 | DVQLQESGGGLVQAGGSLRLSCAASGHTFNNLAMAW |
| | | CRQAPGKEREFVSAIDWNGDNRLYAYSVKGRFTISR |
| | | DNAKSTMYLQMNSLKPEDTAVYYCAADSTYSTNWYL |
| | | DPLNRTTYNYWGQGTPVTVSS |
| VHH8 | SEQ ID NO: 8 | DVQLQESGGGLVQAGGSLRLSCAASGRTFSNDAMGW |
| | | FRQAPGKEREFVAAVSWSLGMTYNADSVKGRFTISR |
| | | DNAKNTVYLQMNSLKPDDTAVYYCAAGPDLSDELYG |
| | | YDYWGQGTQVTVSS |
| VHH9 | SEQ ID NO: 9 | DVQLQESGGGLVQPGESLRLSCTASRSDFSRYTMGW |
| | | YRQAPGKWRELVARITSGGIPHYADSVKGRFTISRD |
| | | NAKNTVTLQMNTLQFEDSAVYYCNTVGVALWAWGQG |
| | | TLVTVSS |
| VHH10 | SEQ ID NO: 10 | DVQLQESGGGLVQAGGSLRLSCAASGLPFSSYAMGW |
| | | FRQAPGKDREFVAGITWSGSSIYYRDSVKGRFTISR |
| | | DNAKNTVWLQMNDLKPEDTAVYYCAAARRRVGLPLA |
| | | DPREYVYWGQGTQVTVSS |
| VHH11 | SEQ ID NO: 11 | DVPLQESGGGLVQPGQSLRLSCAASGSPIRFSVMGW |
| | | FQQAPGKGRDLIARITSGGIVYYTDSVKGRFTISKD |
| | | NAKNTLYLQMNSLNLEDTGVYYCNRLGGALEDWGQG |
| | | TQVTVSS |
| VHH12 | SEQ ID NO: 12 | DVPLQESGGGLVQPGGSLRLSCRASGVGFSIYTMGW |
| | | YRQAPGEQRHLVATVTTGGHINYGDSVQGRFTISRE |
| | | NNGNVVELQMHSLKPEDTAVYFCNAIYFGQDIWGQG |
| | | TQVTVSS |
| VHH13 | SEQ ID NO: 13 | DVQLQESGGGLVQPGGSLRLSCAASGISFRVNVLGW |
| | | YRQAPGKPRELVASISSGDSTNYADSVKGRFTISRD |
| | | NAKSTVYLQMNNLKPEDTAVYYCNARIFARDNWGQG |
| | | TQVTVSS |
| VHH14 | SEQ ID NO: 14 | DVPLQESGGGLVQAGGSLRLSCAASDRTVSKGAMGW |
| | | FRQAPGKEREFVATIGRSSGITYYADSVKGRFTISR |
| | | DNAKNTWYLQMNSLRPEDTAVYYCAADSSGIIRTTS |
| | | WAYDYWGQGTQVTVSS |
| VHH15 | SEQ ID NO: 15 | DVQLQESGGGLVQPGGSLRLSCAASGRFFTINAMGW |
| | | YRQTPGKQRELVAAITSDGSTTYQDSVKGRFTISRD |
| | | NANRTANLYMNSLKSEDTAVYYCNADGRENDNGYVK |
| | | NFNIWGQGTQVTVSS |
| VHH16 | SEQ ID NO: 16 | DVQLQESGGGSVPAGGSLRLSCSASGFTTDDYIIGW |
| | | FRQAPGKKREGVSCISTTDGSAWYADSVKGRFTISS |
| | | DNANNTVTLQMNSLLPEDTSVYYCAEVSTGTGVLYC |
| | | REEFYFGMDSWGKGTPVTVSS |
| VHH17 | SEQ ID NO: 17 | DVPLQESGGGLVQAGGSLRLSCVASGFALDEIAVGW |
| | | FRQAPGKEREGLSCISSSDGSTLYADSVKGRFTISS |
| | | DNAKNTVYLQMNSLKPEDTAVYLCAAVRGSCFGRTT |
| | | HWGQGTQVTVSS |
| VHH18 | SEQ ID NO: 18 | DVQLQESGGGLVQAGGSLRLSCAASGRTFSSYAMGW |
| | | FRQAPGMVREFVAAISWSGGSTYYADSVQGRFTISR |
| | | DNAKNTVYLQMNSLKPEDTAVYYCAADRSLSGSYFG |
| | | ATIDYWGQGTQVTVSS |
| VHH19 | SEQ ID NO: 20 | DVQLQESGGGLVQAGGSLRLSCAASGIRENPERMAW |
| | | YRQAPGKQRELVAAIDSGGSTDYADSVRGRFTISRD |
| | | NAKITVSLQMNSLKPEDTAVYYCNTQIFARDYWGQG |
| | | SQVTVSS |
| VHH20 | SEQ ID NO: 21 | DVQLQESGGGLVQAGGSLRLSCAASVSAFRLKVMGW |
| | | YRQAPGKEREFVAAIDSGDTKSYTNAVKGRFTISGG |
| | | AATNTVVLQMNSLKPEDTAFYYCNAVLEGRDYWGQG |
| | | TQVTVSS |
| VHH21 | SEQ ID NO: 22 | DVQLQESGGGLVQAGGSLRLSCAASGITFSPYVMGW |
| | | YRQAPGKQRELVADMVNGGSTNYADSVKGRFTIERD |
| | | TAANTVTLQMNNLKPDDTAVYYCHIMIFGRASWGQG |
| | | TQVTVSS |
| VHH22 | SEQ ID NO: 22 | DVPLQESGGGLVQPGGSLRLSCAAPGFALEYYAIGW |
| | | FRQAPGKEREGLSCVSSSRSGGSTVYADSVKGRFTI |
| | | SRDNAKNTVYLQIDSLKVEDTAVYYCAGAPAYRSHY |
| | | CRLQADDYTYWGQGTQVTVSS |

Sixth aspect of the present disclosure (also referred to as “Embodiment 213”): According to a sixth aspect, the present disclosure relates to a VHH antibody domain or a fragment thereof, wherein
(E) said VHH antibody domain consists of the amino acid sequence of a VHH selected from the group consisting of VHH12, VHH13, VHH14, VHH15, VHH16, VHH17, VHH18, VHH20, VHH21 and VHH22 as shown in the Table of VHH Sequences;
(F) said VHH antibody domain consists of a VHH sequence as defined in (E) with modification, wherein the modification is that said sequence is humanized;
(G) said VHH antibody domain consists of a VHH sequence as defined in (E) with modification, wherein the modification is the replacement, addition or deletion of up to 25 amino acids; or
(H) said VHH antibody domain consists of a VHH sequence that is at least 75% identical to a VHH sequence referred to in (E).

To the fifth and sixth aspects of the present disclosure and the embodiments referring thereto, the same explanations and definitions apply accordingly as for the first to fourth aspects of the present disclosure and the embodiments referring thereto.

Embodiment 214: The VHH antibody domain or fragment thereof according to any one of embodiments 212 or 213, wherein said fragment of said VHH antibody domain comprises at least 75% of the amino acids of the sequence of said VHH antibody domain. As the skilled person understands, this means that that fragment lacks up to a quarter of the total number of amino acids of said VHH antibody domain, wherein, compared to the “complete” VHH antibody domain sequence said amino acids are lacking either at the N-terminus or at the C-terminus.

Embodiment 215: The VHH antibody domain or fragment thereof according to any one of embodiments 212 or 213, wherein said fragment of said VHH antibody domain comprises at least 80% of the amino acids of the sequence of said VHH antibody domain.

Embodiment 216: The VHH antibody domain or fragment thereof according to any one of embodiments 212 or 213, wherein said fragment of said VHH antibody domain comprises at least 85% of the amino acids of the sequence of said VHH antibody domain.

Embodiment 217: The VHH antibody domain or fragment thereof according to any one of embodiments 212 or 213, wherein said fragment of said VHH antibody domain comprises at least 90% of the amino acids of the sequence of said VHH antibody domain.

Embodiment 218: The VHH antibody domain or fragment thereof according to any one of embodiments 212 or 213, wherein said fragment of said VHH antibody domain comprises at least 95% of the amino acids of the sequence of said VHH antibody domain.

Embodiment 219: The VHH antibody domain or fragment thereof according to any one of embodiments 212 or 213, wherein said fragment of said VHH antibody domain comprises at least 98% of the amino acids of the sequence of said VHH antibody domain.

Embodiment 220: The VHH antibody domain or fragment thereof according to any one of embodiments 212 or 213, wherein said fragment of said VHH antibody domain comprises at least 99% of the amino acids of the sequence of said VHH antibody domain.

Embodiment 221: The VHH antibody domain or fragment thereof according to any one of embodiments 212 to 220, wherein said fragment of said VHH antibody domain comprises the complementarity determining regions CDR1, CDR2 and CDR3.

Embodiment 222: The VHH antibody domain or fragment thereof according to any one of embodiments 212 to 221, wherein said fragment of said VHH antibody domain comprises at least the sequence from the N-terminus of CDR1 to the C-terminus of CDR3 of said VHH antibody domain.

Embodiment 223: The VHH antibody domain or fragment thereof according to any one of embodiments 212 to 222, wherein in (E) said fragment of said VHH antibody domain comprises all the complementarity determining regions (CDRs) of said VHH antibody domain.

Embodiment 224: The VHH antibody domain or fragment thereof according to any one of embodiments 212 to 223, wherein in (F) said humanization of said sequence is by replacing at least one amino acid of said sequence by the corresponding amino acid of a human VH (variable heavy) domain.

Embodiment 225: The VHH antibody domain or fragment thereof according to any one of embodiments 212 to 224, wherein in (F) said humanization of said sequence is by (individually) replacing up to 25 amino acids of said sequence by the corresponding amino acids of a human VH domain.

Embodiment 226: The VHH antibody domain or fragment thereof according to any one of embodiments 212 to 224, wherein in (F) said humanization of said sequence is by replacing up to 20 amino acids of said sequence by the corresponding amino acids of a human VH domain.

Embodiment 227: The VHH antibody domain or fragment thereof according to any one of embodiments 212 to 224, wherein in (F) said humanization of said sequence is by replacing up to 15 amino acids of said sequence by the corresponding amino acids of a human VH domain.

Embodiment 228: The VHH antibody domain or fragment thereof according to any one of embodiments 212 to 224, wherein in (F) said humanization of said sequence is by replacing up to 10 amino acids of said sequence by the corresponding amino acids of a human VH domain.

Embodiment 229: The VHH antibody domain or fragment thereof according to any one of embodiments 212 to 224, wherein in (F) said humanization of said sequence is by replacing up to 5 amino acids of said sequence by the corresponding amino acids of a human VH domain.

Embodiment 230: The VHH antibody domain or fragment thereof according to any one of embodiments 212 to 224, wherein in (F) said humanization of said sequence is by replacing up to 3 amino acids of said sequence by the corresponding amino acids of a human VH domain.

Embodiment 231: The VHH antibody domain or fragment thereof according to any one of embodiments 212 to 224, wherein in (F) said humanization of said sequence is by replacing up to 2 amino acids of said sequence by the corresponding amino acids of a human VH domain.

Embodiment 232: The VHH antibody domain or fragment thereof according to any one of embodiments 212 to 224, wherein in (F) said humanization of said sequence is by replacing one amino acid of said sequence by the corresponding amino acid of a human VH domain.

Embodiment 233: The VHH antibody domain or fragment thereof according to any one of embodiments 212 to 232, wherein in (F) said humanization is within the framework regions of said VHH antibody domain and/or within the CDRs of said VHH antibody domain.

Embodiment 234: The VHH antibody domain or fragment thereof according to any one of embodiments 212 to 232, wherein in (F) said humanization is within the framework regions of said VHH antibody domain, but not within the CDRs of said VHH antibody domain.

Embodiment 235: The VHH antibody domain or fragment thereof according to any one of embodiments 212 to 224, wherein in (F) said humanization is within the CDRs of said VHH antibody domain, but not within the framework regions of said VHH antibody domain.

Embodiment 236: The VHH antibody domain or fragment thereof according to any one of embodiments 212 to 233 or 235, wherein in (F) said humanization within the CDRs of said VHH antibody domain is within CDR1, CDR2 and/or CDR3.

Embodiment 237: The VHH antibody domain or fragment thereof according to any one of embodiments 212 to 233 or 235 to 236, wherein in (F) said humanization within the CDRs of said VHH antibody domain is within CDR1 and/or CDR2.

Embodiment 238: The VHH antibody domain or fragment thereof according to any one of embodiments 212 to 233 or 235 to 237, wherein in (F) said humanization within the CDRs of said VHH antibody domain is within CDR1.

Embodiment 239: The VHH antibody domain or fragment thereof according to any one of embodiments 212 to 233 or 235 to 238, wherein in (F) said humanization within the CDRs of said VHH antibody domain is within CDR2.

Embodiment 240: The VHH antibody domain or fragment thereof according to any one of embodiments 212 to 239, wherein in (F) said humanization within the CDRs of said VHH antibody domain is not within CDR3.

Embodiment 241: The VHH antibody domain or fragment thereof according to any one of embodiments 212 to 240, wherein in (G) the modification is the replacement, addition or deletion of up to 20 amino acids.

Embodiment 242: The VHH antibody domain or fragment thereof according to any one of embodiments 212 to 240, wherein in (G) the modification is the replacement, addition or deletion of up to 15 amino acids.

Embodiment 243: The VHH antibody domain or fragment thereof according to any one of embodiments 212 to 240, wherein in (G) the modification is the replacement, addition or deletion of up to 10 amino acids.

Embodiment 244: The VHH antibody domain or fragment thereof according to any one of embodiments 212 to 240, wherein in (G) the modification is the replacement, addition or deletion of up to 5 amino acids.

Embodiment 245: The VHH antibody domain or fragment thereof according to any one of embodiments 212 to 240, wherein in (G) the modification is the replacement, addition or deletion of up to 3 amino acids.

Embodiment 246: The VHH antibody domain or fragment thereof according to any one of embodiments 212 to 240, wherein in (G) the modification is the replacement, addition or deletion of up to 2 amino acids.

Embodiment 247: The VHH antibody domain or fragment thereof according to any one of embodiments 212 to 240, wherein in (G) the modification is the replacement, addition or deletion of one amino acid.

Embodiment 248: The VHH antibody domain or fragment thereof according to any one of embodiments 212 to 247, wherein the modification in (G) comprises only the replacement, but not the addition or deletion of amino acids.

Embodiment 249: The VHH antibody domain or fragment thereof according to any one of embodiments 212 to 248, wherein in (E) said VHH antibody domain or fragment thereof comprises the complementarity determining regions CDR1, CDR2 and CDR3 of one VHH selected from the group consisting of VHH12, VHH13, VHH14, VHH15, VHH16, VHH17, VHH18, VHH20, VHH21 and VHH22 as shown in the Table of CDRs.

Embodiment 250: The VHH antibody domain or fragment thereof according to any one of embodiments 212 to 249, wherein in (F) to (H)
(d) said VHH antibody domain or fragment thereof comprises the complementarity determining regions CDR1, CDR2 and CDR3 of one VHH selected from the group consisting of VHH12, VHH13, VHH14, VHH15, VHH16, VHH17, VHH18, VHH20, VHH21 and VHH22 as shown in the Table of CDRs;
(e) said VHH antibody domain or fragment thereof comprises the complementarity determining regions CDR1, CDR2 and CDR3 as defined in (d) with modification, wherein the modification is that the sequence of at least one of CDR1, CDR2 and CDR3 is humanized;
(f) said VHH antibody domain or fragment thereof comprises the complementarity determining regions CDR1, CDR2 and CDR3 as defined in (d) with modification, wherein the modification is
   the replacement, addition or deletion of up to three amino acids in CDR1,
   the replacement, addition or deletion of up to three amino acids in CDR2 and/or
   the replacement, addition or deletion of up to three amino acids in CDR3.

Embodiment 251: The VHH antibody domain or fragment thereof according to embodiment 250, wherein the modification in (e) is that the sequence of CDR1 and/or CDR2, but not the sequence of CDR3 is humanized.

Embodiment 252: The VHH antibody domain or fragment thereof according to any one of embodiments 250 or 251, wherein the modification in (e) is that the sequence of CDR1 is humanized, but not the sequence of CDR2 and CDR3.

Embodiment 253: The VHH antibody domain or fragment thereof according to any one of embodiments 250 or 251, wherein the modification in (e) is that the sequence of CDR2 is humanized, but not the sequence of CDR1 and CDR3.

Embodiment 254: The VHH antibody domain or fragment thereof according to any one of embodiments 250 to 253, wherein the modification in (e) is that the sequence of one, but not more than one of CDR1, CDR2 and CDR3 is humanized.

Embodiment 255: The VHH antibody domain or fragment thereof according to any one of embodiments 250 to 254, wherein said humanization of said CDR(s) is by replacing at least one amino acid in the sequence of said CDR by the corresponding amino acid of a human VH domain.

Embodiment 256: The VHH antibody domain or fragment thereof according to any one of embodiments 250 to 255, wherein said humanization of said CDR(s) is by replacing up to three amino acids in the sequence of said CDR by the corresponding amino acid of a human VH domain.

Embodiment 257: The VHH antibody domain or fragment thereof according to any one of embodiments 250 to 255, wherein said humanization of said CDR(s) is by replacing up to three amino acids in the sequence of CDR1 and/or CDR2 and up to one amino acid in the sequence of CDR3 by the corresponding amino acid of a human VH domain.

Embodiment 258: The VHH antibody domain or fragment thereof according to any one of embodiments 250 to 255, wherein said humanization of said CDR(s) is by replacing up to two amino acids in the sequence of said CDR by the corresponding amino acid of a human VH domain.

Embodiment 259: The VHH antibody domain or fragment thereof according to any one of embodiments 250 to 255, wherein said humanization of said CDR(s) is by replacing up to two amino acids in the sequence of CDR1 and/or CDR2 and up to one amino acid in the sequence of CDR3 by the corresponding amino acid of a human VH domain.

Embodiment 260: The VHH antibody domain or fragment thereof according to any one of embodiments 250 to 259, wherein said humanization of said CDR(s) is by replacing one amino acid in the sequence of said CDR by the corresponding amino acid of a human VH domain.

Embodiment 261: The VHH antibody domain or fragment thereof according to any one of embodiments 250 to 260, wherein the modification in (f) is
the replacement, addition or deletion of up to three amino acids in CDR1,
the replacement, addition or deletion of up to three amino acids in CDR2 and/or
the replacement, addition or deletion of up to two amino acids in CDR3.

Embodiment 262: The VHH antibody domain or fragment thereof according to any one of embodiments 250 to 260, wherein the modification in (f) is
the replacement, addition or deletion of up to three amino acids in CDR1,
the replacement, addition or deletion of up to three amino acids in CDR2 and/or
the replacement, addition or deletion of up to one amino acid in CDR3.

Embodiment 263: The VHH antibody domain or fragment thereof according to any one of embodiments 250 to 260, wherein the modification in (f) is
the replacement, addition or deletion of up to three amino acids in CDR1, and/or
the replacement, addition or deletion of up to three amino acids in CDR2, wherein the sequence of CDR3 is unmodified.

Embodiment 264: The VHH antibody domain or fragment thereof according to any one of embodiments 250 to 260, wherein the modification in (f) is
the replacement, addition or deletion of up to two amino acids in CDR1,
the replacement, addition or deletion of up to two amino acids in CDR2 and/or
the replacement, addition or deletion of up to two amino acids in CDR3;

Embodiment 265: The VHH antibody domain or fragment thereof according to any one of embodiments 250 to 260, wherein the modification in (f) is
the replacement, addition or deletion of up to two amino acids in CDR1;
the replacement, addition or deletion of up to two amino acids in CDR2; and/or
the replacement, addition or deletion of up to one amino acid in CDR3.

Embodiment 266: The VHH antibody domain or fragment thereof according to any one of embodiments 250 to 260, wherein the modification in (f) is
the replacement, addition or deletion of up to two amino acids in CDR1 and/or
the replacement, addition or deletion of up to two amino acids in CDR2; wherein the sequence of CDR3 is unmodified.

As the skilled person is aware, “unmodified” means unmodified compared to the sequence in the Table of CDRs.

Embodiment 267: The VHH antibody domain or fragment thereof according to any one of embodiments 250 to 260, wherein the modification in (f) is
the replacement, addition or deletion of up to two amino acids in CDR1, wherein the sequence of CDR2 and CDR3 is unmodified.

Embodiment 268: The VHH antibody domain or fragment thereof according to any one of embodiments 250 to 260, wherein the modification in (f) is
the replacement, addition or deletion of up to two amino acids in CDR2, wherein the sequence of CDR1 and CDR3 is unmodified.

Embodiment 269: The VHH antibody domain or fragment thereof according to any one of embodiments 250 to 260, wherein the modification in (f) is
the replacement, addition or deletion of up to one amino acid in CDR1;
the replacement, addition or deletion of up to one amino acid in CDR2; and/or
the replacement, addition or deletion of up to one amino acid in CDR3.

Embodiment 270: The VHH antibody domain or fragment thereof according to any one of embodiments 250 to 260, wherein the modification in (f) is
the replacement, addition or deletion of one amino acid in CDR1 and/or
the replacement, addition or deletion of one amino acid in CDR2; wherein the sequence of CDR3 is unmodified.

Embodiment 271: The VHH antibody domain or fragment thereof according to any one of embodiments 250 to 260, wherein the modification in (f) is
the replacement, addition or deletion of one amino acid in CDR1, wherein the sequence of CDR2 and CDR3 is unmodified.

Embodiment 272: The VHH antibody domain or fragment thereof according to any one of embodiments 250 to 260, wherein the modification in (f) is
the replacement, addition or deletion of one amino acid in CDR2, wherein the sequence of CDR1 and CDR3 is unmodified.

Embodiment 273: The VHH antibody domain or fragment thereof according to any one of embodiments 250 to 272, wherein the modification in (f) comprises only the replacement, but not the addition or deletion of amino acids.

Embodiment 274: The VHH antibody domain or fragment thereof according to any one of embodiments 250 to 273, wherein said replacement is a conservative amino acid replacement.

Embodiment 275: The VHH antibody domain or fragment thereof according to any one of embodiments 189 to 274, wherein said fragment consists of at least 100 amino acids.

Embodiment 276: The VHH antibody domain or fragment thereof according to any one of embodiments 189 to 274, wherein said fragment consists of at least 105 amino acids.

Embodiment 277: The VHH antibody domain or fragment thereof according to any one of embodiments 189 to 274, wherein said fragment consists of at least 110 amino acids.

Embodiment 278: The VHH antibody domain or fragment thereof according to any one of embodiments 189 to 274, wherein said fragment consists of at least 115 amino acids.

Embodiment 279: The VHH antibody domain or fragment thereof according to any one of embodiments 189 to 278, wherein said VHH antibody domain is an anti-IL-18Rβ VHH antibody domain.

Embodiment 280: The VHH antibody domain or fragment thereof according to any one of embodiments 189 to 279, wherein said VHH antibody domain is specific for IL-18Rβ.

Embodiment 281: The VHH antibody domain or fragment thereof according to any one of embodiments 189 to 280, wherein said VHH antibody domain or fragment thereof binds to IL-18Rβ.

Embodiment 282: The VHH antibody domain or fragment thereof according to any one of embodiments 189 to 281, wherein said VHH antibody domain or fragment thereof binds specifically to IL-18Rβ.

Embodiment 283: The VHH antibody domain or fragment thereof according to any one of embodiments 189 to 282, wherein said VHH antibody domain or fragment thereof does not bind to IL-18Rα.

Embodiment 284: The VHH antibody domain or fragment thereof according to any one of embodiments 189 to 283, wherein said VHH antibody domain or fragment thereof is capable of specifically binding to IL-18RβECD.

Embodiment 285: The VHH antibody domain or fragment thereof according to any one of embodiments 189 to 284, wherein said VHH antibody domain or fragment thereof binds to recombinant human IL-18RβECD with a KD of 1×10⁻⁶ M or stronger.

Embodiment 286 The VHH antibody domain or fragment thereof according to any one of embodiments 189 to 284, wherein said VHH antibody domain or fragment thereof binds to recombinant human IL-18RβECD with a KD of 1×10⁻⁷ M or stronger.

Embodiment 287: The VHH antibody domain or fragment thereof according to any one of embodiments 189 to 284, wherein said VHH antibody domain or fragment thereof binds to recombinant human IL-18RβECD with a KD of 5×10⁻⁸ M or stronger.

Embodiment 288: The VHH antibody domain or fragment thereof according to any one of embodiments 189 to 284, wherein said VHH antibody domain or fragment thereof binds to recombinant human IL-18RβECD with a KD of 1.5×10⁻⁸ M or stronger.

Embodiment 289: The VHH antibody domain or fragment thereof according to any one of embodiments 189 to 284, wherein said VHH antibody domain or fragment thereof binds to recombinant human IL-18RβECD with a KD of 1×10⁻⁸ M or stronger.

Embodiment 290: The VHH antibody domain or fragment thereof according to any one of embodiments 189 to 284, wherein said VHH antibody domain or fragment thereof binds to recombinant human IL-18RβECD with a KD of 5×10⁻⁹ M or stronger.

Embodiment 291: The VHH antibody domain or fragment thereof according to any one of embodiments 189 to 284, wherein said VHH antibody domain or fragment thereof binds to recombinant human IL-18RβECD with a KD of 1×10⁻⁹ M or stronger.

Embodiment 292: The VHH antibody domain or fragment thereof according to any one of embodiments 189 to 291, wherein said fragment of said VHH antibody domain binds to recombinant human IL-18RβECD with at least 90% of the affinity (as determined by KD value) with which said VHH antibody domain binds to recombinant human IL-18RβECD.

Embodiment 293: The VHH antibody domain or fragment thereof according to any one of embodiments 189 to 292, wherein said fragment of said VHH antibody domain binds to IL-18Rα ECD with an affinity (as determined from the KD value) that is at least equal to the affinity with which VHH2 (SEQ ID NO: 2) binds to IL-18Rα ECD or stronger. Such binding can be determined by in vitro binding experiments as described in Example 1 below (by biolayer interferometry).

Embodiment 294: The VHH antibody domain or fragment thereof according to any one of embodiments 189 to 211 or 250 to 293, wherein in (e) and (f) the VHH antibody domain or fragment thereof binds to recombinant human IL-18RβECD with an affinity (KD value) that is by not more than a factor of 10 weaker than the binding of the corresponding VHH antibody domain without modification.

Embodiment 295: The VHH antibody domain or fragment thereof according to any one of embodiments 189 to 211 or 250 to 293, wherein in (e) and (f) the VHH antibody domain or fragment thereof binds to recombinant human IL-18RβECD with an affinity (KD value) that is by not more than a factor of 5 weaker than the binding of the corresponding VHH antibody domain without modification.

Embodiment 296: The VHH antibody domain or fragment thereof according to any one of embodiments 189 to 211 or 250 to 293, wherein in (e) and (f) the VHH antibody domain or fragment thereof binds to recombinant human IL-18RβECD with an affinity (KD value) that is by not more than a factor of 2 weaker than the binding of the corresponding VHH antibody domain without modification.

Embodiment 297: The VHH antibody domain or fragment thereof according to any one of embodiments 189 to 211 or 250 to 293, wherein in (e) and (f) the VHH antibody domain or fragment thereof binds to recombinant human IL-18RβECD with an affinity (KD value) that is by not more than a factor of 1.5 weaker than the binding of the corresponding VHH antibody domain without modification.

Embodiment 298: The VHH antibody domain or fragment thereof according to any one of embodiments 189 to 211 or 250 to 297, wherein in (e) and (f) the VHH antibody domain or fragment thereof binds to recombinant human IL-18RβECD with an affinity (KD value) that is by not more than a factor of 10 stronger than the binding of the corresponding VHH antibody domain without modification.

Embodiment 299: The VHH antibody domain or fragment thereof according to any one of embodiments 189 to 211 or 250 to 297, wherein in (e) and (f) the VHH antibody domain or fragment thereof binds to recombinant human IL-18RβECD with an affinity (KD value) that is by not more than a factor of 5 stronger than the binding of the corresponding VHH antibody domain without modification.

Embodiment 300: The VHH antibody domain or fragment thereof according to any one of embodiments 189 to 211 or 250 to 297, wherein in (e) and (f) the VHH antibody domain or fragment thereof binds to recombinant human IL-18RβECD with an affinity (KD value) that is by not more than a factor of 2 stronger than the binding of the corresponding VHH antibody domain without modification.

Embodiment 301: The VHH antibody domain or fragment thereof according to any one of embodiments 189 to 211 or 250 to 297, wherein in (e) and (f) the VHH antibody domain or fragment thereof binds to recombinant human IL-18RβECD with an affinity (KD value) that is by not more than a factor of 1.5 stronger than the binding of the corresponding VHH antibody domain without modification.

Embodiment 302: The VHH antibody domain or fragment thereof according to any one of embodiments 212 to 301, wherein in (F) and (G) the VHH antibody domain binds to recombinant human IL-18RβECD with an affinity (KD value) that is by not more than a factor of 10 weaker than the binding of the corresponding VHH antibody domain without modification.

Embodiment 303: The VHH antibody domain or fragment thereof according to any one of embodiments 212 to 301, wherein in (F) and (G) the VHH antibody domain binds to recombinant human IL-18RβECD with an affinity (KD value) that is by not more than a factor of 5 weaker than the binding of the corresponding VHH antibody domain without modification.

Embodiment 304: The VHH antibody domain or fragment thereof according to any one of embodiments 212 to 301, wherein in (F) and (G) the VHH antibody domain binds to recombinant human IL-18RβECD with an affinity (KD value) that is by not more than a factor of 2 weaker than the binding of the corresponding VHH antibody domain without modification.

Embodiment 305: The VHH antibody domain or fragment thereof according to any one of embodiments 189 to 301, wherein in (F) and (G) the VHH antibody domain binds to recombinant human IL-18RβECD with an affinity (KD value) that is by not more than a factor of 1.5 weaker than the binding of the corresponding VHH antibody domain without modification.

Embodiment 306: The VHH antibody domain or fragment thereof according to any one of embodiments 212 to 305, wherein in (F) and (G) the VHH antibody domain binds to recombinant human IL-18RβECD with an affinity (KD value) that is by not more than a factor of 10 stronger than the binding of the corresponding VHH antibody domain without modification.

Embodiment 307: The VHH antibody domain or fragment thereof according to any one of embodiments 212 to 305, wherein in (F) and (G) the VHH antibody domain binds to recombinant human IL-18RβECD with an affinity (KD value) that is by not more than a factor of 5 stronger than the binding of the corresponding VHH antibody domain without modification.

Embodiment 308: The VHH antibody domain or fragment thereof according to any one of embodiments 212 to 305, wherein in (F) and (G) the VHH antibody domain binds to recombinant human IL-18RβECD with an affinity (KD value) that is by not more than a factor of 2 stronger than the binding of the corresponding VHH antibody domain without modification.

Embodiment 309: The VHH antibody domain or fragment thereof according to any one of embodiments 212 to 305, wherein in (F) and (G) the VHH antibody domain binds to recombinant human IL-18RβECD with an affinity (KD value) that is by not more than a factor of 1.5 stronger than the binding of the corresponding VHH antibody domain without modification.

Embodiment 310: The VHH antibody domain or fragment thereof according to any one of embodiments 212 to 309, wherein in (H) the affinity (KD value) of the binding of the VHH antibody domain to recombinant human IL-18RβECD is by not more than a factor of 10 weaker than the affinity (KD value) of the binding to recombinant human IL-18Rβ ECD of a VHH antibody domain consisting of the sequence from the Table of VHH Sequences that has the highest degree of sequence identity with the sequence of said VHH antibody domain of (H). The degree of sequence identity can be determined by sequence alignment.

Embodiment 311: The VHH antibody domain or fragment thereof according to any one of embodiments 212 to 309, wherein in (H) the affinity (KD value) of the binding of the VHH antibody domain to recombinant human IL-18RβECD is by not more than a factor of 5 weaker than the affinity (KD value) of the binding to recombinant human IL-18Rβ ECD of a VHH antibody domain consisting of the sequence from the Table of VHH Sequences that has the highest degree of sequence identity with the sequence of said VHH antibody domain of (H). The degree of sequence identity can be determined by sequence alignment.

Embodiment 312: The VHH antibody domain or fragment thereof according to any one of embodiments 212 to 309, wherein in (H) the affinity (KD value) of the binding of the VHH antibody domain to recombinant human IL-18RβECD is by not more than a factor of 2 weaker than the affinity (KD value) of the binding to recombinant human IL-18Rβ ECD of a VHH antibody domain consisting of the sequence from the Table of VHH Sequences that has the highest degree of sequence identity with the sequence of said VHH antibody domain of (H).

Embodiment 313: The VHH antibody domain or fragment thereof according to any one of embodiments 212 to 309, wherein in (H) the affinity (KD value) of the binding of the VHH antibody domain to recombinant human IL-18Rβ ECD is by not more than a factor of 1.5 weaker than the affinity (KD value) of the binding to recombinant human IL-18RβECD of a VHH antibody domain consisting of the sequence from the Table of VHH Sequences that has the highest degree of sequence identity with the sequence of said VHH antibody domain of (H).

Embodiment 314 The VHH antibody domain or fragment thereof according to any one of embodiments 212 to 313, wherein in (H) the affinity (KD value) of the binding of the VHH antibody domain to recombinant human IL-18Rβ ECD is by not more than a factor of 10 stronger than the affinity (KD value) of the binding to recombinant human IL-18RβECD of a VHH antibody domain consisting of the sequence from the Table of VHH Sequences that has the highest degree of sequence identity with the sequence of said VHH antibody domain of (H).

Embodiment 315: The VHH antibody domain or fragment thereof according to any one of embodiments 212 to 313, wherein in (H) the affinity (KD value) of the binding of the VHH antibody domain to recombinant human IL-18Rβ ECD is by not more than a factor of 5 stronger than the affinity (KD value) of the binding to recombinant human IL-18Rβ ECD of a VHH antibody domain consisting of the sequence from the Table of VHH Sequences that has the highest degree of sequence identity with the sequence of said VHH antibody domain of (H).

Embodiment 316: The VHH antibody domain or fragment thereof according to any one of embodiments 212 to 313, wherein in (H) the affinity (KD value) of the binding of the VHH antibody domain to recombinant human IL-18Rβ ECD is by not more than a factor of 2 stronger than the affinity (KD value) of the binding to recombinant human IL-18Rβ ECD of a VHH antibody domain consisting of the sequence from the Table of VHH Sequences that has the highest degree of sequence identity with the sequence of said VHH antibody domain of (H).

Embodiment 317: The VHH antibody domain or fragment thereof according to any one of embodiments 212 to 313, wherein in (H) the affinity (KD value) of the binding of the VHH antibody domain to recombinant human IL-18Rβ ECD is by not more than a factor of 1.5 stronger than the affinity (KD value) of the binding to recombinant human IL-18RβECD of a VHH antibody domain consisting of the sequence from the Table of VHH Sequences that has the highest degree of sequence identity with the sequence of said VHH antibody domain of (H).

Embodiment 318: The VHH antibody domain or fragment thereof according to any one of embodiments 279 to 317, wherein said KD value is determined in in vitro binding experiments through biolayer interferometry.

Embodiment 319: The VHH antibody domain or fragment thereof according to any one of embodiments 279 to 318, wherein said KD value is measured by kinetic measurements by biolayer interferometry at 25 °C and 1000 rpm in KB Buffer (PBS+0.1% Tween-20+1% BSA).

Embodiment 320: The VHH antibody domain or fragment thereof according to any one of embodiments 212 to 319, wherein said VHH antibody domain of (E) comprises one VHH sequence selected from the group consisting of VHH12, VHH13, VHH14, VHH15, VHH17, VHH18, VHH20, VHH21 and VHH22 shown in the Table of VHH Sequences.

Embodiment 321: The VHH antibody domain or fragment thereof according to any one of embodiments 212 to 319, wherein said VHH antibody domain of (E) comprises one VHH sequence selected from the group consisting of VHH12, VHH14, VHH15, VHH17 and VHH22 shown in the Table of VHH Sequences.

Embodiment 322: The VHH antibody domain or fragment thereof according to any one of embodiments 212 to 319, wherein said VHH antibody domain of (E) comprises one VHH sequence selected from the group consisting of VHH6 and VHH11 shown in the Table of VHH Sequences.

Embodiment 323: The VHH antibody domain or fragment thereof according to any one of embodiments 212 to 319, wherein said VHH antibody domain of (E) comprises one VHH sequence selected from the group consisting of VHH13, VHH16 and VHH22 shown in the Table of VHH Sequences.

Embodiment 324: The VHH antibody domain or fragment thereof according to any one of embodiments 212 to 319, wherein said VHH antibody domain of (E) comprises one VHH sequence selected from the group consisting of VHH12, VHH13, VHH15, VHH17, VHH18 and VHH21 shown in the Table of VHH Sequences.

Embodiment 325: The VHH antibody domain or fragment thereof according to any one of embodiments 212 to 319, wherein said VHH antibody domain of (E) comprises one VHH sequence selected from the group consisting of VHH15 and VHH17 shown in the Table of VHH Sequences.

Embodiment 326: The VHH antibody domain or fragment thereof according to any one of embodiments 212 to 319, wherein said VHH antibody domain of (E) comprises the VHH sequence VHH12 shown in the Table of VHH Sequences.

Embodiment 327: The VHH antibody domain or fragment thereof according to any one of embodiments 212 to 319, wherein said VHH antibody domain of (E) comprises the VHH sequence VHH13 shown in the Table of VHH Sequences.

Embodiment 328: The VHH antibody domain or fragment thereof according to any one of embodiments 212 to 319, wherein said VHH antibody domain of (E) comprises the VHH sequence VHH14 shown in the Table of VHH Sequences.

Embodiment 329: The VHH antibody domain or fragment thereof according to any one of embodiments 212 to 319, wherein said VHH antibody domain of (E) comprises the VHH sequence VHH15 shown in the Table of VHH Sequences.

Embodiment 330: The VHH antibody domain or fragment thereof according to any one of embodiments 212 to 319, wherein said VHH antibody domain of (E) comprises the VHH sequence VHH16 shown in the Table of VHH Sequences.

Embodiment 331: The VHH antibody domain or fragment thereof according to any one of embodiments 212 to 319, wherein said VHH antibody domain of (E) comprises the VHH sequence VHH17 shown in the Table of VHH Sequences.

Embodiment 332: The VHH antibody domain or fragment thereof according to any one of embodiments 212 to 319, wherein said VHH antibody domain of (E) comprises the VHH sequence VHH18 shown in the Table of VHH Sequences.

Embodiment 333: The VHH antibody domain or fragment thereof according to any one of embodiments 212 to 319, wherein said VHH antibody domain of (E) comprises the VHH sequence VHH20 shown in the Table of VHH Sequences.

Embodiment 334: The VHH antibody domain or fragment thereof according to any one of embodiments 212 to 319, wherein said VHH antibody domain of (E) comprises the VHH sequence VHH21 shown in the Table of VHH Sequences.

Embodiment 335: The VHH antibody domain or fragment thereof according to any one of embodiments 212 to 319, wherein said VHH antibody domain of (E) comprises the VHH sequence VHH22 shown in the Table of VHH Sequences.

Embodiment 336: The VHH antibody domain or fragment thereof according to any one of embodiments 189 to 211 or 250 to 335, wherein in (d) said VHH antibody domain or fragment thereof comprises the complementarity determining regions CDR1, CDR2 and CDR3 of one VHH selected from the group consisting of VHH12, VHH13, VHH14, VHH15, VHH17, VHH18, VHH20, VHH21 or VHH22 as shown in the Table of CDRs.

Embodiment 337: The VHH antibody domain or fragment thereof according to any one of embodiments 189 to 211 or 250 to 335, wherein in (d) said VHH antibody domain or fragment thereof comprises the complementarity determining regions CDR1, CDR2 and CDR3 of one VHH selected from the group consisting of VHH12, VHH14, VHH15, VHH17 or VHH22 as shown in the Table of CDRs.

Embodiment 338: The VHH antibody domain or fragment thereof according to any one of embodiments 189 to 211 or 250 to 335, wherein in (d) said VHH antibody domain or fragment thereof comprises the complementarity determining regions CDR1, CDR2 and CDR3 of VHH6 or VHH11 as shown in the Table of CDRs.

Embodiment 339: The VHH antibody domain or fragment thereof according to any one of embodiments 189 to 211 or 250 to 335, wherein in (d) said VHH antibody domain or fragment thereof comprises the complementarity determining regions CDR1, CDR2 and CDR3 of one VHH selected from the group consisting of VHH13, VHH16 or VHH22 as shown in the Table of CDRs.

Embodiment 340: The VHH antibody domain or fragment thereof according to any one of embodiments 189 to 211 or 250 to 335, wherein in (d) said VHH antibody domain or fragment thereof comprises the complementarity determining regions CDR1, CDR2 and CDR3 of one VHH selected from the group consisting of VHH12, VHH13, VHH15, VHH17, VHH18 or VHH21 as shown in the Table of CDRs.

Embodiment 341: The VHH antibody domain or fragment thereof according to any one of embodiments 189 to 211 or 250 to 335, wherein in (d) said VHH antibody domain or fragment thereof comprises the complementarity determining regions CDR1, CDR2 and CDR3 of one VHH selected from the group consisting of VHH15 or VHH17 as shown in the Table of CDRs.

Embodiment 342: The VHH antibody domain or fragment thereof according to any one of embodiments 189 to 211 or 250 to 335, wherein in (d) said VHH antibody domain or fragment thereof comprises the complementarity determining regions CDR1, CDR2 and CDR3 of VHH12 as shown in the Table of CDRs.

Embodiment 343: The VHH antibody domain or fragment thereof according to any one of embodiments 189 to 211 or 250 to 335, wherein in (d) said VHH antibody domain or fragment thereof comprises the complementarity determining regions CDR1, CDR2 and CDR3 of VHH13 as shown in the Table of CDRs.

Embodiment 344: The VHH antibody domain or fragment thereof according to any one of embodiments 189 to 211 or 250 to 335, wherein in (d) said VHH antibody domain or fragment thereof comprises the complementarity determining regions CDR1, CDR2 and CDR3 of VHH14 as shown in the Table of CDRs.

Embodiment 345: The VHH antibody domain or fragment thereof according to any one of embodiments 189 to 211 or 250 to 335, wherein in (d) said VHH antibody domain or fragment thereof comprises the complementarity determining regions CDR1, CDR2 and CDR3 of VHH15 as shown in the Table of CDRs.

Embodiment 346: The VHH antibody domain or fragment thereof according to any one of embodiments 189 to 211 or 250 to 335, wherein in (d) said VHH antibody domain or fragment thereof comprises the complementarity determining regions CDR1, CDR2 and CDR3 of VHH16 as shown in the Table of CDRs.

Embodiment 347: The VHH antibody domain or fragment thereof according to any one of embodiments 189 to 211 or 250 to 335, wherein in (d) said VHH antibody domain or fragment thereof comprises the complementarity determining regions CDR1, CDR2 and CDR3 of VHH17 as shown in the Table of CDRs.

Embodiment 348: The VHH antibody domain or fragment thereof according to any one of embodiments 189 to 211 or 250 to 335, wherein in (d) said VHH antibody domain or fragment thereof comprises the complementarity determining regions CDR1, CDR2 and CDR3 of VHH18 as shown in the Table of CDRs.

Embodiment 349: The VHH antibody domain or fragment thereof according to any one of embodiments 189 to 211 or 250 to 335, wherein in (d) said VHH antibody domain or fragment thereof comprises the complementarity determining regions CDR1, CDR2 and CDR3 of VHH20 as shown in the Table of CDRs.

Embodiment 350: The VHH antibody domain or fragment thereof according to any one of embodiments 189 to 211 or 250 to 335, wherein in (d) said VHH antibody domain or fragment thereof comprises the complementarity determining regions CDR1, CDR2 and CDR3 of VHH21 as shown in the Table of CDRs.

Embodiment 351: The VHH antibody domain or fragment thereof according to any one of embodiments 189 to 211 or 250 to 335, wherein in (d) said VHH antibody domain or fragment thereof comprises the complementarity determining regions CDR1, CDR2 and CDR3 of VHH22 as shown in the Table of CDRs.

Embodiment 352: The VHH antibody domain or fragment thereof according to any one of embodiments 189 to 351, wherein said VHH antibody domain or fragment thereof competes with VHH15 for binding to recombinant human IL-18RβECD.

Embodiment 353: The VHH antibody domain or fragment thereof according to any one of embodiments 189 to 351, wherein said VHH antibody domain or fragment thereof partially competes with VHH15 for binding to recombinant human IL-18RβECD.

Embodiment 354: The VHH antibody domain or fragment thereof according to any one of embodiments 189 to 353, wherein said VHH antibody domain or fragment thereof does not compete with VHH2 for binding to recombinant human IL-18Rα ECD.

Embodiment 355: The VHH antibody domain or fragment thereof according to any one of embodiments 189 to 354, wherein said humanization is by germlining.

Embodiment 356: The VHH antibody domain or fragment thereof according to embodiment 355, wherein said germlining involves
identifying the human VH germline sequence that is closest to the sequence to be humanized with respect to its sequence similarity; and
replacing amino acid(s) in the sequence to be humanized by the amino acid(s) at the corresponding sequence position(s) in the closest human VH germline sequence.

Sequence similarity and corresponding sequence positions can be determined by sequence alignment as described above.

Embodiment 357: The VHH antibody domain or fragment thereof according to embodiment 355, wherein said germlining consists of
identifying the human VH germline sequence that is closest to the sequence to be humanized with respect to its sequence similarity; and
replacing amino acid(s) in the sequence to be humanized by the amino acid(s) at the corresponding sequence position(s) in the closest human VH germline sequence.

Embodiment 358: The VHH antibody domain or fragment thereof according to any one of embodiments 189 to 357, wherein said VHH antibody domain (resp. fragment thereof) with humanization binds to human IL-18RβECD with an affinity (KD value) that is not weaker by a factor of more than 10 compared to the binding of a corresponding VHH antibody domain (resp. fragment thereof) without humanization to human IL-18RβECD.

As a skilled person understands, the term “corresponding VHH antibody domain without humanization” refers to a VHH antibody domain that differs only by the amino acid changes introduced in order to carry out said humanization, but that is otherwise identical.

Embodiment 359: The VHH antibody domain or fragment thereof according to any one of embodiments 189 to 357, wherein said VHH antibody domain (resp. fragment thereof) with humanization binds to human IL-18RβECD with an affinity (KD value) that is not weaker by a factor of more than 5 compared to the binding of a corresponding VHH antibody domain (resp. fragment thereof) without humanization to human IL-18RβECD.

Embodiment 360: The VHH antibody domain or fragment thereof according to any one of embodiments 189 to 357, wherein said VHH antibody domain (resp. fragment thereof) with humanization binds to human IL-18RβECD with an affinity (KD value) that is not weaker by a factor of more than 2 compared to the binding of a corresponding VHH antibody domain (resp. fragment thereof) without humanization to human IL-18RβECD.

Embodiment 361: The VHH antibody domain or fragment thereof according to any one of embodiments 189 to 357, wherein said VHH antibody domain (resp. fragment thereof) with humanization binds to human IL-18RβECD with an affinity (KD value) that is not weaker than the binding of a corresponding VHH antibody domain (resp. fragment thereof) without humanization to human IL-18RβECD.

Embodiment 362: The VHH antibody domain or fragment thereof according to any one of embodiments 358 to 361, wherein said affinity is determined by KD measurement.

Embodiment 363: The VHH antibody domain or fragment thereof according to any one of embodiments 212 to 362, wherein said VHH antibody domain comprises a VHH antibody domain according to (E), (F) or (G)/said fragment of a VHH antibody domain comprises a fragment of a VHH antibody domain according to (E), (F) or (G).

Embodiment 364: The VHH antibody domain or fragment thereof according to any one of embodiments 212 to 362, wherein said VHH antibody domain comprises a VHH antibody domain according to (E), (F) or (H)/said fragment of a VHH antibody domain comprises a fragment of a VHH antibody domain according to (E), (F) or (H).

Embodiment 365: The VHH antibody domain or fragment thereof according to any one of embodiments 212 to 362, wherein said VHH antibody domain comprises a VHH antibody domain according to (E), (G) or (H)/said fragment of a VHH antibody domain comprises a fragment of a VHH antibody domain according to (E), (G) or (H).

Embodiment 366: The VHH antibody domain or fragment thereof according to any one of embodiments 212 to 362, wherein said VHH antibody domain comprises a VHH antibody domain according to (E) or (F)/said fragment of a VHH antibody domain comprises a fragment of a VHH antibody domain according to (E) or (F).

Embodiment 367: The VHH antibody domain or fragment thereof according to any one of embodiments 212 to 362, wherein said VHH antibody domain comprises a VHH antibody domain according to (E) or (G)/said fragment of a VHH antibody domain comprises a fragment of a VHH antibody domain according to (E) or (G).

Embodiment 368: The VHH antibody domain or fragment thereof according to any one of embodiments 212 to 362, wherein said VHH antibody domain comprises a VHH antibody domain according to (E) or (H)/said fragment of a VHH antibody domain comprises a fragment of a VHH antibody domain according to (E) or (H).

Embodiment 369: The VHH antibody domain or fragment thereof according to any one of embodiments 212 to 362, wherein said VHH antibody domain comprises a VHH antibody domain according to (E)/said fragment of a VHH antibody domain comprises a fragment of a VHH antibody domain according to (E).

Embodiment 370: The VHH antibody domain or fragment thereof according to any one of embodiments 212 to 362, wherein said VHH antibody domain comprises a VHH antibody domain according to (F)/said fragment of a VHH antibody domain comprises a fragment of a VHH antibody domain according to (F).

Embodiment 371: The VHH antibody domain or fragment thereof according to any one of embodiments 189 to 211 or 250 to 370, wherein said VHH antibody domain or fragment thereof comprises complementarity determining regions according to (d) or (e).

Embodiment 372: The VHH antibody domain or fragment thereof according to any one of embodiments 189 to 211 or 250 to 370, wherein said VHH antibody domain or fragment thereof comprises complementarity determining regions according to (d) or (f).

Embodiment 373: The VHH antibody domain or fragment thereof according to any one of embodiments 189 to 211 or 250 to 370, wherein said VHH antibody domain or fragment thereof comprises complementarity determining regions according to (d).

Embodiment 374: The VHH antibody domain or fragment thereof according to any one of embodiments 189 to 211 or 250 to 370, wherein said VHH antibody domain or fragment thereof comprises complementarity determining regions according to (e).

Seventh aspect of the present disclosure (also referred to as “Embodiment 375”): According to a seventh aspect, the present disclosure relates to a compound comprising
a VHH antibody domain or fragment thereof according to any one of embodiments 1 to 188.

Eighth aspect of the present disclosure (also referred to as “Embodiment 376”): According to a eighth aspect, the present disclosure relates to a compound comprising
a VHH antibody domain or fragment thereof according to any one of claims 189 to 374.

Nineth aspect of the present disclosure (also referred to as “Embodiment 377”): According to a nineth aspect, the present disclosure relates to a compound comprising
a first binding module which is a VHH antibody domain or fragment thereof, wherein
   (a) said VHH antibody domain or fragment thereof comprises the complementarity determining regions CDR1, CDR2 and CDR3 of one VHH selected from the group consisting of VHH1, VHH2, VHH3, VHH4, VHH5, VHH6, VHH8, VHH9, VHH10 and VHH11 as shown in the Table of CDR;
   (b) said VHH antibody domain or fragment thereof comprises the complementarity determining regions CDR1, CDR2 and CDR3 as defined in (a) with modification, wherein the modification is that the sequence of at least one of CDR1, CDR2 and CDR3 is humanized; or
   (c) said VHH antibody domain or fragment thereof comprises the complementarity determining regions CDR1, CDR2 and CDR3 as defined in (a) with modification, wherein the modification is
      the replacement, addition or deletion of up to three amino acids in CDR1,
      the replacement, addition or deletion of up to three amino acids in CDR2 and/or
      the replacement, addition or deletion of up to three amino acids in CDR3;
      a second binding module which is a VHH antibody domain or a fragment thereof, wherein
   (d) said VHH antibody domain or fragment thereof comprises the complementarity determining regions CDR1, CDR2 and CDR3 of one VHH selected from the group consisting of VHH12, VHH13, VHH14, VHH15, VHH16, VHH17, VHH18, VHH20, VHH21 and VHH22 as shown in the Table of CDRs;
   (e) said VHH antibody domain or fragment thereof comprises the complementarity determining regions CDR1, CDR2 and CDR3 as defined in
(d) with modification, wherein the modification is that the sequence of at least one of CDR1, CDR2 and CDR3 is humanized; or
(f) said VHH antibody domain or fragment thereof comprises the complementarity determining regions CDR1, CDR2 and CDR3 as defined in
(d) with modification, wherein the modification is
   the replacement, addition or deletion of up to three amino acids in CDR1,
   the replacement, addition or deletion of up to three amino acids in CDR2 and/or
   the replacement, addition or deletion of up to three amino acids in CDR3.

To the seventh to nineth aspect of the present disclosure and the embodiments referring thereto, the same explanations and definitions apply accordingly as for the first to sixth aspects of the present disclosure and the embodiments referring thereto.

For embodiment 377 and embodiments referring thereto, with respect to said first binding module further embodiments of said VHH antibody domain or fragment thereof of said first binding module are as defined in embodiments 1 to 188.

For embodiment 377 and embodiments referring thereto, with respect to said second binding module further embodiments of said VHH antibody domain or fragment thereof of said second binding module are as defined in embodiments 189 to 374.

Tenth aspect of the present disclosure (also referred to as “Embodiment 378”): According to a tenth aspect, the present disclosure relates to a compound comprising
a first binding module which is a VHH antibody domain or fragment thereof, wherein
   (A) said VHH antibody domain comprises the amino acid sequence of a VHH selected from the group consisting of VHH1, VHH2, VHH3, VHH4, VHH5, VHH6, VHH8, VHH9, VHH10 and VHH11 as shown in the Table of VHH Sequences;
   (B) said VHH antibody domain comprises a VHH sequence as defined in (A) with modification, wherein the modification is that said sequence is humanized;
   (C) said VHH antibody domain comprises a VHH sequence as defined in (A) with modification, wherein the modification is the replacement, addition or deletion of up to 25 amino acids; or
   (D) said VHH antibody domain comprises a VHH sequence that is at least 75% identical to a VHH sequence referred to in (A);
a second binding module which is a VHH antibody domain or a fragment thereof, wherein
   (E) said VHH antibody domain comprises the amino acid sequence of a VHH selected from the group consisting of VHH12, VHH13, VHH14, VHH15, VHH16, VHH17, VHH18, VHH20, VHH21 and VHH22 as shown in the Table of VHH Sequences;
   (F) said VHH antibody domain comprises a VHH sequence as defined in (E) with modification, wherein the modification is that said sequence is humanized;
   (G) said VHH antibody domain comprises a VHH sequence as defined in (E) with modification, wherein the modification is the replacement, addition or deletion of up to 25 amino acids; or
   (H) said VHH antibody domain comprises a VHH sequence that is at least 75% identical to a VHH sequence referred to in (E).

To the tenth aspect of the present disclosure and the embodiments referring thereto, the same explanations and definitions apply accordingly as for the first to nineth aspects of the present disclosure and the embodiments referring thereto.

For embodiment 378 and embodiments referring thereto, with respect to said first binding module further embodiments of said VHH antibody domain or fragment thereof of said first binding module are as defined in embodiments 1 to 188.

For embodiment 378 and embodiments referring thereto, with respect to said second binding module further embodiments of said VHH antibody domain or fragment thereof of said second binding module are as defined in embodiments 189 to 374.

Eleventh aspect of the present disclosure (also referred to as “Embodiment 379”): According to an eleventh aspect, the present disclosure relates to a compound comprising
a first binding module which is a VHH antibody domain or fragment thereof, wherein
   (A) said VHH antibody domain consists of the amino acid sequence of a VHH selected from the group consisting of VHH1, VHH2, VHH3, VHH4, VHH5, VHH6, VHH8, VHH9, VHH10 and VHH11 as shown in the Table of VHH Sequences;
   (B) said VHH antibody domain consists of a VHH sequence as defined in (A) with modification, wherein the modification is that said sequence is humanized;
   (C) said VHH antibody domain consists of a VHH sequence as defined in (A) with modification, wherein the modification is the replacement, addition or deletion of up to 25 amino acids; or
   (D) said VHH antibody domain consists of a VHH sequence that is at least 75% identical to a VHH sequence referred to in (A);
a second binding module which is a VHH antibody domain or a fragment thereof, wherein
   (E) said VHH antibody domain consists of the amino acid sequence of a VHH selected from the group consisting of VHH12, VHH13, VHH14, VHH15, VHH16, VHH17, VHH18, VHH20, VHH21 and VHH22 as shown in the Table of VHH Sequences;
   (F) said VHH antibody domain consists of a VHH sequence as defined in (E) with modification, wherein the modification is that said sequence is humanized;
   (G) said VHH antibody domain consists of a VHH sequence as defined in (E) with modification, wherein the modification is the replacement, addition or deletion of up to 25 amino acids; or
   (H) said VHH antibody domain consists of a VHH sequence that is at least 75% identical to a VHH sequence referred to in (E).

To the eleventh aspect of the present disclosure and the embodiments referring thereto, the same explanations and definitions apply accordingly as for the first to tenth aspects of the present disclosure and the embodiments referring thereto.

For embodiment 379 and embodiments referring thereto, with respect to said first binding module further embodiments of said VHH antibody domain or fragment thereof of said first binding module are as defined in embodiments 1 to 188.

For embodiment 379 and embodiments referring thereto, with respect to said second binding module further embodiments of said VHH antibody domain or fragment thereof of said second binding module are as defined in embodiments 189 to 374.

“Compound”, as used in the present disclosure, is not particularly limited and refers to a chemical entity of any chemical class, provided that it includes the binding module(s) as defined above. Thus, the compound can e.g. be an organic compound or a compound composed of an organic and an inorganic part, it can be a protein composed of a single amino acid chain, a protein composed of multiple amino acid chains that are either non-covalently or covalently associated, or a non-covalent complex including an inorganic component. The compound can consist of the amino acid sequence of the binding module(s) referred to in the above embodiments alone or it can in addition include further amino acid(s) that may be covalently or non-covalently attached, or it can be associated with inorganic components. Preferably, the compound is a molecule.

For example, in embodiment 375, with optional modifications from embodiments 1 to 188 and further embodiments referring to embodiment 375, (resp. in embodiment 376, with optional modifications from embodiments 189 to 374 and further embodiments referring to embodiment 376), the compound can be e.g. a monovalent, monospecific antibody molecule in which an anti-IL-18Rα VHH according to the present disclosure (resp. an anti-IL-18Rβ VHH according to the present disclosure) is linked covalently to an Fc region of an IgG. Alternatively, it could be a bispecific molecule in which an anti-IL-18Rα VHH according to the present disclosure (resp. an anti-IL-18RβVHH according to the present disclosure) is linked covalently to an IgG1 antibody lacking one of its “arms”, such that the molecule has two binding modules, namely the anti-IL-18Rα VHH according to the present disclosure (resp. anti-IL-18RβVHH according to the present disclosure) and the remaining “original” binding module of the “one-armed” IgG1 antibody. Or in another example, the compound can be a bispecific molecule comprising the anti-IL-18Rα VHH according to the present disclosure (resp. anti-IL-18RβVHH according to the present disclosure) and a targeting module.

For example, in embodiments 377 to 379 (with optional modifications for the first binding module defined in embodiments 1 to 188 and optional modifications for the second binding module defined in embodiments 189 to 374, with further optional modifications defined in the embodiments referring to embodiments 377 to 379), the compound may e.g. be an antibody with two binding modules (an anti-IL-18Rα VHH according to the present disclosure and an anti-IL-18RβVHH according to the present disclosure) prepared in the SEED format, resulting in a bispecific antibody with a structure e.g. as shown in FIG. 4A (left). The compound of embodiment 377 to 379 may in addition comprise further molecular components, e.g. further copies of the first and second bindings modules as exemplified in FIG. 4A (middle and right) where molecules with two copies of the first binding module and two copies of the second binding module in different molecular arrangements are shown. As the skilled person will understand, many other formats of the compound are possible, provided that the resulting compound format does not interfere with the function of the VHH antibody domain(s) or fragment(s) thereof according to the invention (i.e. in the case of the compound of embodiment 377 to 379: the molecular format does not interfere with the binding of the first binding module to IL18Rα and the binding of the second binding module to IL18RP, as described in the present disclosure).

The compound of the present disclosure can be prepared by standard methods of genetic engineering and recombinant protein technology known to the skilled person (see e.g. Green and Sambrook, “Molecular Cloning: A Laboratory Manual”, 2014; Coligan et al., “Current Protocols in Protein Science”, 1997). Exemplary methods are also described in the Examples section of the present disclosure.

In cases where the compound cannot be expressed in a single piece, individual parts can be prepared individually and later either covalently coupled, for example by a chemical reaction with appropriate reactive groups (e.g. linkage by maleimide chemistry) or by enzymatic linkage (e.g. transglutaminase-catalyzed linkage). For example, the VHH antibody domain(s) or fragment(s) thereof can be prepared by recombinant protein expression and subsequently linked to an antibody or antibody fragment, resulting in a bispecific antibody compound as described in the Examples section.

If the compound comprises components that are not biomolecules (such as a peptide mimetics or a small molecule), these components may be obtained e.g. by standard methods of synthetic organic chemistry.

Embodiment 380: The compound according to embodiment 377,
wherein said VHH antibody domain or fragment thereof defined in (a) comprises the complementarity determining regions CDR1, CDR2 and CDR3 of one VHH selected from the group consisting of VHH1, VHH2, VHH5, VHH6, VHH8, VHH9 and VHH10 as shown in the Table of CDRs and
wherein said VHH antibody domain or fragment thereof defined in (d) comprises the complementarity determining regions CDR1, CDR2 and CDR3 of one VHH selected from the group consisting of VHH12, VHH13, VHH14, VHH15, VHH17, VHH18, VHH20, VHH21 and VHH22 as shown in the Table of CDRs, wherein said VHH antibody domain or fragment thereof defined in (d) is selected as follows:
   if the VHH antibody domain of (a) or the fragment thereof comprises the complementarity determining regions CDR1, CDR2 and CDR3 of VHH1, then the VHH antibody domain of (d) or the fragment thereof comprises the complementarity determining regions CDR1, CDR2 and CDR3 of one VHH selected from the group consisting of VHH12, VHH13, VHH15, VHH17, VHH20, VHH21 and VHH22 as shown in the Table of CDRs;
   if the VHH antibody domain of (a) or the fragment thereof comprises the complementarity determining regions CDR1, CDR2 and CDR3 of VHH2, then the VHH antibody domain of (d) or the fragment thereof comprises the complementarity determining regions CDR1, CDR2 and CDR3 of one VHH selected from the group consisting of VHH12, VHH13, VHH15, VHH17, VHH18, VHH20, VHH21 and VHH22 as shown in the Table of CDRs;
if the VHH antibody domain of (a) or the fragment thereof comprises the complementarity determining regions CDR1, CDR2 and CDR3 of VHH5, then the VHH antibody domain of (d) or the fragment thereof comprises the complementarity determining regions CDR1, CDR2 and CDR3 of one VHH selected from the group consisting of VHH13 and VHH14 as shown in the Table of CDRs;
   if the VHH antibody domain of (a) or the fragment thereof comprises the complementarity determining regions CDR1, CDR2 and CDR3 of VHH6, then the VHH antibody domain of (d) or the fragment thereof comprises the complementarity determining regions CDR1, CDR2 and CDR3 of one VHH selected from the group consisting of VHH17, VHH20, VHH21 and VHH22 as shown in the Table of CDRs;
   if the VHH antibody domain of (a) or the fragment thereof comprises the complementarity determining regions CDR1, CDR2 and CDR3 of VHH8, then the VHH antibody domain of (d) or the fragment thereof comprises the complementarity determining regions CDR1, CDR2 and CDR3 of one VHH selected from the group consisting of VHH12, VHH13, VHH17, VHH18, VHH20 and VHH21 as shown in the Table of CDRs;
   if the VHH antibody domain of (a) or the fragment thereof comprises the complementarity determining regions CDR1, CDR2 and CDR3 of VHH9, then the VHH antibody domain of (d) or the fragment thereof comprises the complementarity determining regions CDR1, CDR2 and CDR3 of one VHH selected from the group consisting of VHH12, VHH13, VHH15, VHH17, VHH18, VHH20, VHH21 and VHH22 as shown in the Table of CDRs;
   if the VHH antibody domain of (a) or the fragment thereof comprises the complementarity determining regions CDR1, CDR2 and CDR3 of VHH10, then the VHH antibody domain of (d) or the fragment thereof comprises the complementarity determining regions CDR1, CDR2 and CDR3 of one VHH selected from the group consisting of VHH13, VHH20 and VHH21 as shown in the Table of CDRs. Thus, according this embodiment, the compound of embodiment 377 (and the embodiments referring thereto) can comprise any of the combinations VHHα and VHHβ shown in FIG. 4B in green (surrogate agonists triggering either a normalized NFκB reporter activation of more than 50% relative to IL18 or displaying potencies of less than 0.1 nM).

Embodiment 381: The compound according to embodiment 377,
wherein said VHH antibody domain or fragment thereof defined in (a) comprises the complementarity determining regions CDR1, CDR2 and CDR3 of one VHH selected from the group consisting of VHH1, VHH2, VHH3, VHH4, VHH5, VHH6, VHH8, VHH9 and VHH10 as shown in the Table of CDRs and
wherein said VHH antibody domain or fragment thereof defined in (d) comprises the complementarity determining regions CDR1, CDR2 and CDR3 of one VHH selected from the group consisting of VHH12, VHH13, VHH14, VHH15, VHH16, VHH17, VHH18, VHH20, VHH21 and VHH22 as shown in the Table of CDRs, wherein said VHH antibody domain or fragment thereof defined in (d) is selected as follows:
   if the VHH antibody domain of (a) or the fragment thereof comprises the complementarity determining regions CDR1, CDR2 and CDR3 of VHH1, then the VHH antibody domain of (d) or the fragment thereof comprises the complementarity determining regions CDR1, CDR2 and CDR3 of one VHH selected from the group consisting of VHH14, VHH16 and VHH18 as shown in the Table of CDRs;
   if the VHH antibody domain of (a) or the fragment thereof comprises the complementarity determining regions CDR1, CDR2 and CDR3 of VHH2, then the VHH antibody domain of (d) or the fragment thereof comprises the complementarity determining regions CDR1, CDR2 and CDR3 of one VHH selected from the group consisting of VHH14 and VHH16 as shown in the Table of CDRs;
   if the VHH antibody domain of (a) or the fragment thereof comprises the complementarity determining regions CDR1, CDR2 and CDR3 of VHH3, then the VHH antibody domain of (d) or the fragment thereof comprises the complementarity determining regions CDR1, CDR2 and CDR3 of one VHH selected from the group consisting of VHH12, VHH13, VHH14, VHH15, VHH16, VHH17, VHH18, VHH20, VHH21 and VHH22 as shown in the Table of CDRs;
   if the VHH antibody domain of (a) or the fragment thereof comprises the complementarity determining regions CDR1, CDR2 and CDR3 of VHH4, then the VHH antibody domain of (d) or the fragment thereof comprises the complementarity determining regions CDR1, CDR2 and CDR3 of one VHH selected from the group consisting of VHH12, VHH13, VHH14, VHH15, VHH16, VHH17, VHH18, VHH20, VHH21 and VHH22 as shown in the Table of CDRs;
   if the VHH antibody domain of (a) or the fragment thereof comprises the complementarity determining regions CDR1, CDR2 and CDR3 of VHH5, then the VHH antibody domain of (d) or the fragment thereof comprises the complementarity determining regions CDR1, CDR2 and CDR3 of one VHH selected from the group consisting of of VHH12, VHH15, VHH16, VHH17, VHH18, VHH20, VHH21 and VHH22 as shown in the Table of CDRs;
   if the VHH antibody domain of (a) or the fragment thereof comprises the complementarity determining regions CDR1, CDR2 and CDR3 of VHH6, then the VHH antibody domain of (d) or the fragment thereof comprises the complementarity determining regions CDR1, CDR2 and CDR3 of one VHH selected from the group consisting of VHH14, VHH16 and VHH18 as shown in the Table of CDRs;
   if the VHH antibody domain of (a) or the fragment thereof comprises the complementarity determining regions CDR1, CDR2 and CDR3 of VHH8, then the VHH antibody domain of (d) or the fragment thereof comprises the complementarity determining regions CDR1, CDR2 and CDR3 of VHH16 as shown in the Table of CDRs;
   if the VHH antibody domain of (a) or the fragment thereof comprises the complementarity determining regions CDR1, CDR2 and CDR3 of VHH9, then the VHH antibody domain of (d) or the fragment thereof comprises the complementarity determining regions CDR1, CDR2 and CDR3 of one VHH selected from the group consisting of VHH14, VHH16 and VHH18 as shown in the Table of CDRs;
   if the VHH antibody domain of (a) or the fragment thereof comprises the complementarity determining regions CDR1, CDR2 and CDR3 of VHH10, then the VHH antibody domain of (d) or the fragment thereof comprises the complementarity determining regions CDR1, CDR2 and CDR3 of one VHH selected from the group consisting of VHH14, VHH15, VHH16 and VHH18 as shown in the Table of CDRs.

Thus, according to this embodiment, the compound of embodiment 377 (and the embodiments referring thereto) can comprise any of the combinations VHHα and VHHβ shown in FIG. 4B in red.

Embodiment 382: The compound according to embodiment 377,
wherein said VHH antibody domain or fragment thereof defined in (a) comprises the complementarity determining regions CDR1, CDR2 and CDR3 of one VHH selected from the group consisting of VHH6 and VHH11 as shown in the Table of CDRs and
wherein said VHH antibody domain or fragment thereof defined in (d) comprises the complementarity determining regions CDR1, CDR2 and CDR3 of one VHH selected from the group consisting of VHH12, VHH13, VHH14, VHH15, VHH16, VHH17, VHH18, VHH20, VHH21 and VHH22 as shown in the Table of CDRs, wherein said VHH antibody domain or fragment thereof defined in (d) is selected as follows:
   if the VHH antibody domain of (a) or the fragment thereof comprises the complementarity determining regions CDR1, CDR2 and CDR3 of VHH6, then the VHH antibody domain of (d) or the fragment thereof comprises the complementarity determining regions CDR1, CDR2 and CDR3 of one VHH selected from the group consisting of VHH12 and VHH13 as shown in the Table of CDRs;
   if the VHH antibody domain of (a) or the fragment thereof comprises the complementarity determining regions CDR1, CDR2 and CDR3 of VHH11, then the VHH antibody domain of (d) or the fragment thereof comprises the complementarity determining regions CDR1, CDR2 and CDR3 of one VHH selected from the group consisting of VHH12, VHH13, VHH14, VHH15, VHH16, VHH17, VHH18, VHH20, VHH21 and VHH22 as shown in the Table of CDRs.

Thus, according to this embodiment, the compound of embodiment 377 (and the embodiments referring thereto) can comprise any of the combinations VHHα and VHHβ shown in FIG. 4B in black.

Embodiment 383: The compound according to embodiment 377,
wherein said VHH antibody domain or fragment thereof defined in (a) comprises the complementarity determining regions CDR1, CDR2 and CDR3 of one VHH selected from the group consisting of VHH2, VHH6, VHH8 and VHH10 as shown in the Table of CDRs and
wherein said VHH antibody domain or fragment thereof defined in (d) comprises the complementarity determining regions CDR1, CDR2 and CDR3 of one VHH selected from the group consisting of VHH12, VHH14, VHH15, VHH17 and VHH22 as shown in the Table of CDRs, wherein said VHH antibody domain or fragment thereof defined in (d) is selected as follows:
   if the VHH antibody domain of (a) or the fragment thereof comprises the complementarity determining regions CDR1, CDR2 and CDR3 of VHH2, then the VHH antibody domain of (d) or the fragment thereof comprises the complementarity determining regions CDR1, CDR2 and CDR3 of VHH22 as shown in the Table of CDRs;
   if the VHH antibody domain of (a) or the fragment thereof comprises the complementarity determining regions CDR1, CDR2 and CDR3 of VHH6, then the VHH antibody domain of (d) or the fragment thereof comprises the complementarity determining regions CDR1, CDR2 and CDR3 of VHH15 as shown in the Table of CDRs;
   if the VHH antibody domain of (a) or the fragment thereof comprises the complementarity determining regions CDR1, CDR2 and CDR3 of VHH8, then the VHH antibody domain of (d) or the fragment thereof comprises the complementarity determining regions CDR1, CDR2 and CDR3 of one VHH selected from the group consisting of VHH14, VHH15 and VHH22 as shown in the Table of CDRs;
   if the VHH antibody domain of (a) or the fragment thereof comprises the complementarity determining regions CDR1, CDR2 and CDR3 of VHH10, then the VHH antibody domain of (d) or the fragment thereof comprises the complementarity determining regions CDR1, CDR2 and CDR3 of one VHH selected from the group consisting of VHH12, VHH17 and VHH22 as shown in the Table of CDRs.

Thus, according to this embodiment, the compound of embodiment 377 (and the embodiments referring thereto) can comprise any of the combinations VHHα and VHHβ shown in FIG. 4B in orange (active surrogate agonists eliciting less than 50% of NFκB reporter activation normalized to (rh) IL-18 at 1 nM or EC50 higher than 0.1 nM).

Embodiment 384: The compound according to embodiment 377, wherein said VHH antibody domain or fragment thereof defined in (a) comprises the complementarity determining regions CDR1, CDR2 and CDR3 of one VHH selected from the group consisting of VHH2 and VHH8 as shown in the Table of CDRs and wherein said VHH antibody domain or fragment thereof defined in (d) comprises the complementarity determining regions CDR1, CDR2 and CDR3 of one VHH selected from the group consisting of VHH15 and VHH17 as shown in the Table of CDRs.

Embodiment 385: The compound according to embodiment 377,
wherein said VHH antibody domain or fragment thereof defined in (a) comprises the complementarity determining regions CDR1, CDR2 and CDR3 of one VHH selected from the group consisting of VHH2 and VHH8 as shown in the Table of CDRs and
wherein said VHH antibody domain or fragment thereof defined in (d) comprises the complementarity determining regions CDR1, CDR2 and CDR3 of one VHH selected from the group consisting of VHH15 and VHH17 as shown in the Table of CDRs, wherein said VHH antibody domain or fragment thereof defined in (d) is selected as follows:
   if the VHH antibody domain of (a) or the fragment thereof comprises the complementarity determining regions CDR1, CDR2 and CDR3 of VHH2, then the VHH antibody domain of (d) or the fragment thereof comprises the complementarity determining regions CDR1, CDR2 and CDR3 of one VHH selected from the group consisting of VHH15 and VHH17 as shown in the Table of CDRs;
   if the VHH antibody domain of (a) or the fragment thereof comprises the complementarity determining regions CDR1, CDR2 and CDR3 of VHH8, then the VHH antibody domain of (d) or the fragment thereof comprises the complementarity determining regions CDR1, CDR2 and CDR3 of VHH17 as shown in the Table of CDRs.

Embodiment 386: The compound according to embodiment 377, wherein said VHH antibody domain or fragment thereof defined in (a) comprises the complementarity determining regions CDR1, CDR2 and CDR3 of VHH2 as shown in the Table of CDRs and wherein said VHH antibody domain or fragment thereof defined in (d) comprises the complementarity determining regions CDR1, CDR2 and CDR3 of VHH15 as shown in the Table of CDRs.

Embodiment 387: The compound according to embodiment 377,
wherein said VHH antibody domain or fragment thereof defined in (a) comprises the complementarity determining regions CDR1, CDR2 and CDR3 of one VHH selected from the group consisting of VHH1, VHH2, VHH5, VHH6, VHH8 and VHH10 as shown in the Table of CDRs and
wherein said VHH antibody domain or fragment thereof defined in (d) comprises the complementarity determining regions CDR1, CDR2 and CDR3 of one VHH selected from the group consisting of VHH13, VHH16 and VHH22 as shown in the Table of CDRs, wherein said VHH antibody domain or fragment thereof defined in (d) is selected as follows:
   if the VHH antibody domain of (a) or the fragment thereof comprises the complementarity determining regions CDR1, CDR2 and CDR3 of VHH1, then the VHH antibody domain of (d) or the fragment thereof comprises the complementarity determining regions CDR1, CDR2 and CDR3 of VHH16 as shown in the Table of CDRs;
   if the VHH antibody domain of (a) or the fragment thereof comprises the complementarity determining regions CDR1, CDR2 and CDR3 of VHH2, then the VHH antibody domain of (d) or the fragment thereof comprises the complementarity determining regions CDR1, CDR2 and CDR3 of VHH22 as shown in the Table of CDRs;
   if the VHH antibody domain of (a) or the fragment thereof comprises the complementarity determining regions CDR1, CDR2 and CDR3 of VHH5, then the VHH antibody domain of (d) or the fragment thereof comprises the complementarity determining regions CDR1, CDR2 and CDR3 of VHH13 as shown in the Table of CDRs;
   if the VHH antibody domain of (a) or the fragment thereof comprises the complementarity determining regions CDR1, CDR2 and CDR3 of VHH6, then the VHH antibody domain of (d) or the fragment thereof comprises the complementarity determining regions CDR1, CDR2 and CDR3 of VHH22 as shown in the Table of CDRs;
   if the VHH antibody domain of (a) or the fragment thereof comprises the complementarity determining regions CDR1, CDR2 and CDR3 of VHH8, then the VHH antibody domain of (d) or the fragment thereof comprises the complementarity determining regions CDR1, CDR2 and CDR3 of VHH22 as shown in the Table of CDRs;
   if the VHH antibody domain of (a) or the fragment thereof comprises the complementarity determining regions CDR1, CDR2 and CDR3 of VHH10, then the VHH antibody domain of (d) or the fragment thereof comprises the complementarity determining regions CDR1, CDR2 and CDR3 of VHH22 as shown in the Table of CDRs.

Embodiment 388: The compound according to embodiment 377,
wherein said VHH antibody domain or fragment thereof defined in (a) comprises the complementarity determining regions CDR1, CDR2 and CDR3 of one VHH selected from the group consisting of VHH2, VHH6, VHH8 and VHH10 as shown in the Table of CDRs and
wherein said VHH antibody domain or fragment thereof defined in (d) comprises the complementarity determining regions CDR1, CDR2 and CDR3 of one VHH selected from the group consisting of VHH12 and VHH15 as shown in the Table of CDRs, wherein said VHH antibody domain or fragment thereof defined in (d) is selected as follows:
   if the VHH antibody domain of (a) or the fragment thereof comprises the complementarity determining regions CDR1, CDR2 and CDR3 of VHH2, then the VHH antibody domain of (d) or the fragment thereof comprises the complementarity determining regions CDR1, CDR2 and CDR3 of VHH12 as shown in the Table of CDRs;
   if the VHH antibody domain of (a) or the fragment thereof comprises the complementarity determining regions CDR1, CDR2 and CDR3 of VHH6, then the VHH antibody domain of (d) or the fragment thereof comprises the complementarity determining regions CDR1, CDR2 and CDR3 of one VHH selected from the group consisting of VHH12 and VHH15 as shown in the Table of CDRs;
   if the VHH antibody domain of (a) or the fragment thereof comprises the complementarity determining regions CDR1, CDR2 and CDR3 of VHH8, then the VHH antibody domain of (d) or the fragment thereof comprises the complementarity determining regions CDR1, CDR2 and CDR3 of one VHH selected from the group consisting of VHH13 and VHH18 as shown in the Table of CDRs;
   if the VHH antibody domain of (a) or the fragment thereof comprises the complementarity determining regions CDR1, CDR2 and CDR3 of VHH10, then the VHH antibody domain of (d) or the fragment thereof comprises the complementarity determining regions CDR1, CDR2 and CDR3 of VHH12 as shown in the Table of CDRs.

Embodiment 389: The compound according to any one of embodiments 377 or 380 to 388, wherein in the first binding module said VHH antibody domain or fragment thereof comprises complementarity determining regions according to (a) or (b).

If the present disclosure states that “in the first binding module said VHH antibody domain or fragment thereof comprises complementarity determining regions according to (a) or (b)”, then this is meant to designate that in this embodiment only (a) and (b) are considered as options for the VHH antibody domain (or fragment thereof) of the first binding module, whereas option (c) is excluded. If the present disclosure states that “in the first binding module said VHH antibody domain or fragment thereof comprises complementarity determining regions according to (a) or (c)”, then this is meant to designate that in this embodiment only (a) and (c) are considered as options for the VHH antibody domain (or fragment thereof) of the first binding module, whereas option (b) is excluded. The alternative wording below is to be understood accordingly. If the present disclosure states that “in the first binding module said VHH antibody domain or fragment thereof comprises complementarity determining regions according to (a)”, then this is meant to designate that in this embodiment only (a) is considered as option for the VHH antibody domain (or fragment thereof) of the first binding module, whereas options (b) and (c) are excluded. Further variations of this wording below are to be understood accordingly.

Embodiment 390: The compound according to any one of embodiments 377 or 380 to 388, wherein in the first binding module said VHH antibody domain or fragment thereof comprises complementarity determining regions according to (a) or (c).

Embodiment 391: The compound according to any one of embodiments 377 or 380 to 388, wherein in the first binding module said VHH antibody domain or fragment thereof comprises complementarity determining regions according to (a).

Embodiment 392: The compound according to any one of embodiments 377 or 380 to 388, wherein in the first binding module said VHH antibody domain or fragment thereof comprises complementarity determining regions according to (b).

Embodiment 393: The compound according to any one of embodiments 377 or 380 to 392, wherein in the second binding module said VHH antibody domain or fragment thereof comprises complementarity determining regions according to (d) or (e).

Embodiment 394: The compound according to any one of embodiments 377 or 380 to 392, wherein in the second binding module said VHH antibody domain or fragment thereof comprises complementarity determining regions according to (d) or (f).

Embodiment 395: The compound according to any one of embodiments 377 or 380 to 392, wherein in the second binding module said VHH antibody domain or fragment thereof comprises complementarity determining regions according to (d).

Embodiment 396: The compound according to any one of embodiments 377 or 380 to 392, wherein in the second binding module said VHH antibody domain or fragment thereof comprises complementarity determining regions according to (e).

Embodiment 397: The compound according to any one of embodiments 378 or 379,
wherein said VHH antibody domain of (A) comprises the amino acid sequence of one VHH selected from the group consisting of VHH1, VHH2, VHH5, VHH6, VHH8, VHH9 and VHH10 shown in the Table of VHH Sequences and
wherein said VHH antibody domain of (E) comprises the amino acid sequence of one VHH selected from the group consisting of VHH12, VHH13, VHH14, VHH15, VHH17, VHH18, VHH20, VHH21 and VHH22 shown in the Table of VHH Sequences, wherein said VHH antibody domain of (E) is selected as follows:
   if the VHH antibody domain of (A) comprises the amino acid sequence of VHH1, then the VHH antibody domain of (E) comprises the amino acid sequence of one VHH selected from the group consisting of VHH12, VHH13, VHH15, VHH17, VHH20, VHH21 and VHH22 shown in the Table of VHH Sequences;
   if the VHH antibody domain of (A) comprises the amino acid sequence of VHH2, then the VHH antibody domain of (E) comprises the amino acid sequence of one VHH selected from the group consisting of VHH12, VHH13, VHH15, VHH17, VHH18, VHH20, VHH21 and VHH22 shown in the Table of VHH Sequences;
   if the VHH antibody domain of (A) comprises the amino acid sequence of VHH5, then the VHH antibody domain of (E) comprises the amino acid sequence of one VHH selected from the group consisting of VHH13 and VHH14 shown in the Table of VHH Sequences;
   if the VHH antibody domain of (A) comprises the amino acid sequence of VHH6, then the VHH antibody domain of (E) comprises the amino acid sequence of one VHH selected from the group consisting of VHH17, VHH20, VHH21 and VHH22 shown in the Table of VHH Sequences;
   if the VHH antibody domain of (A) comprises the amino acid sequence of VHH8, then the VHH antibody domain of (E) comprises the amino acid sequence of one VHH selected from the group consisting of VHH12, VHH13, VHH17, VHH18, VHH20 and VHH21 shown in the Table of VHH Sequences;
   if the VHH antibody domain of (A) comprises the amino acid sequence of VHH9, then the VHH antibody domain of (E) comprises the amino acid sequence of one VHH selected from the group consisting of VHH12, VHH13, VHH15, VHH17, VHH18, VHH20, VHH21 and VHH22 shown in the Table of VHH Sequences;
   if the VHH antibody domain of (A) comprises the amino acid sequence of VHH10, then the VHH antibody domain of (E) comprises the amino acid sequence of one VHH selected from the group consisting of VHH13, VHH20 and VHH21 shown in the Table of VHH Sequences.

Thus, according to this embodiment, the compound of embodiments 378 to 379 (and the embodiments referring thereto) can comprise any of the combinations VHHα and VHHβ shown in FIG. 4B in green (surrogate agonists triggering either a normalized NFκB reporter activation of more than 50% relative to IL18 or displaying potencies of less than 0.1 nM).

Embodiment 398: The compound according to any one of embodiments 378 or 379,
wherein said VHH antibody domain of (A) comprises the amino acid sequence of one VHH selected from the group consisting of VHH1, VHH2, VHH3, VHH4, VHH5, VHH6, VHH8, VHH9 and VHH10 shown in the Table of VHH Sequences and
wherein said VHH antibody domain of (E) comprises the amino acid sequence of one VHH selected from the group consisting of VHH12, VHH13, VHH14, VHH15, VHH16, VHH17, VHH18, VHH20, VHH21 and VHH22 shown in the Table of VHH Sequences, wherein said VHH antibody domain of (E) is selected as follows:
   if the VHH antibody domain of (A) comprises the amino acid sequence of VHH1, then the VHH antibody domain of (E) comprises the amino acid sequence of one VHH selected from the group consisting of VHH14, VHH16 and VHH18 shown in the Table of VHH Sequences;
   if the VHH antibody domain of (A) comprises the amino acid sequence of VHH2, then the VHH antibody domain of (E) comprises the amino acid sequence of one VHH selected from the group consisting of VHH14 and VHH16 shown in the Table of VHH Sequences;
   if the VHH antibody domain of (A) comprises the amino acid sequence of VHH3, then the VHH antibody domain of (E) comprises the amino acid sequence of one VHH selected from the group consisting of VHH12, VHH13, VHH14, VHH15, VHH16, VHH17, VHH18, VHH20, VHH21 and VHH22 shown in the Table of VHH Sequences;
   if the VHH antibody domain of (A) comprises the amino acid sequence of VHH4, then the VHH antibody domain of (E) comprises the amino acid sequence of one VHH selected from the group consisting of VHH12, VHH13, VHH14, VHH15, VHH16, VHH17, VHH18, VHH20, VHH21 and VHH22 shown in the Table of VHH Sequences;
   if the VHH antibody domain of (A) comprises the amino acid sequence of VHH5, then the VHH antibody domain of (E) comprises the amino acid sequence of one VHH selected from the group consisting of VHH12, VHH15, VHH16, VHH17, VHH18, VHH20, VHH21 and VHH22 shown in the Table of VHH Sequences;
   if the VHH antibody domain of (A) comprises the amino acid sequence of VHH6, then the VHH antibody domain of (E) comprises the amino acid sequence of one VHH selected from the group consisting of VHH14, VHH16 and VHH18 shown in the Table of VHH Sequences;
   if the VHH antibody domain of (A) comprises the amino acid sequence of VHH8, then the VHH antibody domain of (E) comprises the amino acid sequence of VHH16 shown in the Table of VHH Sequences;
   if the VHH antibody domain of (A) comprises the amino acid sequence of VHH9, then the VHH antibody domain of (E) comprises the amino acid sequence of one VHH selected from the group consisting of VHH14, VHH16 and VHH18 shown in the Table of VHH Sequences;
   if the VHH antibody domain of (A) comprises the amino acid sequence of VHH10, then the VHH antibody domain of (E) comprises the amino acid sequence of one VHH selected from the group consisting of VHH14, VHH15, VHH16 and VHH18 shown in the Table of VHH Sequences.

Thus, according to this embodiment, the compound of embodiments 378 to 379 (and the embodiments referring thereto) can comprise any of the combinations VHHα and VHHβ shown in FIG. 4B in red.

Embodiment 399: The compound according to any one of embodiments 378 or 379,
wherein said VHH antibody domain of (A) comprises the amino acid sequence of one VHH selected from the group consisting of VHH6 and VHH11 shown in the Table of VHH Sequences and
wherein said VHH antibody domain of (E) comprises the amino acid sequence of one VHH selected from the group consisting of VHH12, VHH13, VHH14, VHH15, VHH16, VHH17, VHH18, VHH20, VHH21 and VHH22 shown in the Table of VHH Sequences, wherein said VHH antibody domain of (E) is selected as follows:
   if the VHH antibody domain of (A) comprises the amino acid sequence of VHH6, then the VHH antibody domain of (E) comprises the amino acid sequence of one VHH selected from the group consisting of VHH12 and VHH13 shown in the Table of VHH Sequences;
   if the VHH antibody domain of (A) comprises the amino acid sequence of VHH11, then the VHH antibody domain of (E) comprises the amino acid sequence of one VHH selected from the group consisting of VHH12, VHH13, VHH14, VHH15, VHH16, VHH17, VHH18, VHH20, VHH21 and VHH22 shown in the Table of VHH Sequences.

Thus, according to this embodiment, the compound of embodiments 378 to 379 (and the embodiments referring thereto) can comprise any of the combinations VHHα and VHHβ shown in FIG. 4B in black.

Embodiment 400: The compound according to any one of embodiments 378 or 379,
wherein said VHH antibody domain of (A) comprises the amino acid sequence of one VHH selected from the group consisting of VHH2, VHH6, VHH8 and VHH10 shown in the Table of VHH Sequences and
wherein said VHH antibody domain of (E) comprises the amino acid sequence of one VHH selected from the group consisting of VHH12, VHH14, VHH15, VHH17 and VHH22 shown in the Table of VHH Sequences, wherein said VHH antibody domain of (E) is selected as follows:
   if the VHH antibody domain of (A) comprises the amino acid sequence of VHH2, then the VHH antibody domain of (E) comprises the amino acid sequence of VHH22 shown in the Table of VHH Sequences;
   if the VHH antibody domain of (A) comprises the amino acid sequence of VHH6, then the VHH antibody domain of (E) comprises the amino acid sequence of VHH15 shown in the Table of VHH Sequences;
   if the VHH antibody domain of (A) comprises the amino acid sequence of VHH8, then the VHH antibody domain of (E) comprises the amino acid sequence of one VHH selected from the group consisting of VHH14, VHH15 and VHH22 shown in the Table of VHH Sequences;
   if the VHH antibody domain of (A) comprises the amino acid sequence of VHH10, then the VHH antibody domain of (E) comprises the amino acid sequence of one VHH selected from the group consisting of VHH12, VHH17 and VHH22 shown in the Table of VHH Sequences.

Thus, according to this embodiment, the compound of embodiments 378 to 379 (and the embodiments referring thereto) can comprise any of the combinations VHHα and VHHβ shown in FIG. 4B in orange (active surrogate agonists eliciting less than 50% of NFκB reporter activation normalized to (rh) IL-18 at 1 nM or EC50 higher than 0.1 nM).

Embodiment 401: The compound according to any one of embodiments 378 or 379, wherein said VHH antibody domain of (A) comprises the amino acid sequence of one VHH selected from the group consisting of VHH2 and VHH8 shown in the Table of VHH Sequences and wherein said VHH antibody domain of (E) comprises the amino acid sequence of one VHH selected from the group consisting of VHH15 and VHH17 shown in the Table of VHH Sequences.

Embodiment 402: The compound according to any one of embodiments 378 or 379,
wherein said VHH antibody domain of (A) comprises the amino acid sequence of one VHH selected from the group consisting of VHH2 and VHH8 shown in the Table of VHH Sequences and
wherein said VHH antibody domain of (E) comprises the amino acid sequence of one VHH selected from the group consisting of VHH15 and VHH17 shown in the Table of VHH Sequences, wherein said VHH antibody domain of (E) is selected as follows:
   if the VHH antibody domain of (A) comprises the amino acid sequence of VHH2, then the VHH antibody domain of (E) comprises the amino acid sequence of one VHH selected from the group consisting of VHH15 and VHH17 shown in the Table of VHH Sequences;
   if the VHH antibody domain of (A) comprises the amino acid sequence of VHH8, then the VHH antibody domain of (E) comprises the amino acid sequence of VHH17 shown in the Table of VHH Sequences.

Embodiment 403: The compound according to any one of embodiments 378 or 379, wherein said VHH antibody domain of (A) comprises the amino acid sequence of VHH2 shown in the Table of VHH Sequences and wherein said VHH antibody domain of (E) comprises the amino acid sequence of VHH15 shown in the Table of VHH Sequences.

Embodiment 404: The compound according to any one of embodiments 378 or 379,
wherein said VHH antibody domain of (A) comprises the amino acid sequence of one VHH selected from the group consisting of VHH1, VHH2, VHH5, VHH6, VHH8 and VHH10 shown in the Table of VHH Sequences and
wherein said VHH antibody domain of (E) comprises the amino acid sequence of one VHH selected from the group consisting of VHH13, VHH16 and VHH22 shown in the Table of VHH Sequences, wherein said VHH antibody domain of (E) is selected as follows:
   if the VHH antibody domain of (A) comprises the amino acid sequence of VHH1, then the VHH antibody domain of (E) comprises the amino acid sequence of VHH16 shown in the Table of VHH Sequences;
   if the VHH antibody domain of (A) comprises the amino acid sequence of VHH2, then the VHH antibody domain of (E) comprises the amino acid sequence of VHH22 shown in the Table of VHH Sequences;
   if the VHH antibody domain of (A) comprises the amino acid sequence of VHH5, then the VHH antibody domain of (E) comprises the amino acid sequence of VHH13 shown in the Table of VHH Sequences;
   if the VHH antibody domain of (A) comprises the amino acid sequence of VHH6, then the VHH antibody domain of (E) comprises the amino acid sequence of VHH22 shown in the Table of VHH Sequences;
   if the VHH antibody domain of (A) comprises the amino acid sequence of VHH8, then the VHH antibody domain of (E) comprises the amino acid sequence of VHH22 shown in the Table of VHH Sequences;
   if the VHH antibody domain of (A) comprises the amino acid sequence of VHH10, then the VHH antibody domain of (E) comprises the amino acid sequence of VHH22 shown in the Table of VHH Sequences.

Embodiment 405: The compound according to any one of embodiments 378 or 379,
wherein said VHH antibody domain of (A) comprises the amino acid sequence of one VHH selected from the group consisting of VHH2, VHH6, VHH8 and VHH10 shown in the Table of VHH Sequences and
wherein said VHH antibody domain of (E) comprises the amino acid sequence of one VHH selected from the group consisting of VHH12, VHH13, VHH15 and VHH18 shown in the Table of VHH Sequences, wherein said VHH antibody domain of (E) is selected as follows:
   if the VHH antibody domain of (A) comprises the amino acid sequence of VHH2, then the VHH antibody domain of (E) comprises the amino acid sequence of VHH12 shown in the Table of VHH Sequences;
   if the VHH antibody domain of (A) comprises the amino acid sequence of VHH6, then the VHH antibody domain of (E) comprises the amino acid sequence of one VHH selected from the group consisting of VHH15 and VHH12 shown in the Table of VHH Sequences;
   if the VHH antibody domain of (A) comprises the amino acid sequence of VHH8, then the VHH antibody domain of (E) comprises the amino acid sequence of one VHH selected from the group consisting of VHH13 and VHH18 shown in the Table of VHH Sequences;
   if the VHH antibody domain of (A) comprises the amino acid sequence of VHH10, then the VHH antibody domain of (E) comprises the amino acid sequence of VHH12 shown in the Table of VHH Sequences.

Embodiment 406: The compound according to any one of embodiments 378 to 379 or 397 to 405, wherein in the first binding module said VHH antibody domain or fragment thereof comprises a VHH antibody domain or fragment thereof according to (A), (B) or (C).

If the present disclosure states that “in the first binding module said VHH antibody domain or fragment thereof comprises a VHH antibody domain or fragment thereof according to (A), (B) or (C)”, then this is meant to designate that in this embodiment only (A), (B) and (C) are considered as options for the VHH antibody domain (or fragment thereof) of the first binding module, whereas the option (D) is excluded. If the present disclosure states that “in the first binding module said VHH antibody domain or fragment thereof comprises a VHH antibody domain or fragment thereof according to (A), (C) or (D)”, then this is meant to designate that in this embodiment only (A), (C) and (D) are considered as options for the VHH antibody domain (or fragment thereof) of the first binding module, whereas the option (B) is excluded. If the present disclosure states that “in the first binding module said VHH antibody domain or fragment thereof comprises a VHH antibody domain or fragment thereof according to (A)”, then this is meant to designate that in this embodiment only (A) is considered as option for the VHH antibody domain (or fragment thereof) of the first binding module, whereas the options (B), (C) and (D) are excluded. Further variations of this wording below are to be understood accordingly.

Embodiment 407: The compound according to any one of embodiments 378 to 379 or 397 to 405, wherein in the first binding module said VHH antibody domain or fragment thereof comprises a VHH antibody domain or fragment thereof according to (A), (B) or (D).

Embodiment 408: The compound according to any one of embodiments 378 to 379 or 397 to 405, wherein in the first binding module said VHH antibody domain or fragment thereof comprises a VHH antibody domain or fragment thereof according to (A), (C) or (D).

Embodiment 409: The compound according to any one of embodiments 378 to 379 or 397 to 405, wherein in the first binding module said VHH antibody domain or fragment thereof comprises a VHH antibody domain or fragment thereof according to (A) or (B).

Embodiment 410: The compound according to any one of embodiments 378 to 379 or 397 to 405, wherein in the first binding module said VHH antibody domain or fragment thereof comprises a VHH antibody domain or fragment thereof according to (A) or (C).

Embodiment 411: The compound according to any one of embodiments 378 to 379 or 397 to 405, wherein in the first binding module said VHH antibody domain or fragment thereof comprises a VHH antibody domain or fragment thereof according to (A) or (D).

Embodiment 412: The compound according to any one of embodiments 378 to 379 or 397 to 405, wherein in the first binding module said VHH antibody domain or fragment thereof comprises a VHH antibody domain or fragment thereof according to (A).

Embodiment 413: The compound according to any one of embodiments 378 to 379 or 397 to 405, wherein in the first binding module said VHH antibody domain or fragment thereof comprises a VHH antibody domain or fragment thereof according to (B).

Embodiment 414: The compound according to any one of embodiments 378 to 379 or 397 to 413, wherein in the second binding module said VHH antibody domain or fragment thereof comprises a VHH antibody domain or fragment thereof according to (E), (F) or (G).

Embodiment 415: The compound according to any one of embodiments 378 to 379 or 397 to 413, wherein in the second binding module said VHH antibody domain or fragment thereof comprises a VHH antibody domain or fragment thereof according to (E), (F) or (H).

Embodiment 416: The compound according to any one of embodiments 378 to 379 or 397 to 413, wherein in the second binding module said VHH antibody domain or fragment thereof comprises a VHH antibody domain or fragment thereof according to (E), (G) or (H).

Embodiment 417: The compound according to any one of embodiments 378 to 379 or 397 to 413, wherein in the second binding module said VHH antibody domain or fragment thereof comprises a VHH antibody domain or fragment thereof according to (E) or (F).

Embodiment 418: The compound according to any one of embodiments 378 to 379 or 397 to 413, wherein in the second binding module said VHH antibody domain or fragment thereof comprises a VHH antibody domain or fragment thereof according to (E) or (G).

Embodiment 419: The compound according to any one of embodiments 378 to 379 or 397 to 413, wherein in the second binding module said VHH antibody domain or fragment thereof comprises a VHH antibody domain or fragment thereof according to (E) or (H).

Embodiment 420: The compound according to any one of embodiments 378 to 379 or 397 to 413, wherein in the second binding module said VHH antibody domain or fragment thereof comprises a VHH antibody domain or fragment thereof according to (E).

Embodiment 421: The compound according to any one of embodiments 378 to 379 or 397 to 413, wherein in the second binding module said VHH antibody domain or fragment thereof comprises a VHH antibody domain or fragment thereof according to (F).

Embodiment 422: The compound according to any one of embodiments 375 to 421, wherein said compound is a molecule.

Embodiment 423: The compound according to any one of embodiments 375 to 422, wherein said compound comprises or is a protein.

By stating that the compound “comprises” a protein, the present disclosure designates that the compound includes a part within its chemical structure that is a protein. A compound that comprises a protein may or may not comprise a part that is not a protein.

Embodiment 424: The compound according to any one of embodiments 375 to 422, wherein said compound is a protein.

By stating that the compound “is” a protein, the present disclosure designates that the compound consists only of protein and does not comprise a part that is not a protein.

Embodiment 425: The compound according to any one of embodiments 375 to 424, wherein said compound further comprises a targeting moiety.

As used herein, the term “targeting moiety” refers to a moiety (i.e. a molecular group or chemical structure) that is (typically covalently) associated with said compound and that binds a target site, wherein said binding allows to recruit the compound to said target site (e.g. a moiety that specifically binds to EGFR and thus targets the compound to cells expressing EGFR at their cell surface). The target site will typically be a biological molecule or a certain part of a biological molecule. An example of a targeting moiety is an antigen-binding antibody fragment that is covalently linked to an IL-18Rα-binding and/or IL-18Rβ-binding VHH antibody domain to form a compound according to the present disclosure, wherein the antigen-binding fragment binds to a certain receptor present at the surface of a certain cell type (its antigen), and wherein binding of the antigen-binding fragment to this receptor results in recruitment of the compound to this cell.

Non-targeted drugs typically reach their site of action by whole-body distribution and passive diffusion. In contrast, targeted compounds are not distributed evenly across the whole body. Due to the interaction of targeting moiety with its target molecule, a compound including a targeting moiety is concentrated preferentially at its site target site. Therefore, e.g. therapeutic compounds with a targeting moiety require lower dosages to be therapeutically effective, thus improving the therapeutic window.

Embodiment 426: The compound according to embodiment 425, wherein said targeting moiety is a protein, a peptide, a peptide mimetic, a nucleic acid, an oligonucleotide or a small molecule.

As used herein, the term “peptide mimetic” refers to a peptide-like chain which is designed to mimic a peptide. An example of a peptide mimetic is a D-peptide mimetic containing a D-amino acid, but is not limited thereto. As used herein, a “small molecule”, is a molecule with a molecular weight <1000 Da.

Embodiment 427: The compound according to any one of embodiments 375 to 426, wherein said compound is a monospecific molecule.

Embodiment 428: The compound according to any one of embodiments 375 to 426, wherein said compound is a bispecific molecule.

Embodiment 429: The compound according to any one of embodiments 375 to 426 or 428, wherein said compound is a bispecific antibody molecule.

The term “bispecific antibody molecule”, as used in the present disclosure, refers to an antibody molecule that is capable of specifically binding to two different epitopes at the same time. This does not exclude the possibility that said bispecific antibody molecule is linked to further domains or moieties.

The terms “epitope” or “antigenic determinant” are used interchangeably herein and refer to the portion of an antigen that is recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3, and more usually, at least 5 or 8-10 amino acids in a unique spatial conformation.

Typically, in the compounds of the present disclosure, one of the two epitopes to which the compound of the present disclosure binds is an epitope on IL-18Rα whereas the other of the two epitopes to which the compound of the present disclosure binds is an epitope on IL-18Rβ.

Methods for making bispecific antibodies are known in the art. For example, bispecific antibodies can be produced recombinantly using the co-expression of two immunoglobulin heavy chain/light chain pairs (see e.g. Milstein et al., Nature (1983), vol. 305, p. 537-539). Alternatively, bispecific antibodies can be prepared using chemical linkage (see e.g. Brennan et al., Science (1985), vol. 229, p. 81). A bispecific antibody can also for example be prepared by the SEED technology (see below).

Embodiment 430: The compound according to any one of embodiments 375 to 426 or 428 to 429, wherein one binding site of said bispecific antibody molecule is formed by a VHH antibody domain or fragment thereof according to the present disclosure that is specific for IL-18Rα, and one binding site of said bispecific antibody is formed by a VHH antibody domain or fragment thereof according to the present disclosure that is specific for IL-18Rβ.

Embodiment 431: The compound according to any one of embodiments 375 to 426 or 428 to 429, wherein one binding site of said bispecific antibody molecule is a VHH antibody domain or fragment thereof according to the present disclosure and the other binding site of said bispecific antibody molecule is selected from the group consisting of a Fab, a Fab′, a (Fab′)₂, a Fv, a scFv, a diabody and a VHH.

“Fab” fragments are obtained by papain digestion of an antibody, which produces two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (CH1). Each Fab fragment is monovalent with respect to antigen binding, i.e., it has a single antigen-binding site.

“F(ab′)₂” fragments are obtained by pepsin treatment of an antibody, which yields a single large F(ab′)₂ fragment which roughly corresponds to two disulfide linked Fab fragments having different antigen-binding activity and is still capable of cross-linking antigen. “Fab′” fragments differ from Fab fragments by having a few additional residues at the carboxy terminus of the CH1 domain including one or more cysteines from the antibody hinge region. Fab′-SH is the designation for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv”, also abbreviated as “scFv”, are antibody fragments that comprise the VH and VL antibody domains connected into a single polypeptide chain. Preferably, the scFv polypeptide further comprises a polypeptide linker between the VH and VL domains which enables the scFv to form the desired structure for antigen binding. For a review of the scFv, see Pluckthun, in: The Pharmacology of Monoclonal Antibodies, vol. 113 (1994), editors Rosenburg and Moore, Springer-Verlag (New York), p. 269-315.

The term “diabody” refers to a small antibody fragment prepared by constructing scFv fragments (see preceding paragraph) with short linkers (about 5-25 residues) between the VH and VL domains such that inter-chain but not intra-chain pairing of the V domains is achieved, thereby resulting in a bivalent fragment, i.e., a fragment having two antigen-binding sites. Bispecific diabodies are heterodimers of two “crossover” scFv fragments in which the VH and VL domains of the two antibodies are present on different polypeptide chains. Diabodies are described in greater detail in, for example, EP 0404097; WO 93/11161; Hollinger et al., Proc. Natl. Acad. Sci. USA (1993), vol. 90, p. 6444-6448.

Embodiment 432: The compound according to any one of embodiments 375 to 426 or 430 to 431, wherein said compound is a multispecific molecule.

Embodiment 433: The compound according to any one of embodiments 375 to 426 or 428 to 430, wherein said compound is a bispecific molecule that is specific for IL-18Rα and IL-18Rβ.

Embodiment 434: The compound according to any one of embodiments 375 to 426 or 428 to 430 or 433, wherein said compound is a bispecific molecule that binds to IL-18Rα and IL-18RP.

Embodiment 435: The compound according to any one of embodiments 375 to 426 or 428 to 430 or 433 to 434, wherein said compound is a bispecific molecule directed to IL-18Rα and IL-18RP.

Embodiment 436: The compound according to any one of embodiments 375 to 426 or 428 to 430 or 433 to 435, wherein said compound is a bispecific antibody molecule that is specific for IL-18Rα and IL-18Rβ.

If the present disclosure refers to “IL-18Rα” without indicating a specific organism from which said IL-18Rα originates, then this refers to human IL-18Rα. If the present disclosure refers to “IL-18Rβ” without indicating a specific organism from which said IL-18Rβ originates, then this refers to human IL-18Rβ.

According to a twelfth aspect, the present disclosure relates to any of the following (To the twelfth aspect of the present disclosure and the embodiments referring thereto, the same explanations and definitions apply accordingly as for the first to eleventh aspects of the present disclosure and the embodiments referring thereto.)

Embodiment 437: A compound which is a bispecific antibody molecule comprising an IL-18Ra-binding VHH with the amino acid sequence of SEQ ID NO: 1 (First binding module) and an IL18Rβ-binding VHH with the amino acid sequence of SEQ ID NO: 12 (Second binding module).

Embodiment 438: A compound which is a bispecific antibody molecule comprising an IL-18Rα-binding VHH with the amino acid sequence of SEQ ID NO: 1 (First binding module) and an IL18Rβ-binding VHH with the amino acid sequence of SEQ ID NO: 13 (Second binding module).

Embodiment 439: A compound which is a bispecific antibody molecule comprising an IL-18Rα-binding VHH with the amino acid sequence of SEQ ID NO: 1 (First binding module) and an IL18Rβ-binding VHH with the amino acid sequence of SEQ ID NO: 15 (Second binding module).

Embodiment 440: A compound which is a bispecific antibody molecule comprising an IL-18Rα-binding VHH with the amino acid sequence of SEQ ID NO: 1 (First binding module) and an IL18Rβ-binding VHH with the amino acid sequence of SEQ ID NO: 17 (Second binding module).

Embodiment 441: A compound which is a bispecific antibody molecule comprising an IL-18Rα-binding VHH with the amino acid sequence of SEQ ID NO: 1 (First binding module) and an IL18Rβ-binding VHH with the amino acid sequence of SEQ ID NO: 20 (Second binding module).

Embodiment 442: A compound which is a bispecific antibody molecule comprising an IL-18Rα-binding VHH with the amino acid sequence of SEQ ID NO: 1 (First binding module) and an IL18Rβ-binding VHH with the amino acid sequence of SEQ ID NO: 21 (Second binding module).

Embodiment 443: A compound which is a bispecific antibody molecule comprising an IL-18Rα-binding VHH with the amino acid sequence of SEQ ID NO: 1 (First binding module) and an IL18Rβ-binding VHH with the amino acid sequence of SEQ ID NO: 22 (Second binding module).

Embodiment 444: A compound which is a bispecific antibody molecule comprising an IL-18Rα-binding VHH with the amino acid sequence of SEQ ID NO: 2 (First binding module) and an IL18Rβ-binding VHH with the amino acid sequence of SEQ ID NO: 12 (Second binding module).

Embodiment 445: A compound which is a bispecific antibody molecule comprising an IL-18Rα-binding VHH with the amino acid sequence of SEQ ID NO: 2 (First binding module) and an IL18Rβ-binding VHH with the amino acid sequence of SEQ ID NO: 13 (Second binding module).

Embodiment 446: A compound which is a bispecific antibody molecule comprising an IL-18Rα-binding VHH with the amino acid sequence of SEQ ID NO: 2 (First binding module) and an IL18Rβ-binding VHH with the amino acid sequence of SEQ ID NO: 15 (Second binding module).

Embodiment 447: A compound which is a bispecific antibody molecule comprising an IL-18Rα-binding VHH with the amino acid sequence of SEQ ID NO: 2 (First binding module) and an IL18Rβ-binding VHH with the amino acid sequence of SEQ ID NO: 17 (Second binding module).

Embodiment 448: A compound which is a bispecific antibody molecule comprising an IL-18Rα-binding VHH with the amino acid sequence of SEQ ID NO: 2 (First binding module) and an IL18Rβ-binding VHH with the amino acid sequence of SEQ ID NO: 18 (Second binding module).

Embodiment 449: A compound which is a bispecific antibody molecule comprising an IL-18Rα-binding VHH with the amino acid sequence of SEQ ID NO: 2 (First binding module) and an IL18Rβ-binding VHH with the amino acid sequence of SEQ ID NO: 20 (Second binding module).

Embodiment 450: A compound which is a bispecific antibody molecule comprising an IL-18Rα-binding VHH with the amino acid sequence of SEQ ID NO: 2 (First binding module) and an IL18Rβ-binding VHH with the amino acid sequence of SEQ ID NO: 21 (Second binding module).

Embodiment 451: A compound which is a bispecific antibody molecule comprising an IL-18Rα-binding VHH with the amino acid sequence of SEQ ID NO: 5 (First binding module) and an IL18Rβ-binding VHH with the amino acid sequence of SEQ ID NO: 13 (Second binding module).

Embodiment 452: A compound which is a bispecific antibody molecule comprising an IL-18Rα-binding VHH with the amino acid sequence of SEQ ID NO: 5 (First binding module) and an IL18Rβ-binding VHH with the amino acid sequence of SEQ ID NO: 14 (Second binding module).

Embodiment 453: A compound which is a bispecific antibody molecule comprising an IL-18Rα-binding VHH with the amino acid sequence of SEQ ID NO: 6 (First binding module) and an IL18Rβ-binding VHH with the amino acid sequence of SEQ ID NO: 17 (Second binding module).

Embodiment 454: A compound which is a bispecific antibody molecule comprising an IL-18Rα-binding VHH with the amino acid sequence of SEQ ID NO: 6 (First binding module) and an IL18Rβ-binding VHH with the amino acid sequence of SEQ ID NO: 20 (Second binding module).

Embodiment 455: A compound which is a bispecific antibody molecule comprising an IL-18Rα-binding VHH with the amino acid sequence of SEQ ID NO: 6 (First binding module) and an IL18Rβ-binding VHH with the amino acid sequence of SEQ ID NO: 21 (Second binding module).

Embodiment 456: A compound which is a bispecific antibody molecule comprising an IL-18Rα-binding VHH with the amino acid sequence of SEQ ID NO: 6 (First binding module) and an IL18Rβ-binding VHH with the amino acid sequence of SEQ ID NO: 22 (Second binding module).

Embodiment 457: A compound which is a bispecific antibody molecule comprising an IL-18Rα-binding VHH with the amino acid sequence of SEQ ID NO: 8 (First binding module) and an IL18Rβ-binding VHH with the amino acid sequence of SEQ ID NO: 12 (Second binding module).

Embodiment 458: A compound which is a bispecific antibody molecule comprising an IL-18Rα-binding VHH with the amino acid sequence of SEQ ID NO: 8 (First binding module) and an IL18Rβ-binding VHH with the amino acid sequence of SEQ ID NO: 13 (Second binding module).

Embodiment 459: A compound which is a bispecific antibody molecule comprising an IL-18Rα-binding VHH with the amino acid sequence of SEQ ID NO: 8 (First binding module) and an IL18Rβ-binding VHH with the amino acid sequence of SEQ ID NO: 17 (Second binding module).

Embodiment 460: A compound which is a bispecific antibody molecule comprising an IL-18Rα-binding VHH with the amino acid sequence of SEQ ID NO: 8 (First binding module) and an IL18Rβ-binding VHH with the amino acid sequence of SEQ ID NO: 18 (Second binding module).

Embodiment 461: A compound which is a bispecific antibody molecule comprising an IL-18Rα-binding VHH with the amino acid sequence of SEQ ID NO: 8 (First binding module) and an IL18Rβ-binding VHH with the amino acid sequence of SEQ ID NO: 20 (Second binding module).

Embodiment 462: A compound which is a bispecific antibody molecule comprising an IL-18Rα-binding VHH with the amino acid sequence of SEQ ID NO: 8 (First binding module) and an IL18Rβ-binding VHH with the amino acid sequence of SEQ ID NO: 21 (Second binding module).

Embodiment 463: A compound which is a bispecific antibody molecule comprising an IL-18Rα-binding VHH with the amino acid sequence of SEQ ID NO: 9 (First binding module) and an IL18Rβ-binding VHH with the amino acid sequence of SEQ ID NO: 12 (Second binding module).

Embodiment 464: A compound which is a bispecific antibody molecule comprising an IL-18Rα-binding VHH with the amino acid sequence of SEQ ID NO: 9 (First binding module) and an IL18Rβ-binding VHH with the amino acid sequence of SEQ ID NO: 13 (Second binding module).

Embodiment 465: A compound which is a bispecific antibody molecule comprising an IL-18Rα-binding VHH with the amino acid sequence of SEQ ID NO: 9 (First binding module) and an IL18Rβ-binding VHH with the amino acid sequence of SEQ ID NO: 15 (Second binding module).

Embodiment 466: A compound which is a bispecific antibody molecule comprising an IL-18Rα-binding VHH with the amino acid sequence of SEQ ID NO: 9 (First binding module) and an IL18Rβ-binding VHH with the amino acid sequence of SEQ ID NO: 17 (Second binding module).

Embodiment 467: A compound which is a bispecific antibody molecule comprising an IL-18Rα-binding VHH with the amino acid sequence of SEQ ID NO: 9 (First binding module) and an IL18Rβ-binding VHH with the amino acid sequence of SEQ ID NO: 20 (Second binding module).

Embodiment 468: A compound which is a bispecific antibody molecule comprising an IL-18Rα-binding VHH with the amino acid sequence of SEQ ID NO: 9 (First binding module) and an IL18Rβ-binding VHH with the amino acid sequence of SEQ ID NO: 21 (Second binding module).

Embodiment 469: A compound which is a bispecific antibody molecule comprising an IL-18Rα-binding VHH with the amino acid sequence of SEQ ID NO: 9 (First binding module) and an IL18Rβ-binding VHH with the amino acid sequence of SEQ ID NO: 22 (Second binding module).

Embodiment 470: A compound which is a bispecific antibody molecule comprising an IL-18Rα-binding VHH with the amino acid sequence of SEQ ID NO: 10 (First binding module) and an IL18Rβ-binding VHH with the amino acid sequence of SEQ ID NO: 13 (Second binding module).

Embodiment 471: A compound which is a bispecific antibody molecule comprising an IL-18Rα-binding VHH with the amino acid sequence of SEQ ID NO: 10 (First binding module) and an IL18Rβ-binding VHH with the amino acid sequence of SEQ ID NO: 20 (Second binding module).

Embodiment 472: A compound which is a bispecific antibody molecule comprising an IL-18Rα-binding VHH with the amino acid sequence of SEQ ID NO: 10 (First binding module) and an IL18Rβ-binding VHH with the amino acid sequence of SEQ ID NO: 21 (Second binding module).

Embodiment 473: The compound according to any one of embodiments 375 or 377 to 472, wherein said compound is monovalent for binding to IL-18Rα.

Embodiment 474: The compound according to any one of embodiments 375 or 377 to 472, wherein said compound is bivalent for binding to IL-18Rα.

Embodiment 475: The compound according to any one of embodiments 375 or 377 to 472, wherein said compound is multivalent for binding to IL-18Rα.

Embodiment 476: The compound according to any one of embodiments 376 to 475, wherein said compound is monovalent for binding to IL-18Rβ.

Embodiment 477: The compound according to any one of embodiments 376 to 475, wherein said compound is bivalent for binding to IL-18Rβ.

Embodiment 478: The compound according to any one of embodiments 376 to 475, wherein said compound is multivalent for binding to IL-18Rβ.

Embodiment 479: The compound according to any one of embodiments 377 to 478, wherein said compound comprises one copy of said first binding module.

Embodiment 480: The compound according to any one of embodiments 377 to 478, wherein said compound comprises two copies of said first binding module.

Embodiment 481: The compound according to any one of embodiments 377 to 478, wherein said compound comprises two or more copies of said first binding module.

Embodiment 482: The compound according to any one of embodiments 377 to 479, wherein said compound comprises not more than one copy of said first binding module.

Embodiment 483: The compound according to any one of embodiments 377 to 478 or 480, wherein said compound comprises not more than two copies of said first binding module.

Embodiment 484: The compound according to any one of embodiments 377 to 483, wherein said compound comprises one copy of said second binding module.

Embodiment 485: The compound according to any one of embodiments 377 to 483, wherein said compound comprises two copies of said second binding module.

Embodiment 486: The compound according to any one of embodiments 377 to 483, wherein said compound comprises two or more copies of said second binding module.

Embodiment 487: The compound according to any one of embodiments 377 to 484, wherein said compound comprises not more than one copy said second binding module.

Embodiment 488: The compound according to any one of embodiments 377 to 483 or 485, wherein said compound comprises not more than two copies of said second binding module.

Embodiment 489: The compound according to any one of embodiments 375 to 488, wherein said compound comprises only one molecular structure that binds to IL-18Rα.

Embodiment 490: The compound according to any one of embodiments 375 to 489, wherein said compound comprises only one molecular structure that binds to IL-18Rβ.

Embodiment 491: The compound according to any one of embodiments 375 to 490, wherein all components of said compound are covalently linked.

Embodiment 492: The compound according to any one of embodiments 377 to 491, wherein said first binding module is a VHH antibody domain. As the skilled person understands, by stating that the first binding module “is a VHH antibody domain”, the present disclosure indicates that the first binding module is a full VHH antibody domain, not just a fragment thereof.

Embodiment 493: The compound according to any one of embodiments 377 to 492, wherein said second binding module is a VHH antibody domain.

Embodiment 494: The compound according to any one of embodiments 377 to 491, wherein said first binding module consists of a fragment of a VHH antibody domain. As the skilled person understands, by stating that the first binding module “consists of a fragment of a VHH antibody domain”, the present disclosure indicates that the first binding module is a fragment of a VHH antibody domain, not a full-length VHH antibody domain.

Embodiment 495: The compound according to any one of embodiments 377 to 491 or 494, wherein said second binding module consists of a fragment of a VHH antibody domain.

Embodiment 496: The compound according to any one of embodiments 375 to 495, wherein said compound comprises an antibody Fc region.

As used herein, the term “antibody Fc region” refers to the portion of a native immunoglobulin formed by the Fc domains of its two heavy chains (which includes a heavy chain constant region 1 (CH1), a heavy chain constant region 2 (CH2) and a heavy chain constant region 3 (CH3) of an immunoglobulin, but does not include variable regions of the heavy and light chains and a light chain constant region 1 (CL1) of an immunoglobulin). A native Fc region is homodimeric. In some embodiments, the term includes variant Fc regions with one or more alterations relative to a native Fc region. An Fc region may be altered by amino acid substitutions, additions and/or deletions, linkage of additional moieties, and/or alteration of the native glycans. The term encompasses Fc regions wherein each of the constituent Fc domains is different. Examples of heterodimeric Fc regions include, without limitation, Fc regions made using the “knobs into holes” technology as described in, for example U.S. Pat. No. 8,216,805 or by the SEED technology as described in WO 2016/087650.

Embodiment 497: The compound according to any one of embodiments 375 to 495, wherein said compound comprises an antibody Fc region that is competent in Fc receptor binding.

An antibody Fc region is “competent in FC receptor binding” if said antibody Fc region is capable of binding to at least one of the Fc receptors (FcγRI, FcγRII, and FcγRIII subclasses, including allelic variants and alternatively spliced forms of these receptors).

Embodiment 498: The compound according to any one of embodiments 375 to 495, wherein said compound comprises an antibody Fc region that is not competent in Fc receptor binding.

Embodiment 499: The compound according to any one of embodiments 375 to 495, wherein said compound does not comprise an effector-competent antibody Fc region.

An “effector-competent” Fc region is an Fc region having the functional ability to bind proteins and/or cells of the immune system and mediate biological effects normally induced following the binding of an antibody to a corresponding antigen. Such biological effects include e.g. the ability to bind a complement protein (e.g. C1q), resulting in activation of the classical complement system leading to the opsonisation and lysis of cell pathogens (complement-dependent cytotoxicity, CDCC). Other biological effects are endocytosis of immune complexes, engulfment and destruction of antibody-coated particles or microorganisms (also called antibody-dependent phagocytosis, or ADCP), clearance of immune complexes, lysis of antibody-coated target cells by killer cells (called antibody-dependent cell-mediated cytotoxicity, or ADCC), release of inflammatory mediators, regulation of immune system cell activation or control of immunoglobulin production.

Embodiment 500: The compound according to any one of embodiments 375 to 497, wherein said compound comprises an effector-competent antibody Fc region.

Embodiment 501: The compound according to any one of embodiments 375 to 495 or 498 to 499, wherein said compound does not comprise an antibody Fc region capable of inducing ADCC (antibody-dependent cellular cytotoxicity).

Embodiment 502: The compound according to any one of embodiments 375 to 497 or 500, wherein said compound comprises an antibody Fc region capable of inducing ADCC.

Embodiment 503: The compound according to any one of embodiments 375 to 499, wherein said compound comprises an antibody Fc region that is not capable of inducing ADCC.

Embodiment 504: The compound according to any one of embodiments 375 to 503, wherein that antibody Fc region carries a mutation E430G.

This mutation is described in de Jong et al., 2016 and has been reported to drive antibody hexamerization upon target cell binding.

Embodiment 505: The compound according to any one of embodiments 377 to 504, wherein said compound is a bispecific antibody molecule prepared by the SEED technology (i.e. a SEEDbody).

The SEED (strand-exchange engineered domain) technology is an approach for generation of bispecific antibodies in which structurally related sequences within the conserved CH3 domains of human IgA and IgG are exchanged to form two asymmetric but complementary domains (see the Examples section). For details about the SEED technology are described in WO 2016/087650 and Davis et al., 2010. A bispecific antibody prepared by the SEED technology is herein also referred to as “SEEDbody”.

Embodiment 506: The compound according to any one of embodiments 377 to 505, wherein the VHH antibody domain or fragment thereof of the first binding module is formed by a sequence selected from the group consisting of SEQ ID NO: 89, SEQ ID NO: 90, SEQ ID NO: 91, SEQ ID NO: 92, SEQ ID NO: 94, SEQ ID NO: 95, SEQ ID NO: 96, SEQ ID NO: 97, SEQ ID NO: 98 and SEQ ID NO: 99,
and wherein the VHH antibody domain or fragment thereof of the second binding module is formed by a sequence selected from the group consisting of SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109 and SEQ ID NO: 110. SEQ ID NO: 89 to 99 define the amino acid sequences of SEED AG chains based on VHH1 to VHH11 (α1 to β11). SEQ ID NO: 100 to 110 define the amino acid sequences of SEED GA chains based on VHH12 to VHH22 (β12 to β22).

Embodiment 507: The compound according to any one of embodiments 377 to 506, wherein said compound is a bispecific antibody with a strictly monovalent (1+1) bispecific sdAb (single-domain antibody) architecture.

Embodiment 508: The compound according to any one of embodiments 377 to 507, wherein said compound comprises two VHHs of different binding specificity, and each of these VHHs is present in said compound in only one copy. As the skilled person understands, if the compound is a bispecific antibody comprising a first and a second binding module, then typically one VHH forms the first binding module of said compound whereas the other VHH forms the second binding module of said compound.

Embodiment 509: The compound according to any one of embodiments 377 to 504 or 506 to 508, wherein said compound is a single domain-based IgG (sdIgG).

Embodiment 510: The compound according to embodiment 509, wherein a sdIgG is a conventional IgG antibody in which the two VH regions are replaced by VHH domains of one binding specificity, and the two VL regions are replaced by VHH domains of a different binding specificity, resulting in a bispecific, tetravalent antibody.

The single domain-based IgG (sdIgG) format is described in detail in Pekar et al., 2020.

Embodiment 511: The compound according to any one of embodiments 509 to 510, wherein said sdIgG is a conventional IgG antibody in which each of the two VH regions is replaced by a copy of said first binding module, and each of the two VL regions is replaced by a copy of said second binding module, resulting in a bispecific, tetravalent antibody.

Embodiment 512: The compound according to embodiment 511, wherein the first binding module is a VHH that has been grafted onto the CH1 domain of the heavy chain of an IgG1 and the second binding module is a VHH that has been grafted onto the constant region of the lambda light chain of an IgG1.

Embodiment 513: The compound according to any one of embodiments 510 to 512, wherein said IgG/IgG1 is a human IgG1.

Embodiment 514: The compound according to any one of embodiments 377 to 504 or 507 to to 513, wherein the IL-18Rα-specific VHH2 (SEQ ID NO: 2) has been grafted onto the CH1 domain of the heavy chain of an IgG1 and the IL-18Rβ-specific VHH15 (SEQ ID NO: 15) has been grafted onto the constant region of the lambda light chain of an IgG1.

Embodiment 515: The compound according to any one of embodiments 377 to 514, wherein said heavy chain of said IgG is effector-silenced.

Embodiment 516: The compound according to any one of embodiments 377 to 504 or 515, wherein said compound consists of a IgG Fc region to which two copies of a tandem-VHH arrangement are covalently linked at the hinge-region, wherein said tandem-VHH arrangement consists of a first VHH antibody domain that is covalently linked to a second VHH antibody domain, wherein said second VHH antibody domain is covalently linked to the hinge region of said Fc region, resulting in a bispecific, tetravalent compound.

Embodiment 517: The compound according to embodiment 516, wherein said IgG Fc region is an effector-silenced IgG1 Fc region.

Embodiment 518: The compound according to any one of embodiments 516 to 517, wherein said first binding module forms said first VHH antibody domain and said second binding module forms said second VHH antibody domain.

Embodiment 519: The compound according to any one of embodiments 516 to 518, wherein said first VHH antibody domain and said second VHH antibody domain are separated by a linker.

Embodiment 520: The compound according to any one of embodiments 516 to 519, wherein said first VHH antibody domain and said second VHH antibody domain are separated by a five amino acid Gly₄Ser-linker.

Embodiment 521: The compound according to any one of embodiments 516 to 520, wherein said first VHH antibody domain is specific for IL-18Rα and said second VHH antibody domain is specific for IL-18Rβ.

Embodiment 522: The compound according to any one of embodiments 516 to 521, wherein said first VHH antibody domain has the amino acid sequence of VHH2 (SEQ ID NO: 2) and said second VHH antibody domain has the amino acid sequence of VHH15 (SEQ ID NO: 15).

Embodiment 523: The compound according to any one of embodiments 516 to 520, wherein said first VHH antibody domain is specific for IL-18Rβ and said second VHH antibody domain is specific for IL-18Rα.

Embodiment 524: The compound according to any one of embodiments 516 to 520 or 523, wherein said first VHH antibody domain has the amino acid sequence of VHH15 (SEQ ID NO: 15) and said second VHH antibody domain has the amino acid sequence of VHH2 (SEQ ID NO: 2).

Embodiment 525: The compound according to any one of embodiments 516 to 524, wherein the orientation of the first and second VHH antibody domains is the first VHH antibody domains followed by the second VHH antibody domain (from N-terminus to C-terminus).

Embodiment 526: The compound according to any one of embodiments 516 to 525, wherein the orientation of the first and second VHH antibody domains is the second VHH antibody domains followed by the first VHH antibody domain (from N-terminus to C-terminus).

Embodiment 527: The compound according to any one of embodiments 375 to 526, wherein said compound is an IL-18 mimetic.

As used herein, the term “mimetic” refers to a compound that imitates certain aspects of the function of another compound. Provided herein are, among other things, IL-18 mimetics. IL-18 is a proinflammatory cytokine belonging to the IL-1 family of cytokines that mediates signalling through heterodimerization of the receptor subunits IL-18Rα and IL-18Rβ (Yasuda et al., 2019; Dinarello et al., 2013). IL-18 stimulates IFN-γ production in innate lymphoid cells as well as antigen-experienced T cells in synergy with IL-12. The aspects of the function of IL18 that such IL-18 mimetics imitate is the induction of heterodimerization of IL-18R (from IL-18Rα and IL-18Rβ) in the presence of IL-12 (or IL-15). Without being bound by theory, it is speculated that by binding to both IL-18Rα and IL-18Rβ, the IL-18 mimetics of the present disclosure crosslink IL-18Rα and IL-18Rβ, thus leading to dimerization and activation of IL-18R in the presence of IL-12 (or IL-15). Thus, the IL-18 mimetics of the present disclosure imitate the biological function of IL-18, for example the function of IL-18 to induce IFN-γ release (in the presence of IL-12 or IL-15).

Embodiment 528: The compound according to embodiment 527, wherein said IL-18 mimetic is not affected from inhibition by IL-18BP (IL-18 binding protein).

Embodiment 529: The compound according to any one of embodiments 375 to 528, wherein said compound is a IL-18 receptor (IL-18R) agonist.

Embodiment 530: The compound according to any one of embodiments 375 to 529, wherein said compound is a cytokine mimetic targeting IL-18Rα and IL-18Rβ.

Embodiment 531: The compound according to any one of embodiments 375 to 530, wherein said compound binds to IL-18Rα.

Embodiment 532: The compound according to any one of embodiments 375 to 531, wherein said compound specifically binds to IL-18Rα.

Embodiment 533: The compound according to any one of embodiments 375 to 532, wherein said compound is capable of binding to IL-18Rα with an affinity that is at least equal to the affinity with which VHH1 (SEQ ID NO: 1), VHH2 (SEQ ID NO: 2), VHH3 (SEQ ID NO: 3), VHH4 (SEQ ID NO: 4), VHH5 (SEQ ID NO: 5), VHH6 (SEQ ID NO: 6), VHH8 (SEQ ID NO: 8), VHH9 (SEQ ID NO: 9) or VHH10 (SEQ ID NO: 10) binds to IL-18Rα (or stronger).

Embodiment 534: The compound according to any one of embodiments 375 to 533, wherein said compound binds to IL-18Rβ.

Embodiment 535: The compound according to any one of embodiments 375 to 534, wherein said compound specifically binds to IL-18Rβ.

Embodiment 536: The compound according to any one of embodiments 375 to 535, wherein said compound is capable of binding to IL-18Rβ with an affinity that is at least equal to the affinity with which VHH12 (SEQ ID NO: 12), VHH13 (SEQ ID NO: 13), VHH14 (SEQ ID NO: 14), VHH15 (SEQ ID NO: 15), VHH16 (SEQ ID NO: 16), VHH17 (SEQ ID NO: 17), VHH18 (SEQ ID NO: 18), VHH20 (SEQ ID NO: 20), VHH21 (SEQ ID NO: 21) or VHH22 (SEQ ID NO: 22) binds to IL-18Rβ(or stronger).

Embodiment 537: The compound according to any one of embodiments 375 to 536, wherein said compound binds to IL-18Rα and IL-18Rβ.

Embodiment 538: The compound according to any one of embodiments 375 to 537, wherein said compound specifically binds to IL-18Rα and IL-18Rβ.

Embodiment 539: The compound according to any one of embodiments 375 to 538, wherein said compound is capable of crosslinking IL-18Rα and IL-18Rβ.

Embodiment 540: The compound according to any one of embodiments 375 to 539, wherein said compound is capable of inducing heterodimerization of IL-18Rα and IL-18Rβ. Such binding/crosslinking/heterodimerization can be determined by in vitro binding experiments as described in Example 1 below (by biolayer interferometry).

Embodiment 541: The compound according to any one of embodiments 375 to 540, wherein said compound binds to IL-18Rα ECD with a KD of 100 nM or stronger.

Embodiment 542: The compound according to any one of embodiments 375 to 540, wherein said compound binds to IL-18Rα ECD with a KD of 50 nM or stronger.

Embodiment 543: The compound according to any one of embodiments 375 to 540, wherein said compound binds to IL-18Rα ECD with a KD of 25 nM or stronger.

Embodiment 544: The compound according to any one of embodiments 375 to 540, wherein said compound binds to IL-18Rα ECD with a KD of 10 nM or stronger.

Embodiment 545: The compound according to any one of embodiments 375 to 540, wherein said compound binds to IL-18Rα ECD with a KD of 1 nM or stronger.

Embodiment 546: The compound according to any one of embodiments 375 to 540, wherein said compound binds to IL-18Rα ECD with a KD of 500 μM or stronger.

Embodiment 547: The compound according to any one of embodiments 375 to 540, wherein said compound binds to IL-18Rα ECD with a KD of 100 μM or stronger.

Embodiment 548: The compound according to any one of embodiments 375 to 540, wherein said compound binds to IL-18Rα ECD with a KD of 10 μM or stronger.

Embodiment 549: The compound according to any one of embodiments 375 to 548, wherein said compound binds to IL-18Rβ ECD with a KD of 100 nM or stronger.

Embodiment 550: The compound according to any one of embodiments 375 to 548, wherein said compound binds to IL-18Rβ ECD with a KD of 50 nM or stronger.

Embodiment 551: The compound according to any one of embodiments 375 to 548, wherein said compound binds to IL-18RβECD with a KD of 25 nM or stronger.

Embodiment 552: The compound according to any one of embodiments 375 to 548, wherein said compound binds to IL-18Rβ ECD with a KD of 10 nM or stronger.

Embodiment 553: The compound according to any one of embodiments 375 to 548, wherein said compound binds to IL-18RβECD with a KD of 1 nM or stronger.

Embodiment 554: The compound according to any one of embodiments 375 to 548, wherein said compound binds to IL-18Rβ ECD with a KD of 500 μM or stronger.

Embodiment 555: The compound according to any one of embodiments 375 to 548, wherein said compound binds to IL-18Rβ ECD with a KD of 100 μM or stronger.

Embodiment 556: The compound according to any one of embodiments 375 to 548, wherein said compound binds to IL-18Rβ ECD with a KD of 10 μM or stronger.

Embodiment 557: The compound according to any one of embodiments 375 to 556, wherein said compound shows competitive binding with recombinant human IL-18 for targeting IL-18Rα.

Embodiment 558: The compound according to any one of embodiments 375 to 557, wherein said compound shows competitive binding with recombinant human IL-18 for targeting IL-18Rβ.

Embodiment 559: The compound according to any one of embodiments 375 to 558, wherein said compound is capable of activating the IL-18 receptor IL-18R.

Embodiment 560: The compound according to any one of embodiments 375 to 559, wherein said compound is capable of activating the IL-18 receptor IL-18R by binding to IL-18Rα and IL-18Rβ.

Embodiment 561: The compound according to any one of embodiments 375 to 560, wherein said compound is capable of activating the IL-18 receptor IL-18R by cross-linking the IL-18 receptor subunits IL-18Rα and IL-18Rβ.

Embodiment 562: The compound according to any one of embodiments 375 to 558, wherein said compound is does not activate the IL-18 receptor IL-18R.

Embodiment 563: The compound according to any one of embodiments 375 to 562, wherein said compound triggers dose-dependent IL-18R downstream signaling on IL-18 reporter cells. Activation of IL-18R and whether a compound triggers dose-dependent IL-18R downstream signaling on IL-18 reporter cells can for example be determined as described in Example 1, section “Human IL-18 HEK reporter assay”.

Embodiment 564: The compound according to any one of embodiments 375 to 563, wherein said compound induces at a concentration of 50 nM at least 1.5-fold higher NFκB activation than TNF-α at a concentration of 50 nM.

Embodiment 565: The compound according to any one of embodiments 375 to 564, wherein said compound shows a lower potency of IL-18R receptor activation (as determined from the EC₅₀ of NFκB reporter activation) compared with IL-18.

Embodiment 566: The compound according to any one of embodiments 375 to 565, wherein said compound shows a lower magnitude of IL-18 activation (as determined by maximal NFκB activation) compared with IL-18.

Whether a compound shows a higher potency of IL-18R receptor activation (as determined from the EC₅₀ of NFκB reporter activation) compared with IL-18 and/or shows lower magnitude of IL-18 activation (as determined by maximal NFκB activation) compared with IL-18 can be determined based on an assay as described in Example 3 and Example 1, section “Human IL-18 HEK reporter assay”.

Embodiment 567: The compound according to any one of embodiments 375 to 566, wherein said IL-18 is recombinant human IL-18.

Embodiment 568: The compound according to any one of embodiments 375 to 567, wherein said compound induces at a concentration of 1 nM a normalized NFκB reporter activation of more than 50% relative to the NFκB reporter activation induced by recombinant human IL18 at a concentration of 1 nM or wherein said compound has an EC₅₀ for NFκB reporter activation of less than 0.1 nM.

Embodiment 569: The compound according to any one of embodiments 375 to 568, wherein said compound induces at a concentration of 1 nM a normalized NFκB reporter activation of more than 50% relative to the NFκB reporter activation induced by recombinant human IL18 at a concentration of 1 nM.

Embodiment 570: The compound according to any one of embodiments 375 to 569, wherein said compound has an EC₅₀ for NFκB reporter activation of less than 0.1 nM.

Embodiment 571: The compound according to any one of embodiments 375 to 567, wherein said compound induces at a concentration of 1 nM a normalized NFκB reporter activation of less than 50% relative to the NFκB reporter activation induced by recombinant human IL18 at a concentration of 1 nM or wherein said compound has an EC₅₀ for NFκB reporter activation of more than 0.1 nM.

Embodiment 572: The compound according to any one of embodiments 375 to 567 or 571, wherein said compound induces at a concentration of 1 nM a normalized NFκB reporter activation of less than 50% relative to the NFκB reporter activation induced by recombinant human IL18 at a concentration of 1 nM.

Embodiment 573: The compound according to any one of embodiments 375 to 567 or 571 to 572, wherein said compound has an EC₅₀ for NFκB reporter activation of more than 0.1 nM. Whether a compound induces at a concentration of 1 nM a normalized NFκB reporter activation of more or less than 50% relative to the NFκB reporter activation induced by recombinant human IL18 at a concentration of 1 nM and/or has an EC₅₀ for NFκB reporter activation of less or more than 0.1 nM can be determined based on an assay as described in Example 3 and Example 1, section “Human IL-18 HEK reporter assay”.

Embodiment 574: The compound according to any one of embodiments 375 to 573, wherein said compound is capable of inducing IFN-γ release by peripheral blood mononuclear cells (PBMCs) in the presence of low-dose IL-12.

Embodiment 575: The compound according to embodiment 574, wherein said low-dose IL-12 is 10 ng/ml IL-12.

Embodiment 576: The compound according to any one of embodiments 375 to 575, wherein said IL-12 is recombinant human IL-12.

Details how to determine whether a compound is capable of inducing IFN-γ release by peripheral blood mononuclear cells (PBMCs) in the presence of low-dose IL-12 are described in Example 1, section “IFN-γ release assay”.

Embodiment 577: The compound according to any one of embodiments 375 to 576, wherein said compound has an EC50 for the release of IFN-γ from human PBMCs of less than 150 nM.

Embodiment 578: The compound according to any one of embodiments 375 to 576, wherein said compound has an EC50 for the release of IFN-γ from human PBMCs of less than 100 nM.

Embodiment 579: The compound according to any one of embodiments 375 to 576, wherein said compound has an EC50 for the release of IFN-γ from human PBMCs of less than 50 nM.

Embodiment 580: The compound according to any one of embodiments 375 to 576, wherein said compound has an EC50 for the release of IFN-γ from human PBMCs of less than 20 nM.

Embodiment 581: The compound according to any one of embodiments 375 to 576, wherein said compound has an EC50 for the release of IFN-γ from human PBMCs of less than 150 μM.

Embodiment 582: The compound according to any one of embodiments 375 to 576, wherein said compound has an EC50 for the release of IFN-γ from human PBMCs of less than 100 μM.

Embodiment 583: The compound according to any one of embodiments 375 to 576, wherein said compound has an EC50 for the release of IFN-γ from human PBMCs of less than 50 μM.

Embodiment 584: The compound according to any one of embodiments 375 to 576, wherein said compound has an EC50 for the release of IFN-γ from human PBMCs of less than 20 μM.

Embodiment 585: The compound according to any one of embodiments 375 to 584, wherein the capacity of the compound to trigger IFN-γ release on PBMCs (as measured by EC₅₀) is increased compared to recombinant human IL-18. As the skilled person understands, this means that the EC₅₀ of said compound to trigger IFN-γ release on PBMCs is smaller than that of recombinant human IL-18.

Embodiment 586: The compound according to any one of embodiments 375 to 585, wherein the EC50 of said compound for the release of IFN-γ from human PBMCs is less than 50% of the EC50 of (rh) IL-18 for the release of IFN-γ from human PBMCs.

As used herein, “rh” in the context of a molecule stands for “recombinant human”.

Embodiment 587: The compound according to any one of embodiments 375 to 585, wherein the EC50 of said compound for the release of IFN-γ from human PBMCs is less than 200% of the EC50 of (rh) IL-18 for the release of IFN-γ from human PBMCs.

Embodiment 588: The compound according to any one of embodiments 565 to 587, wherein said EC50 is measured in the presence of low dose (rh) IL-12.

Embodiment 589: The compound according to any one of embodiments 375 to 588, wherein said compound causes a release of IFN-γ that is at least 200% of the IFN-γ release caused by (rh) IL-18.

Embodiment 590: The compound according to any one of embodiments 375 to 588, wherein said compound causes a release of IFN-γ that is at least 150% of the IFN-γ release caused by (rh) IL-18.

Embodiment 591: The compound according to any one of embodiments 375 to 588, wherein said compound causes a release of IFN-γ that is at least 100% of the IFN-γ release caused by (rh) IL-18.

Embodiment 592: The compound according to any one of embodiments 375 to 588, wherein said compound causes a release of IFN-γ that is at least 80% of the IFN-γ release caused by (rh) IL-18.

Embodiment 593: The compound according to any one of embodiments 375 to 588, wherein said compound causes a release of IFN-γ that is at least 50% of the IFN-γ release caused by (rh) IL-18.

Embodiment 594: The compound according to any one of embodiments 375 to 588, wherein said compound causes a release of IFN-γ that is at least 10% of the IFN-γ release caused by (rh) IL-18.

Embodiment 595: The compound according to any one of embodiments 574 to 594, wherein said IFN-γ release is measured upon stimulation of human peripheral blood mononuclear cells (PBMCs) with said compound (resp. with IL-18) in the presence of low dose recombinant human IL-12 (10 ng/ml).

Embodiment 596: The compound according to embodiment 595, wherein said compound resp. said IL-18 is used at a concentration of 1 nM.

Embodiment 597: The compound according to any one of embodiments 574 to 596, wherein said release of IFN-γ is the release of IFN-γ at a concentration of 1 nM of said compound resp. said IL-18.

Embodiment 598: The compound according to any one of embodiments 574 to 595, wherein said release of IFN-γ is the maximum release of IFN-γ.

Further details about the assay are described in the Examples section, in particular Example 1, section “IFN-γ release assay”.

Embodiment 599: The compound according to any one of embodiments 375 to 598, wherein said compound is not affected by the presence of IL-18 binding protein (IL-18BP).

Embodiment 600: The compound according to any one of embodiments 375 to 599, wherein said compound does not bind to recombinant human IL-18 binding protein (IL-18BP).

Embodiment 601: The compound according to any one of embodiments 375 to 600, wherein binding of said compound to IL-18Rα and IL-18Rβ is not affected by the presence of IL-18 binding protein (IL-18BP).

Embodiment 602: The compound according to any one of embodiments 375 to 601, wherein said activation of IL-18R by said compound is not affected by the presence of IL-18 binding protein (IL-18BP).

Embodiment 603: The compound according to any one of embodiments 375 to 602, wherein said capacity of said compound to induce IFN-γ release is not affected by the presence of IL-18BP under conditions where the capacity of IL-18 to induce IFN-γ release is inhibited by the presence of IL-18BP.

Embodiment 604: The compound according to any one of embodiments 375 to 603, wherein the EC50 of said compound for the release of IFN-γ from human PBMCs is not increased by the presence of (rh) IL-18BP (compared to the absence of (rh) IL-18BP) under conditions where the EC50 of IL-18 for the release of IFN-γ from human PBMCs is increased by the presence of (rh) IL-18BP (compared to the absence of (rh) IL-18BP).

Embodiment 605: The VHH antibody domain or fragment thereof according to any one of embodiments 1 to 374 or the compound according to any one of embodiments 375 to 604, wherein said binding is determined by biolayer interferometry at 25 °C and 1000 rpm in KB Buffer (PBS+0.1% Tween-20+1% BSA).

Embodiment 606: The VHH antibody domain or fragment thereof according to any one of embodiments 1 to 374 or the compound according to any one of embodiments 375 to 604, wherein said competition is determined by biolayer interferometry at 25 °C and 1000 rpm in KB Buffer (PBS+0.1% Tween-20+1% BSA).

Embodiment 607: The VHH antibody domain or fragment thereof according to any one of embodiments 1 to 374 or the compound according to any one of embodiments 375 to 604, wherein said crosslinking/heterodimerization is determined by biolayer interferometry at 25 °C and 1000 rpm in KB Buffer (PBS+0.1% Tween-20+1% BSA).

Embodiment 608: The VHH antibody domain or fragment thereof according to any one of embodiments 1 to 374 or the compound according to any one of embodiments 375 to 604, wherein said KD value is measured by kinetic measurements by biolayer interferometry at 25 °C and 1000 rpm in KB Buffer (PBS+0.1% Tween-20+1% BSA).

According to a thirteenth aspect, the present disclosure relates to any of the following (To the thirteenth aspect of the present disclosure and the embodiments referring thereto, the same explanations and definitions apply accordingly as for the first to twelfth aspects of the present disclosure and the embodiments referring thereto.)

Embodiment 609: A bispecific antibody molecule prepared by the SEED (strand-exchange engineered domain) technology, said molecule comprising
an AG chain linked to an IL-18Rα-binding VHH, wherein said AG chain linked to said IL-18Rα-binding VHH has the amino acid sequence of SEQ ID NO: 89 (“VHH-SEED-AG-1”) and
a GA chain linked to an IL18Rβ-binding VHH, wherein said GA chain linked to said IL18Rβ-binding VHH has the amino acid sequence of SEQ ID NO: 100 (“VHH-SEED-GA-1”).

Embodiment 610: A bispecific antibody molecule prepared by the SEED (strand-exchange engineered domain) technology, said molecule comprising
an AG chain linked to an IL-18Rα-binding VHH, wherein said AG chain linked to said IL-18Rα-binding VHH has the amino acid sequence of SEQ ID NO: 89 (“VHH-SEED-AG-1”) and
a GA chain linked to an IL18Rβ-binding VHH, wherein said GA chain linked to said IL18Rβ-binding VHH has the amino acid sequence of SEQ ID NO: 101 (“VHH-SEED-GA-2”).

Embodiment 611: A bispecific antibody molecule prepared by the SEED (strand-exchange engineered domain) technology, said molecule comprising
an AG chain linked to an IL-18Rα-binding VHH, wherein said AG chain linked to said IL-18Rα-binding VHH has the amino acid sequence of SEQ ID NO: 89 (“VHH-SEED-AG-i”) and
a GA chain linked to an IL18Rβ-binding VHH, wherein said GA chain linked to said IL18Rβ-binding VHH has the amino acid sequence of SEQ ID NO: 103 (“VHH-SEED-GA-4”).

Embodiment 612: A bispecific antibody molecule prepared by the SEED (strand-exchange engineered domain) technology, said molecule comprising
an AG chain linked to an IL-18Rα-binding VHH, wherein said AG chain linked to said IL-18Rα-binding VHH has the amino acid sequence of SEQ ID NO: 89 (“VHH-SEED-AG-1”) and
a GA chain linked to an IL18Rβ-binding VHH, wherein said GA chain linked to said IL18Rβ-binding VHH has the amino acid sequence of SEQ ID NO: 105 (“VHH-SEED-GA-6”).

Embodiment 613: A bispecific antibody molecule prepared by the SEED (strand-exchange engineered domain) technology, said molecule comprising
an AG chain linked to an IL-18Rα-binding VHH, wherein said AG chain linked to said IL-18Rα-binding VHH has the amino acid sequence of SEQ ID NO: 89 (“VHH-SEED-AG-1”) and
a GA chain linked to an IL18Rβ-binding VHH, wherein said GA chain linked to said IL18Rβ-binding VHH has the amino acid sequence of SEQ ID NO: 108 (“VHH-SEED-GA-9”).

Embodiment 614: A bispecific antibody molecule prepared by the SEED (strand-exchange engineered domain) technology, said molecule comprising

an AG chain linked to an IL-18Rα-binding VHH, wherein said AG chain linked to said IL-18Rα-binding VHH has the amino acid sequence of SEQ ID NO: 89 (“VHH-SEED-AG-1”) and
a GA chain linked to an IL18Rβ-binding VHH, wherein said GA chain linked to said IL18Rβ-binding VHH has the amino acid sequence of SEQ ID NO: 109 (“VHH-SEED-GA-10”).

Embodiment 615: A bispecific antibody molecule prepared by the SEED (strand-exchange engineered domain) technology, said molecule comprising
an AG chain linked to an IL-18Rα-binding VHH, wherein said AG chain linked to said IL-18Rα-binding VHH has the amino acid sequence of SEQ ID NO: 89 (“VHH-SEED-AG-1”) and
a GA chain linked to an IL18Rβ-binding VHH, wherein said GA chain linked to said IL18Rβ-binding VHH has the amino acid sequence of SEQ ID NO: 110 (“VHH-SEED-GA-11”).

Embodiment 616: A bispecific antibody molecule prepared by the SEED (strand-exchange engineered domain) technology, said molecule comprising
an AG chain linked to an IL-18Rα-binding VHH, wherein said AG chain linked to said IL-18Rα-binding VHH has the amino acid sequence of SEQ ID NO: 90 (“VHH-SEED-AG-2”) and
a GA chain linked to an IL18Rβ-binding VHH, wherein said GA chain linked to said IL18Rβ-binding VHH has the amino acid sequence of SEQ ID NO: 100 (“VHH-SEED-GA-1”).

Embodiment 617: A bispecific antibody molecule prepared by the SEED (strand-exchange engineered domain) technology, said molecule comprising
an AG chain linked to an IL-18Rα-binding VHH, wherein said AG chain linked to said IL-18Rα-binding VHH has the amino acid sequence of SEQ ID NO: 90 (“VHH-SEED-AG-2”) and
a GA chain linked to an IL18Rβ-binding VHH, wherein said GA chain linked to said IL18Rβ-binding VHH has the amino acid sequence of SEQ ID NO: 101 (“VHH-SEED-GA-2”).

Embodiment 618: A bispecific antibody molecule prepared by the SEED (strand-exchange engineered domain) technology, said molecule comprising
an AG chain linked to an IL-18Rα-binding VHH, wherein said AG chain linked to said IL-18Rα-binding VHH has the amino acid sequence of SEQ ID NO: 90 (“VHH-SEED-AG-2”) and
a GA chain linked to an IL18Rβ-binding VHH, wherein said GA chain linked to said IL18Rβ-binding VHH has the amino acid sequence of SEQ ID NO: 103 (“VHH-SEED-GA-4”).

Embodiment 619: A bispecific antibody molecule prepared by the SEED (strand-exchange engineered domain) technology, said molecule comprising
an AG chain linked to an IL-18Rα-binding VHH, wherein said AG chain linked to said IL-18Rα-binding VHH has the amino acid sequence of SEQ ID NO: 90 (“VHH-SEED-AG-2”) and
a GA chain linked to an IL18Rβ-binding VHH, wherein said GA chain linked to said IL18Rβ-binding VHH has the amino acid sequence of SEQ ID NO: 105 (“VHH-SEED-GA-6”).

Embodiment 620: A bispecific antibody molecule prepared by the SEED (strand-exchange engineered domain) technology, said molecule comprising
an AG chain linked to an IL-18Rα-binding VHH, wherein said AG chain linked to said IL-18Rα-binding VHH has the amino acid sequence of SEQ ID NO: 90 (“VHH-SEED-AG-2”) and
a GA chain linked to an IL18Rβ-binding VHH, wherein said GA chain linked to said IL18Rβ-binding VHH has the amino acid sequence of SEQ ID NO: 106 (“VHH-SEED-GA-7”).

Embodiment 621: A bispecific antibody molecule prepared by the SEED (strand-exchange engineered domain) technology, said molecule comprising
an AG chain linked to an IL-18Rα-binding VHH, wherein said AG chain linked to said IL-18Rα-binding VHH has the amino acid sequence of SEQ ID NO: 90 (“VHH-SEED-AG-2”) and
a GA chain linked to an IL18Rβ-binding VHH, wherein said GA chain linked to said IL18Rβ-binding VHH has the amino acid sequence of SEQ ID NO: 108 (“VHH-SEED-GA-9”).

Embodiment 622: A bispecific antibody molecule prepared by the SEED (strand-exchange engineered domain) technology, said molecule comprising
an AG chain linked to an IL-18Rα-binding VHH, wherein said AG chain linked to said IL-18Rα-binding VHH has the amino acid sequence of SEQ ID NO: 90 (“VHH-SEED-AG-2”) and
a GA chain linked to an IL18Rβ-binding VHH, wherein said GA chain linked to said IL18Rβ-binding VHH has the amino acid sequence of SEQ ID NO: 109 (“VHH-SEED-GA-10”).

Embodiment 623: A bispecific antibody molecule prepared by the SEED (strand-exchange engineered domain) technology, said molecule comprising
an AG chain linked to an IL-18Rα-binding VHH, wherein said AG chain linked to said IL-18Rα-binding VHH has the amino acid sequence of SEQ ID NO: 93 (“VHH-SEED-AG-5”) and
a GA chain linked to an IL18Rβ-binding VHH, wherein said GA chain linked to said IL18Rβ-binding VHH has the amino acid sequence of SEQ ID NO: 101 (“VHH-SEED-GA-2”).

Embodiment 624: A bispecific antibody molecule prepared by the SEED (strand-exchange engineered domain) technology, said molecule comprising
an AG chain linked to an IL-18Rα-binding VHH, wherein said AG chain linked to said IL-18Rα-binding VHH has the amino acid sequence of SEQ ID NO: 93 (“VHH-SEED-AG-5”) and
a GA chain linked to an IL18Rβ-binding VHH, wherein said GA chain linked to said IL18Rβ-binding VHH has the amino acid sequence of SEQ ID NO: 102 (“VHH-SEED-GA-3”).

Embodiment 625: A bispecific antibody molecule prepared by the SEED (strand-exchange engineered domain) technology, said molecule comprising
an AG chain linked to an IL-18Rα-binding VHH, wherein said AG chain linked to said IL-18Rα-binding VHH has the amino acid sequence of SEQ ID NO: 94 (“VHH-SEED-AG-6”) and
a GA chain linked to an IL18Rβ-binding VHH, wherein said GA chain linked to said IL18Rβ-binding VHH has the amino acid sequence of SEQ ID NO: 105 (“VHH-SEED-GA-6”).

Embodiment 626: A bispecific antibody molecule prepared by the SEED (strand-exchange engineered domain) technology, said molecule comprising
an AG chain linked to an IL-18Rα-binding VHH, wherein said AG chain linked to said IL-18Rα-binding VHH has the amino acid sequence of SEQ ID NO: 94 (“VHH-SEED-AG-6”) and
a GA chain linked to an IL18Rβ-binding VHH, wherein said GA chain linked to said IL18Rβ-binding VHH has the amino acid sequence of SEQ ID NO: 108 (“VHH-SEED-GA-9”).

Embodiment 627: A bispecific antibody molecule prepared by the SEED (strand-exchange engineered domain) technology, said molecule comprising
an AG chain linked to an IL-18Rα-binding VHH, wherein said AG chain linked to said IL-18Rα-binding VHH has the amino acid sequence of SEQ ID NO: 94 (“VHH-SEED-AG-6”) and
a GA chain linked to an IL18Rβ-binding VHH, wherein said GA chain linked to said IL18Rβ-binding VHH has the amino acid sequence of SEQ ID NO: 109 (“VHH-SEED-GA-10”).

Embodiment 628: A bispecific antibody molecule prepared by the SEED (strand-exchange engineered domain) technology, said molecule comprising
an AG chain linked to an IL-18Rα-binding VHH, wherein said AG chain linked to said IL-18Rα-binding VHH has the amino acid sequence of SEQ ID NO: 94 (“VHH-SEED-AG-6”) and
a GA chain linked to an IL18Rβ-binding VHH, wherein said GA chain linked to said IL18Rβ-binding VHH has the amino acid sequence of SEQ ID NO: 110 (“VHH-SEED-GA-11”).

Embodiment 629: A bispecific antibody molecule prepared by the SEED (strand-exchange engineered domain) technology, said molecule comprising
an AG chain linked to an IL-18Rα-binding VHH, wherein said AG chain linked to said IL-18Rα-binding VHH has the amino acid sequence of SEQ ID NO: 96 (“VHH-SEED-AG-8”) and
a GA chain linked to an IL18Rβ-binding VHH, wherein said GA chain linked to said IL18Rβ-binding VHH has the amino acid sequence of SEQ ID NO: 100 (“VHH-SEED-GA-1”).

Embodiment 630: A bispecific antibody molecule prepared by the SEED (strand-exchange engineered domain) technology, said molecule comprising
an AG chain linked to an IL-18Rα-binding VHH, wherein said AG chain linked to said IL-18Rα-binding VHH has the amino acid sequence of SEQ ID NO: 96 (“VHH-SEED-AG-8”) and
a GA chain linked to an IL18Rβ-binding VHH, wherein said GA chain linked to said IL18Rβ-binding VHH has the amino acid sequence of SEQ ID NO: 101 (“VHH-SEED-GA-2”).

Embodiment 631: A bispecific antibody molecule prepared by the SEED (strand-exchange engineered domain) technology, said molecule comprising
an AG chain linked to an IL-18Rα-binding VHH, wherein said AG chain linked to said IL-18Rα-binding VHH has the amino acid sequence of SEQ ID NO: 96 (“VHH-SEED-AG-8”) and
a GA chain linked to an IL18Rβ-binding VHH, wherein said GA chain linked to said IL18Rβ-binding VHH has the amino acid sequence of SEQ ID NO: 105 (“VHH-SEED-GA-6”).

Embodiment 632: A bispecific antibody molecule prepared by the SEED (strand-exchange engineered domain) technology, said molecule comprising
an AG chain linked to an IL-18Rα-binding VHH, wherein said AG chain linked to said IL-18Rα-binding VHH has the amino acid sequence of SEQ ID NO: 96 (“VHH-SEED-AG-8”) and
a GA chain linked to an IL18Rβ-binding VHH, wherein said GA chain linked to said IL18Rβ-binding VHH has the amino acid sequence of SEQ ID NO: 106 (“VHH-SEED-GA-7”).

Embodiment 633: A bispecific antibody molecule prepared by the SEED (strand-exchange engineered domain) technology, said molecule comprising
an AG chain linked to an IL-18Rα-binding VHH, wherein said AG chain linked to said IL-18Rα-binding VHH has the amino acid sequence of SEQ ID NO: 96 (“VHH-SEED-AG-8”) and
a GA chain linked to an IL18Rβ-binding VHH, wherein said GA chain linked to said IL18Rβ-binding VHH has the amino acid sequence of SEQ ID NO: 108 (“VHH-SEED-GA-9”).

Embodiment 634: A bispecific antibody molecule prepared by the SEED (strand-exchange engineered domain) technology, said molecule comprising
an AG chain linked to an IL-18Rα-binding VHH, wherein said AG chain linked to said IL-18Rα-binding VHH has the amino acid sequence of SEQ ID NO: 96 (“VHH-SEED-AG-8”) and
a GA chain linked to an IL18Rβ-binding VHH, wherein said GA chain linked to said IL18Rβ-binding VHH has the amino acid sequence of SEQ ID NO: 109 (“VHH-SEED-GA-10”).

Embodiment 635: A bispecific antibody molecule prepared by the SEED (strand-exchange engineered domain) technology, said molecule comprising
an AG chain linked to an IL-18Rα-binding VHH, wherein said AG chain linked to said IL-18Rα-binding VHH has the amino acid sequence of SEQ ID NO: 97 (“VHH-SEED-AG-9”) and
a GA chain linked to an IL18Rβ-binding VHH, wherein said GA chain linked to said IL18Rβ-binding VHH has the amino acid sequence of SEQ ID NO: 100 (“VHH-SEED-GA-1”).

Embodiment 636: A bispecific antibody molecule prepared by the SEED (strand-exchange engineered domain) technology, said molecule comprising
an AG chain linked to an IL-18Rα-binding VHH, wherein said AG chain linked to said IL-18Rα-binding VHH has the amino acid sequence of SEQ ID NO: 97 (“VHH-SEED-AG-9”) and
a GA chain linked to an IL18Rβ-binding VHH, wherein said GA chain linked to said IL18Rβ-binding VHH has the amino acid sequence of SEQ ID NO: 101 (“VHH-SEED-GA-2”).

Embodiment 637: A bispecific antibody molecule prepared by the SEED (strand-exchange engineered domain) technology, said molecule comprising
an AG chain linked to an IL-18Rα-binding VHH, wherein said AG chain linked to said IL-18Rα-binding VHH has the amino acid sequence of SEQ ID NO: 97 (“VHH-SEED-AG-9”) and
a GA chain linked to an IL18Rβ-binding VHH, wherein said GA chain linked to said IL18Rβ-binding VHH has the amino acid sequence of SEQ ID NO: 103 (“VHH-SEED-GA-4”).

Embodiment 638: A bispecific antibody molecule prepared by the SEED (strand-exchange engineered domain) technology, said molecule comprising
an AG chain linked to an IL-18Rα-binding VHH, wherein said AG chain linked to said IL-18Rα-binding VHH has the amino acid sequence of SEQ ID NO: 97 (“VHH-SEED-AG-9”) and
a GA chain linked to an IL18Rβ-binding VHH, wherein said GA chain linked to said IL18Rβ-binding VHH has the amino acid sequence of SEQ ID NO: 105 (“VHH-SEED-GA-6”).

Embodiment 639: A bispecific antibody molecule prepared by the SEED (strand-exchange engineered domain) technology, said molecule comprising
an AG chain linked to an IL-18Rα-binding VHH, wherein said AG chain linked to said IL-18Rα-binding VHH has the amino acid sequence of SEQ ID NO: 97 (“VHH-SEED-AG-9”) and
a GA chain linked to an IL18Rβ-binding VHH, wherein said GA chain linked to said IL18Rβ-binding VHH has the amino acid sequence of SEQ ID NO: 108 (“VHH-SEED-GA-9”).

Embodiment 640: A bispecific antibody molecule prepared by the SEED (strand-exchange engineered domain) technology, said molecule comprising
an AG chain linked to an IL-18Rα-binding VHH, wherein said AG chain linked to said IL-18Rα-binding VHH has the amino acid sequence of SEQ ID NO: 97 (“VHH-SEED-AG-9”) and
a GA chain linked to an IL18Rβ-binding VHH, wherein said GA chain linked to said IL18Rβ-binding VHH has the amino acid sequence of SEQ ID NO: 109 (“VHH-SEED-GA-10”).

Embodiment 641: A bispecific antibody molecule prepared by the SEED (strand-exchange engineered domain) technology, said molecule comprising
an AG chain linked to an IL-18Rα-binding VHH, wherein said AG chain linked to said IL-18Rα-binding VHH has the amino acid sequence of SEQ ID NO: 97 (“VHH-SEED-AG-9”) and
a GA chain linked to an IL18Rβ-binding VHH, wherein said GA chain linked to said IL18Rβ-binding VHH has the amino acid sequence of SEQ ID NO: 110 (“VHH-SEED-GA-11”).

Embodiment 642: A bispecific antibody molecule prepared by the SEED (strand-exchange engineered domain) technology, said molecule comprising
an AG chain linked to an IL-18Rα-binding VHH, wherein said AG chain linked to said IL-18Rα-binding VHH has the amino acid sequence of SEQ ID NO: 98 (“VHH-SEED-AG-10”) and
a GA chain linked to an IL18Rβ-binding VHH, wherein said GA chain linked to said IL18Rβ-binding VHH has the amino acid sequence of SEQ ID NO: 101 (“VHH-SEED-GA-2”).

Embodiment 643: A bispecific antibody molecule prepared by the SEED (strand-exchange engineered domain) technology, said molecule comprising
an AG chain linked to an IL-18Rα-binding VHH, wherein said AG chain linked to said IL-18Rα-binding VHH has the amino acid sequence of SEQ ID NO: 98 (“VHH-SEED-AG-10”) and
a GA chain linked to an IL18Rβ-binding VHH, wherein said GA chain linked to said IL18Rβ-binding VHH has the amino acid sequence of SEQ ID NO: 108 (“VHH-SEED-GA-9”).

Embodiment 644: A bispecific antibody molecule prepared by the SEED (strand-exchange engineered domain) technology, said molecule comprising
an AG chain linked to an IL-18Rα-binding VHH, wherein said AG chain linked to said IL-18Rα-binding VHH has the amino acid sequence of SEQ ID NO: 98 (“VHH-SEED-AG-10”) and
a GA chain linked to an IL18Rβ-binding VHH, wherein said GA chain linked to said IL18Rβ-binding VHH has the amino acid sequence of SEQ ID NO: 109 (“VHH-SEED-GA-10”).

According to a fourteenth aspect, the present disclosure relates to a pharmaceutical composition comprising the compound or bispecific antibody molecule according to any one of the aspects or embodiments described above.

Methods for preparing pharmaceutical compositions are known to a skilled person in the art (Remington: The Science and Practice of Pharmacy, 22nd ed. (2012), Pharmaceutical Press).

In some embodiments, said pharmaceutical composition comprises a pharmaceutically acceptable carrier, diluent and/or excipient.

The term “pharmaceutically acceptable” designates that said carrier, diluent or excipient is a non-toxic, inert material that is compatible with the other ingredients of the pharmaceutical composition and not harmful to the patient that the pharmaceutical composition is administered to, such that it can be used in a pharmaceutical product. Substances suitable as carriers, diluents or excipients in pharmaceutical compositions are known to a skilled person in the art (Remington: The Science and Practice of Pharmacy, 22nd ed. (2012), Pharmaceutical Press). The pharmaceutical composition may further include e.g. additional adjuvants, antioxidants, buffering agents, bulking agents, colorants, emulsifiers, fillers, flavoring agents, preservatives, stabilizers, suspending agents and/or other customary pharmaceutical auxiliaries.

According to a fifteenth aspect, the present disclosure relates to the use of a compound or bispecific antibody molecule according to any one of the aspects or embodiments described above for activating IL-18R receptor.

According to a sixteenth aspect, the present disclosure relates to the use of a compound or bispecific antibody molecule according to any one of the aspects or embodiments described above for inducing IL-7 release from cells carrying an IL-18R receptor.

Preferably, said use occurs in the presence of IL-12.

Preferably, said cells carrying an IL-18R receptor are T cells.

Preferably, said cells carrying an IL-18R receptor are Th1 T cells.

### EXAMPLES

The following examples describe the preparation and characterization of IL18 mimetics as disclosed in the present disclosure, as well as related compounds and methods, along with comparative disclosure. It is understood that various embodiments of the disclosure reflected 4914 in the examples may be practiced, given the general description provided above. Although the foregoing invention has been described in some detail by way of illustration and example for purposes of clarity of understanding, the description and examples should not be construed as limiting the scope of the invention.

### Example 1

### Camelid Immunization

For the immunization procedure, one llama (Lama glama) with an age of about 12.5 years and one male huarizo (Lama glama×Vicugna pacos) of about 11 years of age were immunized with a cocktail of recombinant human (rh) IL18Rα extracellular domain (ECD) as Fc-fusion (R&D Systems) as well as rh IL18Rβ ECD (Sino Biologics) with a poly histidine-tag. For administration, the antigens were diluted to a stock concentration of 1 mg/mL in PBS and emulsified for initial immunization with Complete Freund's Adjuvant or with Incomplete Freund's Adjuvant for subsequent immunizations. The antigens were injected subcutaneously at three sites with 200 μg material in total (1:1 ratio of IL18Rα and ILR18β). This procedure was conducted four times over a period of 35 days, i.e., administration at d0, d14, d24 and d35. One week after final administration (d42), a volume of 150 ml blood was collected from each specimen for subsequent RNA extraction and cDNA synthesis. Of note, processes involving animals were performed at preclinics GmbH, Germany and in accordance with local regulations and animal welfare protection laws, hence immunized animals remained alive after final blood collection.

### Yeast Strains and Media

For antibody surface display, the yeast Saccharomyces cerevisiae strain EBY100 (MATa URA3-52 trp1 leu2Δ1 his3Δ200 pep4::HIS3 prb1Δ1.6R can1 GAL (pIU211:URA3)) (Thermo Fisher Scientific) was employed. The cells were cultivated in yeast extract-peptone-dextrose (YPD) medium composed of 10 g/L yeast extract, 20 g/L peptone and 20 g/L dextrose, additionally supplemented with 10 mg/mL penicillin-streptomycin (Gibco). Cells harboring the library plasmids (pDisp) after gap repair cloning were cultivated in minimal synthetic defined (SD)-base (Takara Bio) medium supplemented with 5.4 g/L Na2HPO4 and 8.6 g/L NaH2PO4 H2O, also comprising the corresponding dropout mix (Takara Bio) composed of all essential amino acids except for tryptophan (-Trp) for selection. To induce antibody gene expression, dextrose was replaced by galactose as carbon source, thus cells were transferred into SG dropout medium (-Trp) consisting of SG-base medium (Takara Bio) and 10% (w/v) polyethylene glycol 8000 (PEG 8000).

### Plasmids for Yeast Surface Display and Library Generation

Homologous recombination-based cloning, referred to as gap repair cloning, was utilized for the generation of the VHH libraries in yeast. For this, our group already described the specific PCR amplification of VHH fragments as well as library construction detailed elsewhere (Roth et al., 2020). In short, digestion of a stuffer sequence in the pDisp with specific restriction enzyme BsaI allow for the subsequent genetic fusion of VHH library candidates in frame to Aga2p by gap repair cloning, ultimately enabling the sdAb presentation on the surface of the yeast cell. Additionally, monitoring of proper full-length VHH presentation on yeast surface was realized by a HA epitope C-terminally linked to Aga2p on the pDisp backbone.

### Library Sorting

EBY100 cells were grown overnight in SD medium with dropout mix lacking tryptophan (-Trp) at 30 °C and 120 rpm. Next day, cells were transferred into SG medium with dropout mix (-Trp) at 107 cells/mL to induce VHH surface expression and incubated for another 48 h at 20 4964 °C and 120 rpm. The fluorescence activated cell sorting (FACS) procedures were conducted on a BD FACSAria™ Fusion cell sorter (BD Biosciences) device. For library sorting purposes, full-length VHH surface expression was monitored by application of an Alexa Fluor 488 labeled mouse monoclonal anti-HA antibody (R&D Systems, diluted 1:20). A two-dimensional sorting strategy was enabled by simultaneous antigen binding detection using indirect immunofluorescence staining with 250 nM rh his-tagged IL18Rα (Sino Biological) or 250 nM rh his-tagged ILR18Rβ ECD (Sino Biological) in combination with murine anti-his detection antibody (Penta His Alexa Fluor 647 Conjugate, Qiagen, diluted 1:20, for sorting round I or Allophycocyanin anti-His Tag Antibody, BioLegend, diluted 1:20, for sorting round II), respectively (FIG. 1B). Control samples of cells incubated with secondary labeling reagents and an unrelated antigen or cells incubated with secondary labeling only as well as untreated cells were employed in every FACS analysis, enabling an adequate gate adjustment for the desired cell population.

### Protein Expression, Purification and Analytics

After clone selection based on sequencing results obtained from FACS enriched populations, the VHH variants directed against IL18Rα ECD were N-terminally fused to the hinge region of Fc immune effector-silenced (eff-) SEED AG chains, while the VHHs targeting IL18Rβ ECD were fused accordingly to eff-SEED GA chains prior to cloning into pTT5 mammalian expression vector (Durocher, 2002), ultimately enabling the production of eff-monospecific (IL18R_msVHH) and bispecific SEEDbodies (1+1 IL18R_VHHaP). For small-scale production of the proteins (25 mL scale), Expi293 cells were transiently transfected with respective pTT5 vectors according to the manufacturer's instructions (Thermo Fisher Scientific). Six days post transfection the protein containing supernatants were harvested by centrifugation prior purification via MabSelect antibody purification chromatography resin (GE Healthcare). A buffer exchange step to PBS pH 6.8 overnight using Pur-A-Lyzer™ Maxi 3500 Dialysis Kit (Sigma Aldrich) was employed, followed by sterile filtration with Ultrafree®-CL GV 0.22 m centrifugal devices (Merck Millipore) and measurement of resulting molecule concentrations using Nanodrop ND-1000 (Peglab). Protein purities were afterwards determined by analytical size exclusion chromatography (SEC) on a TSKgel UP-SW3000 column (2 μm, 4994 4.6×300 mm, Tosoh Bioscience) using an Agilent HPLC 1260 Infinity system. 7.5 μg protein per sample were injected and run at a flow rate of 0.35 ml/min using 50 mM sodium phosphate, 0.4 M NaClO4 pH 6.3 as mobile phase.

Bispecific proteins generated for the assessment of antibody format engineering on IL-18 cytokine mimetics (FIG. 4A) were generated by the replacement of VH and VLλ of an effector silenced IgG by VHHα and VHHβ, respectively, thereby facilitating the generation of the IL18R_sdIgGαβ architecture (2+2). Tandem arrangement of the respective VHHs was achieved by N-terminal fusion to the hinge region of an effector silenced IgG1 Fc fragment, whereby the VHHs were separated by a five amino acid Gly4Ser linker, consequently leading to the (2+2) IL18R_tanVHHαβ design (2+2). Moreover, tandem arrangement was constructed for both VHH orientations, i.e., αβ and βα. Of note, all four molecules were also produced harboring the E430G mutation for on-target hexamerization.

These antibodies were transiently expressed from ExpiCHO cells (Thermo Fisher Scientific) following a 11-day protocol at 250 mL scale according to the manufacturer's instructions (Max Titer Protocol). The cultivation protocol included a temperature shift from 36.5 °C to 32.0 °C after addition of ExpiFectamine CHO Enhancer and first ExpiCHO Feed at day 1 while incubating at 5% CO2 and 80 rpm. Plasmids for transfections (pTT5 backbone) were used at 0.8 mg/L and were mixed 1:1 HC:LC (IgGs) or 2:1 AG:GA (SEEDs) for constructs composed of multiple antibody chains. Antibody containing supernatants were harvested by centrifugation at 4000×g for 20 min at 4 °C and afterwards sterile filtered. Protein purification was done by affinity capture on Protein A resin (HiTrap MabSelect SuRe, 5 ml) and acidic elution with 50 mM acetic acid pH 3.0 at 5 ml/min using an GE Healthcare AKTAxpress system followed by a desalting step at 10 ml/min (HiPrep 26/10 columns) into PBS pH 6.8. Protein purities were afterwards determined by analytical SEC on a TSKgel UP-SW3000 column (2 μm, 4.6×300 mm, Tosoh Bioscience) using an Agilent HPLC 1260 Infinity system. 7.5 μg protein per sample were injected and run at a flow rate of 0.35 ml/min using 50 mM sodium phosphate, 0.4 M NaClO4 pH 6.3 as mobile phase. For proteins with purities below 90%, a polishing step by preparative SEC (HiLoad 26/600 or 16/600 Superdex 200 μg) was applied. Final samples were sterile filtered over 0.2 μm, flash frozen in liquid nitrogen and stored at −80 °C until further use. To assure no quality loss after freeze/thaw, a final purity determination by analytical SEC was done. Furthermore, thermal unfolding of the antibodies was assessed by differential scanning fluorimetry (DSF) on a Prometheus NT.PLEX nanoDSF instrument (NanoTemper). Samples were measured in duplicates using nanoDSF Standard Capillary Chips. A temperature gradient from 20 °C to 95 °C at a slope of 1 °C/min was used while recording fluorescence at 350 and 330 nm. Unfolding transition midpoints (Tm) were determined from the first derivative of the fluorescence ratio 350 nm/330 nm.

### Biolayer Interferometry

For binding assays, kinetic measurements, competition assays as well as simultaneous binding with recombinant proteins, the Octet RED96 system (ForteBio, Pall Life Science) was employed using 25 °C and 1000 rpm agitation settings. In order to determine binding kinetics of monospecific molecules, human IL18Rα-His (Sino Biological) and human IL18Rβ-His (Sino Biological) were loaded on anti-Penta His (HIS1K) biosensors at 3 g/mL in PBS for 180 s followed by 60 s sensor rinsing in kinetics buffer (KB; PBS+0.1% Tween-20 and 1% bovine serum albumin, BSA). Afterwards, binding to monospecific molecules at 100 nM in KB was measured for 300 s followed by dissociation for 100 s in KB. In each experiment, one negative control using irrelevant antibody and a second reference by incubating the IL-18R subunits in KB instead of the monospecific molecules was measured. Kinetic constants (KD) of bispecific molecules to determine affinities were measured by loading bispecific molecules on anti-human Fc (AHC) biosensors for 180 s at 5 μg/mL in PBS. After sensor rinsing in KB for 45 s, interaction was tested by association of human IL18Rα-His or human IL18Rβ-His in decreasing concentrations from 100 nM to 12.5 nM for top four molecules and 100 nM to 11 nM for engineered constructs in KB for 180 s, followed by dissociation for 300 s in KB. Avidity effects of engineered tandem constructs were shown loading human IL18Rα-His and human IL18RP-His on HIS1K biosensors at 5 g/mL in PBS for 180 s, followed by 45 s sensor rinsing in KB. Association of engineered tandem constructs as well as parental molecule was measured in ranging concentrations from 100 nM to 11 nM for 180 s in KB followed by dissociation for 300 s in KB. Simultaneous binding capacities of IL18R subunits for engineered tandem constructs and parental molecule were measured by loading molecules on AHC biosensors for 180 s at 5 g/mL in PBS. After sensor rinsing a first binding step was performed using human IL18Rα-His or human IL18Rβ-His at 200 nM to saturate the binding capacity of the loaded molecule for 300 s. Afterward, the association was determined against other IL18R subunit for 180 s using varying concentrations from 100 nM to 11 nM. To avoid a dissociation of first bound IL18R subunit in this step, 100 nM of first bound subunit was used for every concentration tested in association. For the following dissociation step (300 s), first bound subunit was also used at 100 nM to assess dissociation against second IL18R subunit only. To determine KD of human IL-18 (R&D Systems) on human IL-18BP His (Acro Biosystems)/human IL-18BP Fc Tag (R&D Systems) was loaded on HIS1K/AHC biosensors at 5 μg/mL in PBS for 180 s. After sensor rinsing in KB for 45 s, association of human IL-18 was measured in decreasing concentrations from 10 nM to 0.3125 nM in KB for 180 s. Dissociation of human IL-18 was measured in KB for 300 s. Parallel control measurements for each association step of biosensors incubated in KB instead were utilized. Data were fitted and analyzed with ForteBio data analysis software 8.0 using a 1:1 binding model after Savitzky-Golay filtering. Epitope binning experiment of top 4 molecules on IL18R subunits were performed by loading human IL18Rα-His or human IL18Rβ-His on HIS1K biosensors at 5 μg/mL in PBS for 180 s, followed by 45 s sensor rinsing in KB. Binding to first bispecific molecule at 200 nM in KB was measured for 300 s to saturate binding capacities of loaded IL18R subunit, followed by a second association step of another bispecific molecule at 200 nM in KB for another 180 s. To avoid a dissociation of first bound bispecific molecule in second association, 100 nM of first bound bispecific molecule was used for every association of second bispecific molecule. To analyze competitive binding of top 4 molecules and engineered tandem constructs vs. human IL-18 on human IL18Rα-His, molecules were loaded at 5 μg/mL in PBS for 180 s on AHC biosensors, followed by 45 s sensor rinsing in KB. Association of the molecules at 200 nM for 300 s in KB was followed by an additional association step with human IL-18 at 100 nM for another 180 s in KB. Control values using an unrelated antibody as well as controls using KB buffer were included. To illustrate that our top 4 molecules and engineered tandem molecules are not affected by human IL-18BP His, molecules were loaded on AHC biosensors at 5 μg/mL for 180 s in PBS. After a rinsing step in KB for 45 s, human IL-18BP His was associated at 1000 nM for 180 s and dissociated in KB for 180 s. To show binding of human IL-18 to human IL-18BP, human IL-18BP His was loaded at 5 μg/mL in PBS for 180 s on HIS1K biosensors, followed by a rinsing step in KB for 45 s. Binding to human IL-18 at 1000 nM in KB was measured for 180 s followed by dissociation for 180 s in KB.

### Human IL-18 HEK Reporter Assay

For detection of the activation of JAK-STAT pathway by bispecific IL18R_VHHaP SEEDbodies the IL-18 HEK-Blue assay (InvivoGen, hkb-hmil18) was performed according to the manufacturer's instructions. In brief, 5×104 cells were seeded into each well of a 96-well plate and stimulated initially with 50 nM of selected IL18R_VHHaP for 24 hours at 37 °C and 5% CO2 including incubation with TNF-α and IL-18 as controls. For EC50 determination, samples were titrated with 1:5 serial dilution with concentrations ranging from 100 nM to 5.12×10−5 nM. Additionally, an IL-18 titration was performed with 1:5 serial dilution with concentrations ranging from 100 nM to 5.24×10-12 nM. Twenty microliters of cell culture supernatant were then taken from each well and mixed with 180 μl QUANTI-Blue medium in a 96-well plate, incubated for 3 h at 37 °C and 5% CO2, and then read in a microplate reader at 640 nm.

### IFN-γ Release Assay

For functional assays to measure IFN-γ release, freshly isolated human peripheral mononuclear cells (PBMCs) from healthy donors were used. Isolation of PBMCs from whole blood samples was performed using SepMate-50 tubes (StemCell Technologies) and Lymphoprep medium (StemCell Technologies) according to StemCell Technologies' SepMate PBMC Isolation protocol (2019). Human PBMCs were used directly for functional assays after isolation and cultivated in AIM V medium (Gibco) at 37 °C and 5% CO2 in a humidity box for 24 h during functional assays. For initial Cytokine Release Assay of top 16 molecules 1×106 human PBMCs per well were stimulated with bispecific molecules (100 nM) or human IL-18 (1 nM, R&D Systems) in AIM V medium in 96-well white-opaque cultivation plates (Perkin&Elmer). All stimulations were performed once in the presence of human IL-12 (10 ng/mL, R&D Systems) and once in the absence of human IL-12. To determine EC50 values of top 4 molecules 1×106 human PBMCs per well were stimulated with a range of 1000 nM to 0.122 nM of bispecific molecules and human IL-12 (10 ng/mL). For first functional test of engineered constructs based on parental molecule IL18R_VHHα2β15 1 ×106 human PBMCs per well were stimulated with two concentrations of 10 nM and 1 nM for engineered constructs and 1 nM/0.1 nM for human IL-18. All stimulations were performed in the presence of human IL-12 (10 ng/ml). To determine EC50 values of top engineered constructs 1×106 human PBMCs were stimulated with ranging concentrations of engineered constructs (1000 nM to 0.000209 nM) and IL-18 (0.457 nM to 0.000209 nM) as well as human IL-12 (10 ng/ml). For human IL-18BP Assay 1×106 human PBMCs were stimulated with a fixed concentration of IL-18 (0.5 nM) or engineered tandem constructs (0.5 nM) and range of IL-18BP concentrations (0.00001 nM to 100 nM). All stimulations were performed in the presence of human IL-12 (10 ng/ml). As controls for every functional assay a non-functional binder that serves as negative control as well as human PBMCs only stimulated with human IL-12 as basal stimulation reference were used. Quantification of IFN-γ released by human PBMCs was performed using human cytokine HTRF kits (Cisbio) as previously described by our group (Pekar et al., 2020). After 24 h incubation, PBMC cells were sedimented in 96-well plates by centrifugation, and cytokine-containing supernatants were further processed according to the manufacturer's instructions. Assay plates were measured with a PHERAstar FSX device (BMG Labtech). HTRF optical entity using excitation at 337 nm and emission at 620 nm as well as 665 nm was used. Analyses and fitting of resulting data were facilitated by MARS software (v3.32, BMG) enabling a four-parameter logistic (4PL 1/y2) model fitting of the standard curve following the kit manufacturer's instructions.

### Data Processing and Statistical Analysis

Graphical and statistical analyses were conducted with GraphPad Prism 8 software. P-values were calculated utilizing repeated measures 2way ANOVA and the Bonferroni as recommended. p<0.05 were regarded as statistically significant.

### Example 2

sdAbs Targeting IL-18Rα and IL-18Rβ

For the isolation of VHHs targeting IL-18Rα and IL-18Rβ one llama and one huarizo (llama×alpaca) were immunized with a cocktail of the extracellular domains (ECDs) of both receptor subunits. Afterwards, for each specimen a YSD library was generated as described previously (Roth et al., 2020; Klausz et al., 2022) and both libraries were sorted individually. A two-dimensional sorting strategy was applied to select for full-length VHH display via HA tag staining simultaneous to the binding functionality by exploiting an antigen concentration of 250 nM for each respective receptor subunit. In the first round of selection, a mixture of both receptor subunits was utilized (FIG. 1B). In the second selection round the enriched libraries were selected against both antigens separately and for each library and each receptor subunit enrichment for an antigen-binding population could be achieved (FIG. 1B, FIG. 5). From each library, 96 clones were sent out for sequencing for each antigen. This resulted in 55 unique clones in total enriched for binding to IL-18Rα and 101 unique clones for IL-18Rβ, respectively (FIG. 1C). By applying a clonotyping strategy based on CDR3 diversity, eleven VHHs targeting each receptor subunit, respectively, were selected for expression.

To this end, all 22 sdAbs were expressed as one-armed (1+0) antibodies by employing the SEED technology which relies on beta-strand exchanges of IgG and IgA CH3 constant domains, resulting in preferential heavy chain heterodimerization (FIG. 6A) (Davis et al., 2010). sdAbs enriched for IL-18Rα binding were grafted onto the hinge region of the AG chain of the SEEDbody and produced with a paratope-less GA chain, while sdAbs targeting IL-18Rβ were fused to the GA chain and combined with a non-targeted AG chain. An effector silenced derivative of the SEEDbody Fc region was exploited in order to abolish the potential of antibody-dependent cell-mediated cytotoxicity (ADCC) in primary cell assays (and to verify that abolishment of ADCC by such an approach also functions in this antibody set-up).

Following expression in ExpiCHO™ cells and protein A purification, we assessed binding to the ECDs of IL-18Rα and IL-18Rβ by biolayer interferometry (BLI) using an antigen concentration of 100 nM (FIG. 6B).

As expected, (given the low sequence identity of approximately 21% between IL-18Rα ECD and IL-18Rβ ECD), monospecific molecules obtained from the IL-18Rα sorts (ms_L18R_VHHα1-11) only showed binding to IL-18Rα ECD but not to IL-18Rβ ECD which was vice versa for monospecific clones selected from the IL-18Rβ enrichments (ms_IL18R_VHHβ12-22). In addition to this, ms_IL18R_VHHα7 only displayed negligible binding to IL-18Rα ECD, whereas IL-18Rβ-directed paratope ms_IL18R_VHHβ19 exhibited no binding at all. Consequently, both sdAbs were excluded from further consideration.

Thus, ten sdAbs specific for IL-18Rα (VHH1, VHH2, VHH3, VHH4, VHH5, VHH6, VHH8, VHH9, VHH10, VHH11) and ten sdAbs specific for IL-18Rβ (VHH12, VHH13, VHH14, VHH15, VHH16, VHH17, VHH18, VHH20, VHH21, VHH22) were obtained.

### Example 3

Identification of IL-18 Receptor Agonists after Combinatorial Reformatting of sdAbs Targeting INDIVIDUAL IL-18 Receptor Subunits as (1+1) bsAbs

For functional characterization, all ten VHHs targeting IL-18Rα (engrafted to the AG chain of the SEED architecture) as well as all ten sdAbs specific for IL-18Rβ (fused to the GA chain) were recombined and expressed as bispecific SEEDbodies, resulting in 100 bispecific sdAb-derived antibody derivatives.

As initial cut-off in terms of biophysical properties, we only considered bispecifics for further investigation that comprised more than 86% target peak in analytical size exclusion chromatography post protein A purification, resulting in twelve molecules that were excluded. Interestingly, this encompassed all bsAbs that harbored IL18R_VHHα11, indicating liabilities of the respective sdAb paratope. Notwithstanding, 88 VHH-derived bsAbs were assessed regarding their agonistic potential exploiting IL-18 reporter cells, stably expressing IL-18Rα and IL-18Rβ (HEK-Blue™ IL-18 cells).

In an initial experiment, bsAbs as well as all monospecific SEEDbodies (either series ms_IL18R_VHHα1-11 or ms_IL18R_VHHβ12-22) were used at a concentration of 50 nM. Recombinant human (rh) TNF-α was employed as negative control as well as (rh) IL-18 as positive control, showing a dose-dependent activation of IL-18 reporter cells (FIG. 7A). As expected, none of the monospecific SEEDbodies was able to agonize IL-18 reporter cells, whereas 44 out of the 88 sdAb-based bispecifics induced NFκB activation that was at least 1.5-fold higher than for TNF-α at this fixed concentration (data not shown). These molecules were further characterized exploiting the reporter cell assay in a dose-dependent manner. To this end, we also included IL18R_VHHα1β16 which did not show agonism potential but specific binding to the respective receptor subunits as negative control (FIG. 2A). Intriguingly, all 44 different bispecifics were able to agonize the IL-18 reporter cells to a certain extent, whereas IL18R_VHHα1β16 did not induce activation, as exemplarily shown for surrogate agonists IL18R_VHHα2β15, IL18R_VHHα8 15, IL18R_VHHα2p 17 and IL18R_VHHα8p 17 in FIG. 2A. However, compared with (rh) IL-18, potencies (EC₅₀ of NFκB reporter activation) as well as the magnitude (maximal NFκB activation) were attenuated for all bispecifics tested (FIG. 7A, B). According to their agonism potential, all bispecifics were ranked into four groups: molecules discarded due to low purity (grey), functionally inactive bispecifics (red), minimally active agonists eliciting less than 50% of NFκB reporter activation normalized to (rh) IL-18 at 1 nM or EC50 higher than 0.1 nM (yellow) and moderately active surrogate agonists triggering either a normalized NFκB reporter activation of more than 50% relative to IL18 or displaying potencies of less than 0.1 nM (green, FIG. 2B).

### Example 4

Dose-Dependent Release of IFN-γ by Bispecific sdAb-Derived IL-18 Mimetics in PBMC-Based Assays To evaluate the functionality of the engineered bispecific cytokine mimetics in primary cell assays, we stimulated human peripheral blood mononuclear cells (PBMCs) either with (rh) IL-18 or with a selection of 16 different surrogate agonists that showed at least some level of agonistic activity in the IL-18 reporter cell assay (ranging from minimally active to moderately active agonists) in combination with low dose (rh) IL-12. Again, IL18R_VHHα1β16 was included as negative control. To this end, we exploited a fixed concentration of 100 nM of each of bsAbs scrutinized, whereas for (rh) IL-18 due to an anticipated much stronger potency, we used a concentration of 1 nM. As functional read out we used PBMC-derived production of IFN-γ (FIG. 3A). Stimulation of PBMCs with (rh) IL-18 triggered a robust release of IFN-γ (mean release at 1 nM of 2135.8 μg/ml), whereas IFN-γ production was negligible for the treatment with IL18R_VHHα1β16 (mean release at 100 nM of 39.5 μg/ml). For the 16 bispecific surrogate agonists we observed a quite diverse release of IFN-γ at 100 nM. While for five bsAbs (IL18R_VHHβ513, IL18R_VHHα2β22, IL18R_VHHα6β22, IL18R_VHHα8β22, IL18R_VHHα10β22), the magnitude of IFN-γ production was not appreciably different from the negative control (IL18R_VHHα1β16), there was a trend for higher proinflammatory cytokine release for the vast majority of bispecific surrogate agonists tested. Most importantly, sdAb-based IL-18 mimetics IL18R_VHHα2β15, IL18R_VHHα2β17 and IL18R_VHHα8β17 evoked a statistically significant higher IFN-γ production compared to IL18R_VHHα1β16, with IL18R_VHHα2β15 triggering the most prominent release (mean release 1390.2 μg/ml). We also assessed IFN-γ production of all the molecules without low-dose (rh) IL-12 (FIG. 8). Neither (rh) IL-18, nor the generated bispecifics triggered IFN-γ production in the absence of (rh) IL-12, which is in accordance with the finding, that IL-18 induces IFN-γ either with IL-12 or IL-15 (Dinarello et al., 2013).

To further narrow down surrogate agonists for in-depth characterization, we only focused on the three molecules that triggered significant release of IFN-γ as well as IL18R_VHHα8β15 which also elicited a moderate production of IFN-γ, albeit not being statistically significant. Remarkably, those four bispecifics were composed of different combinations of two sdAbs targeting IL-18Rα (VHHα2 and VHHα8) as well as two VHHs specific for the IL-18Rβ subunit (VHHβ15 and VHH017). For this, we stimulated human PBMCs isolated from healthy donors with the four bispecific candidates as well as (rh) IL-18 using a range of different concentrations in combination with low dose (rh) IL-12 (FIG. 3B). All four bispecific IL-18 mimetics triggered a dose-dependent release of IFN-γ with potencies (EC50 of IFN-γ production) of 9.1 nM-~107 nM (Table 1). Of note, IL18R_VHHα8β15 did not reach a maximum IFN-γ release at the highest concentration tested (1 μM). Hence, we haven't been able to determine the EC50 of this bsAb accurately.

Besides functional characterization, we also determined affinities of the TOP4 IL-18 mimetics against each receptor subunit (Table 1, FIG. 9). Affinities for binding to IL-18Rα ranged from 5.2 nM for IL18R_VHHα2β15 to 35.6 nM for IL18R_VHHα8β16. Also binding to the IL-18Rβ subunit ranged from the single digit picomolar range to binding in the lower double digit nanomolar range (for IL18R_VHHα2β15). Interestingly, there was a trend towards higher potencies and efficacies in terms of IFN-γ production for cytokine mimetics harboring a VHH with higher binding affinities to IL-18Rα which were mainly driven by an improved off-rate (VHHα2 vs. VHHα8).

TABLE 1
Binding kinetics and functional properties of the herein generated four leading bispecific IL-18 mimetics.

| | | | | | | | Competition | EC50 |
|---|---|---|---|---|---|---|---|---|
| | KD | kon | kdis | KD | kon | kdis | vs. | IFN-γ |
| | IL-18Rα | IL-18Rα | IL-18Rα | IL-18Rβ | IL-18Rβ | IL-18Rβ | IL-18 on | Release |
| Samples | [nM] | [1/Ms] | [1/s] | [nM] | [1/Ms] | [1/s] | IL18Rα | [nM] |
| IL18R_VHHα2β15 | 5.2 | 1.36E+05 | 7.00E−04 | 12.8 | 3.37E+05 | 4.32E−03 | Yes | 9.1 |
| IL18R_VHHα8β15 | 27.7 | 3.31E+05 | 9.17E−03 | 9.9 | 3.77E+05 | 3.71E−03 | Yes | 106.9 |
| IL18R_VHHα2β17 | 4.1 | 1.48E+05 | 6.09E−04 | 9.1 | 2.26E+05 | 2.06E−03 | Yes | 9.9 |
| IL18R_VHHα8β17 | 35.6 | 3.11E+05 | 1.11E−02 | 7.6 | 2.30E+05 | 1.75E−03 | Yes | 42.7 |

### Example 5

IL-18 Cytokine Mimetics with Engineered Antibody Formats

Next, we also set out to further augment the functional properties of the herein generated bispecific surrogate agonists. To this end, we focused on IL18R_VHHα2(315 that triggered the most prominent production of IFN-γ on human PBMCs. In particular, we aimed at investigating the influence of paratope valencies as well as the spatial orientation of individual paratopes within the overall antibody design architecture on triggering a functional IFN-γ response. The different antibody designs are shown in FIG. 4A. Besides the strictly monovalent (1+1) VHH SEED format, that was exploited for initial IL-18 mimetic generation and characterization (IL18R_VHHα2015), we employed two additional designs. In 2020, our group described a VHH-based IgG-like bi- and multispecific antibody platform that relies on the replacement of the VH and the VL regions of a conventional antibody by two independently functioning VHH domains, resulting in a bispecific, tetravalent antibody derivative, herein referred to as single domain-based IgG (sdIgG) (Pekar et al., 2020). We engrafted the IL-18Rα-specific VHHα2 onto the CH1 domain of the effector silenced heavy chain (IgG1) and the IL-18Rβ-targeting VHHβ15 onto the constant region of the lambda light chain (IL18R_sdIgGα2(315). In addition to this IgG-like design, we also constructed tandem-VHH arrangements, grafted onto the hinge-region of an effector-silenced IgG1 Fc region (2+2). The two independent VHH domains were separated by a five amino acid Gly₄Ser-linker. For this, we assessed both orientations, VHHα2 followed by VHHβ15 (from N-terminus to C-terminus, IL18R_tanVHHα2P 15) and vice versa (IL18R_tanVHHβ15α2). In addition to these four molecules in total (including the initial 1+1 SEED design), all formats were also produced harboring the E340G mutation, that was first described by Parren and colleagues (de Jong et al., 2016). This mutation enhances antibody hexamer formation on the target cell surface after antigen binding and was initially utilized to induce conditional complement-dependent cytotoxicity. In this regard, we speculated that on-target hexamerization of the different surrogate agonist formats would result into enhanced receptor clustering and consequently into an improved IFN-γ response.

Expression yields after protein A purification for the different designs ranged from 12 mg/L for IL18R_sdIgGα2315_E430G to up to 375.2 mg/L for IL18R_tanVHHα2β15 (Table 2). Generally, we observed a trend towards lower expression for molecules harboring the E430G mutation. Interestingly, the different formats comprising the VHH tandem arrangements without the E430G amino acid exchanges displayed expression yields of more than 300 mg/L, indicating high production profiles for transient antibody expression. Most of the different bispecific IL-18 mimetic designs also showed quite favorable aggregation properties as indicated by SEC profiles above 90% target peak post protein A purification. Only for two molecules with implemented E430G mutations (IL18R_VHHα2β15_E430G and IL18R_sdIgGα2β15_E430G), we observed SEC purities of 79.3% and 82.6%, respectively. Hence, both molecules were further polished by preparative SEC, yielding final purities of 97.3% and 98.3% (FIG. 10). Additionally, also the thermal stabilities of the engineered designs were monitored by measuring the tₒₙₛₑₜ that is the lowest temperature at which a protein starts to unfold. Generally, all molecules not harboring the E430G exchanges showed thermal stabilities (tₒₙₛₑₜₛ) above 50 °C, indicating favorable biophysical properties, whereas all designs with incorporated E430G mutations showed diminished stabilities by 4 °C-8.6 °C (Table 2, FIG. 11).

TABLE 2
Biophysical, biochemical and functional attributes of engineered cytokine mimetic formats.

| | | | Final SEC | | Mean | Mean | |
|---|---|---|---|---|---|---|---|
| | | | after freeze | | IFN-γ | IFN-γ | EC50 |
| | | SEC after | thaw and | | Release | Release | IFN-γ |
| | Yield | protein A | polishing* | Tonset | [pg/mL] at | [pg/mL] at | Release |
| Samples | [mg/L] | [%] | [%] | [°C] | 10 nM | 1 nM | [nM] |
| IL18R_VHHα2β15 | 64.0 | 94.1 | 94.1 | 56.4 | 418.2 | 115.1 | 2.4 |
| IL18R_VHHα2β15_E430G | 91.6 | 79.3 | 97.3* | 49.7 | 466.4 | 114.9 | |
| IL18R_sdIgGα2β15 | 262.4 | 92.3 | 91.9 | 57.0 | 332.1 | 376.1 | |
| IL18R_sdIgGα2β15_E430G | 142.4 | 82.6 | 98.3* | 48.4 | 374.6 | 385.5 | |
| IL18R_tanVHHα2β15 | 375.2 | 95.5 | 95.2 | 54.3 | 1871.4 | 1946.0 | 0.006 |
| IL18R_tanVHHα2β15_E430G | 161.2 | 97.4 | 95.7 | 50.0 | 1845.9 | 1961.3 | |
| IL18R_tanVHHβ15α2 | 324.4 | 93.5 | 93.3 | 53.3 | 1116.5 | 1238.2 | 0.016 |
| IL18R_tanVHHβ15α2_E430G | 12.0 | 94.2 | 94.3 | 49.3 | 1017.5 | 1061.8 | |

*only for IL18R_VHHα2β15_E430G and IL18R_sdIgGα2β15_E430G preparative SEC was applied for polishing.

Subsequently, all different antibody designs based on VHHα2 and VHHβ15 were analyzed by means of triggering IFN-γ production on human PBMCs isolated from six different donors in total. The different formats were used at two different concentrations (10 nM and 1 nM). Intriguingly, profound differences were unveiled for the different design architectures (FIG. 4B). The engineered sdIgG-based cytokine mimetic (IL18R_sdIgGα2β15) did not induce a substantially improved IFN-γ response compared to the initial (1+1) SEED design termed IL18R_VHHα2β15 (332.1 μg/ml and 376.1 μg/ml for IL18R_sdIgGα2β15 at 10 nM and 1 nM, respectively vs 418.2 μg/ml and 115.1 μg/ml). Hence, we de-prioritized this format for further analysis. In contrast to this, stimulation of PBMCs with both tandem arrangements IL18R_tanVHHβ15α2 and IL18R_tanVHHα2β15 provoked a significantly augmented IFN-γ production with IL18R_tanVHHα2β15 eliciting the strongest release (1116.5 μg/ml and 1238.2 μg/ml at 10 nM and 1 nM, respectively) within the set of engineered IL-18 mimetic architectures. In general, implementation of the E430G amino acid exchanges did not seem to improve IFN-γ release. Consequently, we also discarded all formats harboring the E430G mutation for additional characterization.

Both formats comprising the VHH arranged in tandem (IL18R_tanVHHβ15α2 and IL18R_tanVHHα2β15) as well as the initial 1+1 SEED design (IL18R_VHHα2β 15) were directly compared to (rh) IL-18 regarding their potential to elicit a functional IFN-γ response (FIG. 4C, Table 2). In comparison with (rh) IL-18, the capacity of IL18R_VHHα2β15 to trigger IFN-γ release on PBMCS was clearly attenuated (EC₅₀ of 2.4 nM for IL18R_VHHα2β15 vs 61 pM for (rh) IL-18).

In contrast to this, the potential to evoke IFN-γ production of both mimetics harboring the tandem arrangements were substantially augmented (EC₅₀ of 16 μM for IL18R_tanVHHβ15α2 and 6 μM for IL18R_tanVHHα2β 15), resulting in molecules that were significantly more potent than (rh) IL-18. While potencies were fairly similar between both tandem formats, the magnitude IFN-γ release was quite different. In this regard, IL18R_tanVHHα2β15 caused a maximum release that was quite similar to (rh)IL-18 (2130 μg/ml vs 2097 μg/ml), whereas IL18R_tanVHHβ15α2 elicited a significantly reduced maximum IFN-γ production of 1095 μg/ml. Of note, we observed a strong hooking effect (bell shaped curve) for (rh) IL-18, i.e., reduced IFN-γ release with high and increasing compound concentrations (FIG. 12). This effect was clearly not as pronounced for the different surrogate agonist formats tested. To investigate whether biofunctional differences also translate into differential biochemical attributes, we performed BLI experiments that were aimed at evaluating target affinities as well as binding avidities of IL18R_VHHα2β15, IL18R_tanVHHα2β15 and IL18R_tanVHHβ15α2.

For determining affinities, bispecific cytokine mimetics were captured on the sensor tips and the respective IL-18 receptor subunits were exploited as analyte (Table 3). Compared with IL18R_VHHα2β15, binding affinities for the inner paratopes of tandem IL-18 mimetics were slightly reduced, whereas kinetics for the outer sdAb remained largely unaffected.

To evaluate avidities, the respective receptor subunits were captured on the sensor tips and binding was assessed for the monovalent (for each target) bsAb IL18R_VHHα2β15 as well as for the bivalent (for binding to each receptor chain) surrogate agonists IL18R_tanVHHα2β15 and IL18R_tanVHHβ15α2 (Table 3, FIG. 13). In this experimental setting, IL18R_VHHα2β15 displayed binding to both respective receptor chains in the lower double digit nanomolar range. In contrast to this, apparent binding affinities for both tandem IL-18 mimetics could not be determined, which was driven by the fact that no dissociation from the receptor subunits was observed. This is giving strong evidence for high avidity binding of both tandem surrogate agonists.

TABLE 3
Apparent binding affinities of selected surrogate agonists in an affinity driven BLI experiment and in an avidity driven setting.

| | | | | | | | Avidity driven setting | | |
|---|---|---|---|---|---|---|---|---|---|
| | Affinity driven experimental setting | | | | | | KD | KD | |
| | KD | kon | kdis | KD | kon | kdis | IL-18Rα | IL-18Rβ | Competition |
| | IL-18Rα | IL-18Rα | IL-18Rα | IL-18Rβ | IL-18Rβ | IL-18Rβ | [nM] | [nM] | vs |
| | [nM] | [1/Ms] | [1/s] | [nM] | [1/Ms] | [1/s] | HIS1K | HIS1K | IL-18 |
| IL18R_VHHα2β15 | 5.2 | 1.36E+05 | 7.00E−04 | 12.8 | 3.37E+05 | 4.32E−03 | 19.9 | 29.0 | Yes |
| IL18R_tanVHHα2β15 | 6.6 | 1.67E+05 | 1.10E−03 | 44.6 | 1.96E+05 | 8.73E−03 | <0.001 | <0.001 | Yes |
| IL18R_tanVHHβ15α2 | 12.1 | 8.72E+04 | 1.05E−03 | 11.1 | 3.15E+05 | 3.50E−03 | <0.001 | <0.001 | Yes |

### Example 6

Bispecific IL-18 mimetics are resistant to inhibition by IL-18BP Finally, we wanted to analyze whether the herein generated IL-18 mimetics are inhibited by IL-18BP receptor decoy. To this end, we employed the initially generated TOP4 cytokine mimetics, IL18R_VHHα2β15, IL18R_VHHα2β17, IL18R_VHHα8β17 and IL18R_VHHα8β15 as well as both tandem-engineered surrogate agonists IL18R_tanVHHβ15α2 and IL18R_tanVHHβ15α2 (which are format engineered derivatives of IL18R_VHHα2β15). As determined by BLI, no binding interaction of (rh) IL-18BP was measurable to any of the IL-18 mimetics exploited (FIG. 14A). Opposed to this, (rh) IL-18BP showed high-affinity binding to (rh)IL-18 in the picomolar range (FIG. 14B). Both, IL-18BP and IL-18Rα bind to IL-18 at an overlapping interface (Zhou et al., 2020). Of note, all six surrogate agonists showed competitive binding with (rh) IL-18 for targeting IL-18Rα, indicating a similar or at least a partially overlapping epitope on IL-18Rα (FIG. 15 and Table 1).

Ultimately, we evaluated the resistance of the engineered IL-18 mimetics to IL-18BP inhibition on PBMC stimulation. Therefore, we focused on both tandem IL-18 mimetics, showing potencies at a similar level as (rh)IL-18 in provoking an IFN-γ response. Both cytokine mimetics as well as (rh)IL-18 were utilized at a fixed concentration of 0.5 nM combined with low dose (rh)IL-12 (10 ng/ml) and increasing concentrations of (rh)IL-18BP were titrated (FIG. 4D). Contrary to (rh)IL18 that was efficiently inhibited by (rh)IL-18BP (IC50 of 2.2 nM), both tandem IL-18 mimetics, IL18R_tanVHHβ15α2 and IL18R_tanVHHβ15α2 triggered a robust IFN-γ release, regardless of the IL-18BP receptor decoy concentration tested.

### REFERENCES

Amin, A., and White, R. L. (2014). Interleukin-2 in Renal Cell Carcinoma: A Has-Been or a Still-Viable Option?Journal of Kidney Cancer and VHL 1, 74-83. 10.15586/jkcvhl.2014.18.
Atallah-Yunes, S. A., and Robertson, M. J. (2022). Cytokine Based Immunotherapy for Cancer and Lymphoma: Biology, Challenges and Future Perspectives. Frontiers in Immunology 13. 10.3389/fimmu.2022.872010.
Berraondo, P., Sanmamed, M. F., Ochoa, M. C., Etxeberria, I., Aznar, M. A., Pérez-Gracia, J. L., Rodriguez-Ruiz, M. E., Ponz-Sarvise, M., Castañón, E., and Melero, I. (2019). Cytokines in clinical cancer immunotherapy. British Journal of Cancer 120, 6-15. 10.1038/s41416-018-0328-y.
Chanier, T., and Chames, P. (2019). Nanobody Engineering: Toward Next Generation Immunotherapies and Immunoimaging of Cancer. Antibodies 8, 13. 10.3390/antib8010013.
Davis, J. H., Aperlo, C., Li, Y., Kurosawa, E., Lan, Y., Lo, K.-M., and Huston, J. S. (2010). SEEDbodies: fusion proteins based on strand-exchange engineered domain (SEED) CH3 heterodimers in an Fc analogue platform for asymmetric binders or immunofusions and bispecific antibodiest. Protein Engineering, Design and Selection 23, 195-202. 10.1093/protein/gzp094.
de Jong, R. N., Beurskens, F. J., Verploegen, S., Strumane, K., van Kampen, M. D., Voorhorst, M., Horstman, W., Engelberts, P. J., Oostindie, S. C., Wang, G., et al. (2016). A Novel Platform for the Potentiation of Therapeutic Antibodies Based on Antigen-Dependent Formation of IgG Hexamers at the Cell Surface. PLOS Biology 14, e1002344. 10.1371/journal.pbio.1002344.
Dinarello, C. A., Novick, D., Kim, S., and Kaplanski, G. (2013). Interleukin-18 and IL-18 Binding Protein. Frontiers in Immunology 4. 10.3389/fimmu.2013.00289.
Dolgin, E. (2022). IL-2 upgrades show promise at ASCO. Nature Biotechnology 40, 986-988. 10.1038/s41587-022-01390-3.
Eliason, J. F. (2001). Pegylated Cytokines: Potential Application in Immunotherapy of Cancer. BioDrugs 15, 705-711. 10.2165/00063030-200115110-00001.
Harris, K. E., Lorentsen, K. J., Malik-Chaudhry, H. K., Loughlin, K., Basappa, H. M., Hartstein, S., Ahmil, G., Allen, N. S., Avanzino, B. C., Balasubramani, A., et al. (2021). A bispecific antibody agonist of the IL-2 heterodimeric receptor preferentially promotes in vivo expansion of CD8 and NK cells. Scientific Reports 11. 10.1038/s41598-021-90096-8.
Holder, P. G., Lim, S. A., Huang, C. S., Sharma, P., Dagdas, Y. S., Bulutoglu, B., and Sockolosky, J. T. (2022). Engineering interferons and interleukins for cancer immunotherapy. Advanced Drug Delivery Reviews 182, 114112. 10.1016/j.addr.2022.114112.
Jazayeri, J. A., and Carroll, G. J. (2008). Fc-Based Cytokines: Prospects for Engineering Superior Therapeutics. BioDrugs 22, 11-26. 10.2165/00063030-200822010-00002.
Klausz, K., Pekar, L., Boje, A. S., Gehlert, C. L., Krohn, S., Gupta, T., Xiao, Y., Krah, S., Zaynagetdinov, R., Lipinski, B., et al. (2022). Multifunctional NK Cell-Engaging Antibodies Targeting EGFR and NKp30 Elicit Efficient Tumor Cell Killing and Proinflammatory Cytokine Release. The Journal of Immunology 209, 1724-1735. 10.4049/jimmunol.2100970.
Klein, C., Waldhauer, I., Nicolini, V. G., Freimoser-Grundschober, A., Nayak, T., Vugts, D. J., Dunn, C., Bolijn, M., Benz, J., Stihle, M., et al. (2017). Cergutuzumab amunaleukin (CEA-IL2v), a CEA-targeted IL-2 variant-based immunocytokine for combination cancer immunotherapy: Overcoming limitations of aldesleukin and conventional IL-2-based immunocytokines. OncoImmunology 6, e1277306. 10.1080/2162402X.2016.1277306.
Lipinski, B., Arras, P., Pekar, L., Klewinghaus, D., Boje, A. S., Krah, S., Zimmermann, J., Klausz, K., Peipp, M., Siegmund, V., et al. (2023). NKP46-specific single domain antibodies enable facile engineering of various potent NK cell engager formats. Protein Science. 10.1002/pro.4593.
Moraga, I., Wernig, G., Wilmes, S., Gryshkova, V., Richter, C. P., Hong, W.-J., Sinha, R., Guo, F., Fabionar, H., Wehrman, T. S., et al. (2015). Tuning Cytokine Receptor Signaling by Re-orienting Dimer Geometry with Surrogate Ligands. Cell 160, 1196-1208. 10.1016/j. cell.2015.02.011.
Murer, P., and Neri, D. (2019). Antibody-cytokine fusion proteins: A novel class of biopharmaceuticals for the therapy of cancer and of chronic inflammation. New Biotechnology 52, 42-53. 10.1016/j.nbt.2019.04.002.
Nakamura, K., Bald, T., and Smyth, M. J. (2020). Cancer-killing, decoy-resistant interleukin-18. Immunology & Cell Biology 98, 434-436. 10.1111/imcb.12359.
Neri, D., and Sondel, P. M. (2016). Immunocytokines for cancer treatment: past, present and future. Current Opinion in Immunology 40, 96-102. 10.1016/j.coi.2016.03.006.
Pekar, L., Busch, M., Valldorf, B., Hinz, S. C., Toleikis, L., Krah, S., and Zielonka, S. (2020). Biophysical and biochemical characterization of a VHH-based IgG-like bi- and trispecific antibody platform. mAbs, 1812210. 10.1080/19420862.2020.1812210.
Pires, I. S., Hammond, P. T., and Irvine, D. J. (2021). Engineering Strategies for Immunomodulatory Cytokine Therapies: Challenges and Clinical Progress. Advanced Therapeutics 4, 2100035. 10.1002/adtp.202100035.
Propper, D. J., and Balkwill, F. R. (2022). Harnessing cytokines and chemokines for cancer therapy. Nature Reviews Clinical Oncology 19, 237-253. 10.1038/s41571-021-00588-9.
Robertson, M. J., Mier, J. W., Logan, T., Atkins, M., Koon, H., Koch, K. M., Kathman, S., Pandite, L. N., Oei, C., Kirby, L. C., et al. (2006). Clinical and Biological Effects of Recombinant Human Interleukin-18 Administered by Intravenous Infusion to Patients with Advanced Cancer. Clinical Cancer Research 12, 4265-4273. 10.1158/1078-0432.CCR-06-0121.
Robertson, M. J., Kirkwood, J. M., Logan, T. F., Koch, K. M., Kathman, S., Kirby, L. C., Bell, W. N., Thurmond, L. M., Weisenbach, J., and Dar, M. M. (2008). A Dose-Escalation Study of Recombinant Human Interleukin-18 Using Two Different Schedules of Administration in Patients with Cancer. Clinical Cancer Research 14, 3462-3469. 10.1158/1078-0432.CCR-07-4740.
Robertson, M. J., Kline, J., Struemper, H., Koch, K. M., Bauman, J. W., Gardner, O. S., Murray, S. C., Germaschewski, F., Weisenbach, J., Jonak, Z., et al. (2013). A Dose-Escalation Study of Recombinant Human Interleukin-18 in Combination With Rituximab in Patients With Non-Hodgkin Lymphoma. Journal of Immunotherapy 36, 331-341. 10.1097/CJI.0b013e31829d7e2e.
Robertson, M. J., Stamatkin, C. W., Pelloso, D., Weisenbach, J., Prasad, N. K., and Safa, A. R. (2018). A Dose-escalation Study of Recombinant Human Interleukin-18 in Combination With Ofatumumab After Autologous Peripheral Blood Stem Cell Transplantation for Lymphoma. Journal of Immunotherapy 41, 151-157. 10.1097/CJI.0000000000000220.
Roth, L., Krah, S., Klemm, J., G0nther, R., Toleikis, L., Busch, M., Becker, S., and Zielonka, S. (2020). Isolation of Antigen-Specific VHH Single-Domain Antibodies by Combining Animal Immunization with Yeast Surface Display. Methods Mol. Biol. 2070, 173-189. 10.1007/978-1-4939-9853-1_10.
Saxton, R. A., Glassman, C. R., and Garcia, K. C. (2023). Emerging principles of cytokine pharmacology and therapeutics. Nature Reviews Drug Discovery 22, 21-37. 10.1038/s41573-022-00557-6.
Simpkins, F., Flores, A., Chu, C., Berek, J. S., Lucci, J., Murray, S., Bauman, J., Struemper, H., Germaschewski, F., Jonak, Z., et al. (2013). Chemoimmunotherapy Using Pegylated Liposomal Doxorubicin and Interleukin-18 in Recurrent Ovarian Cancer: A Phase I Dose-Escalation Study. Cancer Immunology Research 1, 168-178. 10.1158/2326-6066.CIR-13-0098.
Tarhini, A. A., Millward, M., Mainwaring, P., Kefford, R., Logan, T., Pavlick, A., Kathman, S. J., Laubscher, K. H., Dar, M. M., and Kirkwood, J. M. (2009). A phase 2, randomized study of SB-485232, rhIL-18, in patients with previously untreated metastatic melanoma. Cancer 115, 859-868. 10.1002/cncr.24100.
Yasuda, K., Nakanishi, K., and Tsutsui, H. (2019). Interleukin-18 in Health and Disease. International Journal of Molecular Sciences 20, 649. 10.3390/ijms20030649.
Yen, M., Ren, J., Liu, Q., Glassman, C. R., Sheahan, T. P., Picton, L. K., Moreira, F. R., Rustagi, A., Jude, K. M., Zhao, X., et al. (2022). Facile discovery of surrogate cytokine agonists. Cell 185, 1414-1430.e19. 10.1016/j.cell.2022.02.025.
Zheng, X., Wu, Y., Bi, J., Huang, Y., Cheng, Y., Li, Y., Wu, Y., Cao, G., and Tian, Z. (2022). The use of supercytokines, immunocytokines, engager cytokines, and other synthetic cytokines in immunotherapy. Cellular & Molecular Immunology 19, 192-209. 10.1038/s41423-021-00786-6.
Zhou, T., Damsky, W., Weizman, O.-E., McGeary, M. K., Hartmann, K. P., Rosen, C. E., Fischer, S., Jackson, R., Flavell, R. A., Wang, J., et al. (2020). IL-18BP is a secreted immune checkpoint and barrier to IL-18 immunotherapy. Nature 583, 609-614. 10.1038/s41586-020-2422-6.

1. A compound, comprising;
a first binding module which is a VHH antibody domain or fragment thereof, wherein
(a) said VHH antibody domain or fragment thereof comprises the complementarity determining regions CDR1, CDR2 and CDR3 of one VHH selected from the group consisting of VHH1, VHH2, VHH5, VHH6, VHH8, VHH9 and VHH10 as shown in the Table of CDRs;
(b) said VHH antibody domain or fragment thereof comprises the complementarity determining regions CDR1, CDR2 and CDR3 as defined in (a) with modification, wherein the modification is that the sequence of at least one of CDR1, CDR2 and CDR3 is humanized; or
(c) said VHH antibody domain or fragment thereof comprises the complementarity determining regions CDR1, CDR2 and CDR3 as defined in (a) with modification, wherein the modification is
the replacement, addition or deletion of up to three amino acids in CDR1,
the replacement, addition or deletion of up to three amino acids in CDR2 and/or
the replacement, addition or deletion of up to three amino acids in CDR3;
a second binding module which is a VHH antibody domain or a fragment thereof, wherein
(d) said VHH antibody domain or fragment thereof comprises the complementarity determining regions CDR1, CDR2 and CDR3 of one VHH selected from the group consisting of VHH12, VHH13, VHH14, VHH15, VHH17, VHH18, VHH20, VHH21 and VHH22 as shown in the Table of CDRs, wherein said VHH antibody domain or fragment thereof defined in (d) is selected as follows:
if the VHH antibody domain of (a) or the fragment thereof comprises the complementarity determining regions CDR1, CDR2 and CDR3 of VHH1, then the VHH antibody domain of (d) or the fragment thereof comprises the complementarity determining regions CDR1, CDR2 and CDR3 of one VHH selected from the group consisting of VHH12, VHH13, VHH15, VHH17, VHH20, VHH21 and VHH22 as shown in the Table of CDRs;
if the VHH antibody domain of (a) or the fragment thereof comprises the complementarity determining regions CDR1, CDR2 and CDR3 of VHH2, then the VHH antibody domain of (d) or the fragment thereof comprises the complementarity determining regions CDR1, CDR2 and CDR3 of one VHH selected from the group consisting of VHH12, VHH13, VHH15, VHH17, VHH18, VHH20, VHH21 and VHH22 as shown in the Table of CDRs;
if the VHH antibody domain of (a) or the fragment thereof comprises the complementarity determining regions CDR1, CDR2 and CDR3 of VHH5, then the VHH antibody domain of (d) or the fragment thereof comprises the complementarity determining regions CDR1, CDR2 and CDR3 of one VHH selected from the group consisting of VHH13 and VHH14 as shown in the Table of CDRs;
if the VHH antibody domain of (a) or the fragment thereof comprises the complementarity determining regions CDR1, CDR2 and CDR3 of VHH6, then the VHH antibody domain of (d) or the fragment thereof comprises the complementarity determining regions CDR1, CDR2 and CDR3 of one VHH selected from the group consisting of VHH17, VHH20, VHH21 and VHH22 as shown in the Table of CDRs;
if the VHH antibody domain of (a) or the fragment thereof comprises the complementarity determining regions CDR1, CDR2 and CDR3 of VHH8, then the VHH antibody domain of (d) or the fragment thereof comprises the complementarity determining regions CDR1, CDR2 and CDR3 of one VHH selected from the group consisting of VHH12, VHH13, VHH17, VHH18, VHH20 and VHH21 as shown in the Table of CDRs;
if the VHH antibody domain of (a) or the fragment thereof comprises the complementarity determining regions CDR1, CDR2 and CDR3 of VHH9, then the VHH antibody domain of (d) or the fragment thereof comprises the complementarity determining regions CDR1, CDR2 and CDR3 of one VHH selected from the group consisting of VHH12, VHH13, VHH15, VHH17, VHH18, VHH20, VHH21 and VHH22 as shown in the Table of CDRs;
if the VHH antibody domain of (a) or the fragment thereof comprises the complementarity determining regions CDR1, CDR2 and CDR3 of VHH10, then the VHH antibody domain of (d) or the fragment thereof comprises the complementarity determining regions CDR1, CDR2 and CDR3 of one VHH selected from the group consisting of VHH13, VHH20 and VHH21 as shown in the Table of CDRs;
(e) said VHH antibody domain or fragment thereof comprises the complementarity determining regions CDR1, CDR2 and CDR3 as defined in (d) with modification, wherein the modification is that the sequence of at least one of CDR1, CDR2 and CDR3 is humanized; or
(f) said VHH antibody domain or fragment thereof comprises the complementarity determining regions CDR1, CDR2 and CDR3 as defined in (d) with modification, wherein the modification is
the replacement, addition or deletion of up to one amino acid in CDR1,
the replacement, addition or deletion of up to one amino acid in CDR2 and/or the replacement, addition or deletion of up to one amino acid in CDR3;
TABLE of
CDRs:

| Description | SEQ ID NO | Amino acid sequence |
|---|---|---|
| CDR1 of VHH1 | SEQ ID NO: 23 | GFTFNSYA |
| CDR2 of VHH1 | SEQ ID NO: 24 | HIGSGGSTR |
| CDR3 of VHH1 | SEQ ID NO: 25 | NVYGATLRS |
| CDR1 of VHH2 | SEQ ID NO: 26 | GFTLLDSYD |
| CDR2 of VHH2 | SEQ ID NO: 27 | ISSSGGKSTY |
| CDR3 of VHH2 | SEQ ID NO: 28 | AADGAEGFWSCQSGGYDY |
| CDR1 of VHH3 | SEQ ID NO: 29 | GFTLGAYA |
| CDR2 of VHH3 | SEQ ID NO: 30 | ITSSGRSAY |
| CDR3 of VHH3 | SEQ ID NO: 31 | AAGAACAAAGTMNY |
| CDR1 of VHH4 | SEQ ID NO: 32 | GFTLGAYA |
| CDR2 of VHH4 | SEQ ID NO: 33 | ITSSGGSAY |
| CDR3 of VHH4 | SEQ ID NO: 34 | AAGGACAAAETMNY |
| CDR1 of VHH5 | SEQ ID NO: 35 | GRTFSNLA |
| CDR2 of VHH5 | SEQ ID NO: 36 | IDWNGDNRL |
| CDR3 of VHH5 | SEQ ID NO: 37 | AADSTYSTDWYLNPLNR |
| | | TRYDY |
| CDR1 of VHH6 | SEQ ID NO: 38 | KSIFSINA |
| CDR2 of VHH6 | SEQ ID NO: 39 | FTSSGRIN |
| CDR3 of VHH6 | SEQ ID NO: 40 | NVGLWEDGLWYDY |
| CDR1 of VHH7 | SEQ ID NO: 41 | GHTFNNLA |
| CDR2 of VHH7 | SEQ ID NO: 42 | IDWNGDNRL |
| CDR3 of VHH7 | SEQ ID NO: 43 | AADSTYSTNWYLDPLNR |
| | | TTYNY |
| CDR1 of VHH8 | SEQ ID NO: 44 | GRTFSNDA |
| CDR2 of VHH8 | SEQ ID NO: 45 | VSWSLGMTY |
| CDR3 of VHH8 | SEQ ID NO: 46 | AAGPDLSDELYGYDY |
| CDR1 of VHH9 | SEQ ID NO: 47 | RSDFSRYT |
| CDR2 of VHH9 | SEQ ID NO: 48 | RITSGGIPH |
| CDR3 of VHH9 | SEQ ID NO: 49 | NTVGVALWA |
| CDR1 of VHH10 | SEQ ID NO: 50 | GLPFSSYA |
| CDR2 of VHH10 | SEQ ID NO: 51 | ITWSGSSIY |
| CDR3 of VHH10 | SEQ ID NO: 52 | AAARRRVGLPLADPREY |
| | | VY |
| CDR1 of VHH11 | SEQ ID NO: 53 | GSPIRFSV |
| CDR2 of VHH11 | SEQ ID NO: 54 | RITSGGIVY |
| CDR3 of VHH11 | SEQ ID NO: 55 | NRLGGALFD |
| CDR1 of VHH12 | SEQ ID NO: 56 | GVGFSIYT |
| CDR2 of VHH12 | SEQ ID NO: 57 | VTTGGHIN |
| CDR3 of VHH12 | SEQ ID NO: 58 | NAIYFGQDI |
| CDR1 of VHH13 | SEQ ID NO: 59 | GISFRVNV |
| CDR2 of VHH13 | SEQ ID NO: 60 | ISSGDSTN |
| CDR3 of VHH13 | SEQ ID NO: 61 | NARIFARDN |
| CDR1 of VHH14 | SEQ ID NO: 62 | DRTVSKGA |
| CDR2 of VHH14 | SEQ ID NO: 63 | IGRSSGITY |
| CDR3 of VHH14 | SEQ ID NO: 64 | AADSSGIIRTTSWAYDY |
| CDR1 of VHH15 | SEQ ID NO: 65 | GRFFTINA |
| CDR2 of VHH15 | SEQ ID NO: 66 | ITSDGSTT |
| CDR3 of VHH15 | SEQ ID NO: 67 | NADGRFNDNGYVKNFNI |
| CDR1 of VHH16 | SEQ ID NO: 68 | GFTTDDYI |
| CDR2 of VHH16 | SEQ ID NO: 69 | ISTTDGSAW |
| CDR3 of VHH16 | SEQ ID NO: 70 | AEVSTGTGVLYCREEFY |
| | | FGMDS |
| CDR1 of VHH17 | SEQ ID NO: 71 | GFALDEIA |
| CDR2 of VHH17 | SEQ ID NO: 72 | ISSSDGSTL |
| CDR3 of VHH17 | SEQ ID NO: 73 | AAVRGSCFGRTTH |
| CDR1 of VHH18 | SEQ ID NO: 74 | GRTFSSYA |
| CDR2 of VHH18 | SEQ ID NO: 75 | ISWSGGSTY |
| CDR3 of VHH18 | SEQ ID NO: 76 | AADRSLSGSYFGATIDY |
| CDR1 of VHH19 | SEQ ID NO: 77 | GIRFNPFR |
| CDR2 of VHH19 | SEQ ID NO: 78 | IDSGGSTD |
| CDR3 of VHH19 | SEQ ID NO: 79 | NTQIFARDY |
| CDR1 of VHH20 | SEQ ID NO: 80 | VSAFRLKV |
| CDR2 of VHH20 | SEQ ID NO: 81 | IDSGDTKS |
| CDR3 of VHH20 | SEQ ID NO: 82 | NAVLFGRDY |
| CDR1 of VHH21 | SEQ ID NO: 83 | GITFSPYV |
| CDR2 of VHH21 | SEQ ID NO: 84 | MVNGGSTN |
| CDR3 of VHH21 | SEQ ID NO: 85 | HIMIFGRAS |
| CDR1 of VHH22 | SEQ ID NO: 86 | GFALEYYA |
| CDR2 of VHH22 | SEQ ID NO: 87 | VSSSRSGGSTV |
| CDR3 of VHH22 | SEQ ID NO: 88 | AGAPAYRSHYCRLQADD |
| | | YTY |

2. The compound according to claim 1, wherein said VHH antibody domain or fragment thereof defined in (a) comprises the complementarity determining regions CDR1, CDR2 and CDR3 of one VHH selected from the group consisting of VHH2 and VHH8 as shown in the Table of CDRs and wherein said VHH antibody domain or fragment thereof defined in (d) comprises the complementarity determining regions CDR1, CDR2 and CDR3 of one VHH selected from the group consisting of VHH15 and VHH17 as shown in the Table of CDRs.

3. The compound according to claim 1, wherein said VHH antibody domain or fragment thereof defined in (a) comprises the complementarity determining regions CDR1, CDR2 and CDR3 of VHH2 as shown in the Table of CDRs and wherein said VHH antibody domain or fragment thereof defined in (d) comprises the complementarity determining regions CDR1, CDR2 and CDR3 of VHH15 as shown in the Table of CDRs.

4. A compound, comprising;
a first binding module which is a VHH antibody domain or fragment thereof, wherein
(A)said VHH antibody domain comprises the amino acid sequence of a VHH selected from the group consisting of VHH1, VHH2, VHH5, VHH6, VHH8, VHH9 and VHH10 as shown in the Table of VHH Sequences;
(B) said VHH antibody domain comprises a VHH sequence as defined in (A) with modification, wherein the modification is that said sequence is humanized;
(C) said VHH antibody domain comprises a VHH sequence as defined in (A) with modification, wherein the modification is the replacement, addition or deletion of up to 25 amino acids; or
(D) said VHH antibody domain comprises a VHH sequence that is at least 75% identical to a VHH sequence referred to in (A);
a second binding module which is a VHH antibody domain or a fragment thereof, wherein
(E) said VHH antibody domain comprises the amino acid sequence of a VHH selected from the group consisting of VHH12, VHH13, VHH14, VHH15, VHH17, VHH18, VHH20, VHH21 and VHH22 as shown in the Table of VHH Sequences, wherein said VHH antibody domain of (E) is selected as follows:
if the VHH antibody domain of (A) comprises the amino acid sequence of VHH1, then the VHH antibody domain of (E) comprises the amino acid sequence of one VHH selected from the group consisting of VHH12, VHH13, VHH15, VHH17, VHH20, VHH21 and VHH22 shown in the Table of VHH Sequences;
if the VHH antibody domain of (A) comprises the amino acid sequence of VHH2, then the VHH antibody domain of (E) comprises the amino acid sequence of one VHH selected from the group consisting of VHH12, VHH13, VHH15, VHH17, VHH18, VHH20, VHH21 and VHH22 shown in the Table of VHH Sequences;
if the VHH antibody domain of (A) comprises the amino acid sequence of VHH5, then the VHH antibody domain of (E) comprises the amino acid sequence of one VHH selected from the group consisting of VHH13 and VHH14 shown in the Table of VHH Sequences;
if the VHH antibody domain of (A) comprises the amino acid sequence of VHH6, then the VHH antibody domain of (E) comprises the amino acid sequence of one VHH selected from the group consisting of VHH17, VHH20, VHH21 and VHH22 shown in the Table of VHH Sequences;
if the VHH antibody domain of (A) comprises the amino acid sequence of VHH8, then the VHH antibody domain of (E) comprises the amino acid sequence of one VHH selected from the group consisting of VHH12, VHH13, VHH17, VHH18, VHH20 and VHH21 shown in the Table of VHH Sequences;
if the VHH antibody domain of (A) comprises the amino acid sequence of VHH9, then the VHH antibody domain of (E) comprises the amino acid sequence of one VHH selected from the group consisting of VHH12, VHH13, VHH15, VHH17, VHH18, VHH20, VHH21 and VHH22 shown in the Table of VHH Sequences;
if the VHH antibody domain of (A) comprises the amino acid sequence of VHH10, then the VHH antibody domain of (E) comprises the amino acid sequence of one VHH selected from the group consisting of VHH13, VHH20 and VHH21 shown in the Table of VHH Sequences;
(F) said VHH antibody domain comprises a VHH sequence as defined in (E) with modification, wherein the modification is that said sequence is humanized;
(G) said VHH antibody domain comprises a VHH sequence as defined in (E) with modification, wherein the modification is the replacement, addition or deletion of up to 10 amino acids; or
(H) said VHH antibody domain comprises a VHH sequence that is at least 90% identical to a VHH sequence referred to in (E);
TABLE of
VHH Sequences:

| VHH | SEQ ID NO | Amino acid sequence |
|---|---|---|
| VHH1 | SEQ ID NO: | DVPLQESGGGLVQTGGSLRLSCAASGFTFN |
| | 1 | SYAMGWYQQAPGKKYRELVAHIGSGGSTRYIDS |
| | | VKGRFTISRDNAKNTVYLQMNSLKPEDTAVYYC |
| | | NVYGATLRSWGQGTQVTVSS |
| VHH2 | SEQ ID NO: | DVQLQESGGGLVQAGGSLRLSCAASGFTLL |
| | 2 | DSYDIGWFRQAPGKKREEVSCISSSGGKSTYYA |
| | | DSVKGRFTISRDNANNTVYLQMNSLKPEDTAVY |
| | | YCAADGAEGFWSCQSGGYDYWGQGTQVTVSS |
| VHH3 | SEQ ID NO: | DVQLQESGGGLVQAGGSLRLSCAASGFTLG |
| | 3 | AYAINWFRQAPGKERARAWDACITSSGRSAYYA |
| | | DSVKGRFTVSRDHAKNTVYLQMNSLKPEDTAFY |
| | | YCAAGAACAAAGTMNYWGKGTLVTVSS |
| VHH4 | SEQ ID NO: | DVQLQESGGGLVQAGGSLRLSCAASGFTLG |
| | 4 | AYAINWFQQAPGKERARAWVLCITSSGGSAYYV |
| | | DSVKGRFTVSRDNAKNTVYLQMNSLKPEDTAFY |
| | | YCAAGGACAAAETMNYWGKGTLVTVSS |
| VHH5 | SEQ ID NO: | DVQLQESGGGLVQAGGSLRLSCAASGRTFS |
| | 5 | NLAMAWFRQAPGKEREFVAAIDWNGDNRLYAAS |
| | | VKGRFTISRDNAKSTMYLQMNSLKPEDTAVYYC |
| | | AADSTYSTDWYLNPLNRTRYDYWGQGTQVTVSS |
| VHH6 | SEQ ID NO: | DVPLQESGGGSVQPGGSLRLSCAASKSIFS |
| | 6 | INAMRWHRQAPGKQRELVATFTSSGRINYADSV |
| | | KGRFTISTDNAKNTIYLQMNSLNPEDTAVYRCN |
| | | VGLWEDGLWYDYWGQGTQVTVSS |
| VHH7 | SEQ ID NO: | DVQLQESGGGLVQAGGSLRLSCAASGHTFN |
| | 7 | NLAMAWCRQAPGKEREFVSAIDWNGDNRLYAYS |
| | | VKGRFTISRDNAKSTMYLQMNSLKPEDTAVYYC |
| | | AADSTYSTNWYLDPLNRTTYNYWGQGTPVTVSS |
| VHH8 | SEQ ID NO: | DVQLQESGGGLVQAGGSLRLSCAASGRTFS |
| | 8 | NDAMGWFRQAPGKEREFVAAVSWSLGMTYNADS |
| | | VKGRFTISRDNAKNTVYLQMNSLKPDDTAVYYC |
| | | AAGPDLSDELYGYDYWGQGTQVTVSS |
| VHH9 | SEQ ID NO: | DVQLQESGGGLVQPGESLRLSCTASRSDFS |
| | 9 | RYTMGWYRQAPGKWRELVARITSGGIPHYADSV |
| | | KGRFTISRDNAKNTVTLQMNTLQFEDSAVYYCN |
| | | TVGVALWAWGQGTLVTVSS |
| VHH10 | SEQ ID NO: | DVQLQESGGGLVQAGGSLRLSCAASGLPFS |
| | 10 | SYAMGWFRQAPGKDREFVAGITWSGSSIYYRDS |
| | | VKGRFTISRDNAKNTVWLQMNDLKPEDTAVYYC |
| | | AAARRRVGLPLADPREYVYWGQGTQVTVSS |
| VHH11 | SEQ ID NO: | DVPLQESGGGLVQPGQSLRLSCAASGSPIR |
| | 11 | FSVMGWFQQAPGKGRDLIARITSGGIVYYTDSV |
| | | KGRFTISKDNAKNTLYLQMNSLNLEDTGVYYCN |
| | | RLGGALFDWGQGTQVTVSS |
| VHH12 | SEQ ID NO: | DVPLQESGGGLVQPGGSLRLSCRASGVGFS |
| | 12 | IYTMGWYRQAPGEQRHLVATVTTGGHINYGDSV |
| | | QGRFTISRENNGNVVELQMHSLKPEDTAVYFCN |
| | | AIYFGQDIWGQGTQVTVSS |
| VHH13 | SEQ ID NO: | DVQLQESGGGLVQPGGSLRLSCAASGISFR |
| | 13 | VNVLGWYRQAPGKPRELVASISSGDSTNYADSV |
| | | KGRFTISRDNAKSTVYLQMNNLKPEDTAVYYCN |
| | | ARIFARDNWGQGTQVTVSS |
| VHH14 | SEQ ID NO: | DVPLQESGGGLVQAGGSLRLSCAASDRTVS |
| | 14 | KGAMGWFRQAPGKEREFVATIGRSSGITYYADS |
| | | VKGRFTISRDNAKNTWYLQMNSLRPEDTAVYYC |
| | | AADSSGIIRTTSWAYDYWGQGTQVTVSS |
| VHH15 | SEQ ID NO: | DVQLQESGGGLVQPGGSLRLSCAASGRFFT |
| | 15 | INAMGWYRQTPGKQRELVAAITSDGSTTYQDSV |
| | | KGRFTISRDNANRTANLYMNSLKSEDTAVYYCN |
| | | ADGRFNDNGYVKNFNIWGQGTQVTVSS |
| VHH16 | SEQ ID NO: | DVQLQESGGGSVPAGGSLRLSCSASGFTTD |
| | 16 | DYIIGWFRQAPGKKREGVSCISTTDGSAWYADS |
| | | VKGRFTISSDNANNTVTLQMNSLLPEDTSVYYC |
| | | AEVSTGTGVLYCREEFYFGMDSWGKGTPVTVSS |
| VHH17 | SEQ ID NO: | DVPLQESGGGLVQAGGSLRLSCVASGFALD |
| | 17 | EIAVGWFRQAPGKEREGLSCISSSDGSTLYADS |
| | | VKGRFTISSDNAKNTVYLQMNSLKPEDTAVYLC |
| | | AAVRGSCFGRTTHWGQGTQVTVSS |
| VHH18 | SEQ ID NO: | DVQLQESGGGLVQAGGSLRLSCAASGRTFS |
| | 18 | SYAMGWFRQAPGMVREFVAAISWSGGSTYYADS |
| | | VQGRFTISRDNAKNTVYLQMNSLKPEDTAVYYC |
| | | AADRSLSGSYFGATIDYWGQGTQVTVSS |
| VHH19 | SEQ ID NO: | DVQLQESGGGLVQAGGSLRLSCAASGIRFN |
| | 20 | PFRMAWYRQAPGKQRELVAAIDSGGSTDYADSV |
| | | RGRFTISRDNAKITVSLQMNSLKPEDTAVYYCN |
| | | TQIFARDYWGQGSQVTVSS |
| VHH20 | SEQ ID NO: | DVQLQESGGGLVQAGGSLRLSCAASVSAFR |
| | 21 | LKVMGWYRQAPGKEREFVAAIDSGDTKSYTNAV |
| | | KGRFTISGGAATNTVVLQMNSLKPEDTAFYYCN |
| | | AVLFGRDYWGQGTQVTVSS |
| VHH21 | SEQ ID NO: | DVQLQESGGGLVQAGGSLRLSCAASGITFS |
| | 22 | PYVMGWYRQAPGKQRELVADMVNGGSTNYADSV |
| | | KGRFTIFRDTAANTVTLQMNNLKPDDTAVYYCH |
| | | IMIFGRASWGQGTQVTVSS |
| VHH22 | SEQ ID NO: | DVPLQESGGGLVQPGGSLRLSCAAPGFALE |
| | 22 | YYAIGWFRQAPGKEREGLSCVSSSRSGGSTVYA |
| | | DSVKGRFTISRDNAKNTVYLQIDSLKVEDTAVY |
| | | YCAGAPAYRSHYCRLQADDYTYWGQGTQVTVSS |

5. The compound according to claim 4, wherein said VHH antibody domain of (A) comprises the amino acid sequence of one VHH selected from the group consisting of VHH2 and VHH8 shown in the Table of VHH Sequences and wherein said VHH antibody domain of (E) comprises the amino acid sequence of one VHH selected from the group consisting of VHH15 and VHH17 shown in the Table of VHH Sequences.

6. The compound according to claim 4, wherein said VHH antibody domain of (A) comprises the amino acid sequence of VHH2 shown in the Table of VHH Sequences and wherein said VHH antibody domain of (E) comprises the amino acid sequence of VHH15 shown in the Table of VHH Sequences.

7. The compound according to claim 1, wherein said compound is a bispecific antibody molecule prepared by the SEED technology.

8. The compound according to claim 1, wherein said compound is a single domain-based IgG (sdIgG), wherein said sdIgG is a conventional IgG antibody in which each of the two VH regions is replaced by a copy of said first binding module, and each of the two VL regions is replaced by a copy of said second binding module, resulting in a bispecific, tetravalent antibody.

9. The compound according to claim 1, wherein said compound consists of a IgG Fc region to which two copies of a tandem-VHH arrangement are covalently linked at the hinge-region, wherein said tandem-VHH arrangement consists of a first VHH antibody domain that is covalently linked to a second VHH antibody domain, wherein said second VHH antibody domain is covalently linked to the hinge region of said Fc region, resulting in a bispecific, tetravalent compound.

10. The compound according to claim 1, wherein said compound binds to IL-18Rα ECD with a KD of 100 nM or stronger.

11. The compound according to claim 1, wherein said compound binds to IL-18RβECD with a KD of 100 nM or stronger.

12. The compound according to claim 1, wherein said compound is capable of activating the IL-18 receptor IL-18R by binding to IL-18Rα and IL-18Rβ.

13. The compound according to claim 1, wherein said compound is capable of triggering dose-dependent IL-18R downstream signaling on IL-18 reporter cells.

14. The compound according to claim 1, wherein said compound is capable of inducing IFN-γ release by peripheral blood mononuclear cells (PBMCs) in the presence of low-dose IL-12 (10 ng/ml).

15. A pharmaceutical composition, comprising:
the compound according to claim 1.


SEQUENCE LISTING (116 sequences)

SEQ ID NO: 1 - protein, 116 aa, synthetic construct
DVPLQESGGGLVQTGGSLRLSCAASGFTFNSYAMGWYQQAPGKKYRELVAHIGSGGSTRYIDSVKGRFTISRDNAKNTVYLQMNSLKPEDTAVYYCNVYGATLRSWGQGTQVTVSS

SEQ ID NO: 2 - protein, 127 aa, synthetic construct
DVQLQESGGGLVQAGGSLRLSCAASGFTLLDSYDIGWFRQAPGKKREEVSCISSSGGKSTYYADSVKGRFTISRDNANNTVYLQMNSLKPEDTAVYYCAADGAEGFWSCQSGGYDYWGQGTQVTVSS

SEQ ID NO: 3 - protein, 123 aa, synthetic construct
DVQLQESGGGLVQAGGSLRLSCAASGFTLGAYAINWFRQAPGKERARAWDACITSSGRSAYYADSVKGRFTVSRDHAKNTVYLQMNSLKPEDTAFYYCAAGAACAAAGTMNYWGKGTLVTVSS

SEQ ID NO: 4 - protein, 123 aa, synthetic construct
DVQLQESGGGLVQAGGSLRLSCAASGFTLGAYAINWFQQAPGKERARAWVLCITSSGGSAYYVDSVKGRFTVSRDNAKNTVYLQMNSLKPEDTAFYYCAAGGACAAAETMNYWGKGTLVTVSS

SEQ ID NO: 5 - protein, 129 aa, synthetic construct
DVQLQESGGGLVQAGGSLRLSCAASGRTFSNLAMAWFRQAPGKEREFVAAIDWNGDNRLYAASVKGRFTISRDNAKSTMYLQMNSLKPEDTAVYYCAADSTYSTDWYLNPLNRTRYDYWGQGTQVTVSS

SEQ ID NO: 6 - protein, 119 aa, synthetic construct
DVPLQESGGGSVQPGGSLRLSCAASKSIFSINAMRWHRQAPGKQRELVATFTSSGRINYADSVKGRFTISTDNAKNTIYLQMNSLNPEDTAVYRCNVGLWEDGLWYDYWGQGTQVTVSS

SEQ ID NO: 7 - protein, 129 aa, synthetic construct
DVQLQESGGGLVQAGGSLRLSCAASGHTFNNLAMAWCRQAPGKEREFVSAIDWNGDNRLYAYSVKGRFTISRDNAKSTMYLQMNSLKPEDTAVYYCAADSTYSTNWYLDPLNRTTYNYWGQGTPVTVSS

SEQ ID NO: 8 - protein, 122 aa, synthetic construct
DVQLQESGGGLVQAGGSLRLSCAASGRTFSNDAMGWFRQAPGKEREFVAAVSWSLGMTYNADSVKGRFTISRDNAKNTVYLQMNSLKPDDTAVYYCAAGPDLSDELYGYDYWGQGTQVTVSS

SEQ ID NO: 9 - protein, 115 aa, synthetic construct
DVQLQESGGGLVQPGESLRLSCTASRSDFSRYTMGWYRQAPGKWRELVARITSGGIPHYADSVKGRFTISRDNAKNTVTLQMNTLQFEDSAVYYCNTVGVALWAWGQGTLVTVSS

SEQ ID NO: 10 - protein, 126 aa, synthetic construct
DVQLQESGGGLVQAGGSLRLSCAASGLPFSSYAMGWFRQAPGKDREFVAGITWSGSSIYYRDSVKGRFTISRDNAKNTVWLQMNDLKPEDTAVYYCAAARRRVGLPLADPREYVYWGQGTQVTVSS

SEQ ID NO: 11 - protein, 115 aa, synthetic construct
DVPLQESGGGLVQPGQSLRLSCAASGSPIRFSVMGWFQQAPGKGRDLIARITSGGIVYYTDSVKGRFTISKDNAKNTLYLQMNSLNLEDTGVYYCNRLGGALFDWGQGTQVTVSS

SEQ ID NO: 12 - protein, 115 aa, synthetic construct
DVPLQESGGGLVQPGGSLRLSCRASGVGFSIYTMGWYRQAPGEQRHLVATVTTGGHINYGDSVQGRFTISRENNGNVVELQMHSLKPEDTAVYFCNAIYFGQDIWGQGTQVTVSS

SEQ ID NO: 13 - protein, 115 aa, synthetic construct
DVQLQESGGGLVQPGGSLRLSCAASGISFRVNVLGWYRQAPGKPRELVASISSGDSTNYADSVKGRFTISRDNAKSTVYLQMNNLKPEDTAVYYCNARIFARDNWGQGTQVTVSS

SEQ ID NO: 14 - protein, 124 aa, synthetic construct
DVPLQESGGGLVQAGGSLRLSCAASDRTVSKGAMGWFRQAPGKEREFVATIGRSSGITYYADSVKGRFTISRDNAKNTWYLQMNSLRPEDTAVYYCAADSSGIIRTTSWAYDYWGQGTQVTVSS

SEQ ID NO: 15 - protein, 123 aa, synthetic construct
DVQLQESGGGLVQPGGSLRLSCAASGRFFTINAMGWYRQTPGKQRELVAAITSDGSTTYQDSVKGRFTISRDNANRTANLYMNSLKSEDTAVYYCNADGRFNDNGYVKNFNIWGQGTQVTVSS

SEQ ID NO: 16 - protein, 129 aa, synthetic construct
DVQLQESGGGSVPAGGSLRLSCSASGFTTDDYIIGWFRQAPGKKREGVSCISTTDGSAWYADSVKGRFTISSDNANNTVTLQMNSLLPEDTSVYYCAEVSTGTGVLYCREEFYFGMDSWGKGTPVTVSS

SEQ ID NO: 17 - protein, 120 aa, synthetic construct
DVPLQESGGGLVQAGGSLRLSCVASGFALDEIAVGWFRQAPGKEREGLSCISSSDGSTLYADSVKGRFTISSDNAKNTVYLQMNSLKPEDTAVYLCAAVRGSCFGRTTHWGQGTQVTVSS

SEQ ID NO: 18 - protein, 124 aa, synthetic construct
DVQLQESGGGLVQAGGSLRLSCAASGRTFSSYAMGWFRQAPGMVREFVAAISWSGGSTYYADSVQGRFTISRDNAKNTVYLQMNSLKPEDTAVYYCAADRSLSGSYFGATIDYWGQGTQVTVSS

SEQ ID NO: 19 - protein, 115 aa, synthetic construct
DVQLQESGGGLVQAGGSLRLSCAASGIRFNPFRMAWYRQAPGKQRELVAAIDSGGSTDYADSVRGRFTISRDNAKITVSLQMNSLKPEDTAVYYCNTQIFARDYWGQGSQVTVSS

SEQ ID NO: 20 - protein, 115 aa, synthetic construct
DVQLQESGGGLVQAGGSLRLSCAASVSAFRLKVMGWYRQAPGKEREFVAAIDSGDTKSYTNAVKGRFTISGGAATNTVVLQMNSLKPEDTAFYYCNAVLFGRDYWGQGTQVTVSS

SEQ ID NO: 21 - protein, 115 aa, synthetic construct
DVQLQESGGGLVQAGGSLRLSCAASGITFSPYVMGWYRQAPGKQRELVADMVNGGSTNYADSVKGRFTIFRDTAANTVTLQMNNLKPDDTAVYYCHIMIFGRASWGQGTQVTVSS

SEQ ID NO: 22 - protein, 129 aa, synthetic construct
DVPLQESGGGLVQPGGSLRLSCAAPGFALEYYAIGWFRQAPGKEREGLSCVSSSRSGGSTVYADSVKGRFTISRDNAKNTVYLQIDSLKVEDTAVYYCAGAPAYRSHYCRLQADDYTYWGQGTQVTVSS

SEQ ID NO: 23 - protein, 8 aa, synthetic construct
GFTFNSYA

SEQ ID NO: 24 - protein, 9 aa, synthetic construct
HIGSGGSTR

SEQ ID NO: 25 - protein, 9 aa, synthetic construct
NVYGATLRS

SEQ ID NO: 26 - protein, 9 aa, synthetic construct
GFTLLDSYD

SEQ ID NO: 27 - protein, 10 aa, synthetic construct
ISSSGGKSTY

SEQ ID NO: 28 - protein, 18 aa, synthetic construct
AADGAEGFWSCQSGGYDY

SEQ ID NO: 29 - protein, 8 aa, synthetic construct
GFTLGAYA

SEQ ID NO: 30 - protein, 9 aa, synthetic construct
ITSSGRSAY

SEQ ID NO: 31 - protein, 14 aa, synthetic construct
AAGAACAAAGTMNY

SEQ ID NO: 32 - protein, 8 aa, synthetic construct
GFTLGAYA

SEQ ID NO: 33 - protein, 9 aa, synthetic construct
ITSSGGSAY

SEQ ID NO: 34 - protein, 14 aa, synthetic construct
AAGGACAAAETMNY

SEQ ID NO: 35 - protein, 8 aa, synthetic construct
GRTFSNLA

SEQ ID NO: 36 - protein, 9 aa, synthetic construct
IDWNGDNRL

SEQ ID NO: 37 - protein, 22 aa, synthetic construct
AADSTYSTDWYLNPLNRTRYDY

SEQ ID NO: 38 - protein, 8 aa, synthetic construct
KSIFSINA

SEQ ID NO: 39 - protein, 8 aa, synthetic construct
FTSSGRIN

SEQ ID NO: 40 - protein, 13 aa, synthetic construct
NVGLWEDGLWYDY

SEQ ID NO: 41 - protein, 8 aa, synthetic construct
GHTFNNLA

SEQ ID NO: 42 - protein, 9 aa, synthetic construct
IDWNGDNRL

SEQ ID NO: 43 - protein, 22 aa, synthetic construct
AADSTYSTNWYLDPLNRTTYNY

SEQ ID NO: 44 - protein, 8 aa, synthetic construct
GRTFSNDA

SEQ ID NO: 45 - protein, 9 aa, synthetic construct
VSWSLGMTY

SEQ ID NO: 46 - protein, 15 aa, synthetic construct
AAGPDLSDELYGYDY

SEQ ID NO: 47 - protein, 8 aa, synthetic construct
RSDFSRYT

SEQ ID NO: 48 - protein, 9 aa, synthetic construct
RITSGGIPH

SEQ ID NO: 49 - protein, 9 aa, synthetic construct
NTVGVALWA

SEQ ID NO: 50 - protein, 8 aa, synthetic construct
GLPFSSYA

SEQ ID NO: 51 - protein, 9 aa, synthetic construct
ITWSGSSIY

SEQ ID NO: 52 - protein, 19 aa, synthetic construct
AAARRRVGLPLADPREYVY

SEQ ID NO: 53 - protein, 8 aa, synthetic construct
GSPIRFSV

SEQ ID NO: 54 - protein, 9 aa, synthetic construct
RITSGGIVY

SEQ ID NO: 55 - protein, 9 aa, synthetic construct
NRLGGALFD

SEQ ID NO: 56 - protein, 8 aa, synthetic construct
GVGFSIYT

SEQ ID NO: 57 - protein, 8 aa, synthetic construct
VTTGGHIN

SEQ ID NO: 58 - protein, 9 aa, synthetic construct
NAIYFGQDI

SEQ ID NO: 59 - protein, 8 aa, synthetic construct
GISFRVNV

SEQ ID NO: 60 - protein, 8 aa, synthetic construct
ISSGDSTN

SEQ ID NO: 61 - protein, 9 aa, synthetic construct
NARIFARDN

SEQ ID NO: 62 - protein, 8 aa, synthetic construct
DRTVSKGA

SEQ ID NO: 63 - protein, 9 aa, synthetic construct
IGRSSGITY

SEQ ID NO: 64 - protein, 17 aa, synthetic construct
AADSSGIIRTTSWAYDY

SEQ ID NO: 65 - protein, 8 aa, synthetic construct
GRFFTINA

SEQ ID NO: 66 - protein, 8 aa, synthetic construct
ITSDGSTT

SEQ ID NO: 67 - protein, 17 aa, synthetic construct
NADGRFNDNGYVKNFNI

SEQ ID NO: 68 - protein, 8 aa, synthetic construct
GFTTDDYI

SEQ ID NO: 69 - protein, 9 aa, synthetic construct
ISTTDGSAW

SEQ ID NO: 70 - protein, 22 aa, synthetic construct
AEVSTGTGVLYCREEFYFGMDS

SEQ ID NO: 71 - protein, 8 aa, synthetic construct
GFALDEIA

SEQ ID NO: 72 - protein, 9 aa, synthetic construct
ISSSDGSTL

SEQ ID NO: 73 - protein, 13 aa, synthetic construct
AAVRGSCFGRTTH

SEQ ID NO: 74 - protein, 8 aa, synthetic construct
GRTFSSYA

SEQ ID NO: 75 - protein, 9 aa, synthetic construct
ISWSGGSTY

SEQ ID NO: 76 - protein, 17 aa, synthetic construct
AADRSLSGSYFGATIDY

SEQ ID NO: 77 - protein, 8 aa, synthetic construct
GIRFNPFR

SEQ ID NO: 78 - protein, 8 aa, synthetic construct
IDSGGSTD

SEQ ID NO: 79 - protein, 9 aa, synthetic construct
NTQIFARDY

SEQ ID NO: 80 - protein, 8 aa, synthetic construct
VSAFRLKV

SEQ ID NO: 81 - protein, 8 aa, synthetic construct
IDSGDTKS

SEQ ID NO: 82 - protein, 9 aa, synthetic construct
NAVLFGRDY

SEQ ID NO: 83 - protein, 8 aa, synthetic construct
GITFSPYV

SEQ ID NO: 84 - protein, 8 aa, synthetic construct
MVNGGSTN

SEQ ID NO: 85 - protein, 9 aa, synthetic construct
HIMIFGRAS

SEQ ID NO: 86 - protein, 8 aa, synthetic construct
GFALEYYA

SEQ ID NO: 87 - protein, 11 aa, synthetic construct
VSSSRSGGSTV

SEQ ID NO: 88 - protein, 20 aa, synthetic construct
AGAPAYRSHYCRLQADDYTY

SEQ ID NO: 89 - protein, 115 aa, synthetic construct
DVPLQESGGGLVQTGGSLRLSCAASGFTFNSYAMGWYQQAPGKKYRELVAHIGSGGSTRYIDSVKGRFTISRDNAKNTVYLQMNSLKPEDTAVYYCNVYGATLRSWGQGTQVTVS

SEQ ID NO: 90 - protein, 126 aa, synthetic construct
DVQLQESGGGLVQAGGSLRLSCAASGFTLLDSYDIGWFRQAPGKKREEVSCISSSGGKSTYYADSVKGRFTISRDNANNTVYLQMNSLKPEDTAVYYCAADGAEGFWSCQSGGYDYWGQGTQVTVS

SEQ ID NO: 91 - protein, 122 aa, synthetic construct
DVQLQESGGGLVQAGGSLRLSCAASGFTLGAYAINWFRQAPGKERARAWDACITSSGRSAYYADSVKGRFTVSRDHAKNTVYLQMNSLKPEDTAFYYCAAGAACAAAGTMNYWGKGTLVTVS

SEQ ID NO: 92 - protein, 122 aa, synthetic construct
DVQLQESGGGLVQAGGSLRLSCAASGFTLGAYAINWFQQAPGKERARAWVLCITSSGGSAYYVDSVKGRFTVSRDNAKNTVYLQMNSLKPEDTAFYYCAAGGACAAAETMNYWGKGTLVTVS

SEQ ID NO: 93 - protein, 128 aa, synthetic construct
DVQLQESGGGLVQAGGSLRLSCAASGRTFSNLAMAWFRQAPGKEREFVAAIDWNGDNRLYAASVKGRFTISRDNAKSTMYLQMNSLKPEDTAVYYCAADSTYSTDWYLNPLNRTRYDYWGQGTQVTVS

SEQ ID NO: 94 - protein, 118 aa, synthetic construct
DVPLQESGGGSVQPGGSLRLSCAASKSIFSINAMRWHRQAPGKQRELVATFTSSGRINYADSVKGRFTISTDNAKNTIYLQMNSLNPEDTAVYRCNVGLWEDGLWYDYWGQGTQVTVS

SEQ ID NO: 95 - protein, 128 aa, synthetic construct
DVQLQESGGGLVQAGGSLRLSCAASGHTFNNLAMAWCRQAPGKEREFVSAIDWNGDNRLYAYSVKGRFTISRDNAKSTMYLQMNSLKPEDTAVYYCAADSTYSTNWYLDPLNRTTYNYWGQGTPVTVS

SEQ ID NO: 96 - protein, 121 aa, synthetic construct
DVQLQESGGGLVQAGGSLRLSCAASGRTFSNDAMGWFRQAPGKEREFVAAVSWSLGMTYNADSVKGRFTISRDNAKNTVYLQMNSLKPDDTAVYYCAAGPDLSDELYGYDYWGQGTQVTVS

SEQ ID NO: 97 - protein, 114 aa, synthetic construct
DVQLQESGGGLVQPGESLRLSCTASRSDFSRYTMGWYRQAPGKWRELVARITSGGIPHYADSVKGRFTISRDNAKNTVTLQMNTLQFEDSAVYYCNTVGVALWAWGQGTLVTVS

SEQ ID NO: 98 - protein, 125 aa, synthetic construct
DVQLQESGGGLVQAGGSLRLSCAASGLPFSSYAMGWFRQAPGKDREFVAGITWSGSSIYYRDSVKGRFTISRDNAKNTVWLQMNDLKPEDTAVYYCAAARRRVGLPLADPREYVYWGQGTQVTVS

SEQ ID NO: 99 - protein, 114 aa, synthetic construct
DVPLQESGGGLVQPGQSLRLSCAASGSPIRFSVMGWFQQAPGKGRDLIARITSGGIVYYTDSVKGRFTISKDNAKNTLYLQMNSLNLEDTGVYYCNRLGGALFDWGQGTQVTVS

SEQ ID NO: 100 - protein, 114 aa, synthetic construct
DVPLQESGGGLVQPGGSLRLSCRASGVGFSIYTMGWYRQAPGEQRHLVATVTTGGHINYGDSVQGRFTISRENNGNVVELQMHSLKPEDTAVYFCNAIYFGQDIWGQGTQVTVS

SEQ ID NO: 101 - protein, 114 aa, synthetic construct
DVQLQESGGGLVQPGGSLRLSCAASGISFRVNVLGWYRQAPGKPRELVASISSGDSTNYADSVKGRFTISRDNAKSTVYLQMNNLKPEDTAVYYCNARIFARDNWGQGTQVTVS

SEQ ID NO: 102 - protein, 123 aa, synthetic construct
DVPLQESGGGLVQAGGSLRLSCAASDRTVSKGAMGWFRQAPGKEREFVATIGRSSGITYYADSVKGRFTISRDNAKNTWYLQMNSLRPEDTAVYYCAADSSGIIRTTSWAYDYWGQGTQVTVS

SEQ ID NO: 103 - protein, 122 aa, synthetic construct
DVQLQESGGGLVQPGGSLRLSCAASGRFFTINAMGWYRQTPGKQRELVAAITSDGSTTYQDSVKGRFTISRDNANRTANLYMNSLKSEDTAVYYCNADGRFNDNGYVKNFNIWGQGTQVTVS

SEQ ID NO: 104 - protein, 128 aa, synthetic construct
DVQLQESGGGSVPAGGSLRLSCSASGFTTDDYIIGWFRQAPGKKREGVSCISTTDGSAWYADSVKGRFTISSDNANNTVTLQMNSLLPEDTSVYYCAEVSTGTGVLYCREEFYFGMDSWGKGTPVTVS

SEQ ID NO: 105 - protein, 119 aa, synthetic construct
DVPLQESGGGLVQAGGSLRLSCVASGFALDEIAVGWFRQAPGKEREGLSCISSSDGSTLYADSVKGRFTISSDNAKNTVYLQMNSLKPEDTAVYLCAAVRGSCFGRTTHWGQGTQVTVS

SEQ ID NO: 106 - protein, 123 aa, synthetic construct
DVQLQESGGGLVQAGGSLRLSCAASGRTFSSYAMGWFRQAPGMVREFVAAISWSGGSTYYADSVQGRFTISRDNAKNTVYLQMNSLKPEDTAVYYCAADRSLSGSYFGATIDYWGQGTQVTVS

SEQ ID NO: 107 - protein, 114 aa, synthetic construct
DVQLQESGGGLVQAGGSLRLSCAASGIRFNPFRMAWYRQAPGKQRELVAAIDSGGSTDYADSVRGRFTISRDNAKITVSLQMNSLKPEDTAVYYCNTQIFARDYWGQGSQVTVS

SEQ ID NO: 108 - protein, 114 aa, synthetic construct
DVQLQESGGGLVQAGGSLRLSCAASVSAFRLKVMGWYRQAPGKEREFVAAIDSGDTKSYTNAVKGRFTISGGAATNTVVLQMNSLKPEDTAFYYCNAVLFGRDYWGQGTQVTVS

SEQ ID NO: 109 - protein, 114 aa, synthetic construct
DVQLQESGGGLVQAGGSLRLSCAASGITFSPYVMGWYRQAPGKQRELVADMVNGGSTNYADSVKGRFTIFRDTAANTVTLQMNNLKPDDTAVYYCHIMIFGRASWGQGTQVTVS

SEQ ID NO: 110 - protein, 128 aa, synthetic construct
DVPLQESGGGLVQPGGSLRLSCAAPGFALEYYAIGWFRQAPGKEREGLSCVSSSRSGGSTVYADSVKGRFTISRDNAKNTVYLQIDSLKVEDTAVYYCAGAPAYRSHYCRLQADDYTYWGQGTQVTVS

SEQ ID NO: 111 - protein, 541 aa, synthetic construct
MNCRELPLTLWVLISVSTAESCTSRPHITVVEGEPFYLKHCSCSLAHEIETTTKSWYKSSGSQEHVELNPRSSSRIALHDCVLEFWPVELNDTGSYFFQMKNYTQKWKLNVIRRNKHSCFTERQVTSKIVEVKKFFQITCENSYYQTLVNSTSLYKNCKKLLLENNKNPTIKKNAEFEDQGYYSCVHFLHHNGKLFNITKTFNITIVEDRSNIVPVLLGPKLNHVAVELGKNVRLNCSALLNEEDVIYWMFGEENGSDPNIHEEKEMRIMTPEGKWHASKVLRIENIGESNLNVLYNCTVASTGGTDTKSFILVRKADMADIPGHVFTRGMIIAVLILVAVVCLVTVCVIYRVDLVLFYRHLTRRDETLTDGKTYDAFVSYLKECRPENGEEHTFAVEILPRVLEKHFGYKLCIFERDVVPGGAVVDEIHSLIEKSRRLIIVLSKSYMSNEVRYELESGLHEALVERKIKIILIEFTPVTDFTFLPQSLKLLKSHRVLKWKADKSLSYNSRFWKNLLYLMPAKTVKPGRDEPEVLPVLSES

SEQ ID NO: 112 - protein, 308 aa, synthetic construct
CTSRPHITVVEGEPFYLKHCSCSLAHEIETTTKSWYKSSGSQEHVELNPRSSSRIALHDCVLEFWPVELNDTGSYFFQMKNYTQKWKLNVIRRNKHSCFTERQVTSKIVEVKKFFQITCENSYYQTLVNSTSLYKNCKKLLLENNKNPTIKKNAEFEDQGYYSCVHFLHHNGKLFNITKTFNITIVEDRSNIVPVLLGPKLNHVAVELGKNVRLNCSALLNEEDVIYWMFGEENGSDPNIHEEKEMRIMTPEGKWHASKVLRIENIGESNLNVLYNCTVASTGGTDTKSFILVRKADMADIPGHVFTR

SEQ ID NO: 113 - protein, 599 aa, synthetic construct
MLCLGWIFLWLVAGERIKGFNISGCSTKKLLWTYSTRSEEEFVLFCDLPEPQKSHFCHRNRLSPKQVPEHLPFMGSNDLSDVQWYQQPSNGDPLEDIRKSYPHIIQDKCTLHFLTPGVNNSGSYICRPKMIKSPYDVACCVKMILEVKPQTNASCEYSASHKQDLLLGSTGSISCPSLSCQSDAQSPAVTWYKNGKLLSVERSNRIVVDEVYDYHQGTYVCDYTQSDTVSSWTVRAVVQVRTIVGDTKLKPDILDPVEDTLEVELGKPLTISCKARFGFERVFNPVIKWYIKDSDLEWEVSVPEAKSIKSTLKDEIIERNIILEKVTQRDLRRKFVCFVQNSIGNTTQSVQLKEKRGVVLLYILLGTIGTLVAVLAASALLYRHWIEIVLLYRTYQSKDQTLGDKKDFDAFVSYAKWSSFPSEATSSLSEEHLALSLFPDVLENKYGYSLCLLERDVAPGGVYAEDIVSIIKRSRRGIFILSPNYVNGPSIFELQAAVNLALDDQTLKLILIKFCYFQEPESLPHLVKKALRVLPTVTWRGLKSVPPNSRFWAKMRYHMPVKNSQGFTWNQLRITSRIFQWKGLSRTETTGRSSQPKEW

SEQ ID NO: 114 - protein, 337 aa, synthetic construct
FNISGCSTKKLLWTYSTRSEEEFVLFCDLPEPQKSHFCHRNRLSPKQVPEHLPFMGSNDLSDVQWYQQPSNGDPLEDIRKSYPHIIQDKCTLHFLTPGVNNSGSYICRPKMIKSPYDVACCVKMILEVKPQTNASCEYSASHKQDLLLGSTGSISCPSLSCQSDAQSPAVTWYKNGKLLSVERSNRIVVDEVYDYHQGTYVCDYTQSDTVSSWTVRAVVQVRTIVGDTKLKPDILDPVEDTLEVELGKPLTISCKARFGFERVFNPVIKWYIKDSDLEWEVSVPEAKSIKSTLKDEIIERNIILEKVTQRDLRRKFVCFVQNSIGNTTQSVQLKEKR

SEQ ID NO: 115 - protein, 193 aa, synthetic construct
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 116 - protein, 194 aa, synthetic construct
MTMRHNWTPDLSPLWVLLLCAHVVTLLVRATPVSQTTTAATASVRSTKDPCPSQPPVFPAAKQCPALEVTWPEVEVPLNGTLSLSCVACSRFPNFSILYWLGNGSFIEHLPGRLWEGSTSRERGSTGTQLCKALVLEQLTPALHSTNFSCVLVDPEQVVQRHVVLAQLWAGLRATLPPTQEALPSSHSSPQQQG